SEQUENCE LISTING (1393 sequences)

SEQ ID NO: 1 - protein, 443 aa, Artificial
  Clostridium septicum alpha-toxin
MSKKSFAKKVICTSMIAIQCAAVVPHVQAYALTNLEEGGYANHNNASSIKIFGYEDNEDLKAKIIQDPEFIRNWANVAHSLGFGWCGGTANPNVGQGFEFKREVGAGGKVSYLLSARYNPNDPYASGYRAKDRLSMKISNVRFVIDNDSIKLGTPKVKKLAPLNSASFDLINESKTESKLSKTFNYTTSKTVSKTDNFKFGEKIGVKTSFKVGLEAIADSKVETSFEFNAKQGWSNTNSTTETKQESTTYTATVSPQTKKRLFLDVLGSQIDIPYEGKIYMEYDIKLMGFLRYTGNAREDHTEDRPTVKLKFGKNGMSAEEHLKDLYSHKNINGYSEWDWKWVDEKFGYLFKNSYDALTSRKLGGIIKGSFTNINGTKIVIREGKEIPLPDKKRRGKRSVDSLDARLQNEGIRIENIETQDVPGFRLNSITYNDKKLILINNI

SEQ ID NO: 2 - protein, 8 aa, Artificial
  furin consensus site
KKRSGSRS

SEQ ID NO: 3 - protein, 6 aa, Artificial
  Peptide
ATSGSR

SEQ ID NO: 4 - DNA, 19 bp, Artificial
  GPAA1 target
tcttcctctacttgctccc

SEQ ID NO: 5 - DNA, 19 bp, Artificial
  PIGT Target
gacactgaccactactttc

SEQ ID NO: 6 - protein, 11 aa, Artificial
  Peptide
YYTGNYDQYVK

SEQ ID NO: 7 - protein, 14 aa, Artificial
  Peptide
IPPPVIMVQNVSFK

SEQ ID NO: 8 - protein, 14 aa, Artificial
  Peptide
LLTGELLPTDGMIR

SEQ ID NO: 9 - protein, 10 aa, Artificial
  Peptide
LVDEEPQLTK

SEQ ID NO: 10 - protein, 16 aa, Artificial
  Peptide
DINQEVYNFLATAGAK

SEQ ID NO: 11 - protein, 12 aa, Artificial
  Peptide
SQFTITPGSQIR

SEQ ID NO: 12 - protein, 15 aa, Artificial
  Peptide
NAVTQEFGPVPDTAR

SEQ ID NO: 13 - protein, 15 aa, Artificial
  Peptide
WVVIGDENYGGGSSR

SEQ ID NO: 14 - protein, 15 aa, Artificial
  Peptide
IGYSSPQTLADQSSK

SEQ ID NO: 15 - protein, 21 aa, Artificial
  Peptide
SVTHFDSLAVIDIPGADTLDK

SEQ ID NO: 16 - protein, 17 aa, Artificial
  Peptide
LSPEPWTPETGLVTDFK

SEQ ID NO: 17 - protein, 10 aa, Artificial
  Peptide
HRPELIEYDK

SEQ ID NO: 18 - protein, 17 aa, Artificial
  Peptide
KDDPVTNLNNAFEVAEK

SEQ ID NO: 19 - protein, 16 aa, Artificial
  Peptide
MLDAEDIVNTARPDEK

SEQ ID NO: 20 - protein, 17 aa, Artificial
  Peptide
VLAVNQENEHLMEDYEK

SEQ ID NO: 21 - protein, 25 aa, Artificial
  Peptide
VEQIAAIAQELNELDYYDSHNVNTR

SEQ ID NO: 22 - protein, 18 aa, Artificial
  Peptide
LSGSNPYTTVTPQIINSK

SEQ ID NO: 23 - protein, 7 aa, Artificial
  Peptide
VQQLVPK

SEQ ID NO: 24 - protein, 28 aa, Artificial
  Peptide
SIVDYKPNLDLLEQQHQLIQEALIFDNK

SEQ ID NO: 25 - protein, 11 aa, Artificial
  Peptide
GISQEQMQEFR

SEQ ID NO: 26 - protein, 16 aa, Artificial
  Peptide
ETTDTDTADQVIASFK

SEQ ID NO: 27 - protein, 9 aa, Artificial
  Peptide
NFITAEELR

SEQ ID NO: 28 - protein, 17 aa, Artificial
  Peptide
MAPYQGPDAVPGALDYK

SEQ ID NO: 29 - protein, 10 aa, Artificial
  Peptide
DQSLQEELAR

SEQ ID NO: 30 - protein, 17 aa, Artificial
  Peptide
KDDPLTNLNTAFDVAEK

SEQ ID NO: 31 - protein, 16 aa, Artificial
  Peptide
MLDAEDIVGTARPDEK

SEQ ID NO: 32 - protein, 17 aa, Artificial
  Peptide
VLAVNQENEQLMEDYEK

SEQ ID NO: 33 - protein, 22 aa, Artificial
  Peptide
VEQIAAIAQELDYYDSPSVNAR

SEQ ID NO: 34 - protein, 14 aa, Artificial
  Peptide
LLETIDQLYLEYAK

SEQ ID NO: 35 - protein, 20 aa, Artificial
  Peptide
IDQLEGDHQLIQEALIFDNK

SEQ ID NO: 36 - protein, 11 aa, Artificial
  Peptide
GISQEQMNEFR

SEQ ID NO: 37 - protein, 16 aa, Artificial
  Peptide
ETADTDTADQVMASFK

SEQ ID NO: 38 - protein, 8 aa, Artificial
  Peptide
ALDFIASK

SEQ ID NO: 39 - protein, 10 aa, Artificial
  Peptide
LASDLLEWIR

SEQ ID NO: 40 - protein, 12 aa, Artificial
  Peptide
VGWEQLLTTIAR

SEQ ID NO: 41 - protein, 12 aa, Artificial
  Peptide
TINEVENQILTR

SEQ ID NO: 42 - protein, 13 aa, Artificial
  Peptide
AGTQIENIEEDFR

SEQ ID NO: 43 - protein, 10 aa, Artificial
  Peptide
DLLLDPAWEK

SEQ ID NO: 44 - protein, 14 aa, Artificial
  Peptide
FAIQDISVEETSAK

SEQ ID NO: 45 - protein, 11 aa, Artificial
  Peptide
GYEEWLLNEIR

SEQ ID NO: 46 - protein, 7 aa, Artificial
  Peptide
LPQTLSR

SEQ ID NO: 47 - protein, 18 aa, Artificial
  Peptide
GWGDQLIWTQTYEEALYK

SEQ ID NO: 48 - protein, 17 aa, Artificial
  Peptide
LAEQFVLLNLVYETTDK

SEQ ID NO: 49 - protein, 11 aa, Artificial
  Peptide
HLSPDGQYVPR

SEQ ID NO: 50 - protein, 11 aa, Artificial
  Peptide
IMFVDPSLTVR

SEQ ID NO: 51 - protein, 17 aa, Artificial
  Peptide
LYAYEPADTALLLDNMK

SEQ ID NO: 52 - protein, 13 aa, Artificial
  Peptide
SVQYDDVPEYKDR

SEQ ID NO: 53 - protein, 14 aa, Artificial
  Peptide
VLHPLEGAVVIIFK

SEQ ID NO: 54 - protein, 11 aa, Artificial
  Peptide
SSNTYTLTDVR

SEQ ID NO: 55 - protein, 14 aa, Artificial
  Peptide
ESLTLIVEGKPQIK

SEQ ID NO: 56 - protein, 14 aa, Artificial
  Peptide
IPPDSEATLVLVGR

SEQ ID NO: 57 - protein, 24 aa, Artificial
  Peptide
GLDLNGGPDDPLQQTGQLFGGLVR

SEQ ID NO: 58 - protein, 17 aa, Artificial
  Peptide
LQEAAELEAVELPVPIR

SEQ ID NO: 59 - protein, 11 aa, Artificial
  Peptide
TYNYNVLMVPK

SEQ ID NO: 60 - protein, 14 aa, Artificial
  Peptide
ASTPGAAAQIQEVK

SEQ ID NO: 61 - protein, 17 aa, Artificial
  Peptide
QAWFIENEEQEYVQTVK

SEQ ID NO: 62 - protein, 24 aa, Artificial
  Peptide
GGPGSAVSPYPTFNPSSDVAALHK

SEQ ID NO: 63 - protein, 13 aa, Artificial
  Peptide
GVDEATIIDILTK

SEQ ID NO: 64 - protein, 16 aa, Artificial
  Peptide
AAYLQETGKPLDETLK

SEQ ID NO: 65 - protein, 15 aa, Artificial
  Peptide
ALTGHLEEVVLALLK

SEQ ID NO: 66 - protein, 11 aa, Artificial
  Peptide
TPAQFDADELR

SEQ ID NO: 67 - protein, 16 aa, Artificial
  Peptide
GLGTDEDTLIEILASR

SEQ ID NO: 68 - protein, 11 aa, Artificial
  Peptide
DITSDTSGDFR

SEQ ID NO: 69 - protein, 8 aa, Artificial
  Peptide
NALLSLAK

SEQ ID NO: 70 - protein, 16 aa, Artificial
  Peptide
SEDFGVNEDLADSDAR

SEQ ID NO: 71 - protein, 8 aa, Artificial
  Peptide
ALYEAGER

SEQ ID NO: 72 - protein, 14 aa, Artificial
  Peptide
GTDVNVFNTILTTR

SEQ ID NO: 73 - protein, 7 aa, Artificial
  Peptide
VLDLELK

SEQ ID NO: 74 - protein, 7 aa, Artificial
  Peptide
PAFFAEK

SEQ ID NO: 75 - protein, 8 aa, Artificial
  Peptide
SIDMNDIK

SEQ ID NO: 76 - protein, 9 aa, Artificial
  Peptide
MLISILTER

SEQ ID NO: 77 - protein, 16 aa, Artificial
  Peptide
GAGTNEDALIEILTTR

SEQ ID NO: 78 - protein, 11 aa, Artificial
  Peptide
DISQAYYTVYK

SEQ ID NO: 79 - protein, 15 aa, Artificial
  Peptide
SLGDDISSETSGDFR

SEQ ID NO: 80 - protein, 9 aa, Artificial
  Peptide
ALLTLADGR

SEQ ID NO: 81 - protein, 7 aa, Artificial
  Peptide
LTFDGYR

SEQ ID NO: 82 - protein, 9 aa, Artificial
  Peptide
NTPAFLAER

SEQ ID NO: 83 - protein, 11 aa, Artificial
  Peptide
GIGTDGFTLNR

SEQ ID NO: 84 - protein, 9 aa, Artificial
  Peptide
SGIDLLDIR

SEQ ID NO: 85 - protein, 13 aa, Artificial
  Peptide
SDTSGDYEITLLK

SEQ ID NO: 86 - protein, 12 aa, Artificial
  Peptide
GTVTDFPGFDGR

SEQ ID NO: 87 - protein, 16 aa, Artificial
  Peptide
GLGTDEESILTLLTSR

SEQ ID NO: 88 - protein, 13 aa, Artificial
  Peptide
DLLDDLKSGLTGK

SEQ ID NO: 89 - protein, 8 aa, Artificial
  Peptide
LYDAYGLK

SEQ ID NO: 90 - protein, 9 aa, Artificial
  Peptide
VLTEIIASR

SEQ ID NO: 91 - protein, 25 aa, Artificial
  Peptide
DPDAGIDEAQVEQDAQALFQAGELK

SEQ ID NO: 92 - protein, 15 aa, Artificial
  Peptide
ETSGNLEQLLLAVVK

SEQ ID NO: 93 - protein, 15 aa, Artificial
  Peptide
SIPAYLAETLYYAMK

SEQ ID NO: 94 - protein, 11 aa, Artificial
  Peptide
GAGTDDHTLIR

SEQ ID NO: 95 - protein, 9 aa, Artificial
  Peptide
SEIDLFNIR

SEQ ID NO: 96 - protein, 23 aa, Artificial
  Peptide
GSIHDFPGFDPNQDAEALYTAMK

SEQ ID NO: 97 - protein, 10 aa, Artificial
  Peptide
EAILDIITSR

SEQ ID NO: 98 - protein, 11 aa, Artificial
  Peptide
DAISGIGTDGK

SEQ ID NO: 99 - protein, 13 aa, Artificial
  Peptide
TNEQMHQLVAAYK

SEQ ID NO: 100 - protein, 16 aa, Artificial
  Peptide
DLEADIIGDTSGHFQK

SEQ ID NO: 101 - protein, 10 aa, Artificial
  Peptide
MLVVLLQGTR

SEQ ID NO: 102 - protein, 25 aa, Artificial
  Peptide
EEDDVVSEDLVQQDVQDLYEAGELK

SEQ ID NO: 103 - protein, 15 aa, Artificial
  Peptide
WGTDEAQFIYILGNR

SEQ ID NO: 104 - protein, 8 aa, Artificial
  Peptide
LVFDEYLK

SEQ ID NO: 105 - protein, 9 aa, Artificial
  Peptide
SELDMLDIR

SEQ ID NO: 106 - protein, 31 aa, Artificial
  Peptide
LSGGDDDAAEQFFPEAAQVAYQMWELSAVAR

SEQ ID NO: 107 - protein, 12 aa, Artificial
  Peptide
PANDENPDADAK

SEQ ID NO: 108 - protein, 16 aa, Artificial
  Peptide
GLGTDEDTIIDIITHR

SEQ ID NO: 109 - protein, 7 aa, Artificial
  Peptide
DLMTDLK

SEQ ID NO: 110 - protein, 9 aa, Artificial
  Peptide
ALIEILATR

SEQ ID NO: 111 - protein, 15 aa, Artificial
  Peptide
SLEDALSSDTSGHFR

SEQ ID NO: 112 - protein, 11 aa, Artificial
  Peptide
EEGGENLDQAR

SEQ ID NO: 113 - protein, 20 aa, Artificial
  Peptide
EDAQVAAEILEIADTPSGDK

SEQ ID NO: 114 - protein, 7 aa, Artificial
  Peptide
VFQEFIK

SEQ ID NO: 115 - protein, 11 aa, Artificial
  Peptide
MTNYDVEHTIK

SEQ ID NO: 116 - protein, 11 aa, Artificial
  Peptide
DAFVAIVQSVK

SEQ ID NO: 117 - protein, 9 aa, Artificial
  Peptide
NKPLFFADK

SEQ ID NO: 118 - protein, 9 aa, Artificial
  Peptide
SEIDLLNIR

SEQ ID NO: 119 - protein, 16 aa, Artificial
  Peptide
SLHQAIEGDTSGDFLK

SEQ ID NO: 120 - protein, 12 aa, Artificial
  Peptide
LGPGLVDAAQAR

SEQ ID NO: 121 - protein, 9 aa, Artificial
  Peptide
AAALAHLDR

SEQ ID NO: 122 - protein, 10 aa, Artificial
  Peptide
NLVTMTTAPR

SEQ ID NO: 123 - protein, 19 aa, Artificial
  Peptide
SPVPPGPAPPKQFYPQGPR

SEQ ID NO: 124 - protein, 10 aa, Artificial
  Peptide
YVRGGFGMGK

SEQ ID NO: 125 - protein, 8 aa, Artificial
  Peptide
YQLAVTQR

SEQ ID NO: 126 - protein, 28 aa, Artificial
  Peptide
IIGFGSALLEEVDPNPANFVGAGIIHYK

SEQ ID NO: 127 - protein, 10 aa, Artificial
  Peptide
FINLFPETLK

SEQ ID NO: 128 - protein, 10 aa, Artificial
  Peptide
YGGAPQATLK

SEQ ID NO: 129 - protein, 15 aa, Artificial
  Peptide
FFQPTEMAAQDFFQR

SEQ ID NO: 130 - protein, 28 aa, Artificial
  Peptide
LLGFGSALLDNVDPNPENFVGAGIIGTK

SEQ ID NO: 131 - protein, 18 aa, Artificial
  Peptide
VGGYILGEFGNLIAGDPR

SEQ ID NO: 132 - protein, 9 aa, Artificial
  Peptide
NADVELQQR

SEQ ID NO: 133 - protein, 14 aa, Artificial
  Peptide
QLSNPQQEVQNIFK

SEQ ID NO: 134 - protein, 8 aa, Artificial
  Peptide
SPVTNIAR

SEQ ID NO: 135 - protein, 9 aa, Artificial
  Peptide
TFITQQGIK

SEQ ID NO: 136 - protein, 9 aa, Artificial
  Peptide
ASENAIVWK

SEQ ID NO: 137 - protein, 11 aa, Artificial
  Peptide
DLATVYVDVLK

SEQ ID NO: 138 - protein, 17 aa, Artificial
  Peptide
DSGRDYVSQFEGSALGK

SEQ ID NO: 139 - protein, 13 aa, Artificial
  Peptide
DYVSQFGGSALGK

SEQ ID NO: 140 - protein, 14 aa, Artificial
  Peptide
LLDNWDSVTSTFSK

SEQ ID NO: 141 - protein, 16 aa, Artificial
  Peptide
EQLGPVTQEFWDNLEK

SEQ ID NO: 142 - protein, 10 aa, Artificial
  Peptide
VQPYLDDFQK

SEQ ID NO: 143 - protein, 8 aa, Artificial
  Peptide
AELQEGAR

SEQ ID NO: 144 - protein, 7 aa, Artificial
  Peptide
LHELQEK

SEQ ID NO: 145 - protein, 11 aa, Artificial
  Peptide
ETEGLRQEMSK

SEQ ID NO: 146 - protein, 9 aa, Artificial
  Peptide
WQEEMELYR

SEQ ID NO: 147 - protein, 9 aa, Artificial
  Peptide
LSPLGEEMR

SEQ ID NO: 148 - protein, 7 aa, Artificial
  Peptide
AHVDALR

SEQ ID NO: 149 - protein, 11 aa, Artificial
  Peptide
THLAPYSDELR

SEQ ID NO: 150 - protein, 7 aa, Artificial
  Peptide
LAEYHAK

SEQ ID NO: 151 - protein, 11 aa, Artificial
  Peptide
ATEHLSTLSEK

SEQ ID NO: 152 - protein, 9 aa, Artificial
  Peptide
AKPALEDLR

SEQ ID NO: 153 - protein, 11 aa, Artificial
  Peptide
QGLLPVLESFK

SEQ ID NO: 154 - protein, 12 aa, Artificial
  Peptide
VSFLSALEEYTK

SEQ ID NO: 155 - protein, 25 aa, Artificial
  Peptide
SSGNSSSSGSGSGSTSAGSSSPGAR

SEQ ID NO: 156 - protein, 18 aa, Artificial
  Peptide
LGIYDADGDGDFDVDDAK

SEQ ID NO: 157 - protein, 36 aa, Artificial
  Peptide
STSEPAVPPEEAEPHTEPEEQVPVEAEPQNIEDEAK

SEQ ID NO: 158 - protein, 14 aa, Artificial
  Peptide
EGIESGDPGTDDGR

SEQ ID NO: 159 - protein, 12 aa, Artificial
  Peptide
WGSNELPAEEGK

SEQ ID NO: 160 - protein, 10 aa, Artificial
  Peptide
NAENAIEALK

SEQ ID NO: 161 - protein, 15 aa, Artificial
  Peptide
VDQSILTGESVSVIK

SEQ ID NO: 162 - protein, 10 aa, Artificial
  Peptide
LDEFGEQLSK

SEQ ID NO: 163 - protein, 9 aa, Artificial
  Peptide
MNVFDTELK

SEQ ID NO: 164 - protein, 9 aa, Artificial
  Peptide
VIMITGDNK

SEQ ID NO: 165 - protein, 13 aa, Artificial
  Peptide
IGIFGQDEDVTSK

SEQ ID NO: 166 - protein, 11 aa, Artificial
  Peptide
EFDELNPSAQR

SEQ ID NO: 167 - protein, 26 aa, Artificial
  Peptide
IVEFLQSFDEITAMTGDGVNDAPALK

SEQ ID NO: 168 - protein, 22 aa, Artificial
  Peptide
TASEMVLADDNFSTIAAVEEGR

SEQ ID NO: 169 - protein, 13 aa, Artificial
  Peptide
ISLPVILMDETLK

SEQ ID NO: 170 - protein, 15 aa, Artificial
  Peptide
DKYEPAAVSEQGDKK

SEQ ID NO: 171 - protein, 8 aa, Artificial
  Peptide
LSLDELHR

SEQ ID NO: 172 - protein, 17 aa, Artificial
  Peptide
DGPNALTPPPTTPEWIK

SEQ ID NO: 173 - protein, 11 aa, Artificial
  Peptide
NMVPQQALVIR

SEQ ID NO: 174 - protein, 15 aa, Artificial
  Peptide
VDNSSLTGESEPQTR

SEQ ID NO: 175 - protein, 13 aa, Artificial
  Peptide
SPDFTNENPLGTR

SEQ ID NO: 176 - protein, 9 aa, Artificial
  Peptide
GIVVYTGDR

SEQ ID NO: 177 - protein, 10 aa, Artificial
  Peptide
TSATWLALSR

SEQ ID NO: 178 - protein, 14 aa, Artificial
  Peptide
AVFQANQENLPILK

SEQ ID NO: 179 - protein, 13 aa, Artificial
  Peptide
AVAGDASESALLK

SEQ ID NO: 180 - protein, 11 aa, Artificial
  Peptide
IVEIPFNSTNK

SEQ ID NO: 181 - protein, 14 aa, Artificial
  Peptide
NPNTSEPQHLLVMK

SEQ ID NO: 182 - protein, 25 aa, Artificial
  Peptide
EQPLDEELKDAFQNAYLELGGLGER

SEQ ID NO: 183 - protein, 16 aa, Artificial
  Peptide
DAFQNAYLELGGLGER

SEQ ID NO: 184 - protein, 13 aa, Artificial
  Peptide
VIMVTGDHPITAK

SEQ ID NO: 185 - protein, 18 aa, Artificial
  Peptide
GVGIISEGNETVEDIAAR

SEQ ID NO: 186 - protein, 11 aa, Artificial
  Peptide
LNIPVSQVNPR

SEQ ID NO: 187 - protein, 12 aa, Artificial
  Peptide
DMTSEQLDDILK

SEQ ID NO: 188 - protein, 19 aa, Artificial
  Peptide
QGAIVAVTGDGVNDSPALK

SEQ ID NO: 189 - protein, 23 aa, Artificial
  Peptide
QAADMILLDDNFASIVTGVEEGR

SEQ ID NO: 190 - protein, 7 aa, Artificial
  Peptide
LIFDNLK

SEQ ID NO: 191 - protein, 9 aa, Artificial
  Peptide
NSVFQQGMK

SEQ ID NO: 192 - protein, 10 aa, Artificial
  Peptide
SLNQSLAEWK

SEQ ID NO: 193 - protein, 14 aa, Artificial
  Peptide
LFIYNPTTGEFLGR

SEQ ID NO: 194 - protein, 11 aa, Artificial
  Peptide
PVTALGYTFSR

SEQ ID NO: 195 - protein, 15 aa, Artificial
  Peptide
SDPTSYAGYIGDLKK

SEQ ID NO: 196 - protein, 11 aa, Artificial
  Peptide
IIGLKPEGVPR

SEQ ID NO: 197 - protein, 15 aa, Artificial
  Peptide
LVLEVAQHLGESTVR

SEQ ID NO: 198 - protein, 12 aa, Artificial
  Peptide
TIAMDGTEGLVR

SEQ ID NO: 199 - protein, 19 aa, Artificial
  Peptide
VLDSGAPIKIPVGPETLGR

SEQ ID NO: 200 - protein, 10 aa, Artificial
  Peptide
IPVGPETLGR

SEQ ID NO: 201 - protein, 12 aa, Artificial
  Peptide
IMNVIGEPIDER

SEQ ID NO: 202 - protein, 10 aa, Artificial
  Peptide
VVDLLAPYAK

SEQ ID NO: 203 - protein, 11 aa, Artificial
  Peptide
IGLFGGAGVGK

SEQ ID NO: 204 - protein, 13 aa, Artificial
  Peptide
TVLIMELINNVAK

SEQ ID NO: 205 - protein, 20 aa, Artificial
  Peptide
TREGNDLYHEMIESGVINLK

SEQ ID NO: 206 - protein, 15 aa, Artificial
  Peptide
VALVYGQMNEPPGAR

SEQ ID NO: 207 - protein, 13 aa, Artificial
  Peptide
VALTGLTVAEYFR

SEQ ID NO: 208 - protein, 16 aa, Artificial
  Peptide
DQEGQDVLLFIDNIFR

SEQ ID NO: 209 - protein, 14 aa, Artificial
  Peptide
FTQAGSEVSALLGR

SEQ ID NO: 210 - protein, 21 aa, Artificial
  Peptide
IPSAVGYQPTLATDMGTMQER

SEQ ID NO: 211 - protein, 19 aa, Artificial
  Peptide
AIAELGIYPAVDPLDSTSR

SEQ ID NO: 212 - protein, 16 aa, Artificial
  Peptide
IMDPNIVGSEHYDVAR

SEQ ID NO: 213 - protein, 24 aa, Artificial
  Peptide
SLQDIIAILGMDELSEEDKLTVSR

SEQ ID NO: 214 - protein, 12 aa, Artificial
  Peptide
AGAAGTAEATAR

SEQ ID NO: 215 - protein, 16 aa, Artificial
  Peptide
LNVFAKPEATEVSPNK

SEQ ID NO: 216 - protein, 15 aa, Artificial
  Peptide
EASGLLSLTSTLYLR

SEQ ID NO: 217 - protein, 13 aa, Artificial
  Peptide
GDGSPSPEYTLFR

SEQ ID NO: 218 - protein, 14 aa, Artificial
  Peptide
VEDYDAADDVQLSK

SEQ ID NO: 219 - protein, 17 aa, Artificial
  Peptide
LSWSQLGGSPAEPIPGR

SEQ ID NO: 220 - protein, 19 aa, Artificial
  Peptide
LVTLISQQATLLASNEAFK

SEQ ID NO: 221 - protein, 9 aa, Artificial
  Peptide
YMEENDQLK

SEQ ID NO: 222 - protein, 9 aa, Artificial
  Peptide
LDVGNAEVK

SEQ ID NO: 223 - protein, 9 aa, Artificial
  Peptide
LKDELASTK

SEQ ID NO: 224 - protein, 9 aa, Artificial
  Peptide
AENQVLAMR

SEQ ID NO: 225 - protein, 11 aa, Artificial
  Peptide
LQAAVDGPMDK

SEQ ID NO: 226 - protein, 15 aa, Artificial
  Peptide
KVEELEGEITTLNHK

SEQ ID NO: 227 - protein, 10 aa, Artificial
  Peptide
LQDASAEVER

SEQ ID NO: 228 - protein, 14 aa, Artificial
  Peptide
SESVPPVTDWAWYK

SEQ ID NO: 229 - protein, 9 aa, Artificial
  Peptide
FFVSSSQGR

SEQ ID NO: 230 - protein, 18 aa, Artificial
  Peptide
SELHIENLNMEADPGQYR

SEQ ID NO: 231 - protein, 10 aa, Artificial
  Peptide
GSDQAIITLR

SEQ ID NO: 232 - protein, 18 aa, Artificial
  Peptide
RKPEDVLDDDDAGSAPLK

SEQ ID NO: 233 - protein, 17 aa, Artificial
  Peptide
KPEDVLDDDDAGSAPLK

SEQ ID NO: 234 - protein, 15 aa, Artificial
  Peptide
SMLDQLGVPLYAVVK

SEQ ID NO: 235 - protein, 11 aa, Artificial
  Peptide
VNLLSVLEAAK

SEQ ID NO: 236 - protein, 11 aa, Artificial
  Peptide
AFVDFLSDEIK

SEQ ID NO: 237 - protein, 14 aa, Artificial
  Peptide
AFVDFLSDEIKEER

SEQ ID NO: 238 - protein, 20 aa, Artificial
  Peptide
VEEQEPELTSTPNFVVEVIK

SEQ ID NO: 239 - protein, 13 aa, Artificial
  Peptide
EVSFQSTGESEWK

SEQ ID NO: 240 - protein, 30 aa, Artificial
  Peptide
GVDNTFADELVELSTALEHQEYITFLEDLK

SEQ ID NO: 241 - protein, 19 aa, Artificial
  Peptide
RPLRPQVVTDDDGQAPEAK

SEQ ID NO: 242 - protein, 27 aa, Artificial
  Peptide
LFENQLVGPESIAHIGDVMFTGTADGR

SEQ ID NO: 243 - protein, 14 aa, Artificial
  Peptide
EPPLLLGVLHPNTK

SEQ ID NO: 244 - protein, 11 aa, Artificial
  Peptide
LENGEIETIAR

SEQ ID NO: 245 - protein, 12 aa, Artificial
  Peptide
LLLSSETPIEGK

SEQ ID NO: 246 - protein, 9 aa, Artificial
  Peptide
LLEYDTVTR

SEQ ID NO: 247 - protein, 7 aa, Artificial
  Peptide
VLLDQLR

SEQ ID NO: 248 - protein, 23 aa, Artificial
  Peptide
FPNGVQLSPAEDFVLVAETTMAR

SEQ ID NO: 249 - protein, 18 aa, Artificial
  Peptide
GGADLFVENMPGFPDNIR

SEQ ID NO: 250 - protein, 9 aa, Artificial
  Peptide
LFSQETVMK

SEQ ID NO: 251 - protein, 12 aa, Artificial
  Peptide
TIYTPGSTVLYR

SEQ ID NO: 252 - protein, 15 aa, Artificial
  Peptide
TVMVNIENPEGIPVK

SEQ ID NO: 253 - protein, 29 aa, Artificial
  Peptide
QDSLSSQNQLGVLPLSWDIPELVNMGQWK

SEQ ID NO: 254 - protein, 18 aa, Artificial
  Peptide
AYYENSPQQVFSTEFEVK

SEQ ID NO: 255 - protein, 14 aa, Artificial
  Peptide
IPIEDGSGEVVLSR

SEQ ID NO: 256 - protein, 10 aa, Artificial
  Peptide
VLLDGVQNPR

SEQ ID NO: 257 - protein, 16 aa, Artificial
  Peptide
SGIPIVTSPYQIHFTK

SEQ ID NO: 258 - protein, 22 aa, Artificial
  Peptide
VPVAVQGEDTVQSLTQGDGVAK

SEQ ID NO: 259 - protein, 12 aa, Artificial
  Peptide
KQELSEAEQATR

SEQ ID NO: 260 - protein, 11 aa, Artificial
  Peptide
QELSEAEQATR

SEQ ID NO: 261 - protein, 22 aa, Artificial
  Peptide
EPGQDLVVLPLSITTDFIPSFR

SEQ ID NO: 262 - protein, 16 aa, Artificial
  Peptide
SNLDEDIIAEENIVSR

SEQ ID NO: 263 - protein, 7 aa, Artificial
  Peptide
LPYSVVR

SEQ ID NO: 264 - protein, 7 aa, Artificial
  Peptide
NEQVEIR

SEQ ID NO: 265 - protein, 13 aa, Artificial
  Peptide
SSLSVPYVIVPLK

SEQ ID NO: 266 - protein, 9 aa, Artificial
  Peptide
TGLQEVEVK

SEQ ID NO: 267 - protein, 20 aa, Artificial
  Peptide
EDIPPADLSDQVPDTESETR

SEQ ID NO: 268 - protein, 20 aa, Artificial
  Peptide
ILLQGTPVAQMTEDAVDAER

SEQ ID NO: 269 - protein, 9 aa, Artificial
  Peptide
GYTQQLAFR

SEQ ID NO: 270 - protein, 10 aa, Artificial
  Peptide
DFDFVPPVVR

SEQ ID NO: 271 - protein, 11 aa, Artificial
  Peptide
VTIKPAPETEK

SEQ ID NO: 272 - protein, 26 aa, Artificial
  Peptide
GDQDATMSILDISMMTGFAPDTDDLK

SEQ ID NO: 273 - protein, 16 aa, Artificial
  Peptide
VHQYFNVELIQPGAVK

SEQ ID NO: 274 - protein, 12 aa, Artificial
  Peptide
SGSDEVQVGQQR

SEQ ID NO: 275 - protein, 9 aa, Artificial
  Peptide
AEFQDALEK

SEQ ID NO: 276 - protein, 13 aa, Artificial
  Peptide
GSFEFPVGDAVSK

SEQ ID NO: 277 - protein, 26 aa, Artificial
  Peptide
VTASDPLDTLGSEGALSPGGVASLLR

SEQ ID NO: 278 - protein, 15 aa, Artificial
  Peptide
VLSLAQEQVGGSPEK

SEQ ID NO: 279 - protein, 14 aa, Artificial
  Peptide
WTYFGPDGENSWSK

SEQ ID NO: 280 - protein, 15 aa, Artificial
  Peptide
LNLPSDMHIQGLQSR

SEQ ID NO: 281 - protein, 9 aa, Artificial
  Peptide
IFSHLQHVK

SEQ ID NO: 282 - protein, 20 aa, Artificial
  Peptide
YKGQEAFVPGFNIEELLPER

SEQ ID NO: 283 - protein, 18 aa, Artificial
  Peptide
GQEAFVPGFNIEELLPER

SEQ ID NO: 284 - protein, 20 aa, Artificial
  Peptide
TLHEWLQQHGIPGLQGVDTR

SEQ ID NO: 285 - protein, 42 aa, Artificial
  Peptide
GAEVTVVPWDHALDSQEYEGLFLSNGPGDPASYPSVVSTLSR

SEQ ID NO: 286 - protein, 13 aa, Artificial
  Peptide
EATAGNPGGQTVR

SEQ ID NO: 287 - protein, 26 aa, Artificial
  Peptide
MAEIGEHVAPSEAANSLEQAQAAAER

SEQ ID NO: 288 - protein, 16 aa, Artificial
  Peptide
AAFALGGLGSGFASNR

SEQ ID NO: 289 - protein, 12 aa, Artificial
  Peptide
ATGYPLAYVAAK

SEQ ID NO: 290 - protein, 10 aa, Artificial
  Peptide
SVGEVMGIGR

SEQ ID NO: 291 - protein, 15 aa, Artificial
  Peptide
VLGTSPEAIDSAENR

SEQ ID NO: 292 - protein, 10 aa, Artificial
  Peptide
FLSSAAAVSK

SEQ ID NO: 293 - protein, 44 aa, Artificial
  Peptide
EIDVDAVASDGVVAAIAISEHVENAGVHSGDATLVTPPQDITAK

SEQ ID NO: 294 - protein, 12 aa, Artificial
  Peptide
TLGVDLVALATR

SEQ ID NO: 295 - protein, 22 aa, Artificial
  Peptide
VIMGEEVEPVGLMTGSGVVGVK

SEQ ID NO: 296 - protein, 9 aa, Artificial
  Peptide
SILEQLAEK

SEQ ID NO: 297 - protein, 16 aa, Artificial
  Peptide
LFVEALGQIGPAPPLK

SEQ ID NO: 298 - protein, 11 aa, Artificial
  Peptide
LYLNETFSELR

SEQ ID NO: 299 - protein, 9 aa, Artificial
  Peptide
LSLDDLLQR

SEQ ID NO: 300 - protein, 14 aa, Artificial
  Peptide
DQMSHLFNVAHTLR

SEQ ID NO: 301 - protein, 11 aa, Artificial
  Peptide
TSSSFAAAMAR

SEQ ID NO: 302 - protein, 21 aa, Artificial
  Peptide
QEEFESIEEALPDTDVLYMTR

SEQ ID NO: 303 - protein, 16 aa, Artificial
  Peptide
APVPTGEVYFADSFDR

SEQ ID NO: 304 - protein, 10 aa, Artificial
  Peptide
GTLSGWILSK

SEQ ID NO: 305 - protein, 10 aa, Artificial
  Peptide
KDDTDDEIAK

SEQ ID NO: 306 - protein, 7 aa, Artificial
  Peptide
WEVEEMK

SEQ ID NO: 307 - protein, 10 aa, Artificial
  Peptide
LNKPFLFDTK

SEQ ID NO: 308 - protein, 12 aa, Artificial
  Peptide
TPELNLDQFHDK

SEQ ID NO: 309 - protein, 11 aa, Artificial
  Peptide
TPYTIMFGPDK

SEQ ID NO: 310 - protein, 7 aa, Artificial
  Peptide
TGIYEEK

SEQ ID NO: 311 - protein, 40 aa, Artificial
  Peptide
THLYTLILNPDNSFEILVDQSVVNSGNLLNDMTPPVNPSR

SEQ ID NO: 312 - protein, 10 aa, Artificial
  Peptide
KPEDWDERPK

SEQ ID NO: 313 - protein, 19 aa, Artificial
  Peptide
IPDPEAVKPDDWDEDAPAK

SEQ ID NO: 314 - protein, 48 aa, Artificial
  Peptide
IPDEEATKPEGWLDDEPEYVPDPDAEKPEDWDEDMDGEWEAPQIANPR

SEQ ID NO: 315 - protein, 9 aa, Artificial
  Peptide
PVIDNPNYK

SEQ ID NO: 316 - protein, 16 aa, Artificial
  Peptide
WKPPMIDNPSYQGIWK

SEQ ID NO: 317 - protein, 15 aa, Artificial
  Peptide
KIPNPDFFEDLEPFR

SEQ ID NO: 318 - protein, 14 aa, Artificial
  Peptide
IPNPDFFEDLEPFR

SEQ ID NO: 319 - protein, 13 aa, Artificial
  Peptide
IVDDWANDGWGLK

SEQ ID NO: 320 - protein, 9 aa, Artificial
  Peptide
AEEDEILNR

SEQ ID NO: 321 - protein, 24 aa, Artificial
  Peptide
ILGGVISAISEAAAQYNPEPPPPR

SEQ ID NO: 322 - protein, 13 aa, Artificial
  Peptide
SMVAVMDSDTTGK

SEQ ID NO: 323 - protein, 7 aa, Artificial
  Peptide
LGFEEFK

SEQ ID NO: 324 - protein, 14 aa, Artificial
  Peptide
YVDSEGHLYTVPIR

SEQ ID NO: 325 - protein, 9 aa, Artificial
  Peptide
AMADELSEK

SEQ ID NO: 326 - protein, 8 aa, Artificial
  Peptide
HLNDDVVK

SEQ ID NO: 327 - protein, 21 aa, Artificial
  Peptide
IDFEDVIAEPEGTHSFDGIWK

SEQ ID NO: 328 - protein, 15 aa, Artificial
  Peptide
LTSFIGAIAIGDLVK

SEQ ID NO: 329 - protein, 22 aa, Artificial
  Peptide
VQDDEVGDGTTSVTVLAAELLR

SEQ ID NO: 330 - protein, 16 aa, Artificial
  Peptide
EALLSSAVDHGSDEVK

SEQ ID NO: 331 - protein, 14 aa, Artificial
  Peptide
QDLMNIAGTTLSSK

SEQ ID NO: 332 - protein, 8 aa, Artificial
  Peptide
LAVEAVLR

SEQ ID NO: 333 - protein, 12 aa, Artificial
  Peptide
GSGNLGAIHIIK

SEQ ID NO: 334 - protein, 18 aa, Artificial
  Peptide
LGGSLADSYLDEGFLLDK

SEQ ID NO: 335 - protein, 19 aa, Artificial
  Peptide
LGGSLADSYLDEGFLLDKK

SEQ ID NO: 336 - protein, 12 aa, Artificial
  Peptide
ILIANTGMDTDK

SEQ ID NO: 337 - protein, 9 aa, Artificial
  Peptide
VAEIEHAEK

SEQ ID NO: 338 - protein, 29 aa, Artificial
  Peptide
QLIYNYPEQLFGAAGVMAIEHADFAGVER

SEQ ID NO: 339 - protein, 20 aa, Artificial
  Peptide
LALVTGGEIASTFDHPELVK

SEQ ID NO: 340 - protein, 11 aa, Artificial
  Peptide
LIEEVMIGEDK

SEQ ID NO: 341 - protein, 12 aa, Artificial
  Peptide
GATQQILDEAER

SEQ ID NO: 342 - protein, 22 aa, Artificial
  Peptide
MLPTIIADNAGYDSADLVAQLR

SEQ ID NO: 343 - protein, 15 aa, Artificial
  Peptide
AAHSEGNTTAGLDMR

SEQ ID NO: 344 - protein, 15 aa, Artificial
  Peptide
QVLLSAAEAAEVILR

SEQ ID NO: 345 - protein, 12 aa, Artificial
  Peptide
QVVLEEGTIAFK

SEQ ID NO: 346 - protein, 34 aa, Artificial
  Peptide
EGRPVYISLPHFLYASPDVSEPIDGLNPNEEEHR

SEQ ID NO: 347 - protein, 31 aa, Artificial
  Peptide
PVYISLPHFLYASPDVSEPIDGLNPNEEEHR

SEQ ID NO: 348 - protein, 12 aa, Artificial
  Peptide
DKVAELENSEFR

SEQ ID NO: 349 - protein, 11 aa, Artificial
  Peptide
VAELENSEEFR

SEQ ID NO: 350 - protein, 14 aa, Artificial
  Peptide
LKQEMGGIVTELIR

SEQ ID NO: 351 - protein, 10 aa, Artificial
  Peptide
GEEDNSLSVR

SEQ ID NO: 352 - protein, 11 aa, Artificial
  Peptide
IGEGTYGVVYK

SEQ ID NO: 353 - protein, 14 aa, Artificial
  Peptide
LESEEEGVPSTAIR

SEQ ID NO: 354 - protein, 16 aa, Artificial
  Peptide
HPNIVSLQDVLMQDSR

SEQ ID NO: 355 - protein, 10 aa, Artificial
  Peptide
SPEVLLGSAR

SEQ ID NO: 356 - protein, 20 aa, Artificial
  Peptide
ALGTPNNEVWPEVESLQDYK

SEQ ID NO: 357 - protein, 8 aa, Artificial
  Peptide
MLIYDPAK

SEQ ID NO: 358 - protein, 14 aa, Artificial
  Peptide
VSEADSSNADWVTK

SEQ ID NO: 359 - protein, 11 aa, Artificial
  Peptide
VKDISEVVTPR

SEQ ID NO: 360 - protein, 8 aa, Artificial
  Peptide
LSENVIDR

SEQ ID NO: 361 - protein, 15 aa, Artificial
  Peptide
YSGAYGASVSDEELK

SEQ ID NO: 362 - protein, 11 aa, Artificial
  Peptide
VAEELALGQAK

SEQ ID NO: 363 - protein, 15 aa, Artificial
  Peptide
QREELGQGLQGVEQK

SEQ ID NO: 364 - protein, 10 aa, Artificial
  Peptide
DLSDGIHVVK

SEQ ID NO: 365 - protein, 14 aa, Artificial
  Peptide
ERDFTSLENTVEER

SEQ ID NO: 366 - protein, 15 aa, Artificial
  Peptide
VASLEESEGNKQDLK

SEQ ID NO: 367 - protein, 10 aa, Artificial
  Peptide
SREWDMEALR

SEQ ID NO: 368 - protein, 7 aa, Artificial
  Peptide
RLEEELR

SEQ ID NO: 369 - protein, 19 aa, Artificial
  Peptide
LEGLGSSEADQDGLASTVR

SEQ ID NO: 370 - protein, 14 aa, Artificial
  Peptide
SVGELPSTVESLQK

SEQ ID NO: 371 - protein, 18 aa, Artificial
  Peptide
VQEQVHTLLSQDQAQAAR

SEQ ID NO: 372 - protein, 11 aa, Artificial
  Peptide
ASVSQVEADLK

SEQ ID NO: 373 - protein, 12 aa, Artificial
  Peptide
TAVDSLVAYSVK

SEQ ID NO: 374 - protein, 12 aa, Artificial
  Peptide
IETNENNLESAK

SEQ ID NO: 375 - protein, 12 aa, Artificial
  Peptide
TTGEVVSGVVSK

SEQ ID NO: 376 - protein, 10 aa, Artificial
  Peptide
GEAVQEELLK

SEQ ID NO: 377 - protein, 11 aa, Artificial
  Peptide
TALAATNPAVR

SEQ ID NO: 378 - protein, 13 aa, Artificial
  Peptide
ALAVMVDHLESGK

SEQ ID NO: 379 - protein, 12 aa, Artificial
  Peptide
IEDLEEGQQVIR

SEQ ID NO: 380 - protein, 28 aa, Artificial
  Peptide
FQEHLQLQNLGINPANIGFSTLTMESDK

SEQ ID NO: 381 - protein, 15 aa, Artificial
  Peptide
RPISADSAIMNPASK

SEQ ID NO: 382 - protein, 12 aa, Artificial
  Peptide
AHTMTDDVTFWK

SEQ ID NO: 383 - protein, 13 aa, Artificial
  Peptide
WLLLTGISAQQNR

SEQ ID NO: 384 - protein, 12 aa, Artificial
  Peptide
VVGAMQLYSVDR

SEQ ID NO: 385 - protein, 18 aa, Artificial
  Peptide
LHIIEVGTPPTGNQPFPK

SEQ ID NO: 386 - protein, 24 aa, Artificial
  Peptide
AVDVFFPPEAQNDDFPVAMQISEK

SEQ ID NO: 387 - protein, 23 aa, Artificial
  Peptide
ISGETIFVTAPHEATAGIIGVNR

SEQ ID NO: 388 - protein, 12 aa, Artificial
  Peptide
NNLAGAEELFAR

SEQ ID NO: 389 - protein, 29 aa, Artificial
  Peptide
FQSVPAQPGQTSPLLQYFGILLDQGQLNK

SEQ ID NO: 390 - protein, 13 aa, Artificial
  Peptide
SVDPTLALSVYLR

SEQ ID NO: 391 - protein, 12 aa, Artificial
  Peptide
VGYTPDWIFLLR

SEQ ID NO: 392 - protein, 12 aa, Artificial
  Peptide
NNRPSEGPLQTR

SEQ ID NO: 393 - protein, 27 aa, Artificial
  Peptide
LLEMNLMHAPQVADAILGNQMFTHYDR

SEQ ID NO: 394 - protein, 6 aa, Artificial
  Peptide
AGLLQR

SEQ ID NO: 395 - protein, 12 aa, Artificial
  Peptide
ALEHFTDLYDIK

SEQ ID NO: 396 - protein, 14 aa, Artificial
  Peptide
GQFSTDELVAEVEK

SEQ ID NO: 397 - protein, 9 aa, Artificial
  Peptide
LLLPWLEAR

SEQ ID NO: 398 - protein, 11 aa, Artificial
  Peptide
IYIDSNNNPER

SEQ ID NO: 399 - protein, 26 aa, Artificial
  Peptide
RPLIDQVVQTALSETQDPEEVSVTVK

SEQ ID NO: 400 - protein, 17 aa, Artificial
  Peptide
AFMTADLPNELIELLEK

SEQ ID NO: 401 - protein, 12 aa, Artificial
  Peptide
IVLDNSVFSEHR

SEQ ID NO: 402 - protein, 12 aa, Artificial
  Peptide
NLQNLLILTAIK

SEQ ID NO: 403 - protein, 21 aa, Artificial
  Peptide
KFDVNTSAVQVLIEHIGNLDR

SEQ ID NO: 404 - protein, 20 aa, Artificial
  Peptide
FDVNTSAVQVLIEHIGNLDR

SEQ ID NO: 405 - protein, 25 aa, Artificial
  Peptide
ADDPSSYMEVVQAANTSGNWEELVK

SEQ ID NO: 406 - protein, 22 aa, Artificial
  Peptide
LAELEEFINGPNNAHIQQVGDR

SEQ ID NO: 407 - protein, 11 aa, Artificial
  Peptide
LLYNNVSNFGR

SEQ ID NO: 408 - protein, 19 aa, Artificial
  Peptide
LASTLVHLGEYQAAVDGAR

SEQ ID NO: 409 - protein, 9 aa, Artificial
  Peptide
VANVELYYR

SEQ ID NO: 410 - protein, 20 aa, Artificial
  Peptide
SVNESLNNLFITEEDYQALR

SEQ ID NO: 411 - protein, 17 aa, Artificial
  Peptide
TSIDAYDNFDNISLAQR

SEQ ID NO: 412 - protein, 7 aa, Artificial
  Peptide
HELIEFR

SEQ ID NO: 413 - protein, 7 aa, Artificial
  Peptide
IAAYLFK

SEQ ID NO: 414 - protein, 10 aa, Artificial
  Peptide
DAMQYASESK

SEQ ID NO: 415 - protein, 17 aa, Artificial
  Peptide
DTELAEELLQWFLQEEK

SEQ ID NO: 416 - protein, 11 aa, Artificial
  Peptide
VDKLDASESLR

SEQ ID NO: 417 - protein, 10 aa, Artificial
  Peptide
TLLSNLEEAK

SEQ ID NO: 418 - protein, 12 aa, Artificial
  Peptide
ASSIIDELFQDR

SEQ ID NO: 419 - protein, 11 aa, Artificial
  Peptide
ELDESLQVAER

SEQ ID NO: 420 - protein, 23 aa, Artificial
  Peptide
VTTVASHTSDSDVPSGVTEVVVK

SEQ ID NO: 421 - protein, 17 aa, Artificial
  Peptide
LFDSDPITVTVPVEVSR

SEQ ID NO: 422 - protein, 12 aa, Artificial
  Peptide
SEDFSLPAYMDR

SEQ ID NO: 423 - protein, 10 aa, Artificial
  Peptide
VNPIQGLASK

SEQ ID NO: 424 - protein, 9 aa, Artificial
  Peptide
FGVYDIDNK

SEQ ID NO: 425 - protein, 8 aa, Artificial
  Peptide
NNLNPVWR

SEQ ID NO: 426 - protein, 9 aa, Artificial
  Peptide
SPLGEVAIR

SEQ ID NO: 427 - protein, 9 aa, Artificial
  Peptide
DIVQFVPFR

SEQ ID NO: 428 - protein, 13 aa, Artificial
  Peptide
STPAITLESPDIK

SEQ ID NO: 429 - protein, 8 aa, Artificial
  Peptide
IDGNLVVR

SEQ ID NO: 430 - protein, 19 aa, Artificial
  Peptide
PYTPISSDDDKGFVDLVIK

SEQ ID NO: 431 - protein, 11 aa, Artificial
  Peptide
GPSGLLVYQGK

SEQ ID NO: 432 - protein, 12 aa, Artificial
  Peptide
DILLRPELEELR

SEQ ID NO: 433 - protein, 18 aa, Artificial
  Peptide
APEAWDYGQGFVNEEMIR

SEQ ID NO: 434 - protein, 11 aa, Artificial
  Peptide
TLVLLDNLNVR

SEQ ID NO: 435 - protein, 11 aa, Artificial
  Peptide
TADDPSLSLIK

SEQ ID NO: 436 - protein, 9 aa, Artificial
  Peptide
SSLNPILFR

SEQ ID NO: 437 - protein, 41 aa, Artificial
  Peptide
GVGMVADPDNPLVLDILTGSSTSYSFFPDKPITQYPHAVGK

SEQ ID NO: 438 - protein, 11 aa, Artificial
  Peptide
NTLLIAGLQAR

SEQ ID NO: 439 - protein, 15 aa, Artificial
  Peptide
WVPFDGDDIQLGFVR

SEQ ID NO: 440 - protein, 11 aa, Artificial
  Peptide
LPDVYGVFQFK

SEQ ID NO: 441 - protein, 9 aa, Artificial
  Peptide
TASQGQWGR

SEQ ID NO: 442 - protein, 22 aa, Artificial
  Peptide
FLPGYVGGIQEGAVTPAGVVNK

SEQ ID NO: 443 - protein, 7 aa, Artificial
  Peptide
AAQLGFK

SEQ ID NO: 444 - protein, 11 aa, Artificial
  Peptide
ADGGTQVIDTK

SEQ ID NO: 445 - protein, 14 aa, Artificial
  Peptide
NLGLEELGIELDPR

SEQ ID NO: 446 - protein, 13 aa, Artificial
  Peptide
SEEQLKEEGIEYK

SEQ ID NO: 447 - protein, 9 aa, Artificial
  Peptide
YGDGIQLTR

SEQ ID NO: 448 - protein, 7 aa, Artificial
  Peptide
GDECILK

SEQ ID NO: 449 - protein, 14 aa, Artificial
  Peptide
ALLQAILQTEDMLK

SEQ ID NO: 450 - protein, 17 aa, Artificial
  Peptide
GFFDPNTEENLTKLQLK

SEQ ID NO: 451 - protein, 25 aa, Artificial
  Peptide
SSSFSDTLEESSPIAAIFDTENLEK

SEQ ID NO: 452 - protein, 10 aa, Artificial
  Peptide
YLANMNYVHR

SEQ ID NO: 453 - protein, 16 aa, Artificial
  Peptide
VLEDDPEATYTTSGGK

SEQ ID NO: 454 - protein, 10 aa, Artificial
  Peptide
FADIVSILDK

SEQ ID NO: 455 - protein, 13 aa, Artificial
  Peptide
LPSTSGSEGVPFR

SEQ ID NO: 456 - protein, 18 aa, Artificial
  Peptide
DKEETLPLEDGWWGPGTR

SEQ ID NO: 457 - protein, 9 aa, Artificial
  Peptide
GFNSVATAR

SEQ ID NO: 458 - protein, 9 aa, Artificial
  Peptide
DVELLYPVK

SEQ ID NO: 459 - protein, 9 aa, Artificial
  Peptide
YLEDGGLER

SEQ ID NO: 460 - protein, 18 aa, Artificial
  Peptide
GGHFAAFEEPELLAQDIR

SEQ ID NO: 461 - protein, 20 aa, Artificial
  Peptide
LALQQDLTSMAPGLVIQAVR

SEQ ID NO: 462 - protein, 12 aa, Artificial
  Peptide
VAQVAEITYGQK

SEQ ID NO: 463 - protein, 12 aa, Artificial
  Peptide
ISEIEDAAFLAR

SEQ ID NO: 464 - protein, 15 aa, Artificial
  Peptide
LSFGLEDEDLETATK

SEQ ID NO: 465 - protein, 12 aa, Artificial
  Peptide
SVQTTLQTDEVK

SEQ ID NO: 466 - protein, 20 aa, Artificial
  Peptide
IEVVNFLTSMAPGLVIQAVR

SEQ ID NO: 467 - protein, 14 aa, Artificial
  Peptide
EALEPSGENVIQNK

SEQ ID NO: 468 - protein, 8 aa, Artificial
  Peptide
TILEWEPK

SEQ ID NO: 469 - protein, 21 aa, Artificial
  Peptide
TILEWEPKPVNQVYTVQISTK

SEQ ID NO: 470 - protein, 13 aa, Artificial
  Peptide
PVNQVYTVQISTK

SEQ ID NO: 471 - protein, 48 aa, Artificial
  Peptide
VFSYPAGNVESTGSAGEPLYENSPEFTPYLETNLGQPTIQSFEQVGTK

SEQ ID NO: 472 - protein, 10 aa, Artificial
  Peptide
DLIYTLYYWK

SEQ ID NO: 473 - protein, 12 aa, Artificial
  Peptide
TNTNEFLIDVDK

SEQ ID NO: 474 - protein, 9 aa, Artificial
  Peptide
KAGVGQSWK

SEQ ID NO: 475 - protein, 11 aa, Artificial
  Peptide
MDASLGNLFAR

SEQ ID NO: 476 - protein, 12 aa, Artificial
  Peptide
MASGAANVVGPK

SEQ ID NO: 477 - protein, 8 aa, Artificial
  Peptide
TGEVLDTK

SEQ ID NO: 478 - protein, 17 aa, Artificial
  Peptide
YFDMWGGDVAPFIEFLK

SEQ ID NO: 479 - protein, 16 aa, Artificial
  Peptide
LIADLGSTSITNLGFR

SEQ ID NO: 480 - protein, 8 aa, Artificial
  Peptide
SPFEQHIK

SEQ ID NO: 481 - protein, 27 aa, Artificial
  Peptide
LPESENLQEFWDNLIGGVDMVTDDDRR

SEQ ID NO: 482 - protein, 11 aa, Artificial
  Peptide
FDASFFGVHPK

SEQ ID NO: 483 - protein, 16 aa, Artificial
  Peptide
VYATILNAGTNTDGFK

SEQ ID NO: 484 - protein, 17 aa, Artificial
  Peptide
EQGVTFPSGDIQEQLIR

SEQ ID NO: 485 - protein, 12 aa, Artificial
  Peptide
VGDPQELNGITR

SEQ ID NO: 486 - protein, 17 aa, Artificial
  Peptide
SNMGHPEPASGLAALAK

SEQ ID NO: 487 - protein, 11 aa, Artificial
  Peptide
LQVVDQPLPVR

SEQ ID NO: 488 - protein, 20 aa, Artificial
  Peptide
DTVTISGPQAPVFEFVEQLR

SEQ ID NO: 489 - protein, 23 aa, Artificial
  Peptide
LHLSGIDANPNALFPPVEFPAPR

SEQ ID NO: 490 - protein, 10 aa, Artificial
  Peptide
GTPLISPLIK

SEQ ID NO: 491 - protein, 26 aa, Artificial
  Peptide
ALGLGVEQLPVVFEDVVLHQATILPK

SEQ ID NO: 492 - protein, 10 aa, Artificial
  Peptide
VYQWDDPDPR

SEQ ID NO: 493 - protein, 10 aa, Artificial
  Peptide
AQVADVVVSR

SEQ ID NO: 494 - protein, 9 aa, Artificial
  Peptide
DPSQQELPR

SEQ ID NO: 495 - protein, 21 aa, Artificial
  Peptide
LQLNGNLQLELAQVLAQERPK

SEQ ID NO: 496 - protein, 17 aa, Artificial
  Peptide
LPEDPLLSGLLDSPALK

SEQ ID NO: 497 - protein, 20 aa, Artificial
  Peptide
IPGLLSPHPLLQLSYTATDR

SEQ ID NO: 498 - protein, 19 aa, Artificial
  Peptide
RPTPQDSPIFLPVDDTSFR

SEQ ID NO: 499 - protein, 15 aa, Artificial
  Peptide
GILADEDSSRPVWLK

SEQ ID NO: 500 - protein, 15 aa, Artificial
  Peptide
WTSQDSLLGMEFSGR

SEQ ID NO: 501 - protein, 10 aa, Artificial
  Peptide
VFTTVGSAEK

SEQ ID NO: 502 - protein, 13 aa, Artificial
  Peptide
FPQLDSTSFANSR

SEQ ID NO: 503 - protein, 15 aa, Artificial
  Peptide
VVVQVLAEEPEAVLK

SEQ ID NO: 504 - protein, 13 aa, Artificial
  Peptide
SLLVNPEGPTLMR

SEQ ID NO: 505 - protein, 21 aa, Artificial
  Peptide
PLFLVHPIEGSTTVFHSLASR

SEQ ID NO: 506 - protein, 9 aa, Artificial
  Peptide
VLEALLPLK

SEQ ID NO: 507 - protein, 23 aa, Artificial
  Peptide
TLLGGSGLESIISIIHSSLAEPR

SEQ ID NO: 508 - protein, 14 aa, Artificial
  Peptide
TASGDYIDSSWELR

SEQ ID NO: 509 - protein, 16 aa, Artificial
  Peptide
VFVGEEDPEAESVTLR

SEQ ID NO: 510 - protein, 13 aa, Artificial
  Peptide
VTGESHIGGVLLK

SEQ ID NO: 511 - protein, 7 aa, Artificial
  Peptide
IVEQINR

SEQ ID NO: 512 - protein, 14 aa, Artificial
  Peptide
KQDWSDHAIWWEQK

SEQ ID NO: 513 - protein, 13 aa, Artificial
  Peptide
VVLAGGVAPALFR

SEQ ID NO: 514 - protein, 8 aa, Artificial
  Peptide
AGDALWLR

SEQ ID NO: 515 - protein, 10 aa, Artificial
  Peptide
YYSFFDLDPK

SEQ ID NO: 516 - protein, 9 aa, Artificial
  Peptide
LTQLYEQAR

SEQ ID NO: 517 - protein, 20 aa, Artificial
  Peptide
LEGSAPTDVLDSLTTIPELK

SEQ ID NO: 518 - protein, 16 aa, Artificial
  Peptide
SQDEAPGDPIQQLNLK

SEQ ID NO: 519 - protein, 15 aa, Artificial
  Peptide
LLVPSPEGMSEIYLR

SEQ ID NO: 520 - protein, 29 aa, Artificial
  Peptide
TGSGGPGNHPHGPDASAEGLNPYGLVAPR

SEQ ID NO: 521 - protein, 19 aa, Artificial
  Peptide
ILEAHQNVAQLSLAEAQLR

SEQ ID NO: 522 - protein, 11 aa, Artificial
  Peptide
KDEILGIANNR

SEQ ID NO: 523 - protein, 10 aa, Artificial
  Peptide
IDLAVGDVVK

SEQ ID NO: 524 - protein, 17 aa, Artificial
  Peptide
AGQSAAGAAPGGGVDTR

SEQ ID NO: 525 - protein, 9 aa, Artificial
  Peptide
DLAEDAPWK

SEQ ID NO: 526 - protein, 8 aa, Artificial
  Peptide
IQQNTFTR

SEQ ID NO: 527 - protein, 14 aa, Artificial
  Peptide
IANLQTDLSDGLDR

SEQ ID NO: 528 - protein, 11 aa, Artificial
  Peptide
LIALLEVLSQK

SEQ ID NO: 529 - protein, 16 aa, Artificial
  Peptide
QMQLENVSVALEFLDR

SEQ ID NO: 530 - protein, 8 aa, Artificial
  Peptide
LLGWIQNK

SEQ ID NO: 531 - protein, 11 aa, Artificial
  Peptide
LPQLPITNFSR

SEQ ID NO: 532 - protein, 9 aa, Artificial
  Peptide
RAEFTVETR

SEQ ID NO: 533 - protein, 22 aa, Artificial
  Peptide
SAGQGEVLVYVEDPAGHQEEAK

SEQ ID NO: 534 - protein, 12 aa, Artificial
  Peptide
VTVLFAGQHIAK

SEQ ID NO: 535 - protein, 9 aa, Artificial
  Peptide
SPFEVYVDK

SEQ ID NO: 536 - protein, 17 aa, Artificial
  Peptide
VTAQGPGLEPSGNIANK

SEQ ID NO: 537 - protein, 10 aa, Artificial
  Peptide
GTVEPQLEAR

SEQ ID NO: 538 - protein, 6 aa, Artificial
  Peptide
SPFEVK

SEQ ID NO: 539 - protein, 13 aa, Artificial
  Peptide
AWGPGLEGGVVGK

SEQ ID NO: 540 - protein, 9 aa, Artificial
  Peptide
LSPFMADIR

SEQ ID NO: 541 - protein, 10 aa, Artificial
  Peptide
DAPQDFHPDR

SEQ ID NO: 542 - protein, 16 aa, Artificial
  Peptide
TGVAVNKPAEFTVDAK

SEQ ID NO: 543 - protein, 11 aa, Artificial
  Peptide
VNVGAGSHPNK

SEQ ID NO: 544 - protein, 9 aa, Artificial
  Peptide
NDNDTFTVK

SEQ ID NO: 545 - protein, 11 aa, Artificial
  Peptide
VKVEPSHDASK

SEQ ID NO: 546 - protein, 10 aa, Artificial
  Peptide
VKAEGPGLSR

SEQ ID NO: 547 - protein, 8 aa, Artificial
  Peptide
AEGPGLSR

SEQ ID NO: 548 - protein, 12 aa, Artificial
  Peptide
GKLDVQFSGLTK

SEQ ID NO: 549 - protein, 10 aa, Artificial
  Peptide
LDVQFSGLTK

SEQ ID NO: 550 - protein, 15 aa, Artificial
  Peptide
DVDIIDHHDNTYTVK

SEQ ID NO: 551 - protein, 22 aa, Artificial
  Peptide
YTPVQQGPVGVNVTYGGDPIPK

SEQ ID NO: 552 - protein, 15 aa, Artificial
  Peptide
SPFSVAVSPSLDLSK

SEQ ID NO: 553 - protein, 12 aa, Artificial
  Peptide
VDVGKDQEFTVK

SEQ ID NO: 554 - protein, 7 aa, Artificial
  Peptide
DQEFTVK

SEQ ID NO: 555 - protein, 13 aa, Artificial
  Peptide
VEPGLGADNSVVR

SEQ ID NO: 556 - protein, 33 aa, Artificial
  Peptide
EEGPYEVEVTYDGVPVPGSPFPLEAVAPTKPSK

SEQ ID NO: 557 - protein, 16 aa, Artificial
  Peptide
AFGPGLQGGSAGSPAR

SEQ ID NO: 558 - protein, 12 aa, Artificial
  Peptide
YGGQPVPNFPSK

SEQ ID NO: 559 - protein, 11 aa, Artificial
  Peptide
EATTEFSVDAR

SEQ ID NO: 560 - protein, 11 aa, Artificial
  Peptide
ALTQTGGPHVK

SEQ ID NO: 561 - protein, 16 aa, Artificial
  Peptide
VANPSGNLTETYVQDR

SEQ ID NO: 562 - protein, 16 aa, Artificial
  Peptide
VHGPGIQSGTTNKPNK

SEQ ID NO: 563 - protein, 18 aa, Artificial
  Peptide
GAGTGGLGLAVEGPSEAK

SEQ ID NO: 564 - protein, 11 aa, Artificial
  Peptide
VPVHDVTDASK

SEQ ID NO: 565 - protein, 13 aa, Artificial
  Peptide
ANLPQSFQVDTSK

SEQ ID NO: 566 - protein, 9 aa, Artificial
  Peptide
AGVAPLQVK

SEQ ID NO: 567 - protein, 24 aa, Artificial
  Peptide
GLVEPVDVVDNADGTQTVNYVPSR

SEQ ID NO: 568 - protein, 17 aa, Artificial
  Peptide
EGPYSISVLYGDEEVPR

SEQ ID NO: 569 - protein, 24 aa, Artificial
  Peptide
ASGPGLNTTGVPASLPVEFTIDAK

SEQ ID NO: 570 - protein, 17 aa, Artificial
  Peptide
DAGEGLLAVQITDPGGK

SEQ ID NO: 571 - protein, 19 aa, Artificial
  Peptide
DAGEGLLAVQITDPEGKPK

SEQ ID NO: 572 - protein, 23 aa, Artificial
  Peptide
KTHIQDNHDGTYTVAYVPDVTGR

SEQ ID NO: 573 - protein, 12 aa, Artificial
  Peptide
YGGDEIPFSPYR

SEQ ID NO: 574 - protein, 9 aa, Artificial
  Peptide
KGEITGEVR

SEQ ID NO: 575 - protein, 10 aa, Artificial
  Peptide
VAQPTITDNK

SEQ ID NO: 576 - protein, 16 aa, Artificial
  Peptide
DAGEGGLSLAIEGPSK

SEQ ID NO: 577 - protein, 14 aa, Artificial
  Peptide
YNEQHVPGSPFTAR

SEQ ID NO: 578 - protein, 29 aa, Artificial
  Peptide
VGSAADIPINISETDLSLLTATVVPPSGR

SEQ ID NO: 579 - protein, 10 aa, Artificial
  Peptide
ETGEHLVHVK

SEQ ID NO: 580 - protein, 8 aa, Artificial
  Peptide
AGGPGLER

SEQ ID NO: 581 - protein, 16 aa, Artificial
  Peptide
EAGAGGLAIAVEGPSK

SEQ ID NO: 582 - protein, 23 aa, Artificial
  Peptide
GQHVPGSPFQFTVGPLGEGGAHK

SEQ ID NO: 583 - protein, 13 aa, Artificial
  Peptide
RLTVSSLQESGLK

SEQ ID NO: 584 - protein, 12 aa, Artificial
  Peptide
LTVSSLQESGLK

SEQ ID NO: 585 - protein, 15 aa, Artificial
  Peptide
VNQPASFAVSLNGAK

SEQ ID NO: 586 - protein, 16 aa, Artificial
  Peptide
VTYTPMAPGSYLISIK

SEQ ID NO: 587 - protein, 13 aa, Artificial
  Peptide
YGGPYHIGGSPFK

SEQ ID NO: 588 - protein, 20 aa, Artificial
  Peptide
LVSNHSLHETSSVFVDSLTK

SEQ ID NO: 589 - protein, 14 aa, Artificial
  Peptide
AGNNMLLVGVHGPR

SEQ ID NO: 590 - protein, 13 aa, Artificial
  Peptide
IGNLQTDLSDGLR

SEQ ID NO: 591 - protein, 10 aa, Artificial
  Peptide
IFAQDGEGQR

SEQ ID NO: 592 - protein, 26 aa, Artificial
  Peptide
VTASGPGLSSYGVPASLPVDFAIDAR

SEQ ID NO: 593 - protein, 10 aa, Artificial
  Peptide
SPPVMVAGGR

SEQ ID NO: 594 - protein, 10 aa, Artificial
  Peptide
AAYDIEVNTR

SEQ ID NO: 595 - protein, 11 aa, Artificial
  Peptide
ISLNTLTLNVK

SEQ ID NO: 596 - protein, 18 aa, Artificial
  Peptide
TEAEIAHIALETLEGHQR

SEQ ID NO: 597 - protein, 14 aa, Artificial
  Peptide
DIHDDQDYLHSLGK

SEQ ID NO: 598 - protein, 13 aa, Artificial
  Peptide
AQQVAVQEQEIAR

SEQ ID NO: 599 - protein, 15 aa, Artificial
  Peptide
SQLIMQAEAEAASVR

SEQ ID NO: 600 - protein, 11 aa, Artificial
  Peptide
GEAEAFAIGAR

SEQ ID NO: 601 - protein, 17 aa, Artificial
  Peptide
LPQVAEEISGPLTSANK

SEQ ID NO: 602 - protein, 11 aa, Artificial
  Peptide
VTGEVLDILTR

SEQ ID NO: 603 - protein, 12 aa, Artificial
  Peptide
VLLVLELQGLQK

SEQ ID NO: 604 - protein, 9 aa, Artificial
  Peptide
FRIDELEPR

SEQ ID NO: 605 - protein, 7 aa, Artificial
  Peptide
IDELEPR

SEQ ID NO: 606 - protein, 15 aa, Artificial
  Peptide
YRVPVVLVADPPIAR

SEQ ID NO: 607 - protein, 13 aa, Artificial
  Peptide
VPVVLVADPPIAR

SEQ ID NO: 608 - protein, 15 aa, Artificial
  Peptide
DENSVELTMAEGPYK

SEQ ID NO: 609 - protein, 7 aa, Artificial
  Peptide
LDLLEDR

SEQ ID NO: 610 - protein, 14 aa, Artificial
  Peptide
AEKDEPGAWEETFK

SEQ ID NO: 611 - protein, 9 aa, Artificial
  Peptide
VTEGGEPYR

SEQ ID NO: 612 - protein, 17 aa, Artificial
  Peptide
MMDYLQGSGETPQTDVR

SEQ ID NO: 613 - protein, 10 aa, Artificial
  Peptide
LVAIVDPHIK

SEQ ID NO: 614 - protein, 27 aa, Artificial
  Peptide
FSFSGNTLVSSSADFEGHFETPIWIER

SEQ ID NO: 615 - protein, 9 aa, Artificial
  Peptide
PAAVVLQTK

SEQ ID NO: 616 - protein, 15 aa, Artificial
  Peptide
LSFQHDPETSVLVLR

SEQ ID NO: 617 - protein, 8 aa, Artificial
  Peptide
MVEGFFDR

SEQ ID NO: 618 - protein, 12 aa, Artificial
  Peptide
DDGSWEVIEGYR

SEQ ID NO: 619 - protein, 11 aa, Artificial
  Peptide
YSTDVSVDEVK

SEQ ID NO: 620 - protein, 19 aa, Artificial
  Peptide
GFIGPGIDVPAPDMSTGER

SEQ ID NO: 621 - protein, 20 aa, Artificial
  Peptide
KGFIGPGIDVPAPDMSTGER

SEQ ID NO: 622 - protein, 9 aa, Artificial
  Peptide
ALASLMTYK

SEQ ID NO: 623 - protein, 10 aa, Artificial
  Peptide
PISQGGIHGR

SEQ ID NO: 624 - protein, 16 aa, Artificial
  Peptide
IIAEGANGPTTPEADK

SEQ ID NO: 625 - protein, 9 aa, Artificial
  Peptide
NLNHVSYGR

SEQ ID NO: 626 - protein, 16 aa, Artificial
  Peptide
HGGTIPIVPTAEFQDR

SEQ ID NO: 627 - protein, 13 aa, Artificial
  Peptide
DIVHSGLAYTMER

SEQ ID NO: 628 - protein, 10 aa, Artificial
  Peptide
TAAYVNAIEK

SEQ ID NO: 629 - protein, 14 aa, Artificial
  Peptide
AMGNLQIDFADPSR

SEQ ID NO: 630 - protein, 17 aa, Artificial
  Peptide
EYQLNDSAAYYLNDLER

SEQ ID NO: 631 - protein, 15 aa, Artificial
  Peptide
IAQSDYIPTQQDVLR

SEQ ID NO: 632 - protein, 12 aa, Artificial
  Peptide
TTGIVETHFTFK

SEQ ID NO: 633 - protein, 8 aa, Artificial
  Peptide
MFDVGGQR

SEQ ID NO: 634 - protein, 11 aa, Artificial
  Peptide
YDEAASYIQSK

SEQ ID NO: 635 - protein, 15 aa, Artificial
  Peptide
NVQFVFDAVTDVIIK

SEQ ID NO: 636 - protein, 27 aa, Artificial
  Peptide
EAIETIVAAMSNLVPPVELANPENQFR

SEQ ID NO: 637 - protein, 12 aa, Artificial
  Peptide
VNFHMFDVGGQR

SEQ ID NO: 638 - protein, 10 aa, Artificial
  Peptide
VLTSGIFETK

SEQ ID NO: 639 - protein, 16 aa, Artificial
  Peptide
YTTPEDATPEPGEDPR

SEQ ID NO: 640 - protein, 11 aa, Artificial
  Peptide
IYAMHWGTDSR

SEQ ID NO: 641 - protein, 10 aa, Artificial
  Peptide
LLVSASQDGK

SEQ ID NO: 642 - protein, 11 aa, Artificial
  Peptide
LIIWDSYTTNK

SEQ ID NO: 643 - protein, 10 aa, Artificial
  Peptide
AGVLAGHDNR

SEQ ID NO: 644 - protein, 11 aa, Artificial
  Peptide
TASTNNIAQAR

SEQ ID NO: 645 - protein, 7 aa, Artificial
  Peptide
LEASIER

SEQ ID NO: 646 - protein, 16 aa, Artificial
  Peptide
SDPLLIGIPTSENPFK

SEQ ID NO: 647 - protein, 9 aa, Artificial
  Peptide
MNLGVGAYR

SEQ ID NO: 648 - protein, 14 aa, Artificial
  Peptide
FVTVQTISGTGALR

SEQ ID NO: 649 - protein, 8 aa, Artificial
  Peptide
IGASFLQR

SEQ ID NO: 650 - protein, 11 aa, Artificial
  Peptide
PTWGNHTPIFR

SEQ ID NO: 651 - protein, 10 aa, Artificial
  Peptide
DAGMQLQGYR

SEQ ID NO: 652 - protein, 12 aa, Artificial
  Peptide
IAAAILNTPDLR

SEQ ID NO: 653 - protein, 14 aa, Artificial
  Peptide
ITPSYVAFTPEGER

SEQ ID NO: 654 - protein, 15 aa, Artificial
  Peptide
NQLTSNPENTVFDAK

SEQ ID NO: 655 - protein, 12 aa, Artificial
  Peptide
TWNDPSVQQDIK

SEQ ID NO: 656 - protein, 15 aa, Artificial
  Peptide
TKPYIQVDIGGGQTK

SEQ ID NO: 657 - protein, 14 aa, Artificial
  Peptide
TFAPEEISAMVLTK

SEQ ID NO: 658 - protein, 11 aa, Artificial
  Peptide
MKETAEAYLGK

SEQ ID NO: 659 - protein, 17 aa, Artificial
  Peptide
VTHAVVTVPAYFNDAQR

SEQ ID NO: 660 - protein, 12 aa, Artificial
  Peptide
DAGTIAGLNVMR

SEQ ID NO: 661 - protein, 17 aa, Artificial
  Peptide
IINEPTAAAIAYGLDKR

SEQ ID NO: 662 - protein, 7 aa, Artificial
  Peptide
VMEHFIK

SEQ ID NO: 663 - protein, 12 aa, Artificial
  Peptide
AKFEELNMDLFR

SEQ ID NO: 664 - protein, 8 aa, Artificial
  Peptide
VLEDSDLK

SEQ ID NO: 665 - protein, 15 aa, Artificial
  Peptide
KSDIDEIVLVGGSTR

SEQ ID NO: 666 - protein, 17 aa, Artificial
  Peptide
SQIFSTASDNQPTVTIK

SEQ ID NO: 667 - protein, 10 aa, Artificial
  Peptide
VYEGERPLTK

SEQ ID NO: 668 - protein, 18 aa, Artificial
  Peptide
DNHLLGTFDLTGIPPAPR

SEQ ID NO: 669 - protein, 18 aa, Artificial
  Peptide
GVPQIEVTFEIDVNGILR

SEQ ID NO: 670 - protein, 9 aa, Artificial
  Peptide
ITITNDQNR

SEQ ID NO: 671 - protein, 7 aa, Artificial
  Peptide
FAEEDKK

SEQ ID NO: 672 - protein, 11 aa, Artificial
  Peptide
NELESYAYSLK

SEQ ID NO: 673 - protein, 16 aa, Artificial
  Peptide
IEWLESHQDADIEDFK

SEQ ID NO: 674 - protein, 12 aa, Artificial
  Peptide
ELEEIVQPIISK

SEQ ID NO: 675 - protein, 11 aa, Artificial
  Peptide
HVVPNEVVVQR

SEQ ID NO: 676 - protein, 20 aa, Artificial
  Peptide
VHVSEEGTEPEAMLQVLGPK

SEQ ID NO: 677 - protein, 12 aa, Artificial
  Peptide
PALPAGTEDTAK

SEQ ID NO: 678 - protein, 25 aa, Artificial
  Peptide
VSNGAGTMSVSLVADENPFAQGALK

SEQ ID NO: 679 - protein, 17 aa, Artificial
  Peptide
QTQVSVLPEGGETPLFK

SEQ ID NO: 680 - protein, 28 aa, Artificial
  Peptide
VPFDAATLHTSTAMAAQHGMDDDGTGQK

SEQ ID NO: 681 - protein, 18 aa, Artificial
  Peptide
TPSAAYLWVGTGASEAEK

SEQ ID NO: 682 - protein, 22 aa, Artificial
  Peptide
DPDQTDGLGLSYLSSHIANVER

SEQ ID NO: 683 - protein, 13 aa, Artificial
  Peptide
AGALNSNDAFVLK

SEQ ID NO: 684 - protein, 8 aa, Artificial
  Peptide
TGAQELLR

SEQ ID NO: 685 - protein, 22 aa, Artificial
  Peptide
AQPVQVAEGSEPDGFWEALGGK

SEQ ID NO: 686 - protein, 15 aa, Artificial
  Peptide
DSQEEEKTEALTSAK

SEQ ID NO: 687 - protein, 7 aa, Artificial
  Peptide
TPITVVK

SEQ ID NO: 688 - protein, 9 aa, Artificial
  Peptide
NVVVVDGVR

SEQ ID NO: 689 - protein, 11 aa, Artificial
  Peptide
TPFLLSGTSYK

SEQ ID NO: 690 - protein, 9 aa, Artificial
  Peptide
LEQDEYALR

SEQ ID NO: 691 - protein, 14 aa, Artificial
  Peptide
AQDEGLLSDVVPFK

SEQ ID NO: 692 - protein, 13 aa, Artificial
  Peptide
DQLLLGPTYATPK

SEQ ID NO: 693 - protein, 14 aa, Artificial
  Peptide
AMDSDWFAENYMGR

SEQ ID NO: 694 - protein, 11 aa, Artificial
  Peptide
HIDLVEGDEGR

SEQ ID NO: 695 - protein, 21 aa, Artificial
  Peptide
TPDGTENGDFLALDLGGTNFR

SEQ ID NO: 696 - protein, 11 aa, Artificial
  Peptide
FNTSDVSAIEK

SEQ ID NO: 697 - protein, 15 aa, Artificial
  Peptide
SANLVAATLGAILNR

SEQ ID NO: 698 - protein, 11 aa, Artificial
  Peptide
GAAMVTAVAYR

SEQ ID NO: 699 - protein, 11 aa, Artificial
  Peptide
LVDEYSLNAGK

SEQ ID NO: 700 - protein, 9 aa, Artificial
  Peptide
FLSQIESDR

SEQ ID NO: 701 - protein, 11 aa, Artificial
  Peptide
GAALITAVGVR

SEQ ID NO: 702 - protein, 12 aa, Artificial
  Peptide
NASDMPETITSR

SEQ ID NO: 703 - protein, 9 aa, Artificial
  Peptide
DPAAVTESK

SEQ ID NO: 704 - protein, 10 aa, Artificial
  Peptide
LVFPQDLLEK

SEQ ID NO: 705 - protein, 13 aa, Artificial
  Peptide
VVLVTGAGAGLGR

SEQ ID NO: 706 - protein, 16 aa, Artificial
  Peptide
ATSTATSGFAGAIGQK

SEQ ID NO: 707 - protein, 12 aa, Artificial
  Peptide
VLQQFADNDVSR

SEQ ID NO: 708 - protein, 14 aa, Artificial
  Peptide
ELISNASDALDKIR

SEQ ID NO: 709 - protein, 13 aa, Artificial
  Peptide
GVVDSDDLPNVSR

SEQ ID NO: 710 - protein, 19 aa, Artificial
  Peptide
FQSSHHPTDITSLDQYVER

SEQ ID NO: 711 - protein, 11 aa, Artificial
  Peptide
FDDAVVQSDMK

SEQ ID NO: 712 - protein, 14 aa, Artificial
  Peptide
SFYPEEVSSMVLTK

SEQ ID NO: 713 - protein, 18 aa, Artificial
  Peptide
TVTNAVVTVPAYFNDSQR

SEQ ID NO: 714 - protein, 12 aa, Artificial
  Peptide
DAGTIAGLNVLR

SEQ ID NO: 715 - protein, 16 aa, Artificial
  Peptide
STAGDTHLGGEDFDNR

SEQ ID NO: 716 - protein, 12 aa, Artificial
  Peptide
ARFEELNADLFR

SEQ ID NO: 717 - protein, 10 aa, Artificial
  Peptide
FEELNADLFR

SEQ ID NO: 718 - protein, 23 aa, Artificial
  Peptide
SINPDEAVAYGAAVQAAILSGDK

SEQ ID NO: 719 - protein, 11 aa, Artificial
  Peptide
NSLESYAFNMK

SEQ ID NO: 720 - protein, 14 aa, Artificial
  Peptide
NQTAEKEEFEHQQK

SEQ ID NO: 721 - protein, 16 aa, Artificial
  Peptide
VAIVKPEVPMEIVLNK

SEQ ID NO: 722 - protein, 8 aa, Artificial
  Peptide
TPVIVTLK

SEQ ID NO: 723 - protein, 8 aa, Artificial
  Peptide
YFQHLLGK

SEQ ID NO: 724 - protein, 19 aa, Artificial
  Peptide
VLQLINDNTATALSYGVFR

SEQ ID NO: 725 - protein, 10 aa, Artificial
  Peptide
TLGGLEMELR

SEQ ID NO: 726 - protein, 9 aa, Artificial
  Peptide
LAGLFNEQR

SEQ ID NO: 727 - protein, 23 aa, Artificial
  Peptide
TVLSANADHMAQIEGLMDDVDFK

SEQ ID NO: 728 - protein, 30 aa, Artificial
  Peptide
VPGPVQQALQSAEMSLDEIEQVILVGGATR

SEQ ID NO: 729 - protein, 21 aa, Artificial
  Peptide
NINADEAAAMGAVYQAAALSK

SEQ ID NO: 730 - protein, 13 aa, Artificial
  Peptide
DAVVYPILVEFTR

SEQ ID NO: 731 - protein, 21 aa, Artificial
  Peptide
VESVFETLVEDSAEEESTLTK

SEQ ID NO: 732 - protein, 18 aa, Artificial
  Peptide
LGNTISSLFGGGTTPDAK

SEQ ID NO: 733 - protein, 16 aa, Artificial
  Peptide
AEAGPEGVAPAPEGEK

SEQ ID NO: 734 - protein, 15 aa, Artificial
  Peptide
LYQPEYQEVSTEEQR

SEQ ID NO: 735 - protein, 19 aa, Artificial
  Peptide
LIPEMDQIFTEVEMTTLEK

SEQ ID NO: 736 - protein, 10 aa, Artificial
  Peptide
LLGIETPLPK

SEQ ID NO: 737 - protein, 11 aa, Artificial
  Peptide
TFLTVYWTPER

SEQ ID NO: 738 - protein, 14 aa, Artificial
  Peptide
VELAPLPSWQPVGK

SEQ ID NO: 739 - protein, 18 aa, Artificial
  Peptide
REPAVGEPAEVTTTVLVR

SEQ ID NO: 740 - protein, 10 aa, Artificial
  Peptide
KVTVNVLSPR

SEQ ID NO: 741 - protein, 14 aa, Artificial
  Peptide
ASVSVTAEDEGTGR

SEQ ID NO: 742 - protein, 16 aa, Artificial
  Peptide
DGTFPLPIGESVTVTR

SEQ ID NO: 743 - protein, 15 aa, Artificial
  Peptide
VAKPVVEMDGDEMTR

SEQ ID NO: 744 - protein, 12 aa, Artificial
  Peptide
PVVEMDGDEMTR

SEQ ID NO: 745 - protein, 17 aa, Artificial
  Peptide
DQTDDQVTIDSALATQK

SEQ ID NO: 746 - protein, 8 aa, Artificial
  Peptide
ATDFVADR

SEQ ID NO: 747 - protein, 11 aa, Artificial
  Peptide
FKDIFQEIFDK

SEQ ID NO: 748 - protein, 11 aa, Artificial
  Peptide
LIDDMVAQVLK

SEQ ID NO: 749 - protein, 13 aa, Artificial
  Peptide
TIEAEAAHGTVTR

SEQ ID NO: 750 - protein, 11 aa, Artificial
  Peptide
GKLDGNQDLIR

SEQ ID NO: 751 - protein, 16 aa, Artificial
  Peptide
LNEHFLNTTDFLDTIK

SEQ ID NO: 752 - protein, 13 aa, Artificial
  Peptide
VEITYTPSDGTQK

SEQ ID NO: 753 - protein, 9 aa, Artificial
  Peptide
NILGGTVFR

SEQ ID NO: 754 - protein, 9 aa, Artificial
  Peptide
SAVQGPPDR

SEQ ID NO: 755 - protein, 9 aa, Artificial
  Peptide
SAVQGPPER

SEQ ID NO: 756 - protein, 15 aa, Artificial
  Peptide
DASGVTFTWTPSSGK

SEQ ID NO: 757 - protein, 9 aa, Artificial
  Peptide
TPLTATLSK

SEQ ID NO: 758 - protein, 10 aa, Artificial
  Peptide
WLQGSQELPR

SEQ ID NO: 759 - protein, 17 aa, Artificial
  Peptide
QEPSQGTTTFAVTSILR

SEQ ID NO: 760 - protein, 16 aa, Artificial
  Peptide
QVGSGVTTDEVEAEAK

SEQ ID NO: 761 - protein, 12 aa, Artificial
  Peptide
YAATSQVLLPSK

SEQ ID NO: 762 - protein, 16 aa, Artificial
  Peptide
QVGSGVTTDQVQAEAK

SEQ ID NO: 763 - protein, 15 aa, Artificial
  Peptide
VFAIPPSFASIFLTK

SEQ ID NO: 764 - protein, 15 aa, Artificial
  Peptide
YVTSAPMPEPQAPGR

SEQ ID NO: 765 - protein, 16 aa, Artificial
  Peptide
VVSVLTVVHQDWLNGK

SEQ ID NO: 766 - protein, 17 aa, Artificial
  Peptide
ATPPMLDSDGSFFLKSK

SEQ ID NO: 767 - protein, 12 aa, Artificial
  Peptide
WYVDGVEVHNAK

SEQ ID NO: 768 - protein, 12 aa, Artificial
  Peptide
GPSVFPLAPSSK

SEQ ID NO: 769 - protein, 14 aa, Artificial
  Peptide
FNWYVDGVEVHNAK

SEQ ID NO: 770 - protein, 17 aa, Artificial
  Peptide
TTPPVLDSDGSFFLYSK

SEQ ID NO: 771 - protein, 22 aa, Artificial
  Peptide
GFYPSDIAVEWESNGQPENNYK

SEQ ID NO: 772 - protein, 7 aa, Artificial
  Peptide
DTLMISR

SEQ ID NO: 773 - protein, 16 aa, Artificial
  Peptide
VVSVLTVLHQDWLNGK

SEQ ID NO: 774 - protein, 8 aa, Artificial
  Peptide
ALPAPIEK

SEQ ID NO: 775 - protein, 10 aa, Artificial
  Peptide
VTHEGSTVEK

SEQ ID NO: 776 - protein, 13 aa, Artificial
  Peptide
SGTSASLAISGLR

SEQ ID NO: 777 - protein, 8 aa, Artificial
  Peptide
LTVLGQPK

SEQ ID NO: 778 - protein, 19 aa, Artificial
  Peptide
AAPSVTLFPPSSEELQANK

SEQ ID NO: 779 - protein, 10 aa, Artificial
  Peptide
AGVETTTPSK

SEQ ID NO: 780 - protein, 19 aa, Artificial
  Peptide
ANPTVTLFPPSSEELQANK

SEQ ID NO: 781 - protein, 15 aa, Artificial
  Peptide
SYSCQVTHEGSTVEK

SEQ ID NO: 782 - protein, 15 aa, Artificial
  Peptide
YAASSYLSLTPEQWK

SEQ ID NO: 783 - protein, 10 aa, Artificial
  Peptide
AGVETTKPSK

SEQ ID NO: 784 - protein, 9 aa, Artificial
  Peptide
RQEEEDLLR

SEQ ID NO: 785 - protein, 10 aa, Artificial
  Peptide
VEEQLGSDTK

SEQ ID NO: 786 - protein, 11 aa, Artificial
  Peptide
LEEEQHALFAR

SEQ ID NO: 787 - protein, 10 aa, Artificial
  Peptide
AVSEIMEMQK

SEQ ID NO: 788 - protein, 12 aa, Artificial
  Peptide
TLEGIQYDNSIK

SEQ ID NO: 789 - protein, 9 aa, Artificial
  Peptide
MQNELDILK

SEQ ID NO: 790 - protein, 9 aa, Artificial
  Peptide
SEAIMEQLK

SEQ ID NO: 791 - protein, 21 aa, Artificial
  Peptide
ISGLVTDVISLTDSVQELENK

SEQ ID NO: 792 - protein, 12 aa, Artificial
  Peptide
NIGDLLSSSIDR

SEQ ID NO: 793 - protein, 11 aa, Artificial
  Peptide
LEPLVNDLTLR

SEQ ID NO: 794 - protein, 8 aa, Artificial
  Peptide
LVTDLLQR

SEQ ID NO: 795 - protein, 9 aa, Artificial
  Peptide
VENQYQLLK

SEQ ID NO: 796 - protein, 12 aa, Artificial
  Peptide
LETNEFQQLQSK

SEQ ID NO: 797 - protein, 17 aa, Artificial
  Peptide
LFEMVLGPAAYNVPLPK

SEQ ID NO: 798 - protein, 28 aa, Artificial
  Peptide
QTASVTLQAIAAQNAAVQAVNAHSNILK

SEQ ID NO: 799 - protein, 12 aa, Artificial
  Peptide
KAVDEAADALLK

SEQ ID NO: 800 - protein, 11 aa, Artificial
  Peptide
AVDEAADALLK

SEQ ID NO: 801 - protein, 9 aa, Artificial
  Peptide
SEIQAEQDR

SEQ ID NO: 802 - protein, 13 aa, Artificial
  Peptide
LHNMIVDLDNVVK

SEQ ID NO: 803 - protein, 9 aa, Artificial
  Peptide
LSEQELQFR

SEQ ID NO: 804 - protein, 17 aa, Artificial
  Peptide
GIEQAVQSHAVAEEEAR

SEQ ID NO: 805 - protein, 19 aa, Artificial
  Peptide
TSSAETPTIPLGSAVEAIK

SEQ ID NO: 806 - protein, 15 aa, Artificial
  Peptide
YEQGFITDPVVLSPK

SEQ ID NO: 807 - protein, 8 aa, Artificial
  Peptide
LVGIISSR

SEQ ID NO: 808 - protein, 14 aa, Artificial
  Peptide
REDLVVAPAGITLK

SEQ ID NO: 809 - protein, 16 aa, Artificial
  Peptide
LPIVNEDDELVAIIAR

SEQ ID NO: 810 - protein, 11 aa, Artificial
  Peptide
NLIDAGVDALR

SEQ ID NO: 811 - protein, 10 aa, Artificial
  Peptide
GMGSLDAMDK

SEQ ID NO: 812 - protein, 12 aa, Artificial
  Peptide
VAQGVSGAVQDK

SEQ ID NO: 813 - protein, 12 aa, Artificial
  Peptide
IFYPETTDIYDR

SEQ ID NO: 814 - protein, 25 aa, Artificial
  Peptide
QSGQTDPLQKEELQSGVDAAQQYQR

SEQ ID NO: 815 - protein, 13 aa, Artificial
  Peptide
LGLAPQIQDLYGK

SEQ ID NO: 816 - protein, 12 aa, Artificial
  Peptide
VDFTEEEINNMK

SEQ ID NO: 817 - protein, 10 aa, Artificial
  Peptide
YGIQMPAFSK

SEQ ID NO: 818 - protein, 11 aa, Artificial
  Peptide
RIPADTFAALK

SEQ ID NO: 819 - protein, 10 aa, Artificial
  Peptide
IPADTFAALK

SEQ ID NO: 820 - protein, 25 aa, Artificial
  Peptide
NPNAMLVNLEEPLASTYQDILYQAK

SEQ ID NO: 821 - protein, 24 aa, Artificial
  Peptide
VNTFSALANIDLALEQEDALALFR

SEQ ID NO: 822 - protein, 10 aa, Artificial
  Peptide
ALQSPALGLR

SEQ ID NO: 823 - protein, 12 aa, Artificial
  Peptide
GLQQQNSDWYLK

SEQ ID NO: 824 - protein, 19 aa, Artificial
  Peptide
EELQSGVDAANSAAQQYQR

SEQ ID NO: 825 - protein, 18 aa, Artificial
  Peptide
ILAIGLINEALDEGDAQK

SEQ ID NO: 826 - protein, 11 aa, Artificial
  Peptide
TLQALQIPAAK

SEQ ID NO: 827 - protein, 18 aa, Artificial
  Peptide
LEGVLAEVAQHYQDTLIR

SEQ ID NO: 828 - protein, 13 aa, Artificial
  Peptide
EQLWLANEGLITR

SEQ ID NO: 829 - protein, 14 aa, Artificial
  Peptide
TLINAEDPPMVVVR

SEQ ID NO: 830 - protein, 20 aa, Artificial
  Peptide
FVHLLDQSDQDFQEELDLMK

SEQ ID NO: 831 - protein, 15 aa, Artificial
  Peptide
SNQQLENDLNLMDIK

SEQ ID NO: 832 - protein, 15 aa, Artificial
  Peptide
SWVNQMESQTGEASK

SEQ ID NO: 833 - protein, 25 aa, Artificial
  Peptide
FQPGETLTEILETPATSEQEAEHQR

SEQ ID NO: 834 - protein, 11 aa, Artificial
  Peptide
ITLQDVVSHSK

SEQ ID NO: 835 - protein, 16 aa, Artificial
  Peptide
VDQIQEIVTGNPTVIK

SEQ ID NO: 836 - protein, 17 aa, Artificial
  Peptide
LPYDVTPEQALAHEEVK

SEQ ID NO: 837 - protein, 11 aa, Artificial
  Peptide
FLSAIVSSVDK

SEQ ID NO: 838 - protein, 14 aa, Artificial
  Peptide
FNVDEYSDLVTLTK

SEQ ID NO: 839 - protein, 14 aa, Artificial
  Peptide
FDVPGDENAEMDAR

SEQ ID NO: 840 - protein, 7 aa, Artificial
  Peptide
LIVDVIR

SEQ ID NO: 841 - protein, 10 aa, Artificial
  Peptide
LTELGTVDPK

SEQ ID NO: 842 - protein, 12 aa, Artificial
  Peptide
ATFYGEQVDYYK

SEQ ID NO: 843 - protein, 21 aa, Artificial
  Peptide
IFSGPSSEQFGYAVQQFINPK

SEQ ID NO: 844 - protein, 17 aa, Artificial
  Peptide
GNWLLVGSPWSGFPENR

SEQ ID NO: 845 - protein, 11 aa, Artificial
  Peptide
FVQGLDIGPTK

SEQ ID NO: 846 - protein, 13 aa, Artificial
  Peptide
TQVGLIQYANNPR

SEQ ID NO: 847 - protein, 9 aa, Artificial
  Peptide
VVFNLNTYK

SEQ ID NO: 848 - protein, 30 aa, Artificial
  Peptide
TKEEMIVATSQTSQYGGDLTNTFGAIQYAR

SEQ ID NO: 849 - protein, 11 aa, Artificial
  Peptide
KYAYSAASGGR

SEQ ID NO: 850 - protein, 11 aa, Artificial
  Peptide
FGIAVLGYLNR

SEQ ID NO: 851 - protein, 8 aa, Artificial
  Peptide
SHLQYFGR

SEQ ID NO: 852 - protein, 7 aa, Artificial
  Peptide
NAQIILK

SEQ ID NO: 853 - protein, 22 aa, Artificial
  Peptide
VDISLGNPGTSPALEAYSETAK

SEQ ID NO: 854 - protein, 17 aa, Artificial
  Peptide
QIPAAQEQPFIVSNQNK

SEQ ID NO: 855 - protein, 13 aa, Artificial
  Peptide
IPLLYDAEIHLTR

SEQ ID NO: 856 - protein, 28 aa, Artificial
  Peptide
STNINFYEISSDGNVPSIVHSFEDVGPK

SEQ ID NO: 857 - protein, 11 aa, Artificial
  Peptide
IGQTSSSVSFK

SEQ ID NO: 858 - protein, 5 aa, Artificial
  Peptide
FLVVK

SEQ ID NO: 859 - protein, 17 aa, Artificial
  Peptide
EAGNPGSLFGYSVALHR

SEQ ID NO: 860 - protein, 9 aa, Artificial
  Peptide
YLLLAGAPR

SEQ ID NO: 861 - protein, 13 aa, Artificial
  Peptide
YTQVLWSGSEDQR

SEQ ID NO: 862 - protein, 11 aa, Artificial
  Peptide
KEWDLSEYSYK

SEQ ID NO: 863 - protein, 10 aa, Artificial
  Peptide
ARPVINTVHK

SEQ ID NO: 864 - protein, 10 aa, Artificial
  Peptide
LELLLMDNLR

SEQ ID NO: 865 - protein, 10 aa, Artificial
  Peptide
AAFVSEQQQK

SEQ ID NO: 866 - protein, 10 aa, Artificial
  Peptide
TVEDVGSPLK

SEQ ID NO: 867 - protein, 19 aa, Artificial
  Peptide
QLDPGGGQGPPPVTLAAAK

SEQ ID NO: 868 - protein, 8 aa, Artificial
  Peptide
LLLVGAPR

SEQ ID NO: 869 - protein, 14 aa, Artificial
  Peptide
IEFDNDADPTSESK

SEQ ID NO: 870 - protein, 16 aa, Artificial
  Peptide
DGWQDIVIGAPQYFDR

SEQ ID NO: 871 - protein, 15 aa, Artificial
  Peptide
LIATFPDTLTYSAYR

SEQ ID NO: 872 - protein, 16 aa, Artificial
  Peptide
LETTSNQDNLAPITAK

SEQ ID NO: 873 - protein, 14 aa, Artificial
  Peptide
SEDEVGSLIEYEFR

SEQ ID NO: 874 - protein, 10 aa, Artificial
  Peptide
SDGIYIINLK

SEQ ID NO: 875 - protein, 17 aa, Artificial
  Peptide
AIVAIENPADVSVISSR

SEQ ID NO: 876 - protein, 13 aa, Artificial
  Peptide
FAAATGATPIAGR

SEQ ID NO: 877 - protein, 15 aa, Artificial
  Peptide
FTPGTFTNQIQAAFR

SEQ ID NO: 878 - protein, 15 aa, Artificial
  Peptide
DPEEIEKEEQAAAEK

SEQ ID NO: 879 - protein, 28 aa, Artificial
  Peptide
GPHLVQSDGTVPFWAHAGNAIPSSDQIR

SEQ ID NO: 880 - protein, 14 aa, Artificial
  Peptide
YQEEFEHFQQELDK

SEQ ID NO: 881 - protein, 20 aa, Artificial
  Peptide
GHPDLQGQPAEEIFESVGDR

SEQ ID NO: 882 - protein, 10 aa, Artificial
  Peptide
QLDMILDEQR

SEQ ID NO: 883 - protein, 11 aa, Artificial
  Peptide
YVSSLTEEISK

SEQ ID NO: 884 - protein, 8 aa, Artificial
  Peptide
DIDNLVQR

SEQ ID NO: 885 - protein, 15 aa, Artificial
  Peptide
GATLALTQVTPQDER

SEQ ID NO: 886 - protein, 19 aa, Artificial
  Peptide
APEEPNIQVNPLGIPVNSK

SEQ ID NO: 887 - protein, 12 aa, Artificial
  Peptide
EVTVPVFYPTEK

SEQ ID NO: 888 - protein, 9 aa, Artificial
  Peptide
EETGQVLER

SEQ ID NO: 889 - protein, 11 aa, Artificial
  Peptide
GPVLQLHDLKR

SEQ ID NO: 890 - protein, 19 aa, Artificial
  Peptide
VAVLGASGGIGQPLSLLLK

SEQ ID NO: 891 - protein, 22 aa, Artificial
  Peptide
LTLYDIAHTPGVAADLSHIETK

SEQ ID NO: 892 - protein, 11 aa, Artificial
  Peptide
IFGVTTLDIVR

SEQ ID NO: 893 - protein, 9 aa, Artificial
  Peptide
ANTFVAELK

SEQ ID NO: 894 - protein, 14 aa, Artificial
  Peptide
VDFPQDQLTALTGR

SEQ ID NO: 895 - protein, 10 aa, Artificial
  Peptide
IQEAGTEVVK

SEQ ID NO: 896 - protein, 16 aa, Artificial
  Peptide
AGAGSATLSMAYAGAR

SEQ ID NO: 897 - protein, 12 aa, Artificial
  Peptide
FVFSLVDAMNGK

SEQ ID NO: 898 - protein, 10 aa, Artificial
  Peptide
MISDAIPELK

SEQ ID NO: 899 - protein, 18 aa, Artificial
  Peptide
LAGSLLTQALESHAEGFR

SEQ ID NO: 900 - protein, 9 aa, Artificial
  Peptide
VPPEFLVQR

SEQ ID NO: 901 - protein, 22 aa, Artificial
  Peptide
PQPQLQYVDALGYVSLFPLLLR

SEQ ID NO: 902 - protein, 9 aa, Artificial
  Peptide
RDGRGGGPR

SEQ ID NO: 903 - protein, 12 aa, Artificial
  Peptide
LGPLLDILADSR

SEQ ID NO: 904 - protein, 15 aa, Artificial
  Peptide
NFINNPLAQADWAAK

SEQ ID NO: 905 - protein, 9 aa, Artificial
  Peptide
SGFEPASLK

SEQ ID NO: 906 - protein, 14 aa, Artificial
  Peptide
NLPIYSEEIVEMYK

SEQ ID NO: 907 - protein, 16 aa, Artificial
  Peptide
HEMPPHIYAITDTAYR

SEQ ID NO: 908 - protein, 13 aa, Artificial
  Peptide
VIQYLAYVASSHK

SEQ ID NO: 909 - protein, 10 aa, Artificial
  Peptide
TDLLLEPYNK

SEQ ID NO: 910 - protein, 11 aa, Artificial
  Peptide
DMFQETMEAMR

SEQ ID NO: 911 - protein, 14 aa, Artificial
  Peptide
IMGIPEEEQMGLLR

SEQ ID NO: 912 - protein, 14 aa, Artificial
  Peptide
VISGVLQLGNIVFK

SEQ ID NO: 913 - protein, 15 aa, Artificial
  Peptide
NTDQASMPDNTAAQK

SEQ ID NO: 914 - protein, 14 aa, Artificial
  Peptide
VSHLLGINVTDFTR

SEQ ID NO: 915 - protein, 19 aa, Artificial
  Peptide
NMDPLNDNIATLLHQSSDK

SEQ ID NO: 916 - protein, 20 aa, Artificial
  Peptide
IIGLDQVAGMSETALPGAFK

SEQ ID NO: 917 - protein, 7 aa, Artificial
  Peptide
VVFQEFR

SEQ ID NO: 918 - protein, 11 aa, Artificial
  Peptide
YEILTPNSIPK

SEQ ID NO: 919 - protein, 10 aa, Artificial
  Peptide
ALELDSNLYR

SEQ ID NO: 920 - protein, 6 aa, Artificial
  Peptide
NWQWWR

SEQ ID NO: 921 - protein, 15 aa, Artificial
  Peptide
LTEMETLQSQLMAEK

SEQ ID NO: 922 - protein, 19 aa, Artificial
  Peptide
MQQNIQELEEQLEEEESAR

SEQ ID NO: 923 - protein, 14 aa, Artificial
  Peptide
IAEFTTNLTEEEEK

SEQ ID NO: 924 - protein, 13 aa, Artificial
  Peptide
NKHEAMITDLEER

SEQ ID NO: 925 - protein, 11 aa, Artificial
  Peptide
HEAMITDLEER

SEQ ID NO: 926 - protein, 24 aa, Artificial
  Peptide
KLEGDSTDLSDQIAELQAQIAELK

SEQ ID NO: 927 - protein, 23 aa, Artificial
  Peptide
LEGDSTDLSDQIAELQAQIAELK

SEQ ID NO: 928 - protein, 17 aa, Artificial
  Peptide
ELESQISELQEDLESER

SEQ ID NO: 929 - protein, 10 aa, Artificial
  Peptide
DLGEELEALK

SEQ ID NO: 930 - protein, 27 aa, Artificial
  Peptide
DLGEELEALKTELEDTLDSTAAQQELR

SEQ ID NO: 931 - protein, 17 aa, Artificial
  Peptide
TELEDTLDSTAAQQELR

SEQ ID NO: 932 - protein, 10 aa, Artificial
  Peptide
THEAQIQEMR

SEQ ID NO: 933 - protein, 16 aa, Artificial
  Peptide
HSQAVEELAEQLEQTK

SEQ ID NO: 934 - protein, 15 aa, Artificial
  Peptide
QTLENERGELANEVK

SEQ ID NO: 935 - protein, 17 aa, Artificial
  Peptide
HSQAVEELAEQLEQTKR

SEQ ID NO: 936 - protein, 8 aa, Artificial
  Peptide
GELANEVK

SEQ ID NO: 937 - protein, 12 aa, Artificial
  Peptide
KVEAQLQELQVK

SEQ ID NO: 938 - protein, 11 aa, Artificial
  Peptide
VEAQLQELQVK

SEQ ID NO: 939 - protein, 18 aa, Artificial
  Peptide
LQVELDNVTGLLSQSDSK

SEQ ID NO: 940 - protein, 21 aa, Artificial
  Peptide
DFSALESQLQDTQELLQEENR

SEQ ID NO: 941 - protein, 13 aa, Artificial
  Peptide
QIATLHAQVADMK

SEQ ID NO: 942 - protein, 8 aa, Artificial
  Peptide
DLEGLSQR

SEQ ID NO: 943 - protein, 16 aa, Artificial
  Peptide
LQQELDDLLVDLDHQR

SEQ ID NO: 944 - protein, 9 aa, Artificial
  Peptide
ALEEAMEQK

SEQ ID NO: 945 - protein, 10 aa, Artificial
  Peptide
TEMEDLMSSK

SEQ ID NO: 946 - protein, 10 aa, Artificial
  Peptide
ALEQQVEEMK

SEQ ID NO: 947 - protein, 9 aa, Artificial
  Peptide
LEVNLQAMK

SEQ ID NO: 948 - protein, 11 aa, Artificial
  Peptide
DLEAHIDSANK

SEQ ID NO: 949 - protein, 8 aa, Artificial
  Peptide
EEILAQAK

SEQ ID NO: 950 - protein, 18 aa, Artificial
  Peptide
SMEAEMIQLQEELAAAER

SEQ ID NO: 951 - protein, 19 aa, Artificial
  Peptide
QAQQERDELADEIANSSGK

SEQ ID NO: 952 - protein, 13 aa, Artificial
  Peptide
DELADEIANSSGK

SEQ ID NO: 953 - protein, 21 aa, Artificial
  Peptide
IAQLEEELEEEQGNTELINDR

SEQ ID NO: 954 - protein, 17 aa, Artificial
  Peptide
KANLQIDQINTDLNLER

SEQ ID NO: 955 - protein, 16 aa, Artificial
  Peptide
ANLQIDQINTDLNLER

SEQ ID NO: 956 - protein, 9 aa, Artificial
  Peptide
LQEMEGTVK

SEQ ID NO: 957 - protein, 9 aa, Artificial
  Peptide
ASITALEAK

SEQ ID NO: 958 - protein, 13 aa, Artificial
  Peptide
IAQLEEQLDNETK

SEQ ID NO: 959 - protein, 12 aa, Artificial
  Peptide
LKDVLLQVDDER

SEQ ID NO: 960 - protein, 12 aa, Artificial
  Peptide
RQLEEAEEEAQR

SEQ ID NO: 961 - protein, 11 aa, Artificial
  Peptide
QLEEAEEEAQR

SEQ ID NO: 962 - protein, 14 aa, Artificial
  Peptide
ELEDATETADAMNR

SEQ ID NO: 963 - protein, 10 aa, Artificial
  Peptide
RGDLPFVVPR

SEQ ID NO: 964 - protein, 9 aa, Artificial
  Peptide
GDLPFVVPR

SEQ ID NO: 965 - protein, 9 aa, Artificial
  Peptide
EQEVTELKK

SEQ ID NO: 966 - protein, 9 aa, Artificial
  Peptide
HGFEAASIK

SEQ ID NO: 967 - protein, 20 aa, Artificial
  Peptide
IVGLDQVTGMTETAFGSAYK

SEQ ID NO: 968 - protein, 24 aa, Artificial
  Peptide
KLDGETTDLQDQIAELQAQIDELK

SEQ ID NO: 969 - protein, 27 aa, Artificial
  Peptide
DLSEELEALKTELEDTLDTTAAQQELR

SEQ ID NO: 970 - protein, 17 aa, Artificial
  Peptide
LQNELDNVSTLLEEAEK

SEQ ID NO: 971 - protein, 21 aa, Artificial
  Peptide
DAASLESQLQDTQELLQEETR

SEQ ID NO: 972 - protein, 10 aa, Artificial
  Peptide
ALEQQVEEMR

SEQ ID NO: 973 - protein, 18 aa, Artificial
  Peptide
SLEAEILQLQEELASSER

SEQ ID NO: 974 - protein, 21 aa, Artificial
  Peptide
IAQLEEELEEEQSNMELLNDR

SEQ ID NO: 975 - protein, 13 aa, Artificial
  Peptide
IGQLEEQLEQEAK

SEQ ID NO: 976 - protein, 16 aa, Artificial
  Peptide
QLLQANPILEAFGNAK

SEQ ID NO: 977 - protein, 11 aa, Artificial
  Peptide
KEEELQAALAR

SEQ ID NO: 978 - protein, 7 aa, Artificial
  Peptide
ADEWLMK

SEQ ID NO: 979 - protein, 8 aa, Artificial
  Peptide
NTNPNFVR

SEQ ID NO: 980 - protein, 11 aa, Artificial
  Peptide
LDPHLVLDQLR

SEQ ID NO: 981 - protein, 11 aa, Artificial
  Peptide
AGVLAHLEEER

SEQ ID NO: 982 - protein, 17 aa, Artificial
  Peptide
TQLEELEDELQATEDAK

SEQ ID NO: 983 - protein, 7 aa, Artificial
  Peptide
TVGQLYK

SEQ ID NO: 984 - protein, 10 aa, Artificial
  Peptide
KFDQLLAEEK

SEQ ID NO: 985 - protein, 9 aa, Artificial
  Peptide
FDQLLAEEK

SEQ ID NO: 986 - protein, 11 aa, Artificial
  Peptide
VIVESASNIPK

SEQ ID NO: 987 - protein, 18 aa, Artificial
  Peptide
VDNELNPVWNEILEFDLR

SEQ ID NO: 988 - protein, 13 aa, Artificial
  Peptide
GPVGTVSEAQLAR

SEQ ID NO: 989 - protein, 15 aa, Artificial
  Peptide
WLLLNDPEDTSSGSK

SEQ ID NO: 990 - protein, 16 aa, Artificial
  Peptide
AEDIPQMDDAFSQTVK

SEQ ID NO: 991 - protein, 13 aa, Artificial
  Peptide
NDVVGTTYLHLSK

SEQ ID NO: 992 - protein, 11 aa, Artificial
  Peptide
ILVELATFLEK

SEQ ID NO: 993 - protein, 13 aa, Artificial
  Peptide
LDAVNTLLAMAER

SEQ ID NO: 994 - protein, 9 aa, Artificial
  Peptide
LQTNIEALK

SEQ ID NO: 995 - protein, 12 aa, Artificial
  Peptide
IPANQLAELWLK

SEQ ID NO: 996 - protein, 10 aa, Artificial
  Peptide
LIDEVIEDTR

SEQ ID NO: 997 - protein, 11 aa, Artificial
  Peptide
ANVTVLDTQIR

SEQ ID NO: 998 - protein, 11 aa, Artificial
  Peptide
SLSQIHEAAVR

SEQ ID NO: 999 - protein, 12 aa, Artificial
  Peptide
STLAEIEDWLDK

SEQ ID NO: 1000 - protein, 18 aa, Artificial
  Peptide
IPAHQVLYSTSGENASGK

SEQ ID NO: 1001 - protein, 11 aa, Artificial
  Peptide
DLTQTASSTAR

SEQ ID NO: 1002 - protein, 19 aa, Artificial
  Peptide
VIMELFDNDQVGKDEFLGR

SEQ ID NO: 1003 - protein, 10 aa, Artificial
  Peptide
LNSEMDITPK

SEQ ID NO: 1004 - protein, 16 aa, Artificial
  Peptide
DIVIEMEDTKPLLASK

SEQ ID NO: 1005 - protein, 14 aa, Artificial
  Peptide
SDENEDPSVVGEFK

SEQ ID NO: 1006 - protein, 15 aa, Artificial
  Peptide
IYPLPDDPSVPAPPR

SEQ ID NO: 1007 - protein, 11 aa, Artificial
  Peptide
ISVYDYDTFTR

SEQ ID NO: 1008 - protein, 11 aa, Artificial
  Peptide
VGETIIDLENR

SEQ ID NO: 1009 - protein, 14 aa, Artificial
  Peptide
DQLRPTQLLQNVAR

SEQ ID NO: 1010 - protein, 12 aa, Artificial
  Peptide
ILHQHLGAPEER

SEQ ID NO: 1011 - protein, 14 aa, Artificial
  Peptide
SLGPPGPPFNITPR

SEQ ID NO: 1012 - protein, 11 aa, Artificial
  Peptide
SLDGEGNFNWR

SEQ ID NO: 1013 - protein, 12 aa, Artificial
  Peptide
TFIAIKPDGVQR

SEQ ID NO: 1014 - protein, 10 aa, Artificial
  Peptide
DRPFFPGLVK

SEQ ID NO: 1015 - protein, 17 aa, Artificial
  Peptide
VMLGETNPADSKPGTIR

SEQ ID NO: 1016 - protein, 10 aa, Artificial
  Peptide
NIIHGSDSVK

SEQ ID NO: 1017 - protein, 21 aa, Artificial
  Peptide
IESPGYLTSPGYPHSYHPSEK

SEQ ID NO: 1018 - protein, 16 aa, Artificial
  Peptide
IAPPPVVSSGPFLFIK

SEQ ID NO: 1019 - protein, 15 aa, Artificial
  Peptide
FVSDYETHGAGFSIR

SEQ ID NO: 1020 - protein, 13 aa, Artificial
  Peptide
FVTAVGTQGAISK

SEQ ID NO: 1021 - protein, 14 aa, Artificial
  Peptide
SFEGNNNYDTPELR

SEQ ID NO: 1022 - protein, 23 aa, Artificial
  Peptide
TGPIQDHTGDGNFIYSQADENQK

SEQ ID NO: 1023 - protein, 17 aa, Artificial
  Peptide
NLSALENYNFELVDGVK

SEQ ID NO: 1024 - protein, 23 aa, Artificial
  Peptide
YDAMALGNHEFDNGVELIEPLLK

SEQ ID NO: 1025 - protein, 17 aa, Artificial
  Peptide
VLPVGDEVVGIVGYTSK

SEQ ID NO: 1026 - protein, 29 aa, Artificial
  Peptide
ETPFLSNPGTNLVFEDEDEITALQPEVDK

SEQ ID NO: 1027 - protein, 11 aa, Artificial
  Peptide
YPFIVTSDDGR

SEQ ID NO: 1028 - protein, 8 aa, Artificial
  Peptide
VPSYDPLK

SEQ ID NO: 1029 - protein, 17 aa, Artificial
  Peptide
VIPNFLANGGDGFQMIK

SEQ ID NO: 1030 - protein, 17 aa, Artificial
  Peptide
HDSGDQDINVVSTYISK

SEQ ID NO: 1031 - protein, 9 aa, Artificial
  Peptide
VIYPAVEGR

SEQ ID NO: 1032 - protein, 11 aa, Artificial
  Peptide
SNFAEALAAHK

SEQ ID NO: 1033 - protein, 16 aa, Artificial
  Peptide
VDATEESDLAQQYGVR

SEQ ID NO: 1034 - protein, 42 aa, Artificial
  Peptide
EADDIVNWLKKRTGPAATTLPDGAAAESLVESSEVAVIGFFK

SEQ ID NO: 1035 - protein, 30 aa, Artificial
  Peptide
TGPAATTLPDGAAAESLVESSEVAVIGFFK

SEQ ID NO: 1036 - protein, 25 aa, Artificial
  Peptide
QFLQAAEAIDDIPFGITSNSDVFSK

SEQ ID NO: 1037 - protein, 9 aa, Artificial
  Peptide
NNFEGEVTK

SEQ ID NO: 1038 - protein, 12 aa, Artificial
  Peptide
YQLDKDGVVLFK

SEQ ID NO: 1039 - protein, 7 aa, Artificial
  Peptide
DGVVLFK

SEQ ID NO: 1040 - protein, 8 aa, Artificial
  Peptide
ENLLDFIK

SEQ ID NO: 1041 - protein, 17 aa, Artificial
  Peptide
HNQLPLVIEFTEQTAPK

SEQ ID NO: 1042 - protein, 9 aa, Artificial
  Peptide
THILLFLPK

SEQ ID NO: 1043 - protein, 8 aa, Artificial
  Peptide
ILEFFGLK

SEQ ID NO: 1044 - protein, 10 aa, Artificial
  Peptide
LITLEEEMTK

SEQ ID NO: 1045 - protein, 12 aa, Artificial
  Peptide
YKPESEELTAER

SEQ ID NO: 1046 - protein, 10 aa, Artificial
  Peptide
NFEDVAFDEK

SEQ ID NO: 1047 - protein, 15 aa, Artificial
  Peptide
LGETYKDHENIVIAK

SEQ ID NO: 1048 - protein, 9 aa, Artificial
  Peptide
DHENIVIAK

SEQ ID NO: 1049 - protein, 12 aa, Artificial
  Peptide
MDSTANEVEAVK

SEQ ID NO: 1050 - protein, 9 aa, Artificial
  Peptide
TVIDYNGER

SEQ ID NO: 1051 - protein, 8 aa, Artificial
  Peptide
MGGDIANR

SEQ ID NO: 1052 - protein, 11 aa, Artificial
  Peptide
GDAMIMEETGK

SEQ ID NO: 1053 - protein, 8 aa, Artificial
  Peptide
SDQDYILK

SEQ ID NO: 1054 - protein, 17 aa, Artificial
  Peptide
LVKPGNQNTQVTEAWNK

SEQ ID NO: 1055 - protein, 11 aa, Artificial
  Peptide
TIIQNPTDQQK

SEQ ID NO: 1056 - protein, 21 aa, Artificial
  Peptide
AEFEVHEVYAVDVLVSSGEGK

SEQ ID NO: 1057 - protein, 8 aa, Artificial
  Peptide
AFFSEVER

SEQ ID NO: 1058 - protein, 9 aa, Artificial
  Peptide
FDAMPFTLR

SEQ ID NO: 1059 - protein, 13 aa, Artificial
  Peptide
HELLQPFNVLYEK

SEQ ID NO: 1060 - protein, 12 aa, Artificial
  Peptide
FTVLLMPNGPMR

SEQ ID NO: 1061 - protein, 12 aa, Artificial
  Peptide
ITSGPFEPDLYK

SEQ ID NO: 1062 - protein, 11 aa, Artificial
  Peptide
SEMEVQDAELK

SEQ ID NO: 1063 - protein, 9 aa, Artificial
  Peptide
ALLQSSASR

SEQ ID NO: 1064 - protein, 14 aa, Artificial
  Peptide
TGEAIVDAALSALR

SEQ ID NO: 1065 - protein, 13 aa, Artificial
  Peptide
KDVIELTDDSFDK

SEQ ID NO: 1066 - protein, 14 aa, Artificial
  Peptide
NLEPEWAAAASEVK

SEQ ID NO: 1067 - protein, 15 aa, Artificial
  Peptide
LAAVDATVNQVLASR

SEQ ID NO: 1068 - protein, 24 aa, Artificial
  Peptide
ALDLFSDNAPPPELLEIINEDIAK

SEQ ID NO: 1069 - protein, 9 aa, Artificial
  Peptide
NSYLEVLLK

SEQ ID NO: 1070 - protein, 13 aa, Artificial
  Peptide
GSFSEQGINEFLR

SEQ ID NO: 1071 - protein, 17 aa, Artificial
  Peptide
GSTAPVGGGAFPTIVER

SEQ ID NO: 1072 - protein, 22 aa, Artificial
  Peptide
DGELPVEDDIDLSDVELDDLGK

SEQ ID NO: 1073 - protein, 12 aa, Artificial
  Peptide
DSPSVWAAVPGK

SEQ ID NO: 1074 - protein, 16 aa, Artificial
  Peptide
TFVNITPAEVGVLVGK

SEQ ID NO: 1075 - protein, 14 aa, Artificial
  Peptide
SSFYVNGLTLGGQK

SEQ ID NO: 1076 - protein, 14 aa, Artificial
  Peptide
DSLLQDGEFSMDLR

SEQ ID NO: 1077 - protein, 14 aa, Artificial
  Peptide
STGGDPTFNVTVTK

SEQ ID NO: 1078 - protein, 8 aa, Artificial
  Peptide
TLVLLMGK

SEQ ID NO: 1079 - protein, 19 aa, Artificial
  Peptide
GLGAGAGAGEESPATSLPR

SEQ ID NO: 1080 - protein, 14 aa, Artificial
  Peptide
FYGPAGPYGIFAGR

SEQ ID NO: 1081 - protein, 18 aa, Artificial
  Peptide
DEYDDLSDLNAVQMESVR

SEQ ID NO: 1082 - protein, 20 aa, Artificial
  Peptide
GVQDIVVGEGTHFLIPWVQK

SEQ ID NO: 1083 - protein, 10 aa, Artificial
  Peptide
DLQNVNITLR

SEQ ID NO: 1084 - protein, 8 aa, Artificial
  Peptide
PVASQLPR

SEQ ID NO: 1085 - protein, 12 aa, Artificial
  Peptide
ILFRPVASQLPR

SEQ ID NO: 1086 - protein, 12 aa, Artificial
  Peptide
IFTSIGEDYDER

SEQ ID NO: 1087 - protein, 10 aa, Artificial
  Peptide
FDAGELITQR

SEQ ID NO: 1088 - protein, 11 aa, Artificial
  Peptide
AAIISAEGDSK

SEQ ID NO: 1089 - protein, 20 aa, Artificial
  Peptide
AAELIANSLATAGDGLIELR

SEQ ID NO: 1090 - protein, 14 aa, Artificial
  Peptide
KLEAAEDIAYQLSR

SEQ ID NO: 1091 - protein, 12 aa, Artificial
  Peptide
NTLFNLSNFLDK

SEQ ID NO: 1092 - protein, 13 aa, Artificial
  Peptide
SGLQGYDMSTFIR

SEQ ID NO: 1093 - protein, 9 aa, Artificial
  Peptide
VAEQVGIDR

SEQ ID NO: 1094 - protein, 27 aa, Artificial
  Peptide
GDIPDLSQAPSSLLDALEQHLASLEGK

SEQ ID NO: 1095 - protein, 20 aa, Artificial
  Peptide
ATTLSNAVSSLASTGLSLTK

SEQ ID NO: 1096 - protein, 15 aa, Artificial
  Peptide
LASGGADLALGSIEK

SEQ ID NO: 1097 - protein, 24 aa, Artificial
  Peptide
VVEYLLPADKEESAPAPGHQQAQK

SEQ ID NO: 1098 - protein, 18 aa, Artificial
  Peptide
GVTDNVVDTVVHYVPLPR

SEQ ID NO: 1099 - protein, 24 aa, Artificial
  Peptide
SAQSPGAPPGPGLGDEVATPAAPR

SEQ ID NO: 1100 - protein, 10 aa, Artificial
  Peptide
VGALTNTLSR

SEQ ID NO: 1101 - protein, 14 aa, Artificial
  Peptide
ASGAPSAGPEPAPR

SEQ ID NO: 1102 - protein, 11 aa, Artificial
  Peptide
GVVQGGLDTTR

SEQ ID NO: 1103 - protein, 11 aa, Artificial
  Peptide
GAVQGGLDTSK

SEQ ID NO: 1104 - protein, 11 aa, Artificial
  Peptide
GTVQAGVDTTK

SEQ ID NO: 1105 - protein, 11 aa, Artificial
  Peptide
GAVQGGLDTTK

SEQ ID NO: 1106 - protein, 11 aa, Artificial
  Peptide
GAVQTGVDTAK

SEQ ID NO: 1107 - protein, 11 aa, Artificial
  Peptide
GTVQTGVDASK

SEQ ID NO: 1108 - protein, 8 aa, Artificial
  Peptide
ANLNALDR

SEQ ID NO: 1109 - protein, 18 aa, Artificial
  Peptide
VAPEEHPVLLTEAPLNPK

SEQ ID NO: 1110 - protein, 30 aa, Artificial
  Peptide
TTGIVMDSGDGVTHTVPIYEGYALPHAILR

SEQ ID NO: 1111 - protein, 10 aa, Artificial
  Peptide
GYSFTTTAER

SEQ ID NO: 1112 - protein, 21 aa, Artificial
  Peptide
DLYANTVLSGGTTMYPGIADR

SEQ ID NO: 1113 - protein, 13 aa, Artificial
  Peptide
QEYDESGPSIVHR

SEQ ID NO: 1114 - protein, 16 aa, Artificial
  Peptide
YPIEHGIITNWDDMEK

SEQ ID NO: 1115 - protein, 18 aa, Artificial
  Peptide
VAPEEHPTLLTEAPLNPK

SEQ ID NO: 1116 - protein, 10 aa, Artificial
  Peptide
GYSFVTTAER

SEQ ID NO: 1117 - protein, 11 aa, Artificial
  Peptide
HQGVMVGMGQK

SEQ ID NO: 1118 - protein, 11 aa, Artificial
  Peptide
DSYVGDEAQSK

SEQ ID NO: 1119 - protein, 8 aa, Artificial
  Peptide
DLTDYLMK

SEQ ID NO: 1120 - protein, 11 aa, Artificial
  Peptide
EITALAPSTMK

SEQ ID NO: 1121 - protein, 10 aa, Artificial
  Peptide
AGFAGDDAPR

SEQ ID NO: 1122 - protein, 9 aa, Artificial
  Peptide
AVFPSIVGR

SEQ ID NO: 1123 - protein, 16 aa, Artificial
  Peptide
SYELPDGQVITIGNER

SEQ ID NO: 1124 - protein, 9 aa, Artificial
  Peptide
VSFELFADK

SEQ ID NO: 1125 - protein, 9 aa, Artificial
  Peptide
FEDENFILK

SEQ ID NO: 1126 - protein, 7 aa, Artificial
  Peptide
TEWLDGK

SEQ ID NO: 1127 - protein, 11 aa, Artificial
  Peptide
EGMNIVEAMER

SEQ ID NO: 1128 - protein, 8 aa, Artificial
  Peptide
IGDEDVGR

SEQ ID NO: 1129 - protein, 8 aa, Artificial
  Peptide
VIFGLFGK

SEQ ID NO: 1130 - protein, 13 aa, Artificial
  Peptide
TVDNFNALATGEK

SEQ ID NO: 1131 - protein, 11 aa, Artificial
  Peptide
DFMIQGGDFTR

SEQ ID NO: 1132 - protein, 7 aa, Artificial
  Peptide
FPDENFK

SEQ ID NO: 1133 - protein, 14 aa, Artificial
  Peptide
HYGPGWVSMANAGK

SEQ ID NO: 1134 - protein, 13 aa, Artificial
  Peptide
DTNGSQFFITTVK

SEQ ID NO: 1135 - protein, 9 aa, Artificial
  Peptide
VLEGMEVVR

SEQ ID NO: 1136 - protein, 11 aa, Artificial
  Peptide
IEVEKPFAIAK

SEQ ID NO: 1137 - protein, 10 aa, Artificial
  Peptide
IELFADVVPK

SEQ ID NO: 1138 - protein, 6 aa, Artificial
  Peptide
HVVFGK

SEQ ID NO: 1139 - protein, 20 aa, Artificial
  Peptide
LGEHNIEVLEGNEQFINAAK

SEQ ID NO: 1140 - protein, 20 aa, Artificial
  Peptide
LGEHNIKVLEGNEQFINAAK

SEQ ID NO: 1141 - protein, 13 aa, Artificial
  Peptide
VLEGNEQFINAAK

SEQ ID NO: 1142 - protein, 6 aa, Artificial
  Peptide
PGVYTK

SEQ ID NO: 1143 - protein, 21 aa, Artificial
  Peptide
VELSDVQNPAISITENVLHFK

SEQ ID NO: 1144 - protein, 12 aa, Artificial
  Peptide
WLDESDAEMELR

SEQ ID NO: 1145 - protein, 14 aa, Artificial
  Peptide
LESEGSPETLTNLR

SEQ ID NO: 1146 - protein, 11 aa, Artificial
  Peptide
VVLIGDSGVGK

SEQ ID NO: 1147 - protein, 11 aa, Artificial
  Peptide
AQIWDTAGQER

SEQ ID NO: 1148 - protein, 9 aa, Artificial
  Peptide
HLTYENVER

SEQ ID NO: 1149 - protein, 10 aa, Artificial
  Peptide
HESQMDSVVK

SEQ ID NO: 1150 - protein, 11 aa, Artificial
  Peptide
KATVNLLGEEK

SEQ ID NO: 1151 - protein, 10 aa, Artificial
  Peptide
ATVNLLGEEK

SEQ ID NO: 1152 - protein, 10 aa, Artificial
  Peptide
TVDLSSHLAK

SEQ ID NO: 1153 - protein, 17 aa, Artificial
  Peptide
VTAEVVLAHLGGGSTSR

SEQ ID NO: 1154 - protein, 15 aa, Artificial
  Peptide
ATSFLLALEPELEAR

SEQ ID NO: 1155 - protein, 12 aa, Artificial
  Peptide
GEDEEENNLEVR

SEQ ID NO: 1156 - protein, 11 aa, Artificial
  Peptide
SEDLLDYGPFR

SEQ ID NO: 1157 - protein, 13 aa, Artificial
  Peptide
TILPAAAQDVYYR

SEQ ID NO: 1158 - protein, 17 aa, Artificial
  Peptide
FVDHVFDEQVIDSLTVK

SEQ ID NO: 1159 - protein, 12 aa, Artificial
  Peptide
NIEIDSPYEISR

SEQ ID NO: 1160 - protein, 15 aa, Artificial
  Peptide
APDELHYTYLDTFGR

SEQ ID NO: 1161 - protein, 22 aa, Artificial
  Peptide
APDELHYTYLDTFGRPVIVAYK

SEQ ID NO: 1162 - protein, 7 aa, Artificial
  Peptide
PVIVAYK

SEQ ID NO: 1163 - protein, 8 aa, Artificial
  Peptide
HFDETVNR

SEQ ID NO: 1164 - protein, 12 aa, Artificial
  Peptide
ALTSEIALLQSR

SEQ ID NO: 1165 - protein, 12 aa, Artificial
  Peptide
LFLVEDLVDSLK

SEQ ID NO: 1166 - protein, 11 aa, Artificial
  Peptide
TQIDHYVGIAR

SEQ ID NO: 1167 - protein, 34 aa, Artificial
  Peptide
KPAAGLSAAPVPTAPAAGAPLMDFGNDFVPPAPR

SEQ ID NO: 1168 - protein, 13 aa, Artificial
  Peptide
GPLPAAPPVAPER

SEQ ID NO: 1169 - protein, 28 aa, Artificial
  Peptide
QPSWDPSPVSSTVPAPSPLSAAAVSPSK

SEQ ID NO: 1170 - protein, 45 aa, Artificial
  Peptide
LPEDDEPPARPPPPPPASVSPQAEPVMTPPAPAPAAPPSTPAAPK

SEQ ID NO: 1171 - protein, 8 aa, Artificial
  Peptide
GVIQAIQK

SEQ ID NO: 1172 - protein, 12 aa, Artificial
  Peptide
LFLVDDLVDSLK

SEQ ID NO: 1173 - protein, 14 aa, Artificial
  Peptide
HQAQIDHYLGLANK

SEQ ID NO: 1174 - protein, 11 aa, Artificial
  Peptide
GLGLDESGLAK

SEQ ID NO: 1175 - protein, 12 aa, Artificial
  Peptide
AVLLAGPPGTGK

SEQ ID NO: 1176 - protein, 14 aa, Artificial
  Peptide
TALALAIAQELGSK

SEQ ID NO: 1177 - protein, 11 aa, Artificial
  Peptide
LDPSIFESLQK

SEQ ID NO: 1178 - protein, 16 aa, Artificial
  Peptide
PQEEQDILSMMGQLMK

SEQ ID NO: 1179 - protein, 18 aa, Artificial
  Peptide
GTEDITSPHGIPLDLLDR

SEQ ID NO: 1180 - protein, 26 aa, Artificial
  Peptide
DQAVENILVSPVVVASSLGLVSLGGK

SEQ ID NO: 1181 - protein, 22 aa, Artificial
  Peptide
AVLSAEQLRDEEVHAGLGELLR

SEQ ID NO: 1182 - protein, 9 aa, Artificial
  Peptide
AVLSAEQLR

SEQ ID NO: 1183 - protein, 13 aa, Artificial
  Peptide
DEEVHAELEELLR

SEQ ID NO: 1184 - protein, 15 aa, Artificial
  Peptide
LYGPSSVSFADDFVR

SEQ ID NO: 1185 - protein, 17 aa, Artificial
  Peptide
SALQSINEWAAQTTDGK

SEQ ID NO: 1186 - protein, 13 aa, Artificial
  Peptide
TDGALLVNAMFFK

SEQ ID NO: 1187 - protein, 13 aa, Artificial
  Peptide
TGLYNYYDDEKEK

SEQ ID NO: 1188 - protein, 11 aa, Artificial
  Peptide
LQIVEMPLAHK

SEQ ID NO: 1189 - protein, 8 aa, Artificial
  Peptide
AVAISLPK

SEQ ID NO: 1190 - protein, 11 aa, Artificial
  Peptide
GVVEVTHDLQK

SEQ ID NO: 1191 - protein, 13 aa, Artificial
  Peptide
HLAGLGLTEAIDK

SEQ ID NO: 1192 - protein, 13 aa, Artificial
  Peptide
LFYADHPFIFLVR

SEQ ID NO: 1193 - protein, 12 aa, Artificial
  Peptide
DTQSGSLLFIGR

SEQ ID NO: 1194 - protein, 13 aa, Artificial
  Peptide
LSAFVFPGELLLR

SEQ ID NO: 1195 - protein, 11 aa, Artificial
  Peptide
EVLDSFLDLAR

SEQ ID NO: 1196 - protein, 10 aa, Artificial
  Peptide
LGPEGELLIR

SEQ ID NO: 1197 - protein, 13 aa, Artificial
  Peptide
IVEMEDVGLLFAR

SEQ ID NO: 1198 - protein, 10 aa, Artificial
  Peptide
STEPELIQVK

SEQ ID NO: 1199 - protein, 20 aa, Artificial
  Peptide
SELPLDPLPVPTEEGNPLLK

SEQ ID NO: 1200 - protein, 12 aa, Artificial
  Peptide
GPAGDATVASEK

SEQ ID NO: 1201 - protein, 10 aa, Artificial
  Peptide
VHLQTQQEVK

SEQ ID NO: 1202 - protein, 11 aa, Artificial
  Peptide
LFSGATMASSR

SEQ ID NO: 1203 - protein, 16 aa, Artificial
  Peptide
DYFLFNPVTDIEEIIR

SEQ ID NO: 1204 - protein, 13 aa, Artificial
  Peptide
QLLAGGIAGAVSR

SEQ ID NO: 1205 - protein, 9 aa, Artificial
  Peptide
HEGLGAFYK

SEQ ID NO: 1206 - protein, 9 aa, Artificial
  Peptide
IMMQVHGSK

SEQ ID NO: 1207 - protein, 13 aa, Artificial
  Peptide
DFLAGGVAAAISK

SEQ ID NO: 1208 - protein, 10 aa, Artificial
  Peptide
QIFLGGVDKR

SEQ ID NO: 1209 - protein, 11 aa, Artificial
  Peptide
AAYFGIYDTAK

SEQ ID NO: 1210 - protein, 13 aa, Artificial
  Peptide
DFLAGGIAAAISK

SEQ ID NO: 1211 - protein, 11 aa, Artificial
  Peptide
AAYFGVYDTAK

SEQ ID NO: 1212 - protein, 8 aa, Artificial
  Peptide
TAVAPIER

SEQ ID NO: 1213 - protein, 10 aa, Artificial
  Peptide
LLLQVQHASK

SEQ ID NO: 1214 - protein, 9 aa, Artificial
  Peptide
EQGVLSFWR

SEQ ID NO: 1215 - protein, 8 aa, Artificial
  Peptide
GNLANVIR

SEQ ID NO: 1216 - protein, 12 aa, Artificial
  Peptide
YFPTQALNFAFK

SEQ ID NO: 1217 - protein, 9 aa, Artificial
  Peptide
QIFLGGVDK

SEQ ID NO: 1218 - protein, 8 aa, Artificial
  Peptide
GAWSNVLR

SEQ ID NO: 1219 - protein, 13 aa, Artificial
  Peptide
VAEDEAEAAAAAK

SEQ ID NO: 1220 - protein, 11 aa, Artificial
  Peptide
FTGLSKEELLK

SEQ ID NO: 1221 - protein, 9 aa, Artificial
  Peptide
VAGSPGWVR

SEQ ID NO: 1222 - protein, 9 aa, Artificial
  Peptide
WWHTGALYR

SEQ ID NO: 1223 - protein, 19 aa, Artificial
  Peptide
IGDLQAFQGHGAGNLAGLK

SEQ ID NO: 1224 - protein, 10 aa, Artificial
  Peptide
GRLDYLSSLK

SEQ ID NO: 1225 - protein, 8 aa, Artificial
  Peptide
LDYLSSLK

SEQ ID NO: 1226 - protein, 9 aa, Artificial
  Peptide
GLVLGPIHK

SEQ ID NO: 1227 - protein, 11 aa, Artificial
  Peptide
EDFDSLLQSAK

SEQ ID NO: 1228 - protein, 10 aa, Artificial
  Peptide
VILDLTPNYR

SEQ ID NO: 1229 - protein, 16 aa, Artificial
  Peptide
GENSWFSTQVDTVATK

SEQ ID NO: 1230 - protein, 20 aa, Artificial
  Peptide
DLLLTSSYLSDSGSTGEHTK

SEQ ID NO: 1231 - protein, 12 aa, Artificial
  Peptide
LLTSFLPAQLLR

SEQ ID NO: 1232 - protein, 14 aa, Artificial
  Peptide
GQSEDPGSLLSLFR

SEQ ID NO: 1233 - protein, 11 aa, Artificial
  Peptide
ADLLLSTQPGR

SEQ ID NO: 1234 - protein, 21 aa, Artificial
  Peptide
ADLLLSTQPGREEGSPLELER

SEQ ID NO: 1235 - protein, 10 aa, Artificial
  Peptide
EEGSPLELER

SEQ ID NO: 1236 - protein, 12 aa, Artificial
  Peptide
LKLEPHEGLLLR

SEQ ID NO: 1237 - protein, 13 aa, Artificial
  Peptide
KGTVEGFGPADNK

SEQ ID NO: 1238 - protein, 12 aa, Artificial
  Peptide
GTVEGFEPADNK

SEQ ID NO: 1239 - protein, 19 aa, Artificial
  Peptide
VQNATLAVANITNADSATR

SEQ ID NO: 1240 - protein, 21 aa, Artificial
  Peptide
EEIAHNMQSTLDDATDAWGIK

SEQ ID NO: 1241 - protein, 10 aa, Artificial
  Peptide
AMAAEAEASR

SEQ ID NO: 1242 - protein, 12 aa, Artificial
  Peptide
VIAAEGEMNASR

SEQ ID NO: 1243 - protein, 16 aa, Artificial
  Peptide
EASMVITESPAALQLR

SEQ ID NO: 1244 - protein, 12 aa, Artificial
  Peptide
YLQTLTTIAAEK

SEQ ID NO: 1245 - protein, 20 aa, Artificial
  Peptide
NSTIVFPLPIDMLQGIIGAK

SEQ ID NO: 1246 - protein, 17 aa, Artificial
  Peptide
NTVVLFVPQQEAWVVER

SEQ ID NO: 1247 - protein, 15 aa, Artificial
  Peptide
ILEPGLNILIPVLDR

SEQ ID NO: 1248 - protein, 21 aa, Artificial
  Peptide
ASYGVEDPEYAVTQLAQTTMR

SEQ ID NO: 1249 - protein, 10 aa, Artificial
  Peptide
ATVLESEGTR

SEQ ID NO: 1250 - protein, 17 aa, Artificial
  Peptide
AEQINQAAEEASAVLAK

SEQ ID NO: 1251 - protein, 30 aa, Artificial
  Peptide
DSNTILLPSNPGDVTSMVAQAMGVYGALTK

SEQ ID NO: 1252 - protein, 16 aa, Artificial
  Peptide
APVPGTPDSLSSGSSR

SEQ ID NO: 1253 - protein, 15 aa, Artificial
  Peptide
FESVIHEFDPYFNYR

SEQ ID NO: 1254 - protein, 9 aa, Artificial
  Peptide
FLAEEGFYK

SEQ ID NO: 1255 - protein, 11 aa, Artificial
  Peptide
LNPQQFEVLFR

SEQ ID NO: 1256 - protein, 11 aa, Artificial
  Peptide
FYSLLDPSYAK

SEQ ID NO: 1257 - protein, 11 aa, Artificial
  Peptide
VGQAMASTEEK

SEQ ID NO: 1258 - protein, 12 aa, Artificial
  Peptide
ENDYYTPTGEFR

SEQ ID NO: 1259 - protein, 12 aa, Artificial
  Peptide
GQDMETEAHQNK

SEQ ID NO: 1260 - protein, 16 aa, Artificial
  Peptide
LEEMINELAVAMTAVK

SEQ ID NO: 1261 - protein, 9 aa, Artificial
  Peptide
HEQEYMEVR

SEQ ID NO: 1262 - protein, 16 aa, Artificial
  Peptide
SAFSNLFGGEPLSYTR

SEQ ID NO: 1263 - protein, 14 aa, Artificial
  Peptide
LAVDEEENADNNTK

SEQ ID NO: 1264 - protein, 9 aa, Artificial
  Peptide
RLYWDDLKR

SEQ ID NO: 1265 - protein, 10 aa, Artificial
  Peptide
LLNENSYVPR

SEQ ID NO: 1266 - protein, 19 aa, Artificial
  Peptide
EAGSQKDENLALYVENQFR

SEQ ID NO: 1267 - protein, 13 aa, Artificial
  Peptide
DENLALYVENQFR

SEQ ID NO: 1268 - protein, 12 aa, Artificial
  Peptide
DSAQNSVIIVDK

SEQ ID NO: 1269 - protein, 16 aa, Artificial
  Peptide
LVYLVENPGGYVAYSK

SEQ ID NO: 1270 - protein, 14 aa, Artificial
  Peptide
SSGLPNIPVQTISR

SEQ ID NO: 1271 - protein, 9 aa, Artificial
  Peptide
ILNIFGVIK

SEQ ID NO: 1272 - protein, 15 aa, Artificial
  Peptide
GFVEPDHYVVVGAQR

SEQ ID NO: 1273 - protein, 11 aa, Artificial
  Peptide
LAQMFSDMVLK

SEQ ID NO: 1274 - protein, 9 aa, Artificial
  Peptide
AFTYINLDK

SEQ ID NO: 1275 - protein, 13 aa, Artificial
  Peptide
VSASPLLYTLIEK

SEQ ID NO: 1276 - protein, 17 aa, Artificial
  Peptide
HPVTGQFLYQDSNWASK

SEQ ID NO: 1277 - protein, 12 aa, Artificial
  Peptide
AAAEVAGQFVIK

SEQ ID NO: 1278 - protein, 13 aa, Artificial
  Peptide
LTHDVELNLDYER

SEQ ID NO: 1279 - protein, 6 aa, Artificial
  Peptide
DLNQYR

SEQ ID NO: 1280 - protein, 13 aa, Artificial
  Peptide
VEYHFLSPYVSPK

SEQ ID NO: 1281 - protein, 19 aa, Artificial
  Peptide
HVFWGSGSHTLPALLENLK

SEQ ID NO: 1282 - protein, 22 aa, Artificial
  Peptide
VVMVNGTPMEDVLHSFAVQQLR

SEQ ID NO: 1283 - protein, 8 aa, Artificial
  Peptide
LGSQIFVK

SEQ ID NO: 1284 - protein, 12 aa, Artificial
  Peptide
DGNLHEGDIILK

SEQ ID NO: 1285 - protein, 12 aa, Artificial
  Peptide
AEQMASVQNAQR

SEQ ID NO: 1286 - protein, 25 aa, Artificial
  Peptide
NKEPPAPAQQLQPQPVAVQGPEPAR

SEQ ID NO: 1287 - protein, 18 aa, Artificial
  Peptide
MSPDEGQEELEEVQAELK

SEQ ID NO: 1288 - protein, 11 aa, Artificial
  Peptide
SQLTTIVLAAR

SEQ ID NO: 1289 - protein, 34 aa, Artificial
  Peptide
QLSPTEAFPVLVEDMDNSGSLNAQVIHQLGPGLR

SEQ ID NO: 1290 - protein, 9 aa, Artificial
  Peptide
MAIQTQQSK

SEQ ID NO: 1291 - protein, 14 aa, Artificial
  Peptide
RPGEEGTVMSLAGK

SEQ ID NO: 1292 - protein, 6 aa, Artificial
  Peptide
ANLLFK

SEQ ID NO: 1293 - protein, 15 aa, Artificial
  Peptide
GSVDSNWIVGATLGK

SEQ ID NO: 1294 - protein, 14 aa, Artificial
  Peptide
LIGQIVSSITASLR

SEQ ID NO: 1295 - protein, 15 aa, Artificial
  Peptide
AVFVDLEPTVIDEIR

SEQ ID NO: 1296 - protein, 15 aa, Artificial
  Peptide
AVFVDLEPTVIDEVR

SEQ ID NO: 1297 - protein, 9 aa, Artificial
  Peptide
EIIDLVLDR

SEQ ID NO: 1298 - protein, 10 aa, Artificial
  Peptide
DVNAAIATIK

SEQ ID NO: 1299 - protein, 14 aa, Artificial
  Peptide
LISQIVSSITASLR

SEQ ID NO: 1300 - protein, 14 aa, Artificial
  Peptide
NLDIERPTYTNLNR

SEQ ID NO: 1301 - protein, 21 aa, Artificial
  Peptide
FDGALNVDLTEFQTNLVPYPR

SEQ ID NO: 1302 - protein, 16 aa, Artificial
  Peptide
IHFPLATYAPVISAEK

SEQ ID NO: 1303 - protein, 18 aa, Artificial
  Peptide
VGINYQPPTVVPGGDLAK

SEQ ID NO: 1304 - protein, 8 aa, Artificial
  Peptide
PTYTNLNR

SEQ ID NO: 1305 - protein, 17 aa, Artificial
  Peptide
SFGGGTGSGFTSLLMEK

SEQ ID NO: 1306 - protein, 20 aa, Artificial
  Peptide
TIGGGDDSFNTFFSETGAGK

SEQ ID NO: 1307 - protein, 20 aa, Artificial
  Peptide
AFVHWYVGEGMEEGEFSEAR

SEQ ID NO: 1308 - protein, 7 aa, Artificial
  Peptide
FDLMYAK

SEQ ID NO: 1309 - protein, 14 aa, Artificial
  Peptide
LLDAVDTYIPVPAR

SEQ ID NO: 1310 - protein, 19 aa, Artificial
  Peptide
DLEKPFLLPVEAVYSVPAR

SEQ ID NO: 1311 - protein, 15 aa, Artificial
  Peptide
PFLLPVEAVYSVPGR

SEQ ID NO: 1312 - protein, 13 aa, Artificial
  Peptide
TVVTGIGIEMFHK

SEQ ID NO: 1313 - protein, 12 aa, Artificial
  Peptide
AEAGDNLGALVR

SEQ ID NO: 1314 - protein, 19 aa, Artificial
  Peptide
ATAAPAGAPPQPQDLEFTK

SEQ ID NO: 1315 - protein, 18 aa, Artificial
  Peptide
LPNGLVIASLENYSPVSR

SEQ ID NO: 1316 - protein, 14 aa, Artificial
  Peptide
YEDFSNLGTTHLLR

SEQ ID NO: 1317 - protein, 11 aa, Artificial
  Peptide
WEVADLQPQLK

SEQ ID NO: 1318 - protein, 21 aa, Artificial
  Peptide
AVAFQNPQTHVIENLHAAAYR

SEQ ID NO: 1319 - protein, 14 aa, Artificial
  Peptide
MALIGLGVSHPVLK

SEQ ID NO: 1320 - protein, 10 aa, Artificial
  Peptide
QVAEQFLNMR

SEQ ID NO: 1321 - protein, 16 aa, Artificial
  Peptide
TIAQGNLSNTDVQAAK

SEQ ID NO: 1322 - protein, 16 aa, Artificial
  Peptide
TVQSNSPISALAPTGK

SEQ ID NO: 1323 - protein, 14 aa, Artificial
  Peptide
HEQILVLDPPTDLK

SEQ ID NO: 1324 - protein, 14 aa, Artificial
  Peptide
FKGPFTDVVTTNLK

SEQ ID NO: 1325 - protein, 20 aa, Artificial
  Peptide
FMVQTIFAPPNTSDMEAVWK

SEQ ID NO: 1326 - protein, 12 aa, Artificial
  Peptide
PHSVSLNDTETR

SEQ ID NO: 1327 - protein, 16 aa, Artificial
  Peptide
VAHSDKPGSTSTASFR

SEQ ID NO: 1328 - protein, 12 aa, Artificial
  Peptide
GPFTDVVTTNLK

SEQ ID NO: 1329 - protein, 27 aa, Artificial
  Peptide
TEAASDPQHPAASEGAAAAAASPPLLR

SEQ ID NO: 1330 - protein, 15 aa, Artificial
  Peptide
PAAPPAPGPGQLTLR

SEQ ID NO: 1331 - protein, 18 aa, Artificial
  Peptide
TWWNQFSVTALQLLQANR

SEQ ID NO: 1332 - protein, 26 aa, Artificial
  Peptide
LPPLPVTPGMEGAGVVIAVGEGVSDR

SEQ ID NO: 1333 - protein, 16 aa, Artificial
  Peptide
GVDIVMDPLGGSDTAK

SEQ ID NO: 1334 - protein, 14 aa, Artificial
  Peptide
VVTYGMANLLTGPK

SEQ ID NO: 1335 - protein, 9 aa, Artificial
  Peptide
VLLVPGPEK

SEQ ID NO: 1336 - protein, 21 aa, Artificial
  Peptide
AIPDLTAPVAAVQAAVSNLVR

SEQ ID NO: 1337 - protein, 10 aa, Artificial
  Peptide
MSAEINEIIR

SEQ ID NO: 1338 - protein, 15 aa, Artificial
  Peptide
DPSASPGDAGEQAIR

SEQ ID NO: 1339 - protein, 13 aa, Artificial
  Peptide
MLGQMTDQVADLR

SEQ ID NO: 1340 - protein, 14 aa, Artificial
  Peptide
AQQVSQGLDVLTAK

SEQ ID NO: 1341 - protein, 12 aa, Artificial
  Peptide
GVATALQNLQTK

SEQ ID NO: 1342 - protein, 12 aa, Artificial
  Peptide
MTGLVDEAIDTK

SEQ ID NO: 1343 - protein, 10 aa, Artificial
  Peptide
GDDLSTAILK

SEQ ID NO: 1344 - protein, 20 aa, Artificial
  Peptide
LIVDEAINEDNSVVSLSQPK

SEQ ID NO: 1345 - protein, 8 aa, Artificial
  Peptide
MDELQLFR

SEQ ID NO: 1346 - protein, 8 aa, Artificial
  Peptide
KGDIFLVR

SEQ ID NO: 1347 - protein, 7 aa, Artificial
  Peptide
GDIFLVR

SEQ ID NO: 1348 - protein, 10 aa, Artificial
  Peptide
LAGESESNLR

SEQ ID NO: 1349 - protein, 17 aa, Artificial
  Peptide
NAPAIIFIDELDAIAPK

SEQ ID NO: 1350 - protein, 13 aa, Artificial
  Peptide
IVSQLLTLMDGLK

SEQ ID NO: 1351 - protein, 12 aa, Artificial
  Peptide
EVDIGIPDATGR

SEQ ID NO: 1352 - protein, 12 aa, Artificial
  Peptide
WALSQSNPSALR

SEQ ID NO: 1353 - protein, 21 aa, Artificial
  Peptide
ETVVEVPQVTWEDIGGLEDVK

SEQ ID NO: 1354 - protein, 15 aa, Artificial
  Peptide
ELQELVQYPVEHPDK

SEQ ID NO: 1355 - protein, 13 aa, Artificial
  Peptide
GGNIGDGGGAADR

SEQ ID NO: 1356 - protein, 15 aa, Artificial
  Peptide
VINQILTEMDGMSTK

SEQ ID NO: 1357 - protein, 13 aa, Artificial
  Peptide
LDQLIYIPLPDEK

SEQ ID NO: 1358 - protein, 9 aa, Artificial
  Peptide
DVDLEFLAK

SEQ ID NO: 1359 - protein, 19 aa, Artificial
  Peptide
QTNPSAMEVEEDDPVPEIR

SEQ ID NO: 1360 - protein, 8 aa, Artificial
  Peptide
GYGFGLIK

SEQ ID NO: 1361 - protein, 11 aa, Artificial
  Peptide
WTEYGLTFTEK

SEQ ID NO: 1362 - protein, 13 aa, Artificial
  Peptide
LTFDSSFSPNTGK

SEQ ID NO: 1363 - protein, 22 aa, Artificial
  Peptide
GALVLGYEGWLAGYQMNFETAK

SEQ ID NO: 1364 - protein, 11 aa, Artificial
  Peptide
VTQSNFAVGYK

SEQ ID NO: 1365 - protein, 20 aa, Artificial
  Peptide
VNNSSLIGLGYTQTLKPGIK

SEQ ID NO: 1366 - protein, 11 aa, Artificial
  Peptide
LSQNNFALGYK

SEQ ID NO: 1367 - protein, 8 aa, Artificial
  Peptide
GFGFGLVK

SEQ ID NO: 1368 - protein, 13 aa, Artificial
  Peptide
LTFDTTFSPNTGK

SEQ ID NO: 1369 - protein, 12 aa, Artificial
  Peptide
YQLDPTASISAK

SEQ ID NO: 1370 - protein, 10 aa, Artificial
  Peptide
LTLSALVDGK

SEQ ID NO: 1371 - protein, 6 aa, Artificial
  Peptide
FGIAAK

SEQ ID NO: 1372 - protein, 19 aa, Artificial
  Peptide
LLQDSVDFSLADAINTEFK

SEQ ID NO: 1373 - protein, 9 aa, Artificial
  Peptide
VELQELNDR

SEQ ID NO: 1374 - protein, 7 aa, Artificial
  Peptide
FANYIDK

SEQ ID NO: 1375 - protein, 10 aa, Artificial
  Peptide
ILLAELEQLK

SEQ ID NO: 1376 - protein, 10 aa, Artificial
  Peptide
LGDLYEEEMR

SEQ ID NO: 1377 - protein, 10 aa, Artificial
  Peptide
RQVDQLTNDK

SEQ ID NO: 1378 - protein, 9 aa, Artificial
  Peptide
DNLAEDIMR

SEQ ID NO: 1379 - protein, 8 aa, Artificial
  Peptide
LQEEMLQR

SEQ ID NO: 1380 - protein, 11 aa, Artificial
  Peptide
EEAENTLQSFR

SEQ ID NO: 1381 - protein, 10 aa, Artificial
  Peptide
QDVDNASLAR

SEQ ID NO: 1382 - protein, 13 aa, Artificial
  Peptide
KVESLQEEIAFLK

SEQ ID NO: 1383 - protein, 10 aa, Artificial
  Peptide
NLQEAEEWYK

SEQ ID NO: 1384 - protein, 10 aa, Artificial
  Peptide
FADLSEAANR

SEQ ID NO: 1385 - protein, 8 aa, Artificial
  Peptide
GTNESLER

SEQ ID NO: 1386 - protein, 19 aa, Artificial
  Peptide
EMEENFAVEAANYQDTIGR

SEQ ID NO: 1387 - protein, 9 aa, Artificial
  Peptide
LQDEIQNMK

SEQ ID NO: 1388 - protein, 14 aa, Artificial
  Peptide
ISLPLPNFSSLNLR

SEQ ID NO: 1389 - protein, 15 aa, Artificial
  Peptide
ETNLDSLPLVDTHSK

SEQ ID NO: 1390 - protein, 14 aa, Artificial
  Peptide
AVTEQGHELSNEER

SEQ ID NO: 1391 - protein, 8 aa, Artificial
  Peptide
NLLSVAYK

SEQ ID NO: 1392 - protein, 9 aa, Artificial
  Peptide
EMQPTHPIR

SEQ ID NO: 1393 - protein, 10 aa, Artificial
  Peptide
DSTLIMQLLR

(19) United States
(12) Patent Application Publication
(10) Pub. No.: US 2014/0315212 A1
(43) Pub. Date: Oct. 23, 2014

(54) ALPHA TOXIN DETECTION OF GPI ANCHORED PROTEINS

(71) Applicant: University of Georgia Research Foundation, Inc., Athens, GA (US)

(72) Inventors: James Michael Pierce, Athens, GA (US); Karen Lynn Abbott, Statham, GA (US)

(73) Assignee: University of Georgia Research Foundation, Inc., Athens, GA (US)

(21) Appl. No.: 14/166,034

(22) Filed: Jan. 28, 2014

Related U.S. Application Data
(63) Continuation-in-part of application No. PCT/US12/48581, filed on Jul. 27, 2012.
(60) Provisional application No. 61/512,976, filed on Jul. 29, 2011.
(60) Provisional application No. 61/579,957, filed on Dec. 23, 2011.

(51) Int. Cl. G01N 33/68

(52) U.S. Cl. CPC G01N 33/6893
USPC 435/7.1; 435/34; 530/395; 530/387.9

(57) ABSTRACT

The present invention relates to a method for the purification, concentration and identification of glycosylphosphatidylinositol anchored proteins (GPI-APs) from a biological sample (cells, tissues and/or blood/serum) in a patient or subject, including a human patient or subject. A new method to separate GPI-anchored glycoproteins, a class of glycoproteins found in all animal cells and fluids including serum, from other glycoproteins and proteins for the purpose of identifying potential biomarkers for various diseases, including cancer, especially breast cancer, vaginal cancer, endometrial cancer, uterine cancer, cervical cancer, pancreatic cancer and prostate cancer. The method uses the alpha-toxin from Clostridium septicum to separate GPI-anchored glycoproteins for identification and optionally quantification. The GPI-APs so obtained may be used to raise antibodies for inclusion in an immunosorbent assay for the diagnosis or the monitoring of therapy of cancer in a patient.

### RELATED APPLICATIONS AND GRANT SUPPORT

This application claims the benefit of priority of international application PCT/US2012/048581, filed 27 Jun. 2012, entitled “Alpha Toxin Detection of GPI Anchored Proteins”, which claims the benefit of priority of claims the benefit of priority of provisional application Ser. No. 61/512,976, filed 29 Jul. 2011 of identical title and Ser. No. 61/579,957, filed Dec. 23, 2011, also of identical title, all of said applications being incorporated by reference in their entirety herein.

This invention was made with government support under grant nos. W81XWH-08-1-0565; BC075534, UO1CA128454 and P41RR018502 awarded by the United States Department of Defense. Consequently, the government has certain rights in the invention.

### FIELD OF THE INVENTION

The present invention relates to a method for the purification, concentration and identification of glycosylphosphatidylinositol anchored proteins (GPI-APs) from biological samples (cells, tissues and/or blood/serum) in a patient or subject, including a human patient or subject to be diagnosed for a disease. A new method to separate GPI-anchored glycoproteins, a class of glycoproteins found in all animal cells and fluids including serum, from other glycoproteins and proteins for the purpose of identifying potential biomarkers for various diseases, including cancer, especially breast cancer, vaginal cancer, endometrial cancer, cervical cancer, ovarian cancer, uterine cancer, pancreatic cancer and prostate cancer, represent alternative embodiments according to the present invention. The method uses the alpha-toxin from Clostridium septicum to separate GPI-anchored glycoproteins for identification and optionally quantification. The GPI-APs so obtained may be used to raise antibodies for inclusion in an immunosorbent assay for the diagnosis and/or the monitoring of therapy of cancer in a patient.

### BACKGROUND OF THE INVENTION

GPI anchoring of proteins to the cell surface was initially discovered in 1976 when it was revealed that alkaline phosphatase could be released from cells using the enzyme phospholipase C [6]. Since this discovery, scientists have been characterizing GPI-APs from several species such as plants, parasites, and humans [7, 8]. The GPI anchor consists of a core structure (FIG. 1) that is well conserved among different species [9].

The species and cell type diversity of GPI-anchors occurs with the different glycan substitutions (X and Y FIG. 1) that can be found attached to the conserved mannose residues of the core [10]. Synthesis of GPI anchored proteins begins in the endoplasmic reticulum with approximately 20 enzymes identified as participating in the production of the GPI anchor [11]. After several sequential glycan additions, the GPI anchor is transferred en bloc to the C-terminus of the protein in the endoplasmic reticulum by the action of the GPI transamidase complex, which recognizes a pattern of amino acids at the C-terminus of the protein. The GPI-transamidase simultaneously cleaves off the C-terminal sequence that serves as a signal for GPI attachment and adds the GPI anchor at an amino acid termed the omega site. Therefore, the final GPI-AP does not have the original C-terminus sequence. Once the GPI anchor is added to the glycoprotein it is transferred to the cell surface through Golgi transport [12].

Proteomic studies characterizing GPI-APs from human cells have focused on cleavage of the GPI-APs from isolated detergent resistant membranes using recombinant phospholipase C or recombinant GPI-specific phospholipase D [7]. This approach has been modestly successful; however, a method that captures endogenously released GPI-APs from solution, rather than from the membranes themselves, would be more compatible with downstream mass spectrometry techniques and more informative for high-throughput serum-based disease profiling, including cancer proteomic profiling.

Posttranslational addition of a glycosylphosphatidylinositol anchor (GPI), is performed in eukaryotic cells via the activity of the GPI transamidase (GPIT) (1). GPIT is a multisubunit enzyme complex required for the expression of GPI anchored proteins on the cell surface. GPI anchored proteins are predicted to comprise approximately 1-2% of translated proteins in mammals (2). Several GPI anchored proteins identified to date are tumor antigens such as carcinoembryonic antigen (3), mesothelin (4), prostate specific stem cell antigen (5), and urokinase plasminogen activator receptor (6), suggesting possible roles for this class of proteins in promoting tumorigenesis.

The predictive annotation of GPI anchoring in mammalian protein databases is difficult as there are no common consensus sequences that clearly indicate that a protein will receive a GPI anchor. There are several amino acid features that have been characterized in the C-terminus of proteins that receive the GPI anchor (7). The discovery of these common characteristics led to the development of algorithms to predict the probability of GPI anchor addition such as FragAnchor (8), GPI SOM (9), and Big-PI (10). Furthermore, the experimental isolation and identification of GPI anchored proteins from mammalian cells is often hampered due to the lower expression levels of GPI anchored proteins in many cell types coupled with difficulty in extracting these proteins due to the presence of both lipid and glycan structures (FIG. 1A, core GPI structure). GPI anchored proteins can be released into a soluble form using the bacterial enzyme GPI-specific phospholipase C (PI-PLC) (11). However, certain GPI anchored proteins may be phospholipase C insensitive due to acylation within the GPI anchor (12). In an effort to overcome these obstacles, we are employing the use of the bacterial toxin known as alpha toxin (AT), isolated from Clostridium septicum, to capture and enrich GPI anchored proteins from breast carcinoma for identification by mass spectrometry (See the fractionation scheme in FIG. 1B). AT is a member of the aerolysin-like pore forming toxins that bind with GPI anchored proteins (13). The diversity of GPI anchored proteins that AT can bind with suggests that the binding occurs via the GPI anchor without peptide requirements.

Human breast carcinomas express elevated levels of several GPIT subunits such as GPAA1 (GPI Anchor Attachment Protein 1) and PIGT (GPI Class T) due to gain of chromosome copy number (14). Increased expression of these subunits has been shown to induce tumorigenicity in vitro and in vivo (14). In our study, we document that increased expression of GPIT subunits results in increased levels of GPI anchored proteins in breast cancer epithelial cells evidenced by binding of AT from C. septicum. The present inventors isolate and identify proteins binding to AT using nano ESI-RPLC-MS/MS analysis. The data indicate that the membrane abundance of several cell surface receptors that are also found in mesenchymal stem cell populations is dependent on the expression of GPAA1 and PIGT. We report that increased expression of GPAA1 and PIGT positively regulates the expression of the embryonic Forkhead/Fox transcription factor FOXC2. Elevated expression of GPI anchored proteins also increases the expression of several mitochondrial membrane proteins that may promote the growth and survival of breast cancer. We also provide evidence that AT binds with GPI anchored proteins released into serum allowing the capture and detection of potential markers for the detection of breast cancer. Overall, these results indicate that GPI anchored proteins are abundant in breast cancer cells with functions that promote tumor growth and spread, making these proteins ideal diagnostic and therapeutic targets.

The overall abundance of GPI anchored proteins is estimated to comprise 1-2% of all translated proteins in the human proteome. The abundance in specific tissues may be lower due to the regulation of specific GPI anchored proteins that are expressed. In breast cancer, there are amplifications of the GPI anchor biosynthetic pathway that leads to increased levels of GPI anchor addition to proteins. In the present invention, the inventors have been utilizing the bacterial toxin known as alpha toxin to capture and identify GPI anchored proteins by mass spectrometry. Pursuant to the present invention, the studies have revealed that alpha toxin binds with the GPI glycan region due to the retained binding of the toxin after removal of the lipid portion of the GPI anchor with GPI-specific phospholipase C. Furthermore, the diversity of GPI anchored proteins that bind the toxin indicates that the binding occurs via the GPI glycan without peptide requirements. Consequently, the inventors have discovered that alpha toxin can be used as a lectin specific for the GPI anchor.

The primary mechanism that allows GPI anchored proteins to enter the circulatory system from tumors is not well understood. GPI anchored proteins can potentially be released from cells by proteolysis (6, second set of references), GPI-specific phospholipase activities (7, 8), or by exosome vesicular transport from the cell (6) (FIG. 8). The GPI glycan would remain attached to the GPI anchored proteins if the proteins were released by exosome or GPI-specific phospholipase cleavage (FIG. 1). The present invention is directed to the use of alpha toxin to determine if GPI anchored proteins are present at elevated levels with a GPI anchor glycan in serum samples obtained for various human cancers.

### OBJECTS OF THE INVENTION

It is an object of the invention to show that purified alpha toxin from Clostridium septicum can specifically recognize GPI anchored proteins in solution.

It is another object of the invention to show that purified alpha toxin from Clostridium septicum can recognize GPI anchored proteins in the serum of a patient or subject.

It is a further object of the invention to provide a method for identifying GPI anchored proteins from a biological sample obtained from a patient or subject, especially the blood/serum of the subject or patient.

It is yet another object of the invention to provide a method of identifying GPI anchored proteins from a biological sample obtained from a subject or patient and through analysis of the identified GPI anchored proteins determine (diagnosing) whether or not the subject or patient has a disease or condition reflective of that analysis.

Any one or more of these and/or other objects of the invention may be readily gleaned from a review of the description of the invention that follows.

### BRIEF DESCRIPTION OF THE FIGURES

FIG. 1 shows the core structure of the GPI anchor. X and Y represent currently known potential/possible glycan substitutions on the conserved mannose residues.

FIG. 2 shows the results of biotin-labeled alpha toxin binding of membrane proteins extracted from the invasive breast cancer cell line MDAMB231. Fractions of proteins from the extraction were sampled as indicated and presented: 2A shows the results of the pre-PI-PLC fraction containing all membrane proteins solubilized in SDS-loading buffer; 2B shows the results of PI-PLC release which contained the proteins that were released into the soluble phase after PI-PLC enzyme incubation; 2C shows the post-PI-PLC fraction which contained membrane proteins remaining that were solubilized in SDS-loading buffer. The PI-PLC digestion released GPI anchored proteins into the soluble phase as shown in FIG. 2B. The biotinylated alpha toxin reacted with more PI-PLC released proteins than the anti-CRD antibody, indicating a higher sensitivity. Several bands were concentrated in the PI-PLC released fraction compared with the pre-PI-PLC lane shown by the asterisk in FIG. 2A. Equivalent amounts of protein were loaded into each lane as evidenced by the equal staining of various bands in the Sypro ruby stained gel FIG. 2C.

FIG. 3 shows silver stained gel showing toxin captured proteins isolated from membrane extracts from breast cancer cells MDAMB231 expressing control shRNA, Gpaa1 shRNA, PigT shRNA, and MCF10A normal mammary cells.

FIG. 4 shows silver stain gel showing proteins bound to alpha toxin from patient matched normal or breast tumor tissue.

FIG. 5 shows silver stain gel showing proteins bound to alpha toxin from pooled normal non-diseased patient serum (NL) or serum from patients with invasive ductal breast carcinoma (BC).

FIG. 6A shows that alpha toxin binds at high levels to malignant epithelial cells in tissue from ductal invasive breast carcinoma.

FIG. 6B shows that alpha toxin binds at a very low level to stromal areas of patient matched normal breast tissue.

FIG. 7 shows that alpha toxin binds can specifically capture phospholipase released GPI anchored proteins. Alpha toxin Selectively captured proteins from the PIPLC treated cells. Equivalent levels of proteins were present in the reactions Indicated by the 10% input.

FIG. 8. (A) Schematic of the GPI biosynthetic pathway. The core GPI anchor structure is assembled stepwise in the ER and added en bloc to the C-terminus of proteins that display a C-terminal signal sequence by the multisubunit enzyme complex GPIT (blue asterisk). Enzymes increased in breast carcinoma are labeled. (B) Schematic of the sample processing and isolation of GPI anchored proteins. Patient matched tissue samples and cells from the indicated cell lines were used for extraction of total membrane proteins. Membrane extractions were analyzed by nano-ESI-MS/MS to verify equivalent protein levels and quality. Biotinylated AT was added to membrane extractions and serum obtained from women with ductal invasive breast carcinoma or women with benign breast disease. GPI anchored proteins with bound AT were captured with streptavidin magnetic beads and analyzed by nano-ESI-MS/MS.

FIG. 9. AT capture of membrane proteins requires the expression of GPAA1 and PIGT (A) Real-time PCR measurement of GPAA1 mRNA levels in human breast cancer MDAMB231 cells stably expressing control siRNA or GPAA1 siRNA. GPAA1 levels were normalized to RPL4 and the control level set to 1.0 for comparison. Error bars represent the standard error of the mean (SEM) for triplicate measurements. (B) Real-time PCR measurement of PIGT mRNA levels standardized and measured as described for GPAA1. (C) Silver stained gel of AT bound proteins obtained using equivalent inputs of membrane proteins isolated from MDAMB231 stable cell lines and MCF10A cells.

FIG. 10. GPI biosynthetic enzyme transcript levels and GPI anchored proteins are elevated in human breast cancer. (A) Real-time PCR analysis of enzymes in the GPI biosynthetic pathway using RNA isolated from MDAMB231 cells and non-transformed human mammary MCF10A cells. Transcript levels are normalized to RPL4 and the MCF10A levels are set to 1.0 for comparison. Error bars represent the SEM for triplicate measurements. (B) Immunohistochemistry analysis of AT binding to GPI anchored proteins in ductal invasive breast carcinoma tissue and patient matched adjacent normal tissue, magnification 40×.

FIG. 11. Alpha toxin capture enriches for membrane proteins. (A) Cell compartment analysis of proteins identified from nano-ESI-RPLC-MS/MS analysis of total membrane proteins from human breast cancer before AT capture. (B) Cell compartment analysis of proteins identified from nano-ESI-RPLC-MS/MS analysis of human breast cancer after AT capture. The percentage of proteins in the membrane compartment increases to 82%, and from this 82%, >50% of the proteins are found in the plasma membrane.

FIG. 12. GPAA1 and PIGT expression levels regulate FOXC2 transcript levels. (A) Real-time PCR analysis of FOXC2 transcript levels in MDAMB231 cells stably expressing the indicated siRNAs. Error bars represent the SEM of data from 2 separate experiments with 5 replicates each normalized to RPL4. (B) Real-time PCR analysis of FOXC2 transcript levels in MCF10A cells stably expressing GPAA1 or PIGT cDNA. Error bars represent data from 2 separate experiments with 5 replicates each normalized to RPL4. (C) Representative Western blot analysis of FOXC2 protein levels in MCF10A cells for control (vector), GPAA1, or PIGT. Densitometry graph represents band density normalized to ERK levels.

FIG. 13. GPI anchored proteins from serum bind AT. (A) Silver stained gel of AT bound proteins from pooled serum (n=5) obtained from non-diseased patients (Normal) and pooled serum (n=5) from patients with ductal invasive breast carcinoma (Breast Cancer). (B) Western blot analysis of serum from patients with ductal invasive breast carcinoma following AT capture. The blot was probed using the indicated antibodies. (C) Western blot analysis of serum from women with benign polycystic breast disease following AT capture. Blots were probed using the indicated antibodies. Gelsolin binds with AT in serum obtained from patients with breast carcinoma and non-malignant serum samples and serves as a control for serum quality and input.

FIG. 14, Table 1, shows all cluster of differentiation (CD) receptors that show GPIT dependence as described in Appendix A. Overwhelmingly the CD markers listed in Table 1 are associated with mesenchymal stem cell (MSC) populations. These are CD markers which are reduced by Gpaa1 or PigT suppression. All of these proteins have tumor-specific expression in breast cancer tissue with the exception of CD36.

FIG. 15 (a-g), Table 2 lists all proteins, in alphabetical order with respect to official gene code (2+ peptides minimum found in at least 2 separate samples) detected that bind to alpha toxin (AT) from breast tissues (T), serum (S) and cell lines (CL). The proteins listed in Table 1 binding with AT are likely presenting in the membrane of breast cancer cells with a GPI linkage making these novel biomarkers for breast carcinoma.

FIG. 16 (a-bbb) Table 3, shows the complete peptide coverage and complete peptide list for each peptide in Table 2, described above.

FIG. 17, Table 4, provides a list of alpha toxin bound proteins identified in normal MCF10A cells, normal breast tissue or normal serum. These mesenchymal genes are induced in malignant breast epithelial cells.

FIG. 18, Table 5, provides a list of serum proteins that were found to be binding AT from cancer patient serum that were never found in tissues or cells.

FIG. 19, Table 6, shows primer sequences used in qRT-PCR analysis.

FIG. 20 shows a diagram illustrating various mechanisms that can result in the release of GPI anchored proteins from the cell surface. GPI anchored proteins released from membrane-derived vesicles or via GPI-specific phospholipase enzymes would be expected to have an intact GPI anchor (lipid and glycan) or partial GPI anchor (glycan). GPI anchored proteins released by proteolysis will not have the GPI anchor.

FIG. 21 shows that alpha toxin capture of CEA5 requires the presence of the GPI anchor. (A) LS174T human colon adenocarcinoma cells were divided into equal fractions, one fraction received buffer only (−) and one fraction received GPI-specific PI-PLC (1.5 units/ml) (+). Following incubation for one hour at 37° C., the supernatant was subjected to alpha toxin capture followed by Western blot detection of carcinoembryonic antigen 5 (CEA5). Input equals 10% of the supernatant used for alpha toxin capture. (B) Ten percent protein inputs and alpha toxin captured proteins were separated on a 4-12% polyacrylamide gel and stained with silver.

FIG. 22 shows that alpha toxin reacts with sera from human cancers and not with control (non-malignant) sera. (A) Representative slot blot analysis of human serum (5 μL) from patients with the indicated cancers detected with biotin labeled alpha toxin (2 μg/ml) followed by streptavidin conjugated peroxidase and chemiluminescent substrate. (B) Cumulative results from slot blot analysis. Alpha toxin binding levels were normalized to alpha-1-acid glycoprotein levels for each case. Error bars indicate the +SEM for normalized densities obtained from all serum samples analyzed for each cancer.

FIG. 23 shows (A) LS174T cells were divided into equal fractions, one fraction received 1×PBS (−) and one fraction received PL-PLC (+) 1.5 units/ml. Following incubation for 30 min. at 37 C, the supernatant was subjected to alpha toxin capture followed by Western blot detection of carcinoembryonic antigen 5 (CEA5). (B) Ten percent of the protein inputs and alpha toxin captured proteins were separated on 4-12% PAG and stained with silver.

FIG. 24 shows (A) HEK cells stably expressing vector or GPAA1 (GPAA1 cells shown in A) were transfected with GFP-FERMT3 or GFP-DAF. One day post transfection, the cells were collected by pipet and divided into two equal fractions, one for mock (−) and one for PI-PLC treatment 1.5 units/ml for 30 minutes. The supernatant was removed to a clean tube and the cells were subjected to immunofluorescence. (B) The supernatants were subjected to alpha toxin capture followed by SDS-PAGE and Western blot. (C) Triton X-114 (we-condensed) was used to isolate the detergent fraction from the mock treated GPAA1 cells expressing GFP-FERMT3 and GFP-DAF. The detergent fraction was divided into two equal fractions and treated with 1×TBS or PI-PLC for 30 minutes at 37 C. The samples were phase partitioned and the aqueous fractions were precipitated prior to SDS-PAGE and Western blot.

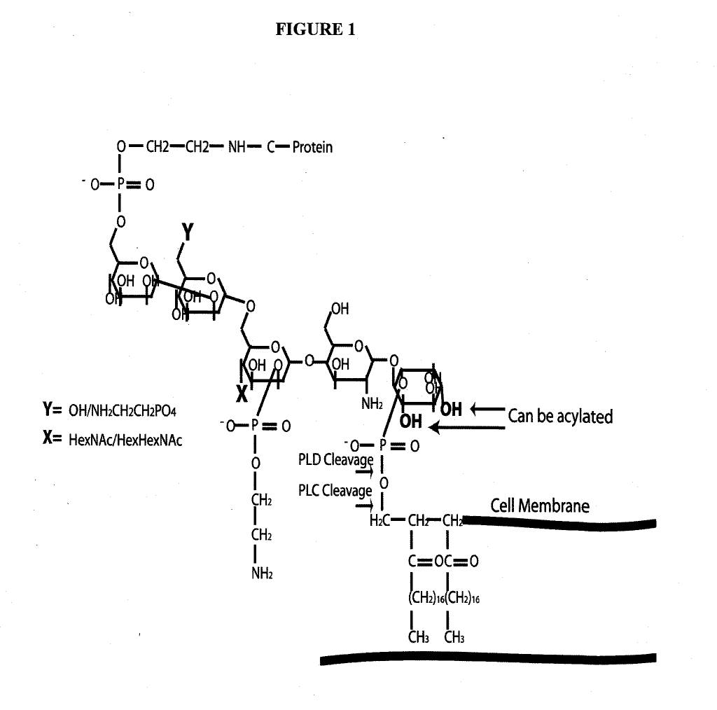

### BRIEF DESCRIPTION OF THE INVENTION

This application is directed to methods developed using alpha toxin from Clostridium septicum (C. septicum) to enable the detection, capture, identification, and targeting of glycosylphosphatidylinositol anchored proteins (GPI-APs). The identification of GPI-APs enables improved detection and treatment for cancers that have elevated expression of GPI-APs, such as breast cancer, endometrial cancer, uterine cancer, ovarian cancer, vaginal cancer, pancreatic cancer and prostate cancer, among numerous others, including most cancers, as otherwise described herein.

Many of the existing circulating tumor markers that have been identified such as carcinoembryonic antigen, urokinase plasminogen activator, and prostate stem cell antigen, were once linked to the cell through a glycosylphosphatidylinositol anchor. GPI-APs are a component of lipid-rich microdomains at the cell surface known as lipid rafts [1, 2]. The associations of GPI-APs within lipid rafts are important for maintaining signaling pathways that control cellular growth and differentiation. Although GPI-APs are concentrated in the lipid rafts at the cell surface, they can be cleaved by enzymes into a soluble form allowing release from the cell [3]. Therefore, the inventors have found that soluble released GPI-APs found in serum can be useful as diagnostic markers and intact GPI-APs on the surface of tumor cells as potential targets for therapeutic targets, facilitating anticancer therapy. Also possible for use in imaging cancers in vivo.

Alpha toxin from Clostridium septicum binds to GPI-APs through the GPI glycan region [4, 5]. The toxin binding and the specificity of the binding is favorable for cancer diagnostics, because alpha toxin binds preferentially to GPI-APs that appear on the surface of cancer cells. In particular, preliminary data presented in the present application indicate that the toxin binds to GPI-APs from cancer, especially human breast carcinoma tissue and serum with very little binding to normal human breast tissue and serum. These studies provide evidence that the toxin can be used as a detection or therapeutic targeting molecule for a number of forms of cancer, especially including breast cancer.

In one aspect, the present invention is directed to a method of isolating GPI anchored glycoproteins from cells and/or cell preparations, including solubilized cellular membranes, using labeled alpha toxin from Clostridium septicum as otherwise described herein, the method comprising the steps of exposing a population of cells and/or cell preparations to alpha toxin (in certain aspects, preferably containing a fluorescent label) from Clostridium septicum, allowing binding of GPI-APs to occur to produce an alpha toxin GPI-AP complex, isolating the alpha toxin GPI-AP complex(es) from the cells and/or cell preparation and optionally identifying and further optionally, quantifying the bound GPI-APs. This method allows the identification, purification and/or concentration of specific GPI-APs from a cell surface of a tissue or cell sample and identifying GPI-APs associated therewith from which one may establish one or more target biomarker(s) from which the diagnosis and/or treatment progress of a condition or disease state (e.g. cancer, especially breast cancer) associated with a particular cell and target biomarker may be established. This method is useful for identifying GPI-APs that are found on certain types of cells, especially cancer cells, such that selectivity may be established regarding the GPI-APs that are expressed on those cells. From this information, providing diagnostic assays to assist in the diagnosis and/or treatment of a disease in which the identification of the existence and amount of GPI-AP found in a cell or tissue sample is relevant is straightforward.

In a related method, the present invention is directed to the above method wherein the GPI-AP complex obtained above is treated to remove the alpha-toxin from the GPI-AP and the GPI-AP is identified and optionally quantified to provide a standard biomarker in identity and preferably quantity associated with a disease state or condition to diagnosed or monitored for effectiveness of therapy in a patient.

In a further aspect of the invention, the present method relates to the diagnosis of a disease state or condition comprising obtaining a biological sample (cells, tissue and/or a blood sample or a fluid such as sputum, urine or cerebrospinal fluid) from a patient or subject suspected of having a disease state or condition, exposing the biological sample to an antibody specific for a cancer biomarker or to Clostridium septicum alpha-toxin to form a GPI-AP alpha-toxin complex; measuring the amount of antibody bound GPI-AP complex or GPI-AP alpha-toxin complex and comparing the amount of said GPI-AP antibody or said GPI-AP alpha-toxin to one or more predetermined value(s) or control(s), wherein an amount of alpha-toxin complex obtained from said biological sample which is above or below the predetermined value(s) or control(s) is evidence of the presence or absence of a disease state or condition in the biological sample. This analysis can be performed in an immunoassay as otherwise described herein (including a multiplexed assay such as a bead assay) in order to rapidly diagnose a patient suspected of having cancer or alternatively, to monitor therapy of cancer in a patient to determine the progress of such therapy on the disease state or condition.

In an alternative embodiment, the present method provides a GPI-AP alpha-toxin complex as prepared above, wherein the glycoprotein alpha-toxin complex so obtained is treated to remove and isolate said alpha-toxin from said GPI anchored glycoprotein and said GPI-AP obtained therewith is optionally identified.

The present invention relates to a GPI-AP protein that is obtained by any one or more methods according to the present invention.

In a related aspect of the present invention, Clostridum septicum alpha-toxin can be purified, for example, by coupling to a resin, such as a magnetic or other resin (or other method) and used to capture GPI anchored proteins for mass spectrometry identification from human cells, tissue, and serum (blood). Alternatively, the purified alpha-toxin may be coupled to an antibody that has been adapted for use in an immunoassay, including an enzyme-linked immunosorbent assay (ELISA), which may include fluorescent or colorimetric means to quantify binding. The immunosorbent assay may be used to identify and quantify select GPI-APs in a biological sample such as blood, serum or urine (or other biological liquid such as sputum or cerebrospinal fluid), to determine their presence or absence and the existence or absence of a disease state associated the GPI-APs in the biological sample.

In the present invention, alpha toxin can be used to distinguish cancer tissue from non-cancer tissue, including breast cancer tissue from non-cancer tissue, the method comprising exposing tissue to labeled alpha toxin and determining the presence and/or concentration of cancer biomarkers in the tissue and comparing the presence and/or concentration of biomarkers in the tissue sample to a predetermined value from non-cancer tissue and/or cancer tissue, wherein the presence of biomarkers in the tissue which is greater than normal tissue or is the same as or approximately the same as cancer tissue is evidence that the tissue is cancer tissue (as opposed to non-cancer tissue).

The present invention also relates to variants of alpha toxin, peptides derived from alpha toxin, optimized mutant forms of alpha toxin and antibodies raised against alpha toxin (including the alpha toxin binding epitope) which may be used as diagnostic and/or therapeutic agents in the diagnosis and/or treatment of a disease or disease state, especially cancer, including breast, endometrial, cervical, vaginal, ovarian, pancreatic and prostate cancer, among others as described herein. It has unexpectedly been discovered that each type of cancer produces a selective GPI-AP profile which can be used to identify (diagnose) the existence of cancer in a patient, to identify the extent of cancer in a patient, to establish the progression (monitoring) of cancer therapy in a patient and identify effective therapies, including compounds which may be useful for cancer therapy by identifying the impact (often, the inhibition) on the expression of the biomarker by a compound having an unknown or not fully appreciated biological activity (drug screening).

In a further method according to the present invention, alpha toxin bound proteins which have been released from alpha toxin (using standard methods known in the art) are identified by mass spectrometry from cells, tissues, and serum (including cancer cells and tissue, especially breast cancer cells and tissue) that may be used as diagnostic or therapeutic targets via the GPI anchor.

In the case of breast cancer, the inventors have identified the biomarkers isoform 2 of Fermitin family homolog 3 (FERMT3 or Kindlin 3) and filamin A (FLNA) as being expressed at high levels in breast cancer tissue. GPI-anchor proteins of both of these biomarkers are found in the plasma of breast cancer-afflicted patients. Identifying and qualifying/quantifying the expression levels of one or both of FERMT3 and filamin A (FLNA) may be used to diagnose breast cancer in a patient, identify the extent of breast cancer in a patient or subject, establish the progression of breast cancer therapy in a patient undergoing such therapy. Additionally, the (inhibition or upregulation, usually the inhibition of) expression levels of FERMT3/Kindlin3 and/or filamen A (FLNA) in in vitro and/or in vivo test systems may be used to identify potential therapies, especially including potential drug therapies for breast cancer. An immune assay system that includes labeled C. septicum alpha toxin in conjunction with at least one antibody which binds to FERMT3 and/or filamen A (FLNA) represents an alternative embodiment of the present invention.

In one embodiment of the invention, a method is directed to the identification of at least one biomarker (GPI-AP) on a cancer cell, wherein said biomarker is expressed selectively on said cancer cells by virtue of such a biomarker being absent on said normal cells or said biomarker is expressed in greater numbers on cancer cells in comparison to normal cells, the method comprising:
1. Providing a sample of cancer cells and normal cells from the same tissue (usually from cancer tissue of a subject or patient);
2. Lysing said cells, optionally in the presence of a non-ionic detergent (preferably, polyethylene glycol tert-octylphenyl ether or Triton X-114), to obtain a mixture of cellular proteins, preferably a mixture of membrane proteins;
3. Exposing said cellular proteins or said membrane proteins to phospholipase (preferably phospholipase C) to release membrane proteins from membranes of said cells to produce a population of membrane-released GPI-anchor proteins;
4. Exposing said population of GPI-anchor proteins to labeled C. septicum alpha toxin to produce alpha toxin bound GPI-anchor protein;
5. Isolating and/or purifying said alpha-toxin bound GPI-anchor protein and releasing alpha toxin from GPI-anchor protein to produce isolated GPI-anchored proteins;
6. Optionally, releasing protein from said GPI-anchor protein by deaminating said protein from the GPI moiety (using hydrofluoric acid—see Dagdanova, et al., J. Biol. Chem., 285, 30489-30495, Oct. 1, 2010 and Mehlert and Ferguson, Glycoconjugate Journal, 2009 November; 26(8): 915-921 and below) to provide a population of released (from GPI moieties to which they are attached) proteins to be analyzed;
7. Analyzing (preferably by mass spectrometry) said GPI-anchor proteins or optionally, said released proteins to determine the content and quantity of proteins in such population of GPI-anchor proteins for each of said cancer cells and normal cells; and
8. Comparing the content and quantity of proteins in said population of GPI-anchor proteins from said cancer cells with said normal cells, wherein a population of GPI-anchor proteins which is found exclusively on said cancer cells or at an identifiably higher concentration on said cancer cells compared to said normal cells identifies that GPI-anchor protein(s) as a potential selective biomarker for cancer diagnosis and/or treatment. Once the potential selective biomarker is identified, an antibody (polyclonal or monoclonal) may be raised to that protein/peptide using standard methods well known in the art.

Once the cancer specific biomarkers are identified from cancer cells (either because of selective expression in contrast to normal cells or because of heightened expression, e.g., hyperproducer, of one or more biomarkers in the cancer cells compared to normal cells, the biomarker(s) is then identified in a biological sample (blood, serum or urine, preferably serum/plasma or other bodily fluid) from a group of cancer patients to determine whether or not that biomarker may be useful as a potential diagnostic biomarker. In this method aspect, plasma is obtained from a patient or subject, optionally, a protein sample is enriched from the plasma sample and the plasma and/or enriched protein sample is exposed directly to labeled C. septicum alpha toxin to produce alpha toxin bound GPI-anchor proteins. These alpha toxin bound GPI-anchor proteins are then separated (generally, using the label such as biotin on the alpha toxin to provide isolated alpha toxin bound GPI-anchor proteins which also separates the alpha toxin from the GPI-APs using standard elution methods with the alpha toxin remaining with the particles to which the label, such as biotin, is bound) and the isolated GPI-anchor proteins (which may be optionally further separated from the GPI portion of the complex to provide a population of polypeptides) are then identified (using, for example, mass spectrometry as discussed above or by binding to labeled polyclonal or monoclonal antibodies which have been raised to biomarkers identified in the method above) to determine whether the GPI-anchor proteins identified in the plasma sample match those which were identified from cancer cells. The biomarkers which match are potential cancer biomarkers found in the bloodstream of a patient to be diagnosed and these may be readily monitored in plasma using a standard assay system (utilizing at least one antibody in an immune assay or standard bead assay, including a multiplex bead assay) to determine whether or not a patient has cancer and the type of cancer afflicting the patient. Immunoassays (e.g. ELISA, other) based upon labeled C. septicum alpha toxin which capture GPI-APs in conjunction with at least one peptide (biomarker) specific polyclonal or monoclonal antibody which is labeled with a reporter (preferably, a fluorescent reporter as otherwise described herein) or an enzyme (in conjunction with a substrate which produces a colorimetric response) is used to identify the presence and/or concentration/quantity of a biomarker. Alternatively, the present method can be used in multiplex immunoassays (bead based or other) to assess the presence of more than one cancer biomarker.

In an alternative embodiment, the present invention is directed to a method of identifying whether or not a compound of unknown cancer activity is a potential anticancer agent, said method comprising exposing cancer cells which express GPI-APs either selectively or at a level which is substantially higher than normal cells to a compound of unknown cancer activity, measuring the expression of said GPI-APs by binding said GPI-APs to labeled alpha toxin and comparing said measurement obtained with a predetermined value, wherein a measurement which is below the level of expression of said GPI-APs for said cancer cells identifies the compound as a potential anticancer agent. In this method, the cancer cells may be breast cancer cells and the breast cancer cells express a population of GPI-APs which comprise FERMT3/kindling 3 and a population of GPI-APs which comprise, FilamenA (FLNA),

### DETAILED DESCRIPTION OF THE INVENTION

The term “patient” or “subject” is used throughout the specification to describe a mammal, preferably a human, to whom the present method of diagnosing the likelihood of the existence of a disease state or condition or monitoring the therapy of a disease state or condition in a patient or subject is directed or from whom a sample of tissue or cells to be analyzed is obtained. In preferred, non-veterinary aspects of the present invention, the term patient refers to a human patient or subject.

The term “GPI-AP” is used to describe glycosylphosphatidylinositol anchored proteins (GPI-APs) that are identified in methods of the present invention. These are proteins that are found at the cell surface in a variety of cells and through the present invention, may be used as biomarkers in the diagnosis and treatment of disease states, especially including cancer. They are a functionally and structurally diverse family of post-translationally modified membrane proteins found mostly in the outer leaflet of the plasma membrane in a variety of eukaryotic cells. Although the general role of GPI-APs remains unclear, they act as enzymes and receptors in cell adhesion, differentiation, and host-pathogen interactions. GPI-APs represent potential diagnostic and therapeutic targets and are presented in attached Table 1 of Appendix A and further described in Supplemental Table 1 of Appendix A. Particularly preferred GPI-APs include those wherein the anchored protein is Filamin A (FLNA) or kindlin 3 (FERMT3).

Proteins may be released from GPI-APs using methods for cleaving off the GPI group from the peptides that are covalently linked. GPI glycan extraction generally follows alpha toxin capture from tissues, cells, or serum. In this approach, a minimum amount (e.g. 20 mg) of captured GPI anchored proteins are applied to polyvinylidene fluoride (PVDF) membrane. The membrane is excised and placed in a microcentrifuge tube and covered with ice-cold hydrofluoric acid (e.g., 48% HF) for a sufficient period of time at low temperature (e.g., 48 hours at −20 C). The released GPI glycans are removed from the PVDF and placed in a new tube with 1 mL water prior to lyophilization of the glycans. Water is added again and the lyophilization is repeated.

The term “predetermined value” refers to a concentration or amount of a GPI-AP which is a standard obtained from a statistically significant sample obtained from a normal patient group (i.e., one or more patients without disease) or a patient group with disease pursuant to clinical results and to which a measurement of GPI-AP in a measured sample is compared in order to determine whether a patient is normal or has a disease, and in certain instances, the extent of disease, as otherwise described herein. The predetermined value may be obtained from a group of nonnal patients or subjects to establish a “normal” predetermined value or from a group of patients with a diagnosed disease wherein the predetermined value is a “diseased” predetermined value. Comparison of GPI-APs from a biological sample obtained from a patient or subject to be tested with the predetermined value(s) will allow a diagnostician or clinician to determine the existence or absence of a disease state and the relative severity (including the relative degree of healing) of the disease.

The term “Clostridum septicum” is used to describe a spore-forming gram positive rod bacterium that produces an alpha toxin that is used in the present invention. Clostridium septicum are found in virtually all anoxic habitats where organic compounds are present, including aquatic sediments, soils, animals and humans guts. Like C. botulinum, C. septicum produces a number of toxins, most notably the alpha toxin used in the present invention. Alpha toxin is a pore-forming toxin responsible for gas gangrene in humans and animals.

The term “alpha toxin” is used to describe alpha toxin from Clostridium septicum. Clostridium septicum alpha-toxin is secreted as an inactive 46,450-Da protoxin, the polypeptide sequence of which includes the following:

(SEQ ID NO: 1)
MSKKSFAKKVICTSMIAIQCAAVVPHVQAYALTNLEEG
GYANHNNASSIKIFGYEDNEDLKAKIIQDPEFIRNWAN
VAHSLGFGWCGGTANPNVGQGFEFKREVGAGGKVSYLL
SARYNPNDPYASGYRAKDRLSMKISNVRFVIDNDSIKL
GTPKVKKLAPLNSASFDLINESKTESKLSKTFNYTTSK
TVSKTDNFKFGEKIGVKTSFKVGLEAIADSKVETSFEF
NAKQGWSNTNSTTETKQESTTYTATVSPQTKKRLFLDV
LGSQIDIPYEGKIYMEYDIKLMGFLRYTGNAREDHTED
RPTVKLKFGKNGMSAEEHLKDLYSHKNINGYSEWDWKW
VDEKFGYLFKNSYDALTSRKLGGIIKGSFTNINGTKIV
IREGKEIPLPDKKRRGKRSVDSLDARLQNEGIRIENIE
TQDVPGFRLNSITYNDKKLILINNI

(See, inter alia GenBank: EU482197.1; EU482189; EU482196.1)
which sequences are incorporated by reference herein. From the various polypeptides, antibodies may be prepared for use in diagnostic and/or therapeutic monitoring assays. Alpha toxin of SEQ ID NO: 1 is the preferred alpha toxin for use in the present invention, although it is understood that various alternative alpha toxins, including protoxin as described hereinbelow and mutants of the alpha toxin may also be used in the present invention. Any alpha toxin which is capable of binding GPI-APs may be used in the present invention.

The protoxin of alpha toxin is activated by proteolytic cleavage near the C terminus of the above polypeptide, which eventually causes the release of a 45-amino-acid fragment. Proteolytic activation and loss of the propeptide allow alpha-toxin to oligomerize and form pores on the plasma membrane, which results in colloidal-osmotic lysis. Activation may be accomplished in vitro by cleavage with trypsin at Arg367 (J. Ballard, Y. Sokolov, W. L. Yuan, B. L. Kagan, and R. K. Tweten, Mol. Microbiol. 10:627-634, 1993), which is located within the sequence KKRRGKR367S. A conspicuous feature of this site is a recognition site (RGKR) for the eukaryotic protease furin. Pro-alpha-toxin (AT[pro]) that was digested with trypsin or recombinant soluble furin yields the 41,327-Da active form (AT[act]). While the proteolytic form of alpha toxin is a less preferred version of the wild-type alpha toxin which is preferably used in the present invention, principally because the proteolytic form exhibits biological activity inconsistent with and in some cases which complicates the use of the present invention, the proteolytic form of alpha toxin described above also may be used in the present invention. Mutated forms of alpha toxin may also be used in the present invention provided that they can bind to GPI-anchlor proteins as otherwise described herein. A mutated alpha-toxin in which the furin consensus site is altered to KKRSGSRS at the cleavage site (AT[SGSR]) is cleaved and activated by trypsin but not by furin. Furin is involved in the activation of C. septicum alpha-toxin on the cell surface but that alternate eukaryotic proteases can also activate the toxin. Regardless of the activating protease, the furin consensus site appears to be essential for the activation of alpha-toxin on the cell surface. Thus, pursuant to the present invention, the term alpha toxin includes the full native polypeptide, the protoxin, a mutant (including mutants described in U.S. Pat. No. 7,179,888, which is incorporated by reference herein) or variant or a polypeptide region thereof, each of which binds to GPI-APs. In preferred aspects of the invention, alpha toxin is labeled, preferably with biotin and/or a fluorescent label for identifying and/or isolating GPI-APs or for diagnostic and/or monitoring therapy aspects of the present invention. See also, Ballard, et al., Infection and Immunity, January, 1995, pp. 340-344 and Knapp, et al., Toxicon, 55, 1, pp. 61-72 (January, 2010), relevant portions of which are incorporated by reference herein.

In order to prepare the preferred alpha toxin (AT) according to the present invention, the plasmid pBRS10 encoding native (wild-type) AT expressing a histidine tag (see Sellman, et al., Mol. Microbiol. 23(3)551-558, 1997) may be transformed into E. coli as expression vector, wild-type alpha toxin is produced and isolated. The procedure is rather facile. Alternatively, the peptide sequence, which is known, may be readily synthesized using standard peptide synthesis, a cDNA is produced from the synthesized peptide, incorporated into a plasmid vector, transformed into an expression vector and produced in sufficient quantities, all using methods well known in the art.

The term “cancer” refers to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri; corpus uteri, ovary, prostate, testis, bladder, renal, brain/CNS, head and neck, throat, Hodgkin's disease, multiple myeloma, leukemia, melanoma, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, including benign and malignant lymphomas, particularly Burkitt's lymphoma and Non-Hodgkin's lymphoma, benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line and non-germ line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas which may be treated by one or more compounds according to the present invention. In preferred aspects of the invention, cancer shall refer to breast, ovarian, endometrial, vaginal, uterine, prostate or pancreatic cancer.

The term “antibody” shall mean an antibody, or an antigen-binding portion thereof, that binds to alpha toxin and/or GPI-AP and/or an alpha toxin GPI-AP complex and is used in an assay, to measure the binding of a GPI-APs to alpha toxin (GPI-AP/alpha toxin complex), especially including GPI-APs of the proteins which have been identified in Table 1 of the attached Appendix A. Alternatively, an antibody may bind to any one or more of the peptides which have been identified and presented in the accompanying tables which have been attached to the present application. A combination of antibodies which bind to GPI and to a specific peptide may be useful in diagnostic and/or therapeutic aspects according to the present invention, although the use of alpha toxin in lieu of an antibody in diagnostic assays, including immunoassays, may be preferred. An antibody as used herein may be polyclonal or monoclonal and is usually a murine, rat or rabbit antibody, more preferably a murine antibody which binds to a protein/peptide, especially a human protein/peptide for biological assays. [Could also include other species—lamprey] Methods for making polyclonal and monoclonal antibodies are well known to the art. Monoclonal antibodies can be prepared, for example, using hybridoma techniques, recombinant, and phage display technologies, or a combination thereof. See, for example, Golub et al., U.S. Patent Application Publication No. 2003/0134300, published Jul. 17, 2003, for a detailed description of the preparation and use of antibodies as diagnostic or therapeutic agents. Antibodies to alpha toxin mutants, peptides and/or derivatives can be generated by standard means as described, for example, in “Antibodies: A Laboratory Manual” by Harlow and Lane (Cold Spring Harbor Press, 1988), using methods which are well known in the art.

Preferably, the antibody is a monoclonal antibody to provide the desired specificity for binding to a peptide portion of a GPI-AP. Useful antibodies that selectively bind Clostridum septicum alpha toxin and/or GPI-AP and/or anchor protein/GPI-AP complex include those disclosed in U.S. Pat. No. 7,179,888, which is incorporated by reference herein. Numerous other antibodies can also be used. The antibodies are quite useful for analyzing, isolating and identifying GPI-APs which bind to alpha toxin in the method of present invention. Once the GPI-APs are isolated, antibodies may be readily raised to the isolated GPI-APs, or to the peptide portion of the GPI-AP for use to determine the present of specific GPI-APs in a biological sample such as blood, urine, or plasma of a patient to be diagnosed for disease. While any method for determining the concentration of a particular GPI-AP in the blood, serum or urine of a patient may be used in the present method, including mass spectrometry, etc., preferably, a colorimetric or chemiluminescent antibody-based assay such as a sandwich assay or bead based assay (all of which may be multiplexed) to identify the presence and concentration of more than one particular type of GPI-AP in a sample, especially for the diagnosis of a disease state or condition, especially including cancer, or for monitoring the treatment of a disease state. This technology is well-known in the art. In certain aspects of the invention, a combination of antibodies are used with specificities to the GPI portion of a GPI-AP and at least one additional antibody, preferably a monoclonal antibody which is specific for the peptide portion of the GPI-AP. In alternative embodiments, anchor protein that binds to GPI-AP may be used in combination with one or more antibodies (preferably monoclonal) which are specific for a peptide portion of a GPI-AP to be identified in a sample in order to provide an immunoassay effective to diagnose a disease state such as cancer.

Antibodies are employed in immunoassays (ELISA) in methods according to the present invention in order to measure the concentration of GPI-APs in the serum, whole blood or cell lysate (preferably serum). The assay employed is most preferably a simple colorimetric sandwich assay, as described herein below, which indicates a concentration of GPI-APs of a predetermined value, based upon a colorimetric or chemiluminescent readout which evidences the likelihood of a subject or patient being afflicted with a disease state in the event the intensity of the readout is above a predetermined value. However, any assay which utilizes an anti-GPI-AP antibody or a fluorescently labeled alpha toxin in a plate assay to measure GPI-APs to a concentration that may be compared to a predetermined value may be used provided that it is fast and accurate. The preferred assay for use in the present invention is a qualitative colorimetric or chemiluminescent nitrocellulose based monoclonal sandwich assay as described in greater detail herein which can be used to qualitatively measure the target GPI-APs in whole blood or serum above or below a predetermined value. The assay is sufficiently sensitive to measure GPI-APs associated with cancer cells (especially including breast, vaginal, cervical, endometrial, uterine, pancreatic or prostate cancer, among numerous others as otherwise described herein). To determine whether a patient has cancer, the patient's blood or serum levels of a target GPI-AP is measured, the result providing an indication of the likelihood of the presence of cancer in the patient. In certain embodiments this method may also be used to determine the impact of therapy on cancer in a patient, including by comparing measurements of one or more target GPI-AP(s), for example, before and after commencement of therapy.

As a first step in the method, a patient or subject suspected of having cancer is tested to determine whether or not that patient or subject has cancer. The preferred method for testing the existence of cancer is a GPI-AP sandwich assay which measures one or more GPI-APs which are hyperexpressed in cancer cells using antibodies which are raised to the GPI-APs or to a GPI-AP-anchor protein complex and determining the concentration of specific GPI-APs in the serum of a patient or subject and comparing that concentration to a predetermined value, wherein a measure of the concentration of GPI-APs above or below a predetermined value is indicative of that a patient has cancer or is cancer-free.

As described above, labeled (including fluorescently labeled) alpha toxin may be used in an assay for cancer, including breast cancer, which measures GPI anchor proteins in the serum of a patient to determine the likelihood of the existence of breast cancer in that patient) that relies on the chemical removal of GPI glycans from the serum glycoproteins in the serum from a patient or subject in a first step followed by an incubation step of the serum glycoproteins with a fluorescently labeled alpha toxin in a plate assay which specifically binds to GPI-APs which are exclusively produced or hyperproduced in cancer cells. In many instances, the GPI-APs do not have to be separated and the serum glycoproteins may be measured directly in an assay to determine the existence and concentration of the relevant GPI-APs. The assay will bind to those GPI-APs which are indicative of the existence of cancer cells (they are exclusively produced or hyperproduced by cancer cells) in the patient and the fluorescent signal which is produced from the exposure of the serum glycoproteins to the fluorescently labeled alpha toxin may be compared to a predetermined value as otherwise described herein wherein a fluorescent signal above or below a predetermined value is indicative of the existence or absence of cancer cells in the patient or subject. Further variations on this assay will be readily apparent to those of ordinary skill in the art. Immunoassays that utilize alpha toxin for detection and/or capture of the GPI-APs to be identified and measured in combination with antibodies (especially monoclonal antibodies) which are specific for a particular GPI-AP (in the case of breast cancer FERMT3/Kindlin3 and/or FilamenA FLNA GPI-APs) can provide immunoassays (e.g. ELISA or bead based assays) which can be used as an immunoassay using a biological sample, especially including a serum based immunoassay for the detection of particular cancer cells in patient or subject and a diagnosis of cancer.

Numerous polyclonal and/or monoclonal antibodies to FERMT3/Kindlin 3 and FilamenA (FLNA) are readily available commercially or can be readily produced from the peptide sequence, which is known. Polyclonal and monoclonal antibodies (anti-rabbit, anti-goat and anti-mouse) for FERMT3/Kindlin 3 are readily available commercially from one or more ABnova, Taipei, Taiwan, ProSci, ProSci., Inc. San Diego, Calif., USA, Thermo Fisher Scientific Pierce Antibodies, Waltham Mass., USA, from Santa Cruz Biotechnology, Inc., Santa Cruz, Calif., USA, as well as described in Swenseon, et al., Nature Medicine, 2009, March 15 (3) 306-312. In the case of polyclonal and/or monoclonal antibodies (rabbit, mouse and goat) to FilamenA (FLNA), 108 FilamenA antibodies are available from 17 suppliers, including EMD Millipore, Billerica, Mass., USA, Lifespan Biosciences, Inc., Seattle, Wash., AMS Biotechnology, LLC, Lake Forest, Calif., USA, Acris Antibodies, Inc., San Diego, Calif., USA, Atlas Antibodies, Stockholm Sweden and ThermoFisher Scientific, Pierce Antibodies, Waltham Mass., USA, among numerous others. These antibodies are commercially available and may be readily adapted to provide immunoassays for breast cancer based upon the measurement of FERMT3/kindling 3 and/or FilamenA (FLNA) in the serum of patients suspected of having breast cancer.

The term “immunoassay” is used to describe well-known biochemical tests that measure the presence or concentration of a substance (generally, referred to as the “analyte”) in solutions that frequently contain a complex mixture of substances. Analytes in blood serum and urine are assayed using immunoassay methods. In essence, the method depends upon the fact that the analyte in question is known to undergo a unique immune reaction with a second substance, which is used to determine the presence and amount of the analyte. This type of reaction involves the binding of one type of molecule, the peptide or antigen with a second type, in the present invention, alpha toxin and/or an antibody Immunoassays can be carried out using either the antigen or the antibody in order to test for the other member of the antigen/antibody pair, however in the present invention, serum containing GPI-APs (as antigen) are presented to the alpha toxin and/or antibody. For GPI-AP peptide analytes pursuant to the present invention, an antibody that specifically binds to a particular GPI-AP which is biomarker for a disease state, in particular, cancer, is prepared or obtained for use as an analytical reagent. The specificity of the assay depends on the degree to which the analytical reagent is able to bind to its specific binding partner to the exclusion of all other substances that might be present in the sample to be analyzed. In addition to the need for specificity, a binding partner must be selected that has a sufficiently high affinity for the analyte to permit an accurate measurement. The affinity requirements depend on the particular assay format that is used.

In addition to binding specificity, the other key feature of immunoassays is a means to produce a measurable signal in response to a specific binding. Historically this was accomplished by measuring a change in some physical characteristic such as light scattering or changes in refractive index. With modern instrumentation such methods are again becoming increasingly popular. Most immunoassays depend on the use of an analytical reagent that is associated with a detectable label. A large variety of labels have been demonstrated including radioactive elements used in radioimmunoassay, enzymes, coenzymes, fluorescent labels, phosphorescent, and chemiluminescent dyes, among numerous others. Such labels serve for detection and quantitation of binding events either after separating free and bound labeled reagents or by designing the system in such a way that a binding event effects a change in the signal produced by the label Immunoassays requiring a separation step, often called separation immunoassays or heterogeneous immunoassays, are popular because they are easy to design, but they frequently require multiple steps including careful washing of a surface onto which the labeled reagent has bound Immunoassays in which the signal is affected by binding can often be run without a separation step. Such assays can frequently be carried out simply by mixing the reagents with sample and making a physical measurement. Such assays are called homogenous immunoassays or less frequently non-separation immunoassays.

Regardless of the method used, interpretation of the signal produced in an immunoassay requires reference to a calibrator that mimics the characteristics of the sample medium. For qualitative assays the calibrators may consist of a negative sample with no analyte and a positive sample having the lowest concentration of the analyte that is considered detectable. Quantitative assays require additional calibrators with known analyte concentrations. Comparison of the assay response of a real sample to the assay responses produced by the calibrators makes it possible to interpret the signal strength in terms of the presence or concentration of analyte in the sample.

The term “label” is used to describe a component which is introduced onto alpha toxin or an antibody as otherwise described herein, in order to function as a way of separating and/or detecting GPI-APs in a sample. Labels which find use according to the present invention include radioactive elements used in radioimmunoassay, enzymes, coenzymes, fluorescent labels, including fluorescent peptides, phosphorescent, and chemiluminescent dyes, among numerous others. In the present invention preferred labels including complexing agent (e.g., biotin which complexes with streptavidin for purposes of isolating GPI-APs from samples), enzymes, especially in ELISA assays (where alpha toxin and/or antibody, preferably a monoclonal antibody which is which may be linked to an agent, which when acted upon by the enzyme converts to a compound which produces color for use in a colorimetric assay (e.g., peroxidase action on an appropriate substrate such as ABTS or 3,3′,5,5′-tetramethylbenzidine to produce a colorimetric signal which can be compared to a predetermined signal or value) or a fluorescent agent which provides a fluorescent signal which can be measured and the quantified to provide a direct indication of analyte (GPI-AP) binding to an antibody and/or alpha toxin.

Fluorescent labels for use in the present invention may be linked to alpha toxin and/or a polyclonal or monoclonal antibody through use of a convention linker, a large number of which are known in the art. Linkers are generally bifunctional agents which can link a moiety from a polypeptide (such as alpha toxin) to a label and include such linkers as Exemplary fluorescent agents which may be used as labels pursuant to the present invention include, for example, Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester (532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moities which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids as chimeric proteins (especially in the case of alpha toxin) and include polypeptide reporters such as fluorescent green protein and fluorescent red protein. Use of more than one fluorescent label in immunoassays according to the present invention in a multiplexing approach for identifying the presence and quantity of more than one type of GPI-AP in a sample (such as a blood, serum, urine or other biological fluid test) for cancer, especially breast cancer which identifies and quantifies the amount of FERMT3/Kindling3 and/or FilamenA (FLNA) to determine the presence of breast cancer cells in a patient) to determine the existence of a disease state such as cancer in a patient represents another embodiment of the present invention.

The term “phospholipase” is used to describe a phospholipase enzyme (preferably, phospholipase C or GPI specific phospholipase D) which may be used to isolate (cleave) GPI-AP from the cell membranes to which they are attached. These enzymes are readily available commercially and phospholipase enzymes from a number of different species may be employed during processing and isolation of GPI-APs from cell membranes in processing and identifying GPI-APs according to the present invention.

The term “proteomics” is used to describe the large-scale study of proteins, in particular, their structures and their functions which relies on protein purification and mass spectrometry to provide details as to identity (when known) and structure (when known or unknown) of proteins which are identified.

In the present invention, glycoproteomic identification of GPI anchored proteins from tissues or cells is performed with a critical step of complexing alpha toxin to the GPI anchor protein. In an exemplary method of the present invention, membrane fractions (e.g., 600 μg) isolated from 100 mg of cells or tissue are combined with 10 μg biotin labeled alpha toxin at 4 C overnight in 1×PBS. GPI anchored glycoproteins bound to alpha toxin labeled with biotin are captured using streptavidin magnetic beads. Proteins are eluted using urea, reduced with DTT, and carboxyamidomethylated with iodoacetamide prior to trypsin digestion (to reduce all disulfide linkages to SH residues (cysteine). Tryptic peptides are acidified and desalted on micro C18 spin columns. Peptides are dried and resuspended in Buffer A (0.1% formic acid) 19.5 μL and Buffer B (80% acetonitrile/0.1% formic acid) 0.5 μL and filtered through a 0.2 micron filter. Samples are nitrogen bomb loaded into capillary C18 columns and eluted via a linear gradient of increasing Buffer B over 160 minutes directly into a linear ion trap mass spectrometer (LTQ XL ETD, Thermo). The top eight ions from the full MS (300-2000 m/z) were selected from MS/MS in CID at 34% normalized collision energy with a dynamic exclusion of 2 repeat counts for 30 seconds. Raw spectra are converted to mzData format and searched against the International Protein Index database using Myrimatch software. Proteins (minimum of 2 peptides assigned were grouped are grouped using IDPicker software. Tumor-specific GPI anchored proteins are selected by comparison against patient matched normal tissue or non-transformed cells analyzed. See also, Mehlert and Ferguson, Glyconate Journal, 2009 November; 26(8): 915-921.

In addition to GPI-APs, proteins released from the GPI groups may also be analyzed by mass spectrometry as described above. In particular, proteins may be released from GPI-APs using methods for cleaving off the GPI group from the peptides which are covalently linked. By way of example, GPI glycan extraction generally follows alpha toxin capture from tissues, cells, or serum. In this approach, a minimum amount (e.g. 20 mg) of captured GPI anchored proteins are applied to polyvinylidene fluoride (PVDF) membrane. The membrane is excised and placed in a microcentrifuge tube and covered with ice-cold hydrofluoric acid (e.g., 48% HF) for a sufficient period of time at low temperature (e.g., 48 hours at −20 C). The released GPI glycans are removed from the PVDF and placed in a new tube with 1 mL water prior to lyophilization of the glycans. Water is added again and the lyophilization is repeated. Glycans are labeled using permethylation techniques to introduce methyl group substitutions enabling enhanced ionization in LTQ-FT-MS/MS detection and analyzed according to the above-described method. Spectra are manually interpreted to determine glycan composition and linkage.

The following examples are provided to further describe various aspects of the present invention. The examples should not be taken to limit the scope of the present invention in any way.

### Example 1

### Method

Alpha toxin was expressed and purified from the PET22 vector using E. Coli BL21 cells. The purified alpha toxin was labeled with biotin using the Pierce EZ-link sulfo-NHS-LC Biotin labeling reagent according to the manufacturers instructions. Membrane proteins were extracted from the invasive breast cancer cell line MDAMB231 using a triton X-114 phase separation protocol. Fractions of proteins from the extraction were sampled as follows: pre-PI-PLC fraction contained all membrane proteins solubilized in SDS-loading buffer, PI-PLC released containing the proteins that were released into the soluble phase after PI-PLC enzyme incubation, the post-PI-PLC fraction contained membrane proteins remaining that were solubilized in SDS-loading buffer. Proteins corresponding to twenty micrograms per fraction were separated by polyacrylamide gel electrophoresis on 4-12% Bis Tris gel. The gel was stained with Sypro-Ruby stain and imaged under a UV light source. Proteins on a duplicate gel were transferred to PVDF membrane for 1 hour at 25 volts. The membrane was blocked for 1 hour in 5% non-fat milk in 1×TBST buffer. The membrane was then washed 3×10 minutes each in 1×TBST buffer. The rabbit polyclonal CRD-antibody (Glyko) which recognizes certain PI-PLC cleaved proteins was diluted 1:40 in 1×TBST and incubated with the membrane for 1 hour at room temperature. After washing the membrane 3×5 minutes in 1×TBST the CRD reactive bands were detected using anti-rabbit IgG HRP conjugated secondary antibody followed by enhanced chemiluminescent detection. The signal was allowed to decay and the blot was then incubated with the biotin-labeled alpha toxin (0.1 mg/ml) diluted in 1×TBST for 1 hour. After washing as described above, the membrane was incubated in streptavidin conjugated to HRP. The membrane was washed and the bound streptavidin HRP was detected by enhanced chemiluminescent detection.

### Results:

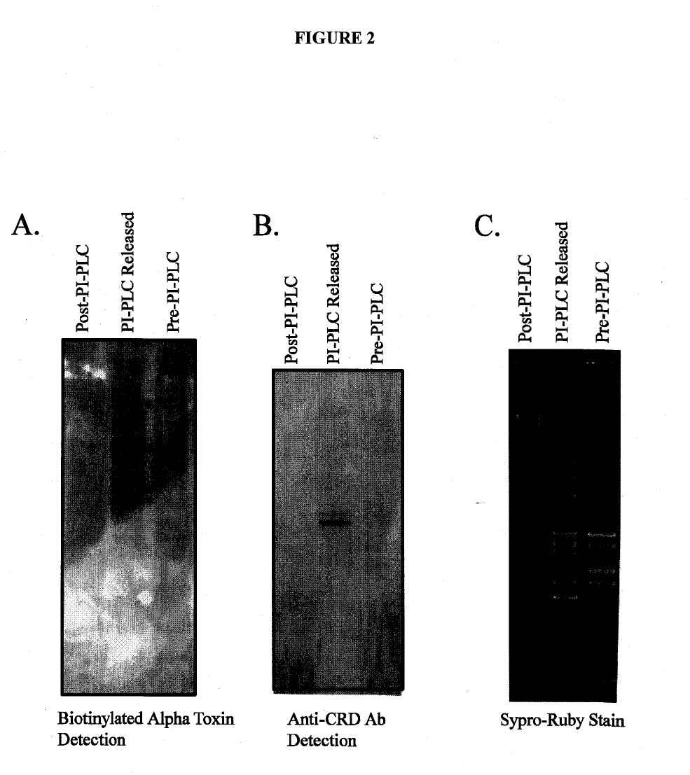

The PI-PLC digestion released GPI anchored proteins into the soluble phase as evidenced by the reactivity of the anti-CRD antibody shown in FIG. 2B. The biotinylated alpha toxin reacted with more PI-PLC released proteins than the anti-CRD antibody, indicating a higher sensitivity. Several bands are concentrated in the PI-PLC released fraction compared with the pre-PI-PLC lane shown by the asterisk in FIG. 2A (compare the pre-PI-PLC lane with the PI-PLC released lane). Equivalent amounts of protein were loaded into each lane as evidenced by the equal staining of various bands in the sypro ruby stained gel (FIG. 2C). We are in the process of identifying the alpha toxin reactive bands by MS-MS to verify that they are either known GPI anchored proteins or predicted GPI anchored proteins.

### Application:

The following uses are shown or flow from the experimental results provided: the use of biotinylated alpha toxin to capture GPI anchored proteins from a variety of sources such as cells, tissues, and serum; the enrichment of GPI anchored proteins by alpha toxin can facilitate protein identification by MS/MS leading to the discovery of novel markers for diseases such as breast cancer. The specificity of the toxin for GPI glycans can be useful in a sandwich assay as a diagnostic tool or in a therapeutic approaches targeting tumor cells for chemotherapy.

### Example 2

The development of techniques to capture and enrich GPI-APs released from mammalian cells would greatly enhance the characterization of human GPI-APs. One very promising methodology is the use of carbohydrate binding proteins known as lectins. Toxins such as aerolysin from A. hydrophila and alpha toxin from C. septicum have been shown to act as GPI-specific lectins, binding to the conserved glycan core of the GPI anchor. Recent studies have shown that aerolysin requires interactions with the protein N-glycan as well as the GPI core [13]. Studies using alpha toxin from C. Septicum have demonstrated that this protein recognizes with very broad specificity the core GPI anchor structure [4]. Researchers have been utilizing these toxins to discover new CHO cell mutants with defects in GPI biosynthesis. Recently, a mutant form of the alpha toxin from C. Septicum that is non-toxic to cells was shown to have potential value as an agent to differentiate GPI-positive cells from GPI-negative cells for the diagnosis of paroxysmal nocturnal hemoglobinuria [14]. This example presents data indicating that alpha toxin can bind GPI-APs from breast tissue and serum in a tumor-specific manner suggesting that alpha toxin or derivatives of alpha toxin can be useful for the development of diagnostics and therapeutics for breast cancer, among others. There may be other types of cancer that display elevated levels of GPI-APs in serum that can potentially be detected by alpha toxin.

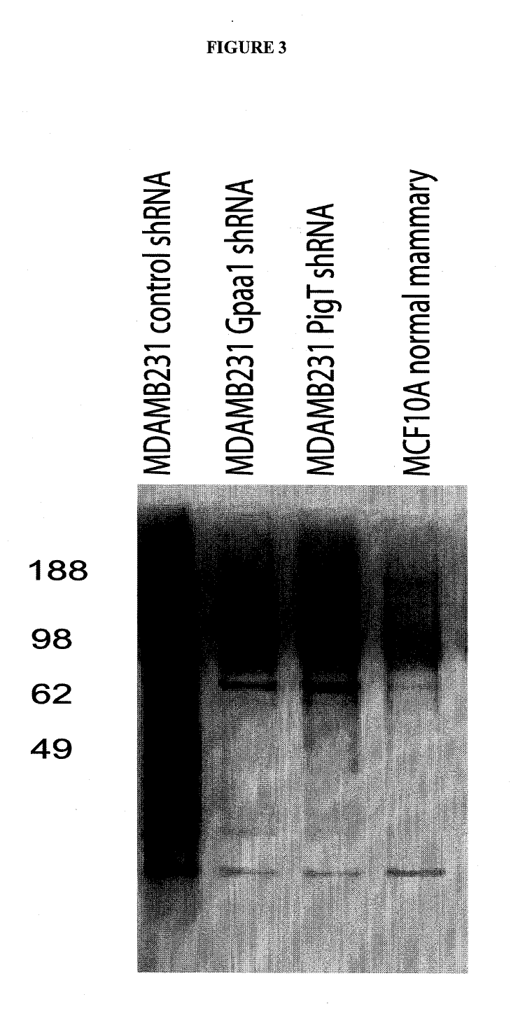

Breast carcinoma cells have elevated expression levels for GPI transamidase subunits due to a gain of chromosomal copy [15] and are therefore predicted to have elevated levels of GPI-APs. We extracted membrane proteins from human breast cancer cells (MDAMB231) or human normal mammary cells (MCF10A) and compared the level of proteins binding to alpha toxin (FIG. 3). Alpha toxin binds many more proteins extracted from the cancer cells compared with the normal mammary cells.

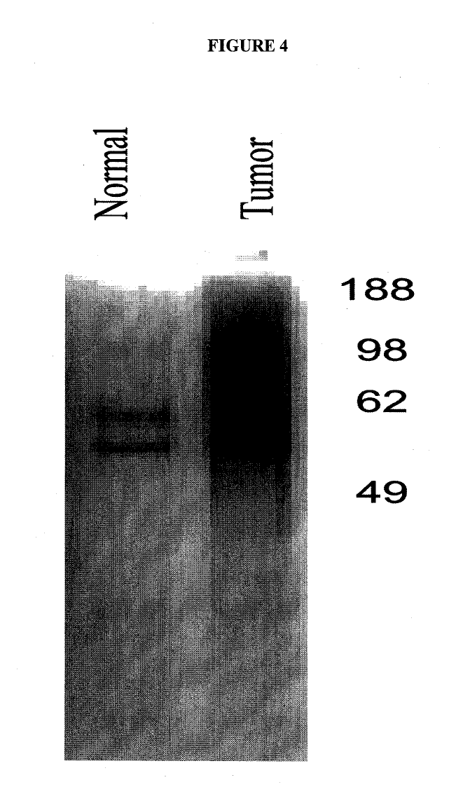

To demonstrate that alpha toxin binding was GPI-AP-specific the inventors employed short hairpin interfering RNA to suppress expression of GPI transamidase subunits Gpaa1 or PigT. Expression of Gpaa1 or PigT shRNA in the MDAMB231 breast cancer cells reduces the levels of GPI anchored proteins binding alpha toxin in the breast cancer cell line MDAMB231 back to levels observed for normal mammary cells (FIG. 3). Therefore, alpha toxin is binding to proteins in a GPI-dependent manner. Next, the inventors tested the binding specificity of alpha toxin for breast cancer tissue or matched normal breast tissue. Again, the toxin binds more proteins in the tumor tissue relative to the normal indicating that the level of toxin binding can discriminate cancer tissue from normal tissue (FIG. 4).

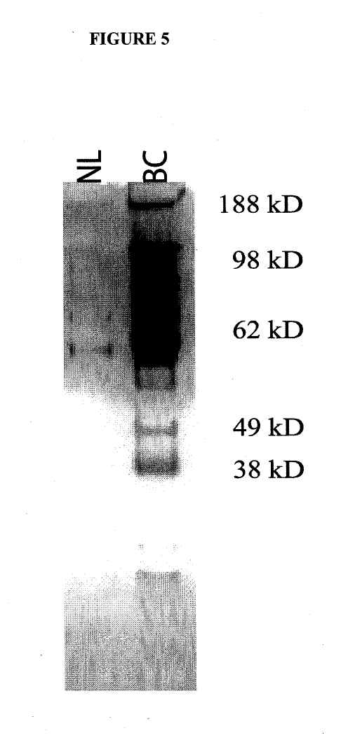

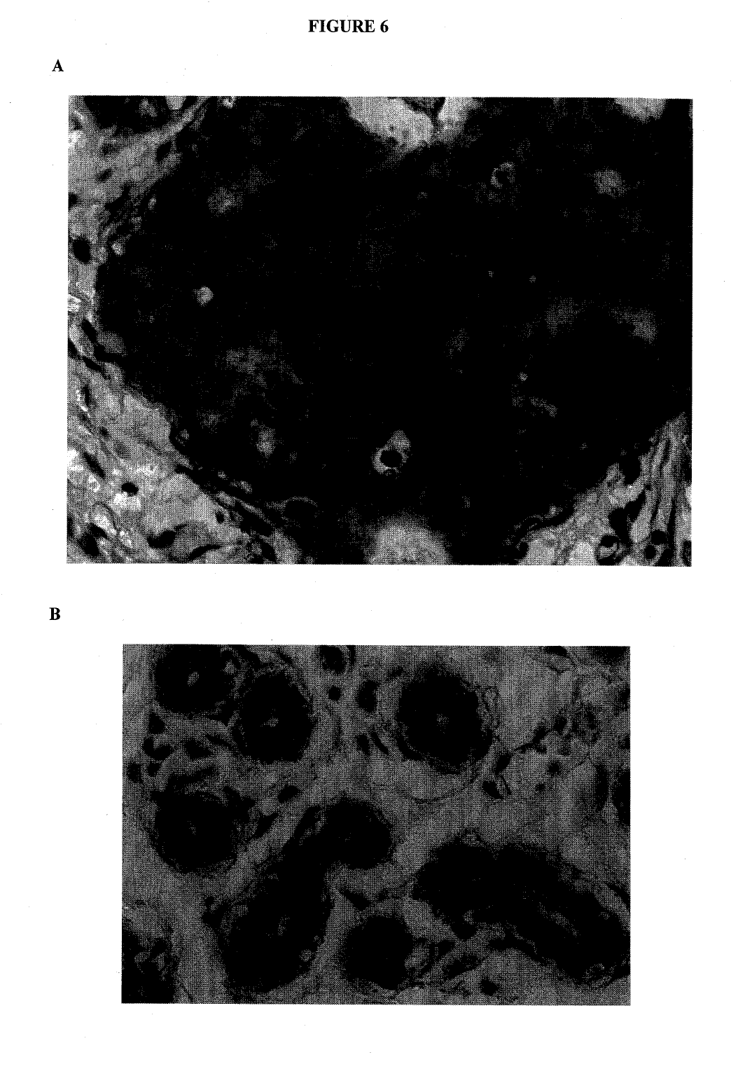

Serum from non-diseased patients or patients with breast carcinoma were pooled (n=5 cases each) and analyzed for the level of protein binding to alpha toxin. Alpha toxin binds significantly more proteins in the serum from breast cancer patients relative to the pooled normal serum (FIG. 5). Therefore, the toxin can be used to discriminate breast cancer serum from normal serum. Biotinylated alpha toxin was used to bind to paraffin embedded tissue sections from ductal invasive breast carcinoma tissue and the matched normal tissue (FIG. 6A and FIG. 6B). Alpha toxin binds to the epithelial breast carcinoma cells in the tumor with high affinity, while normal breast tissue shows low levels of stromal staining. This data demonstrates that alpha toxin can be used to differentiate between normal and malignant breast cancer tissue sections.

### Example 3

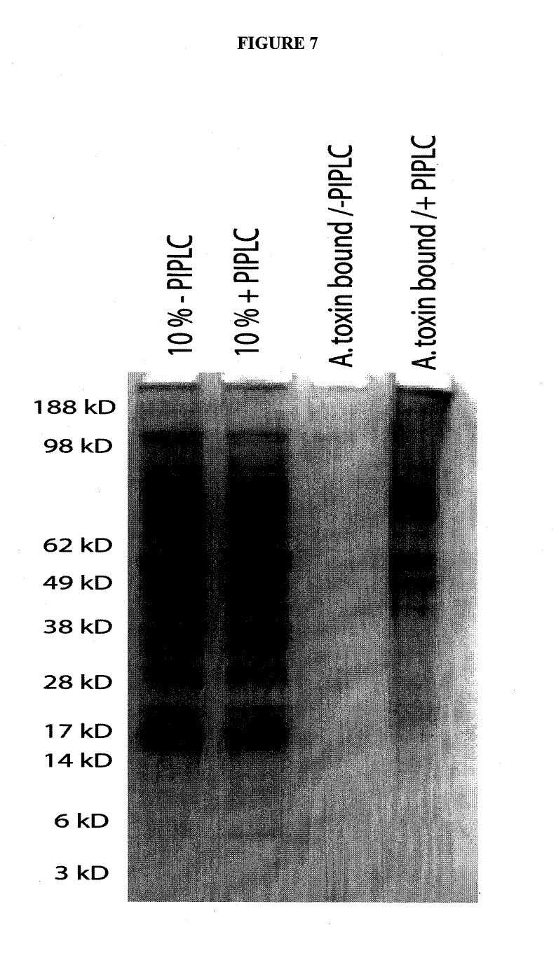

To show that alpha toxin can be used to bind colon cancer GPI-APs, intact LS174T colon cancer cells were incubated with phospholipase-1×PBS (PIPLC) or 1 U/ml PI-PLC in 1×PBS (+PIPLC) for 1 hour at 37 C. Ten percent of the proteins released from the cells were loaded for each sample (lanes 1 and 2, see FIG. 7). The remaining 90% was incubated with alpha toxin beads (lanes 3 and 4 of FIG. 7). The captured proteins were separated on 4-12% Bis-Tris gel before being silver stained. FIG. 7 indicates that alpha toxin can specifically capture phospholipase released GPI anchored proteins. Alpha toxin Selectively captured proteins from the PIPLC treated cells. Equivalent levels of proteins were present in the reactions indicated by the 10% input.

### Examples

### Proteomic Identification of Glycosylphosphatidylinositol (GPI) Anchor-Dependent Membrane Proteins Elevated in Breast Carcinoma

### Experimental Procedures (Second Set of References Applies)

Posttranslational addition of a glycosylphosphatidylinositol anchor (GPI), is performed in eukaryotic cells via the activity of the GPI transamidase (GPIT) (1). GPIT is a multisubunit enzyme complex required for the expression of GPI anchored proteins on the cell surface. GPI anchored proteins are predicted to comprise approximately 1-2% of translated proteins in mammals (2). Several GPI anchored proteins identified to date are tumor antigens such as carcinoembryonic antigen (3), mesothelin (4), prostate specific stem cell antigen (5), and urokinase plasminogen activator receptor (6), suggesting possible roles for this class of proteins in promoting tumorigenesis.

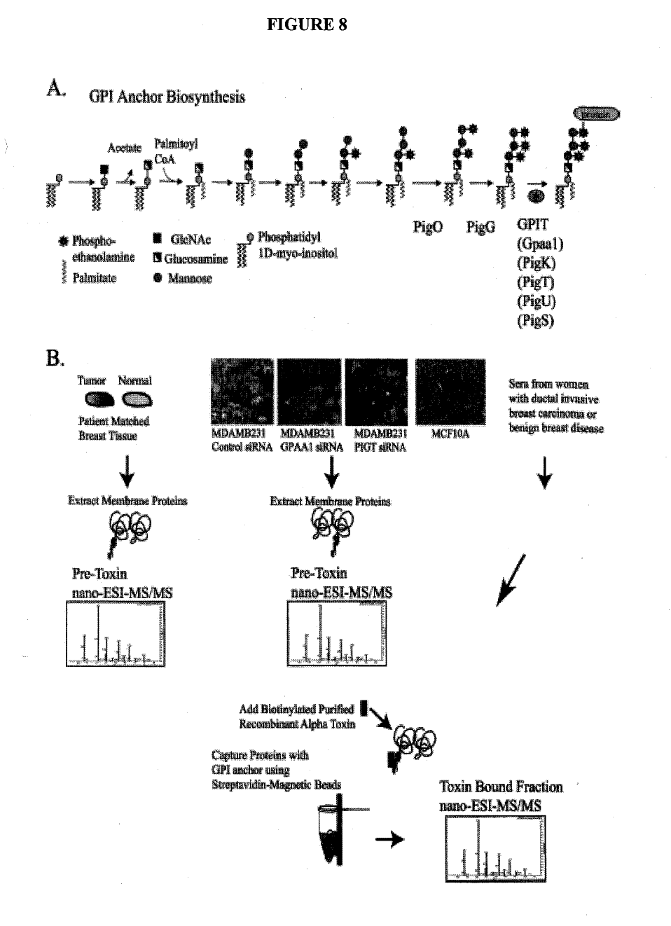

The predictive annotation of GPI anchoring in mammalian protein databases is difficult as there are no common consensus sequences that clearly indicate that a protein will receive a GPI anchor. There are several amino acid features that have been characterized in the C-terminus of proteins that receive the GPI anchor (7). The discovery of these common characteristics led to the development of algorithms to predict the probability of GPI anchor addition such as FragAnchor (8), GPI SOM (9), and Big-PI (10). Furthermore, the experimental isolation and identification of GPI anchored proteins from mammalian cells is often hampered due to the lower expression levels of GPI anchored proteins in many cell types coupled with difficulty in extracting these proteins due to the presence of both lipid and glycan structures (FIG. 8A, core GPI structure). GPI anchored proteins can be released into a soluble form using the bacterial enzyme GPI-specific phospholipase C (PI-PLC) (11). However, certain GPI anchored proteins may be phospholipase C insensitive due to acylation within the GPI anchor (12). In an effort to overcome these obstacles, we are employing the use of the bacterial toxin known as alpha toxin (AT), isolated from Clostridium septicum, to capture and enrich GPI anchored proteins from breast carcinoma for identification by mass spectrometry (See the fractionation scheme in FIG. 8B). AT is a member of the aerolysin-like pore forming toxins that bind with GPI anchored proteins (13). The diversity of GPI anchored proteins that AT can bind with suggests that the binding occurs via the GPI anchor without peptide requirements.

Human breast carcinomas express elevated levels of several GPIT subunits such as GPAA1 (GPI Anchor Attachment Protein 1) and PIGT (GPI Class T) due to gain of chromosome copy number (14). Increased expression of these subunits has been shown to induce tumorigenicity in vitro and in vivo (14). In our study, we document that increased expression of GPIT subunits results in increased levels of GPI anchored proteins in breast cancer epithelial cells evidenced by binding of AT from C. septicum. The inventors isolate and identify proteins binding to AT using nano ESI-RPLC-MS/MS analysis. The data indicate that the membrane abundance of several cell surface receptors that are also found in mesenchymal stem cell populations are dependent on the expression of GPAA1 and PIGT. We report that increased expression of GPAA1 and PIGT positively regulates the expression of the embryonic Forkhead/Fox transcription factor FOXC2. Elevated expression of GPI anchored proteins also increases the expression of several mitochondrial membrane proteins that may promote the growth and survival of breast cancer. The inventors also provide evidence that AT binds with GPI anchored proteins released into serum allowing the capture and detection of potential markers for the detection of breast cancer. Overall, these results indicate that GPI anchored proteins are abundant in breast cancer cells and other cancer cells with functions that promote tumor growth and spread, making these proteins ideal diagnostic and therapeutic targets.

### EXPERIMENTAL PROCEDURES

### Antibodies and Reagents

The following reagents were purchased from Sigma (St. Louis, Mo.): Puromycin, Polybrene, DTT, Iodoacetamide, Immidazole, Urea, and anti-kindlin-3 rabbit polyclonal. All secondary antibodies and the following primary antibodies were purchased from Santa Cruz Biotechnology: anti-ERK, anti-FoxC2, anti-filamin A.

### Specimens and Cell Line Information

Tissue specimens, matched adjacent normal and tumor, from patients with histologically proven invasive ductal breast carcinoma were collected in accordance with approved institutional review board Human Subject's guidelines at Georgia Health Sciences University (GHSU) in Augusta, Ga. and Emory University in Atlanta, Ga. Board certified clinical oncologists and pathologists carried out all clinical and histological analysis of the biopsies. Each tumor contained >75% tumor cells by H and E stain and was of high grade. All specimens for this study were fast frozen at −70° C. until proteomic analysis. Blood from non-diseased and patients with ductal invasive breast carcinoma was collected pre-operatively and in accordance with approved institutional review board Human Subject guidelines at GHSU. Serum fractions were stored at −70° C. The MDAMB231, MCF10A, and 293 cell lines were obtained from the ATCC and cultured as recommended.

### Immunohistochemistry Staining

Paraffin-embedded tissue sections from patient matched tissue sets of normal and invasive ductal breast carcinoma were de-waxed and re-hydrated. Tissues were blocked with 0.5% hydrogen peroxide for 30 minutes. Tissues were rinsed in PBS with 0.2% tween 20 prior to blocking in 2.5% blocking serum (Vector Labs) for 20 minutes. Biotin labeled AT (2 μg/ml) diluted in PBS/0.2% tween 20 was added for 2 hours at room temperature. Following washes in PBS/0.2% tween 20 the tissues were incubated with the ABC reagent (Vector Labs) for 30 minutes. Positive staining was detected using DAB substrate followed by hematoxylin counterstain.

### AT Purification and Labeling

The plasmid pBRS 10 encoding native AT expressing a histidine-tag (15) was transformed into BL21(DE3) pLysS E. coli. Bacteria were grown in 2×YT media supplemented with 100 μg/ml ampicillin and 34 μg/ml chloramphenical at 37° C. overnight. The culture was diluted 20 fold, at 1.0 OD protein expression was induced for 4 hours at room temperature using 0.2 mM IPTG. Bacterial pellets were resuspended in 0.5×PBS with protease inhibitors and lysed using a French Press. AT was purified using Talon cobalt resin (Clontech). Bound toxin was eluted using step immidazole gradients in 25 mM MES pH 6.5 buffer supplemented with 150 mM NaCl. AT fractions were pooled and dialyzed to remove immidazole prior to SP cation-exchange chromatography. AT fractions were concentrated and buffer exchanged into 25 mM MES pH 6.5, 150 mM NaCl before storage at −80° C. AT was dialyzed into 1×PBS pH 9.0 prior to biotin labeling using Sulfo-NHS-LC-Biotin (Pierce) as recommended followed by buffer exchange using a 10,000 MWCO membrane.

### Membrane Protein Extraction/AT Binding/Peptide Preparation.

Tissue (100 mg) or cell pellet (100 mg) was resuspended in 10 mM Hepes pH 7.5 μlus protease inhibitors using a polytron (1 ml volume). The slurry was placed in a glass dounce homogenizer and cells lysed using 10 strokes each of the large and fine pestle. The solution was incubated on ice for 1 hour. Nuclei were removed by transferring to a microcentrifuge tube and centrifuging at 3,000 rpm for 5 minutes. The supernatant was removed to a fresh tube and the centrifugation was repeated twice. The final supernatant was placed in a Beckman ultracentrifuge tube and centrifuged at 100,000×g for 1 hour at 4° C. The pellet containing a total membrane preparation was rinsed in 40 mM ammonium bicarbonate. The pellet was resuspended by sonication in 300 μl of 40 mM ammonium bicarbonate/10 mM DTT and rotated at room temperature for 2 hours to reduce proteins. An equal volume of iodoacetamide (10 mg/ml in 40 mM ammonium bicarbonate) was added and the tubes were vortexed before incubating in the dark for 45 minutes at room temperature. The protein solution was dialyzed overnight at 4° C. into 10 mM ammonium bicarbonate using 4000 MWCO tube-O-dialyzer (G-Biosciences). Proteins were dried in the speed vacuum for long term storage at −80° C. or used directly. Membrane proteins were also extracted from the cells using Triton X-114 (16). The detergent fractions were treated with 10 units of PI-PLC (Invitrogen) for 1 hour at 37° C. The aqueous fraction containing the GPI anchored proteins was precipitated with cold acetone. Proteins were resuspended in 1×PBS for binding to alpha toxin as described below for the total membrane extractions. Digest containing 60 μg of membrane proteins was prepared with 5 μg of sequencing grade modified trypsin (Promega) for sample analysis. Dried proteins or proteins PI-PLC released from Triton X-114 extractions were resuspended in 1×PBS by sonication. A protein assay was performed and 600 μg were incubated with 10 μg of biotinylated AT overnight at 4° C. For the analysis of serum samples, 10 μL of patient serum was incubated with 10 μg of biotinylated AT overnight at 4° C. in a 300 microliter volume of 1×PBS. Bound toxin reactive proteins were captured using 100 μl paramagnetic streptavidin particles (Promega) at 4° C. for 2 hours. After washing in 1×PBS, captured proteins were eluted with 200 μl of 4M Urea/4 mM DTT/40 mM ammonium bicarbonate at 52° C. for 1 hour. The eluted fraction was separated from the paramagnetic streptavidin particles using a magnetic stand. Alpha toxin bound proteins from Triton X-114 extractions were separated on 4-12% polyacrylamide gels and each sample lane was cut into 6 gel slices. Proteins were reduced, carboxyamidomethylated, and released using standard in-gel trypsin digest protocols. Proteins eluted from streptavidin beads from the total membrane isolation protocol were digested with 5 μg of sequencing grade trypsin at 37° C. overnight. Tryptic peptides were acidified with 200 μl of 1% trifluoroacetic acid and desalting was performed using C18 spin columns (Vydac Silica C18, The Nest Group, Inc.). Peptides were dried in the speed vac and resuspended in 19.5 μl buffer A (0.1% formic acid) and 0.5 μl of buffer B (80% acetonitrile/0.1% formic acid) and filtered through a 0.2 μm filter (nanosep, PALL). Samples were loaded off-line onto a nanospray column/emitter (75 μm×13.5 cm, New Objective) self-packed with C18 reverse-phase resin in a nitrogen pressure bomb for 10 minutes. Peptides were eluted via a 160-minute linear gradient of increasing B at a flow rate of approximately 250 nl/min. directly into a linear ion trap mass spectrometer (LTQ XL ETD, Thermo Fisher Scientific, San Jose, Calif.) equipped with a nanoelectrospray ion source). The top eight ions from the full MS (300-2000 m/z) were selected for MS/MS in CID at 34% normalized collision energy with a dynamic exclusion of 2 repeat counts at 30 seconds duration.

### Proteomic Data Analysis

The raw peptide data was converted to mzXML using ReAdW, a software written at the Institute for Systems Biology in Seattle, Wash. (www.systemsbiology.org). MS/MS spectra were searched against the International Protein Index (IPI) human sequence database (IPI.HUMAN.v.3.71) using MyriMatch (17). The MyriMatch search criteria included only tryptic peptides, all cysteines were presumed carboxyamidomethylated, and methionines were allowed to be oxidized. MyriMatch searches allowed a precursor error of up to 1.25 m/z and a fragment ion limit of within 0.5 m/z. All ambiguous identifications that matched to multiple peptide sequences were excluded. The identified proteins (2 or more peptides assigned) from each individual tumor and normal sample were filtered and grouped using IDPicker software. IDPicker software incorporates searches against a separate reverse database, probability match obtained from MyriMatch, and DeltCN scores to achieve false discovery rates of <5%. Information about IDPicker tools can be found at http://www.mc.vanderbilt.edu/msrc/bioinformatics/.

### Biological Function Annotation

Proteins (Defined by 2 or More Peptides Assigned in at Least 2 biological samples) binding to AT were converted to gene symbols and uploaded to DAVID 2009 (the Database for Annotation, Visualization and Integrated Discovery) for analysis.

RNA interference Constructs

RNA interference (RNAi) target sequences were chosen using the Oligoengine software. Two target sequences for each gene were tested and the following sequences were chosen based on >80% knockdown of mRNA following infection in MDAMB231 cells. GPAA1 target: (NM_{—}003801) 5′ TCTTCCTCTACTTGCTCCC 3′ and PIGT target: (NM_{—}015937) 5′ GACACTGACCACTACTTTC 3′ were synthesized in oligos as recommended by the manufacturer for cloning into pSIH-H1shRNA vector (SBI System Biosciences).

### Construction of GPAA1 and PIGT Lentiviral Expression Vectors

Full length GPAA1 and PIGT were PCR amplified using primers with flanking sequence matching the pCDH1-MSCV-MCS-EF1-Puro cDNA expression vector (SBI System Biosciences). PCR products (gel purified) mixed with gel purified NotI/EcoRI cut vector were ligated and transformed using the Cold Fusion Kit (SBI System Biosciences).

### Lentivirus Production and Cell Transduction

Lentivirus was produced by transfection of 293T cells using Lipofectamine 2000 with the following amounts of plasmids: 8 μg envelope (pMD.G), 5 μg lentiviral siRNA vector or lentiviral expression clone, and 8 μg of packaging plasmid. Approximately 5.5 ml of 293T cell suspension (1.2×10⁶) in growth media were seeded onto lipofectamine 2000 complexes (formed in Opti-MEM I media) in 10 cm tissue culture plates. The next day the cells were replenished with fresh media and infectious lentivirus supernatant was collected at 48 hours and 72 hours posttransfection. Polybrene was added to lentiviral supernatants at a final concentration of 8 μg/ml and the virus was placed on cells to be infected. Virus entered cells during centrifugation at 2,500 rpm for 30 minutes at 37° C. Cells were fed with normal growth media between infections. Two rounds of infection were performed prior to selection of infected cells using puromycin 1 μg/ml.

Quantitative RT-PCR

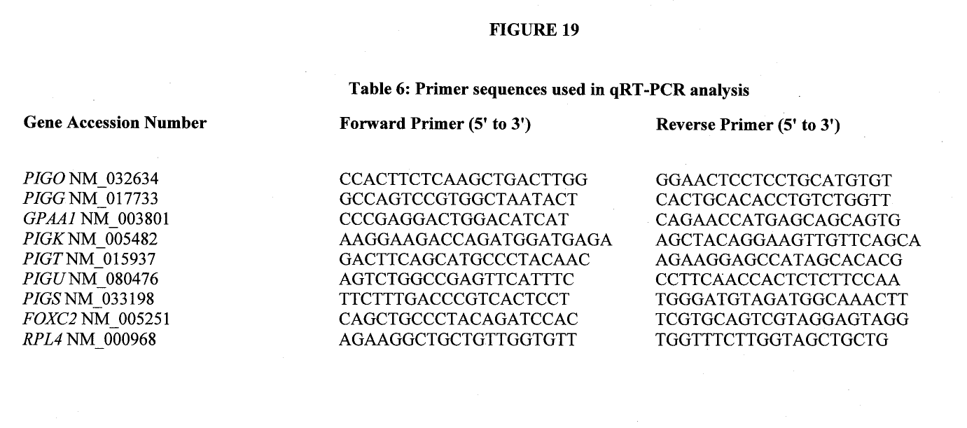

Samples (50 μl packed cells) were extracted using TriZol (Invitrogen, Carlsbad, Calif.) according to the manufactuer's instructions. After DNase treatment, RNA (2 μg) was reverse transcribed using Superscript III (Invitrogen) with random hexamers and Oligo (dT). Primer pairs for assay genes and control genes were designed within a single exon using conditions described (18,19). Primers were validated with respect to primer efficiency and single product detection. Primer sequences are included in Table 6 (FIG. 19). The control gene, Ribosomal Protein L4 (RPL4, NM_{—}024212) was included on each plate to control for run variation and to normalize individual gene expression. Samples were run with negative control templates prepared without reverse transcription to ensure amplification is specific to cDNA. Triplicate Ct values for each gene were averaged and the standard deviation from the mean was calculated. Data was converted to linear values and normalized as described previously (18,19).

### Results

Specificity of AT for GPI Anchored Proteins—

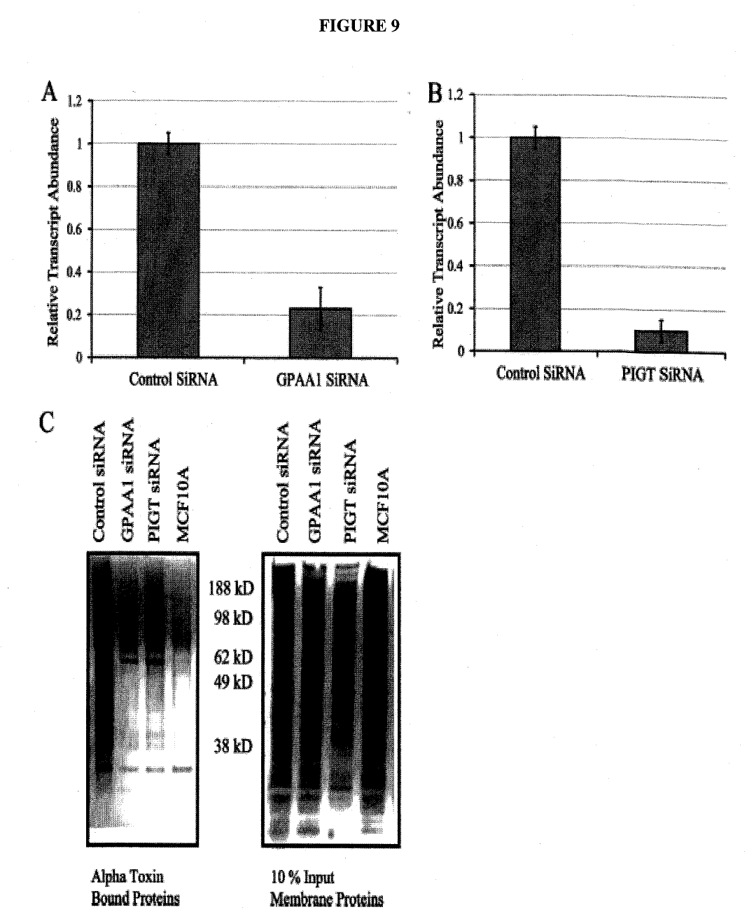

AT has been used previously by researchers to screen for cells that carry mutations of enzymes in the GPI biosynthetic pathway (20). Despite evidence that AT binds to cells that display GPI anchored proteins, in vitro binding of proteins to AT has not been described. Therefore, to establish the specificity of AT binding for GPI anchored proteins in vitro, we created breast cancer cell lines using MDAMB231 breast cancer cells that express a control short interfering RNA that does not target any human genes (control siRNA) or short interfering RNA targeting the GPI anchor attachment protein 1 (GPAA1) and the GPI Class T (PIGT) subunits of the GPIT. The addition of the GPI anchor to the C-terminus of proteins by GPIT is required for the surface expression of GPI anchored protein receptors such as PrP (21). The subunits GPAA1 and PIGT are each essential for the addition of the GPI anchor to proteins by the GPIT (22,23). The MDAMB231 cells expressing short interfering RNA targeting the GPAA1 and PIGT genes reduced the level of mRNA expression for each subunit by at least 80% (FIG. 9A, 9B). We used these cell lines to establish that AT binding to proteins requires the GPI anchor. Membrane proteins from these cells were extracted and used in a binding assay with biotin labeled AT and streptavidin magnetic beads in vitro (See Scheme in FIG. 8B). The silver stained gel shown in FIG. 9C demonstrates that several membrane proteins isolated from MDAMB231 cells expressing control siRNA are bound by AT. However, in MDAMB231 cells expressing siRNA that targets the GPAA1 or PIGT gene there is a dramatic reduction in the levels of membrane proteins binding to the toxin, similar to the low levels observed for non-transformed mammary MCF10A cells. The total protein amounts present in each AT binding reaction were equivalent verified by protein assay and nano-ESI-RPLC-MS/MS analysis prior to AT binding (see FIG. 8B) and a silver stained gel representing 10% of the protein inputs are shown in FIG. 9C. Therefore, we report that AT can be used to bind with extracted membrane proteins in vitro and binding requires the presence of a GPI anchor verified by the dependence of AT binding on GPAA1 and PIGT gene expression.

Elevated Expression Levels of Enzymes in the GPI Biosynthetic Pathway Result in Increased Levels of GPI Anchored Proteins in Breast Carcinoma Cells.

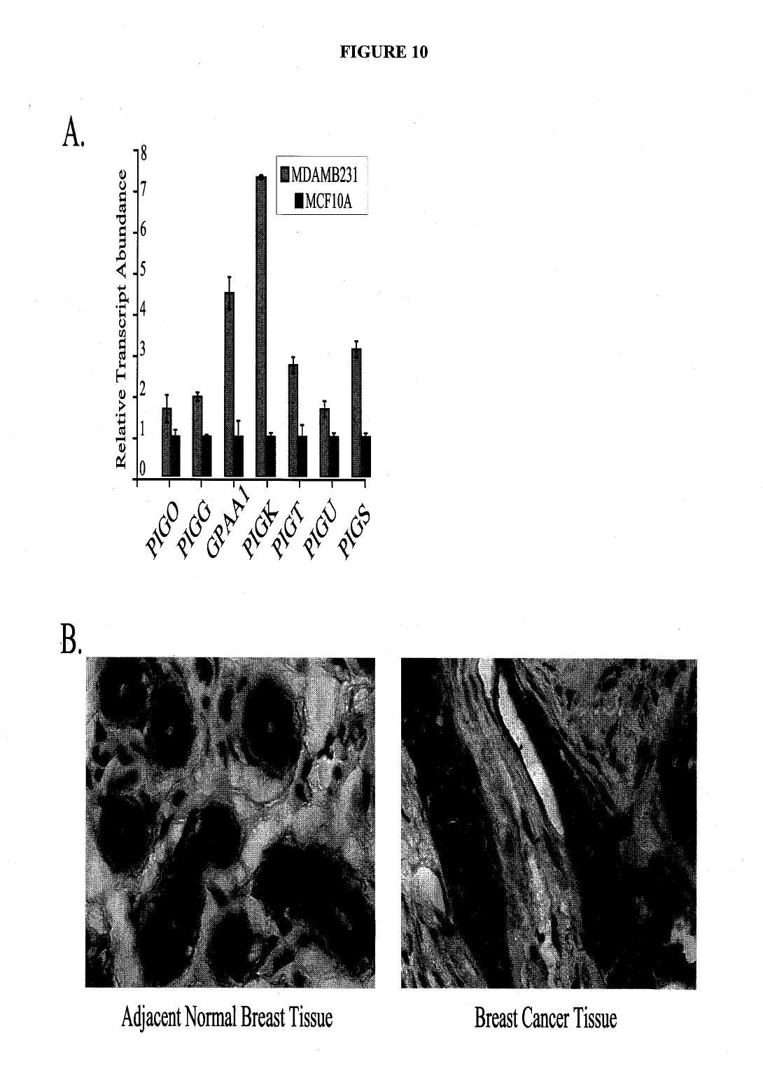

The GPI anchor is assembled by stepwise assembly in the ER membrane prior to addition to the C-terminus of proteins by the GPIT (FIG. 8A, blue asterisk). We have compared the mRNA expression levels for each enzyme in this pathway in malignant MDAMB231 cells and non-transformed mammary MCF10A cells, using quantitative real-time PCR. Our data indicate that the enzymes involved in the last 3 steps of the GPI biosynthetic pathway, additions of phosphoethanolamine by PigO and PigG and enzymes that comprise the GPIT, are significantly increased in breast carcinoma cells relative to non-transformed mammary cells (FIG. 10A). MCF10A expression levels were set to 1 to demonstrate the fold increase of each enzyme in the MDAMB231 cells. The mRNA levels of enzymes participating in the earlier steps of this pathway were not significantly different between MCF10A and MDAMB231 (data not shown).

AT Shows High Levels of Binding to Human Breast Carcinoma Tissue Compared with Adjacent Normal Breast Tissue.

Next, we wanted to determine the cell types that may be expressing GPI anchored proteins in human breast cancer tissue. Therefore, we stained human ductal breast carcinoma and adjacent normal breast tissue with biotin labeled AT. Results shown in FIG. 10B demonstrate that AT binds very weakly to the stromal cell compartment of normal breast tissue (FIG. 10B left), however, the toxin binds with high affinity to malignant breast epithelial cells in the ductal breast carcinoma tissue (FIG. 10B right). These results verify that GPI anchored proteins are increased in breast cancer. This data also reveals that GPI anchored proteins are expressed in cell types different between normal and malignant breast tissue. The stromal cells express low levels of GPI anchored proteins in normal breast; while the epithelial cells are expressing high levels of GPI anchored proteins in the tumor.

Identification of Membrane Proteins Binding AT from Breast Carcinoma Tissue, Serum, and Cells.

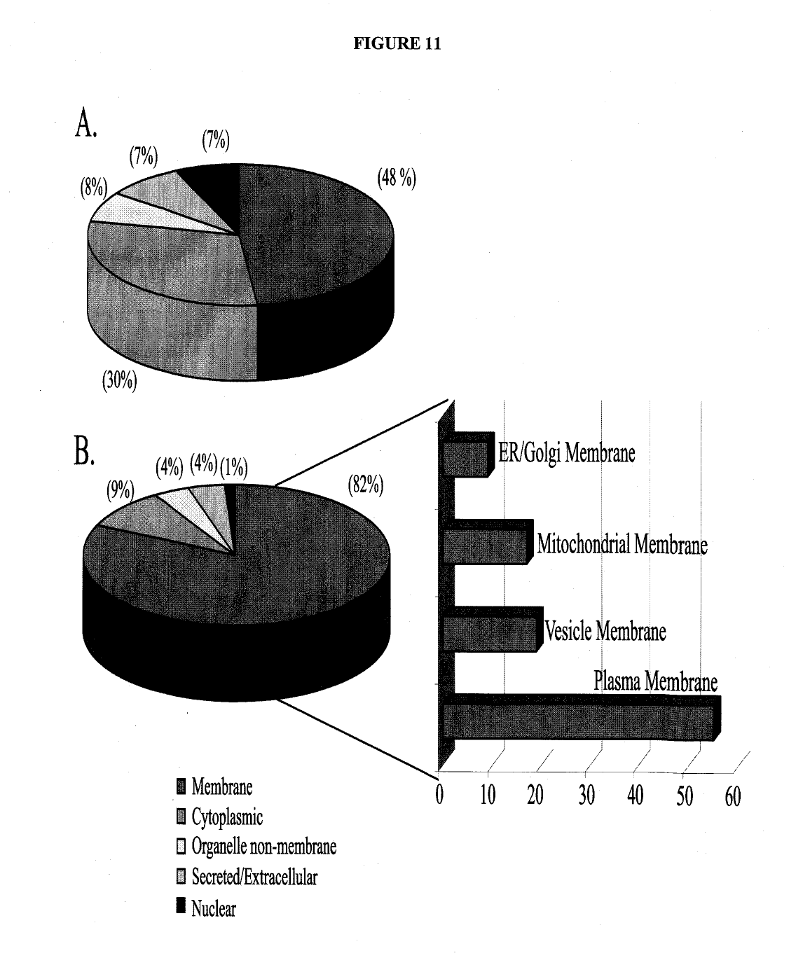

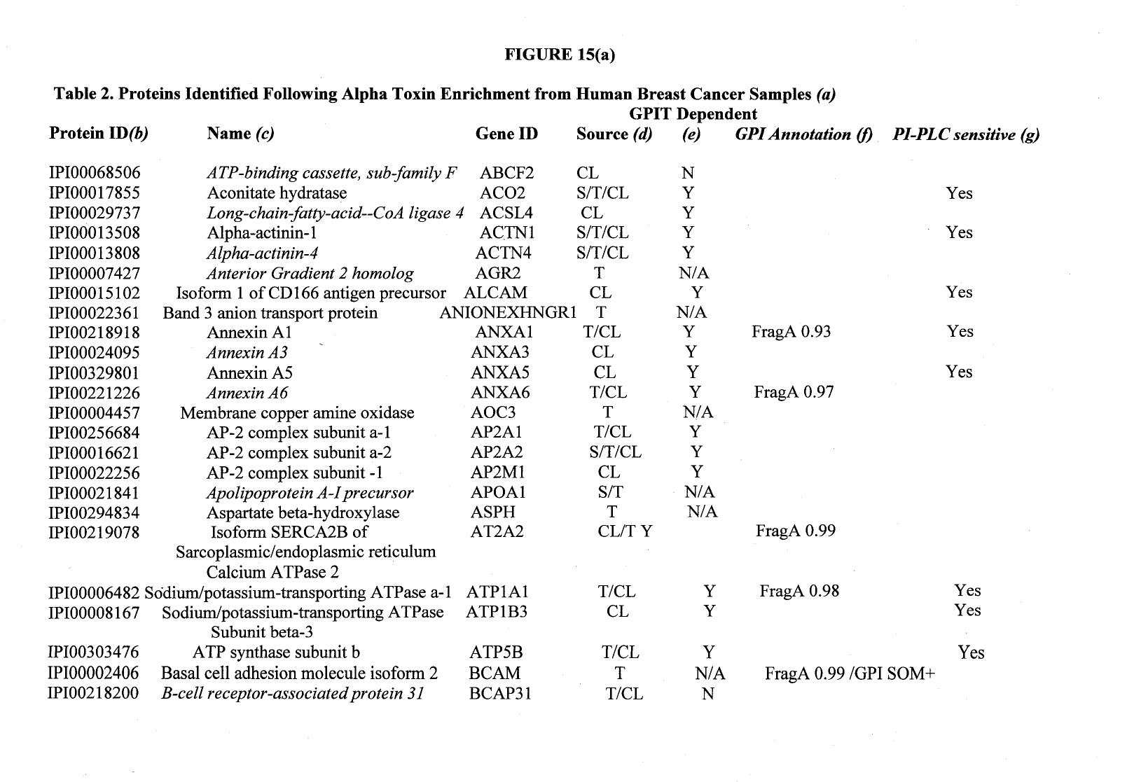

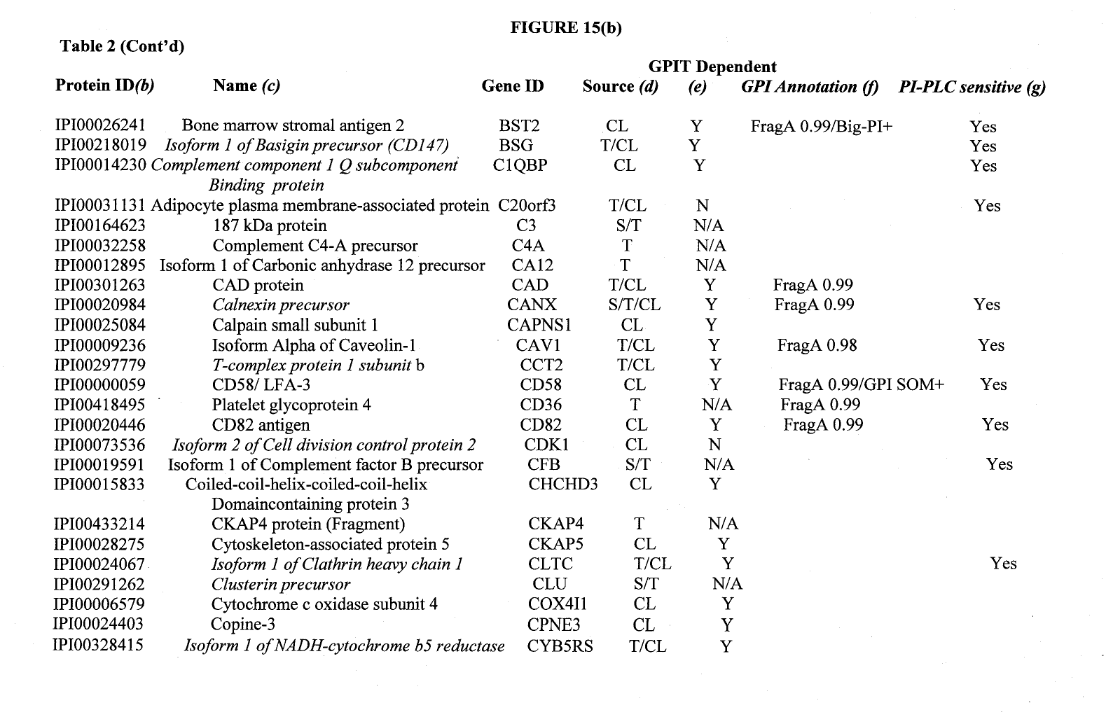

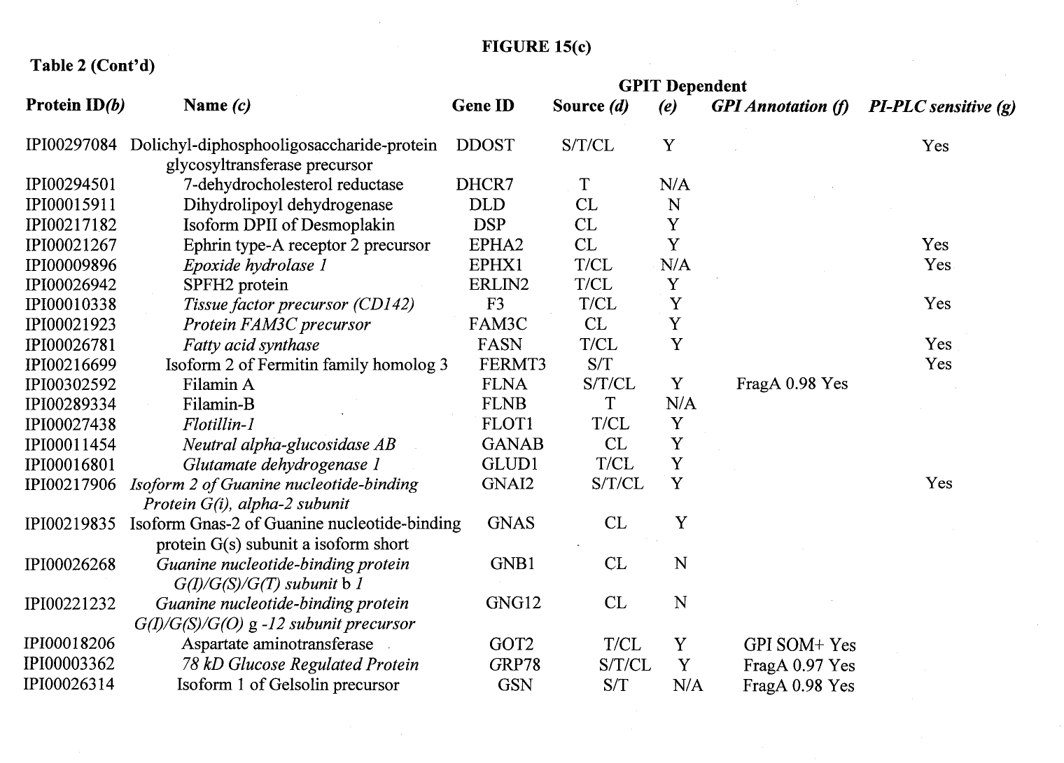

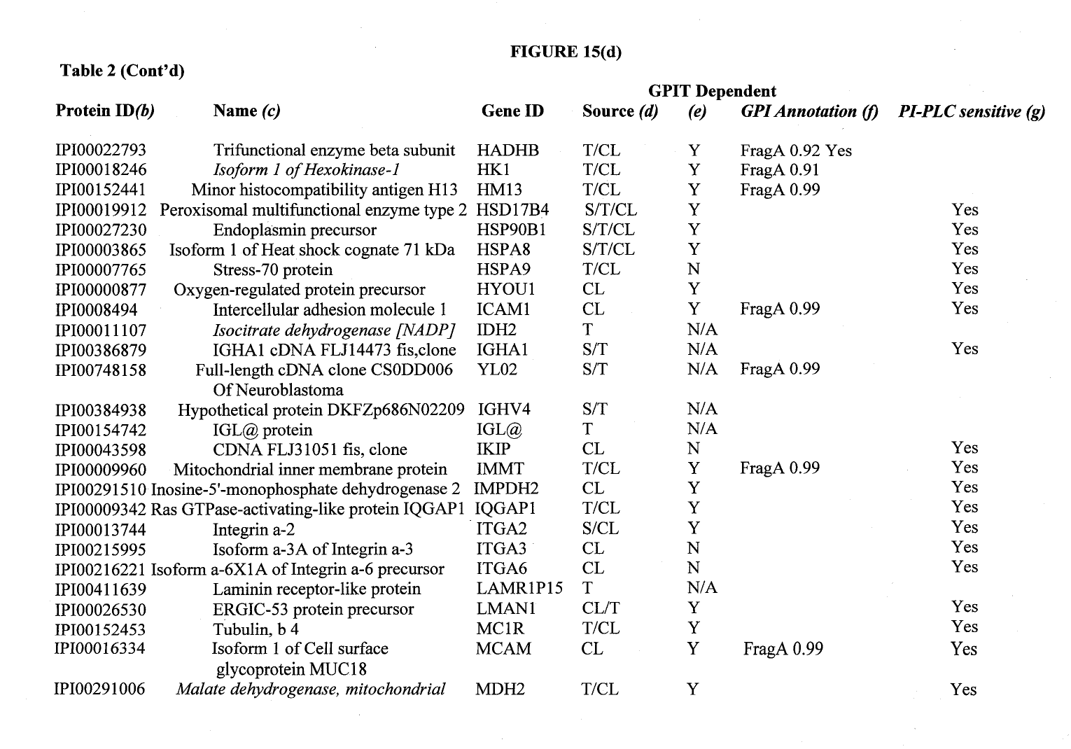

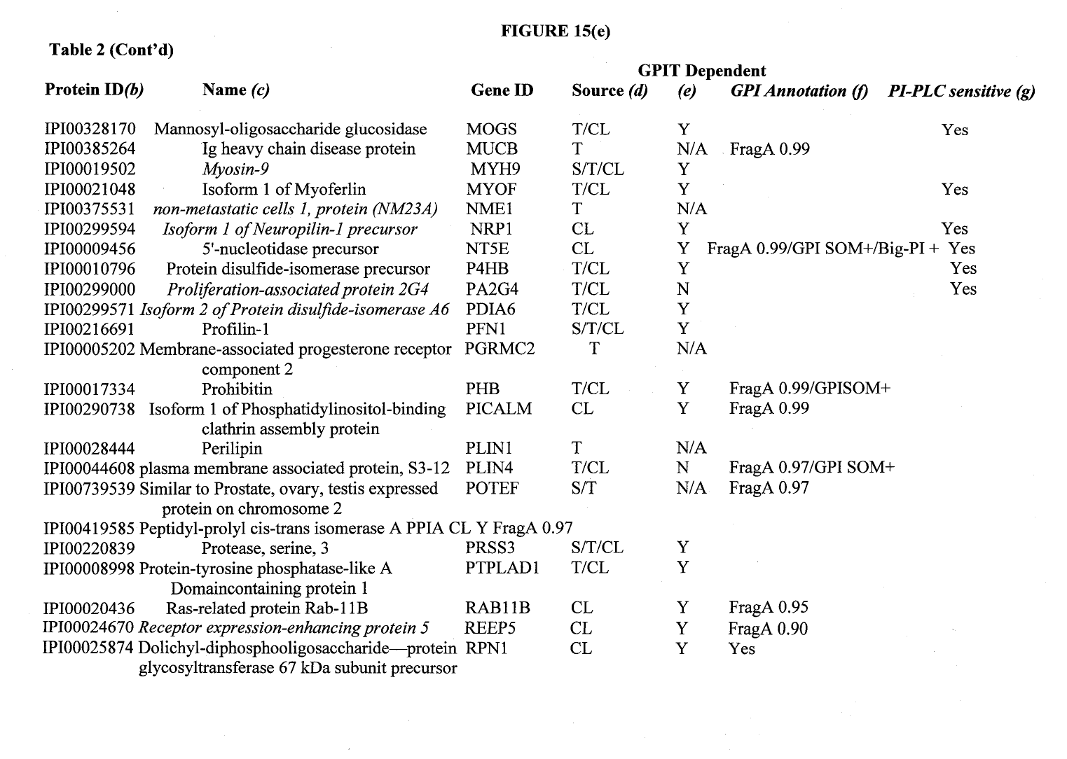

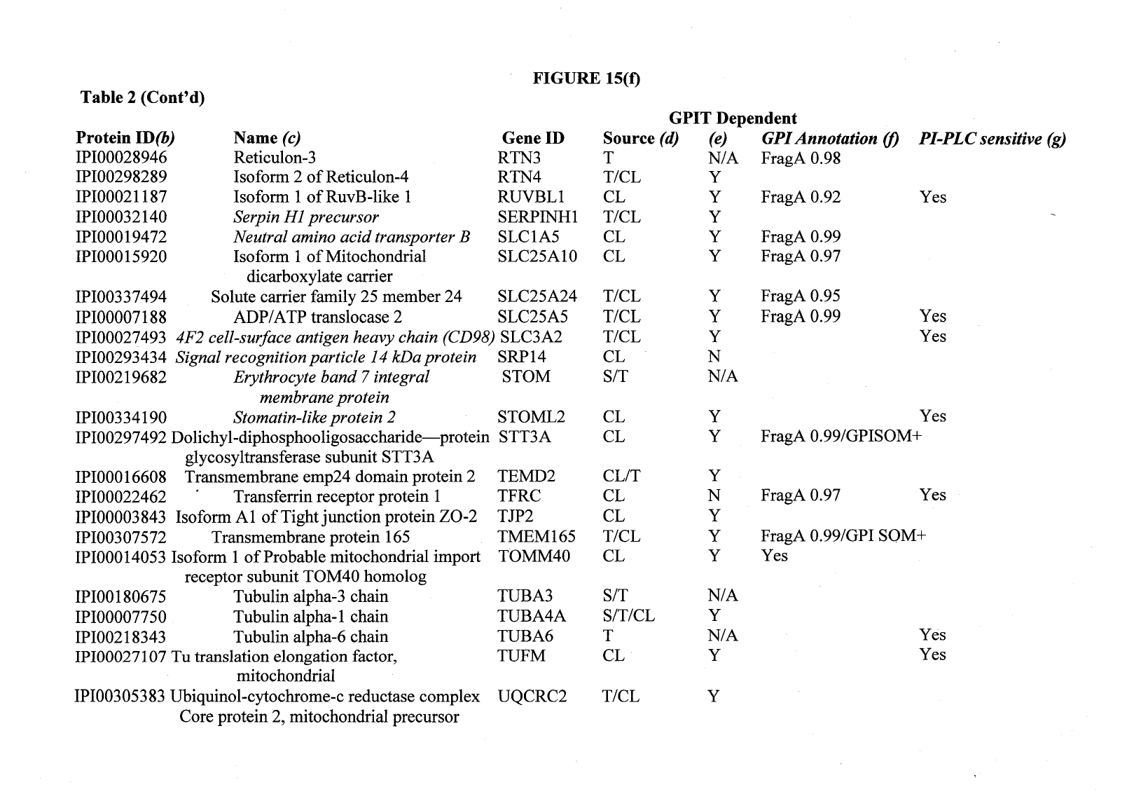

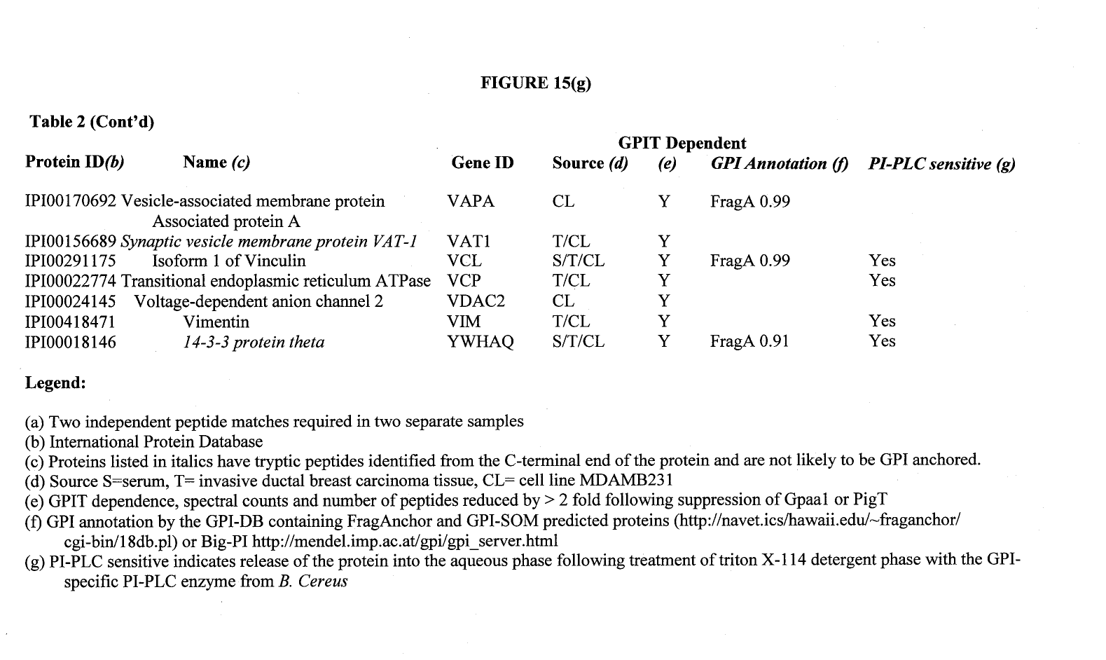

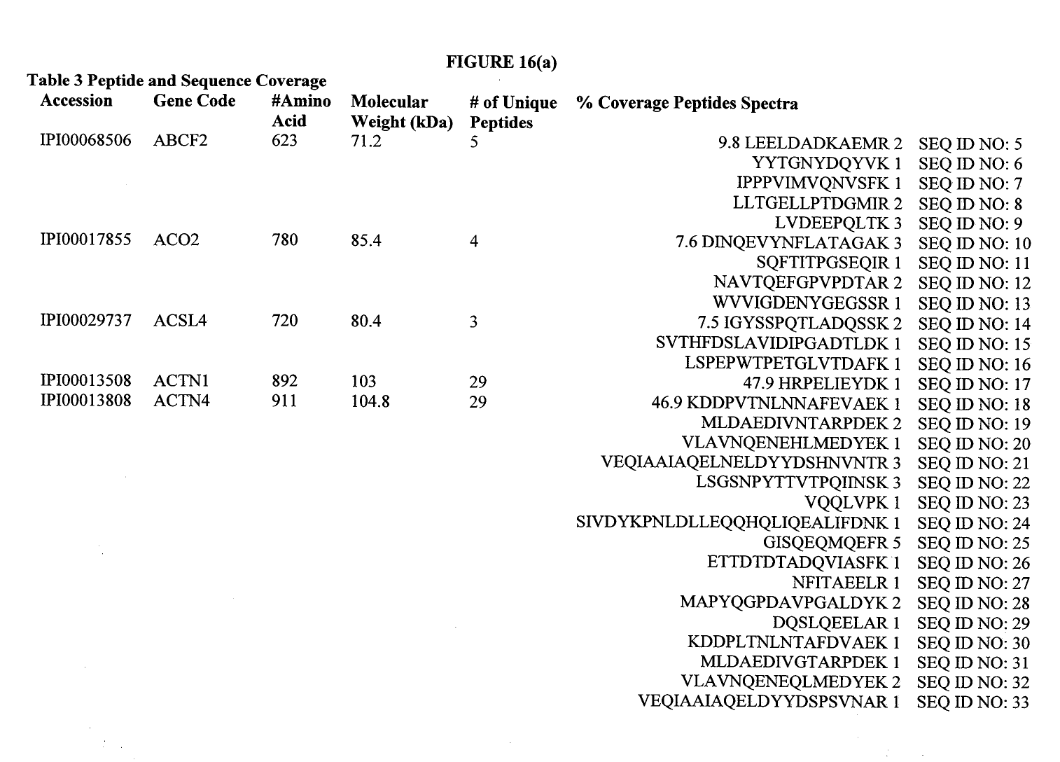

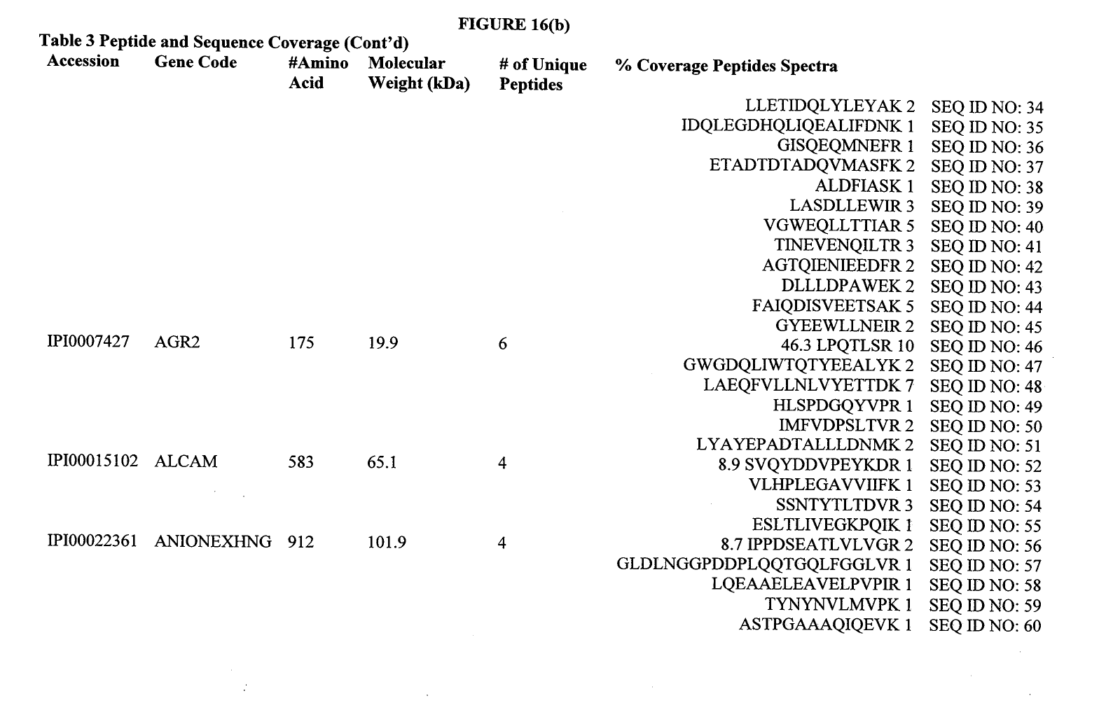

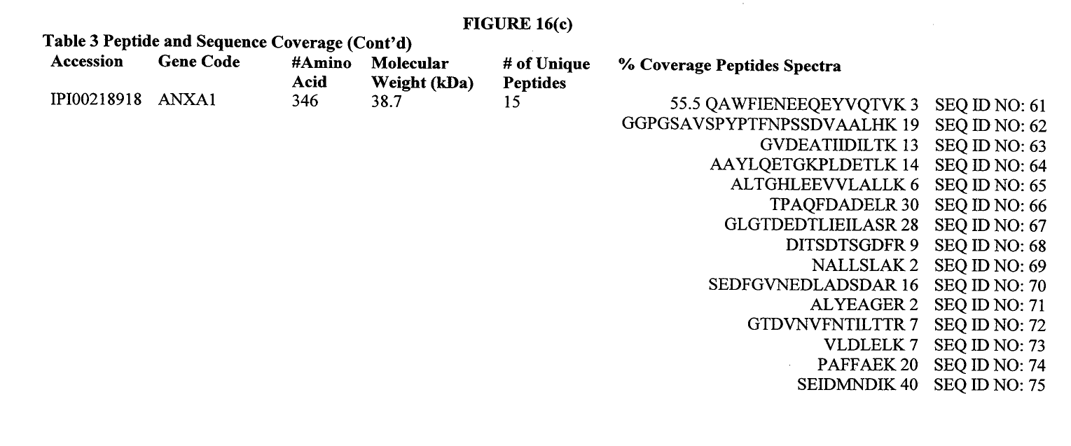

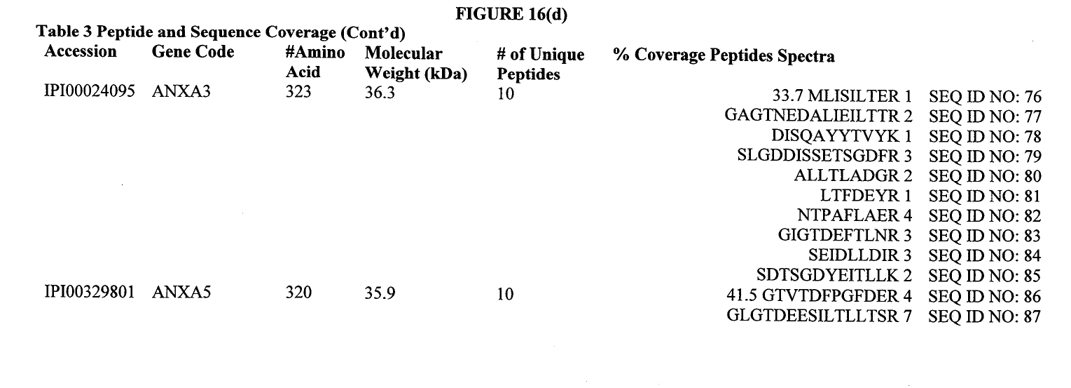

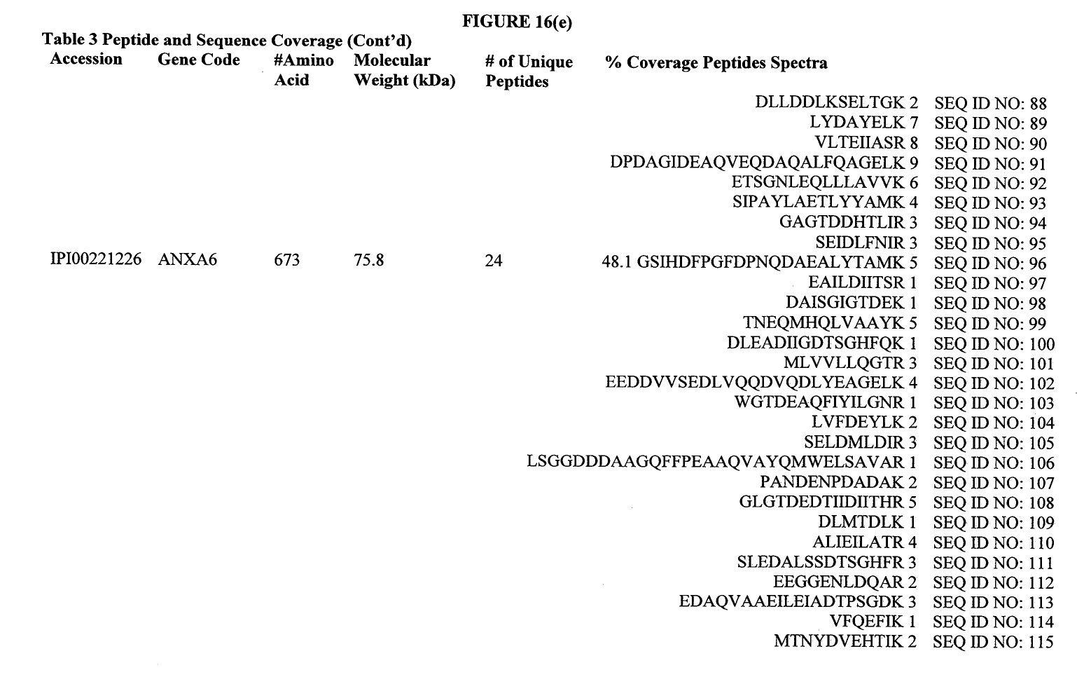

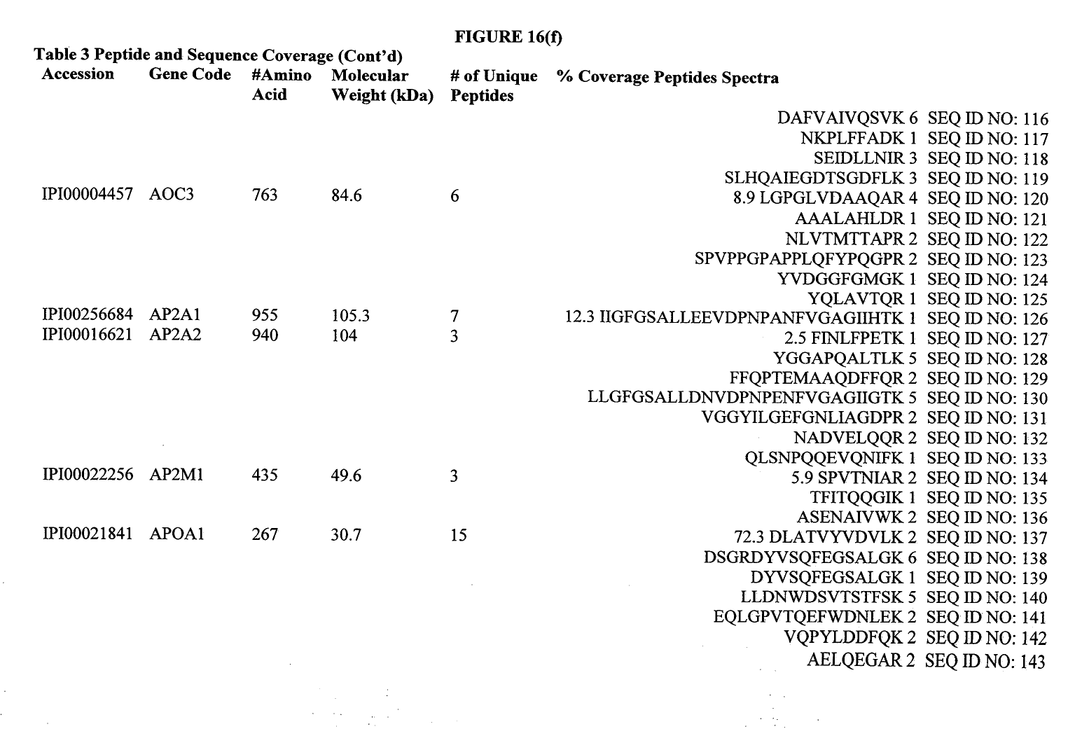

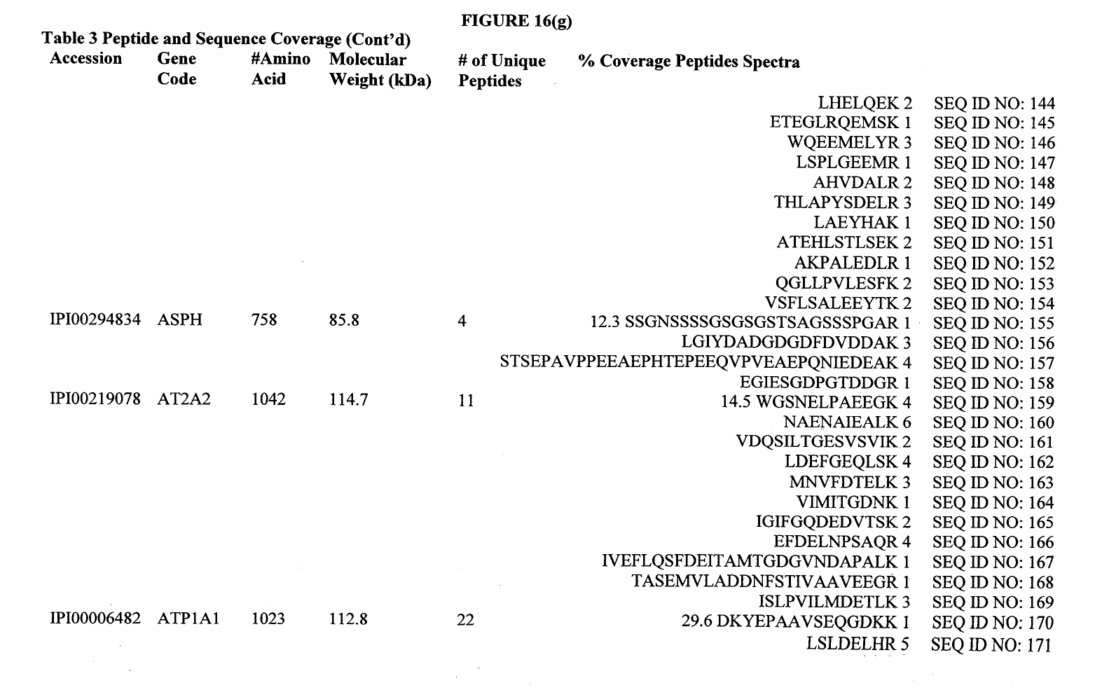

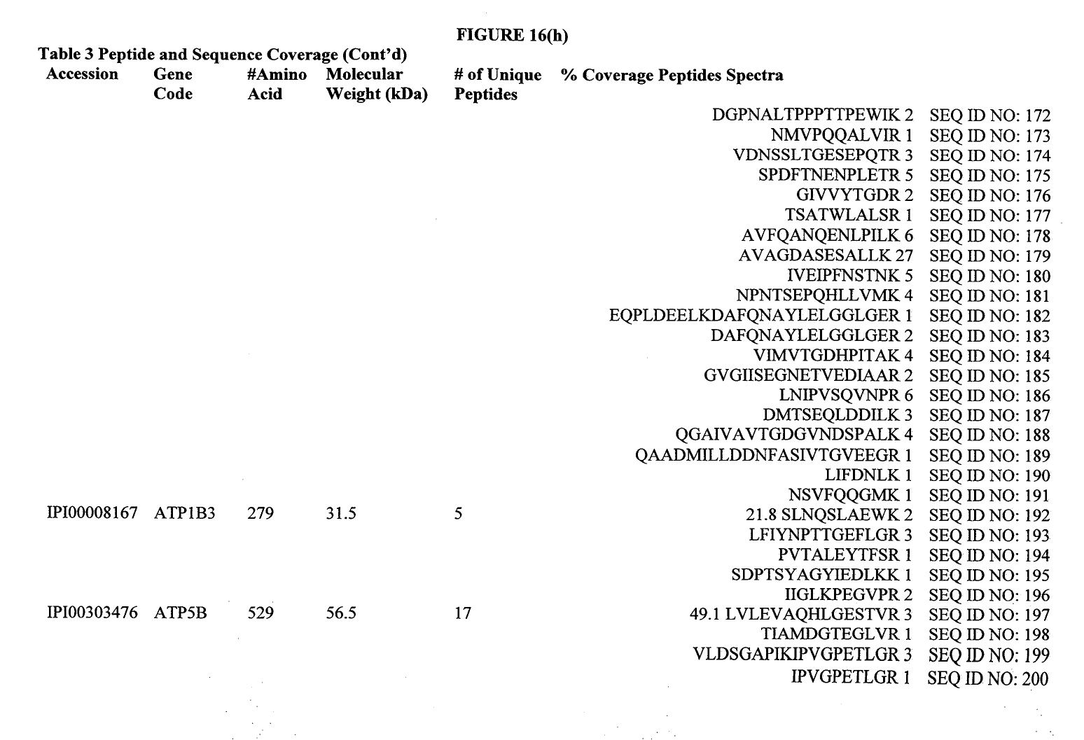

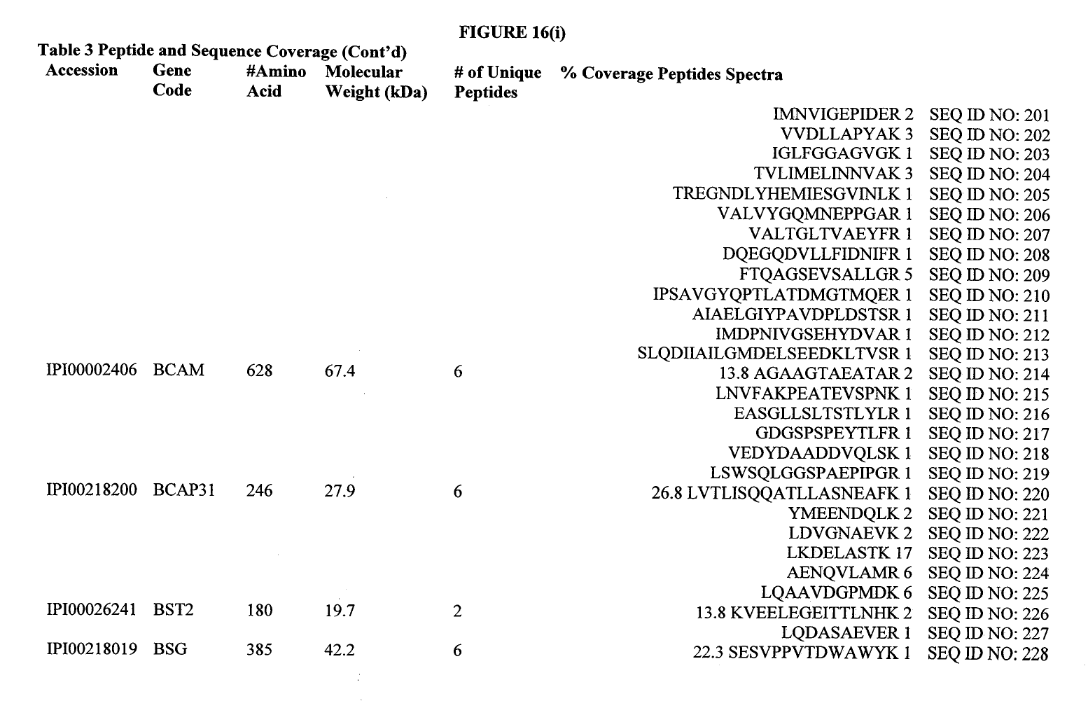

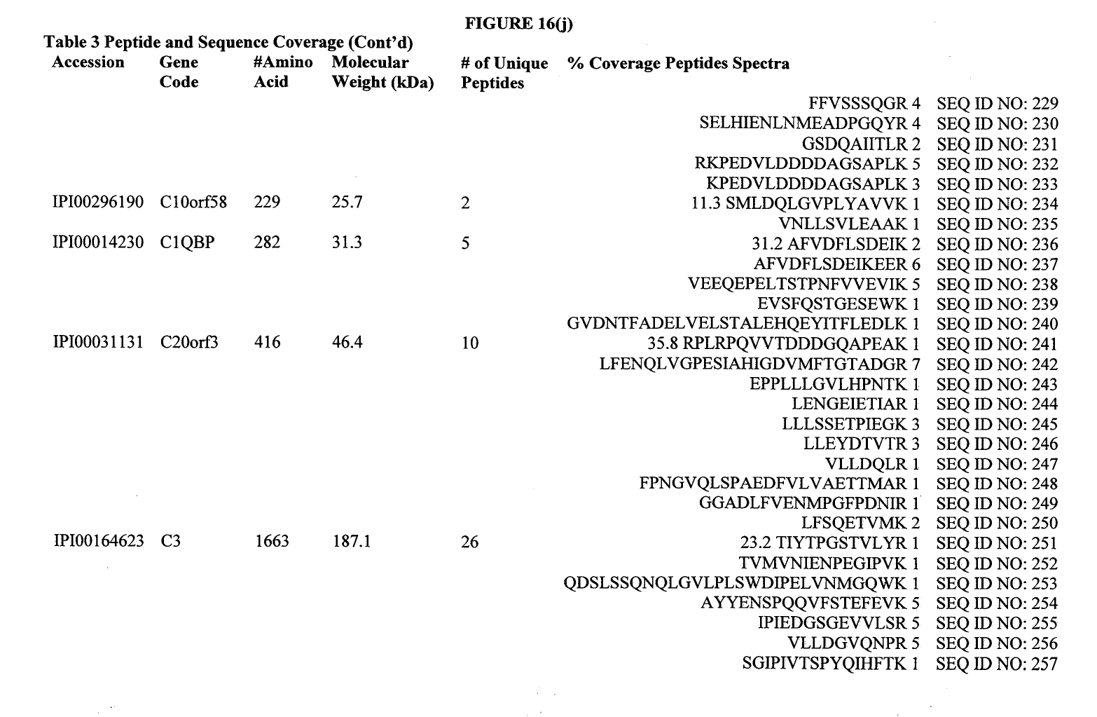

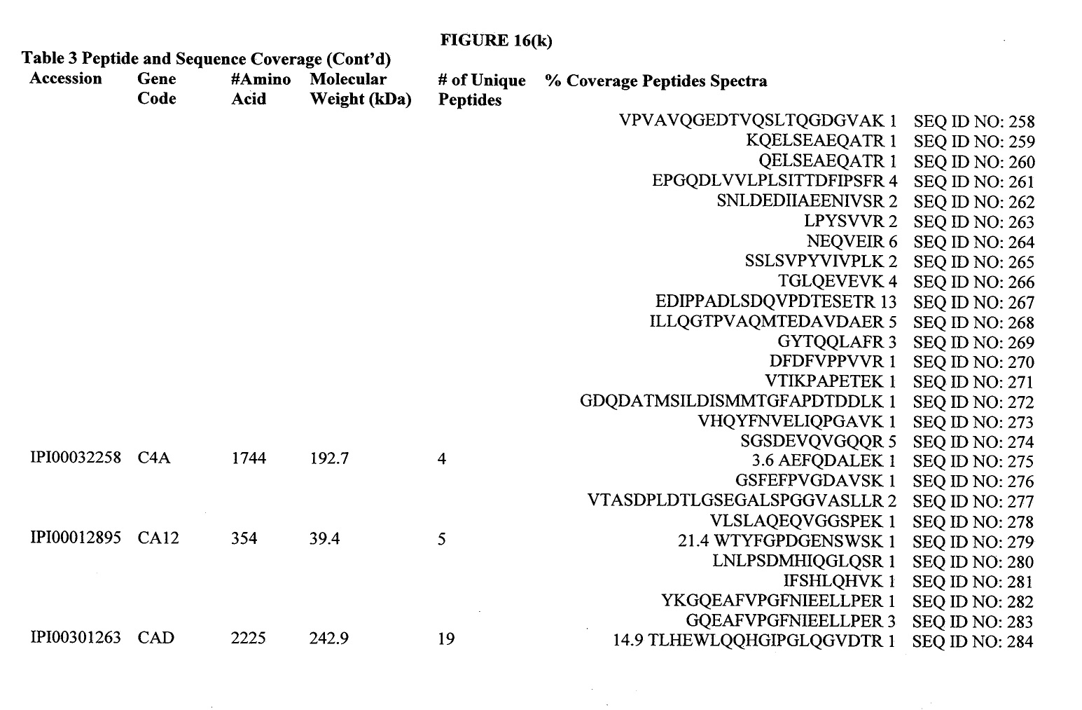

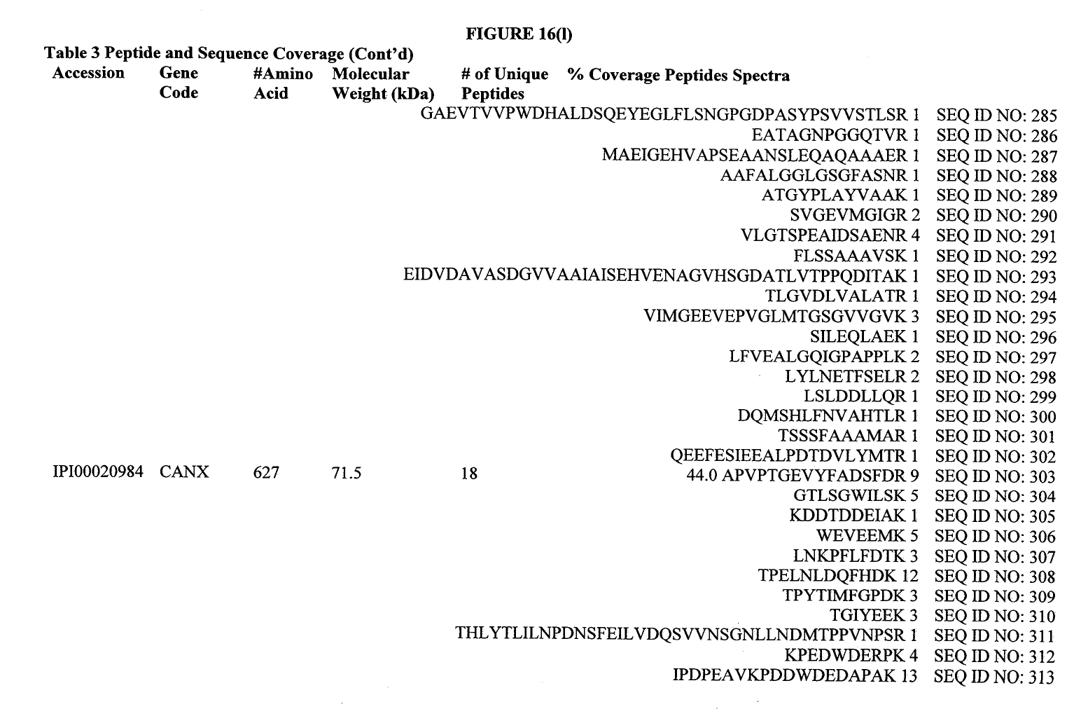

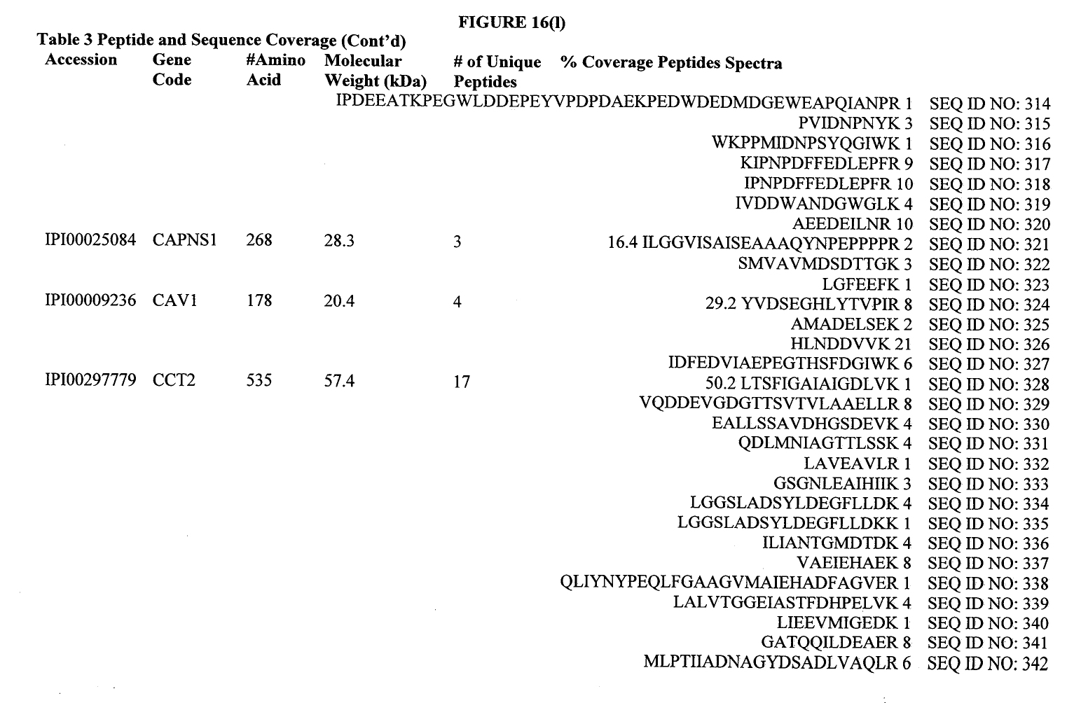

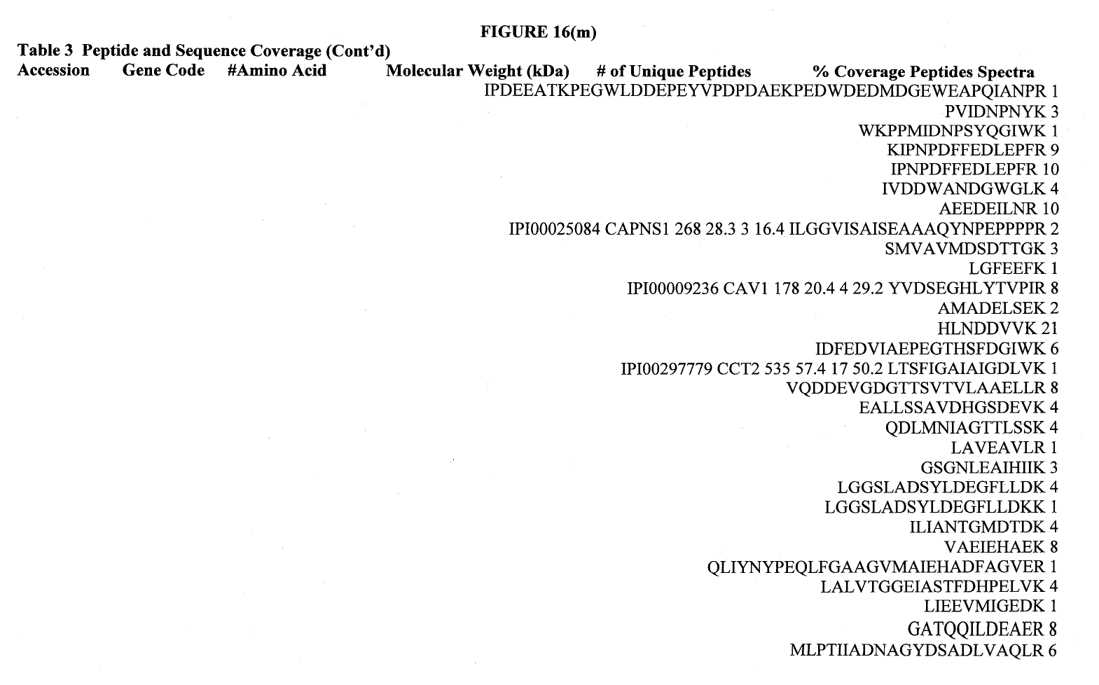

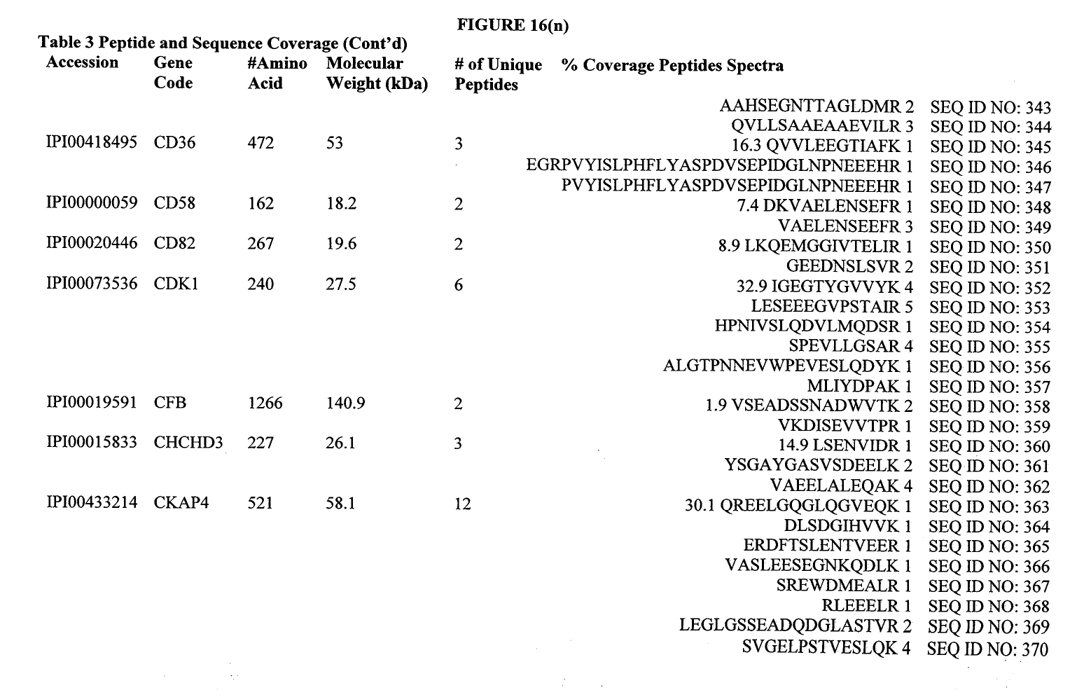

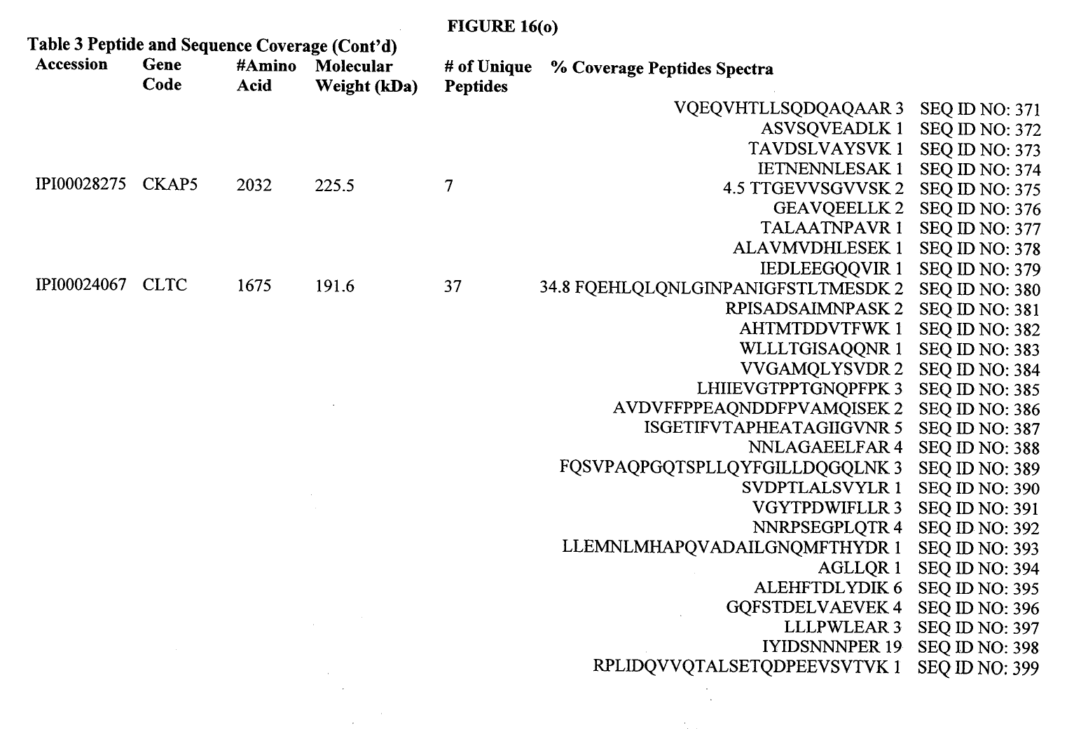

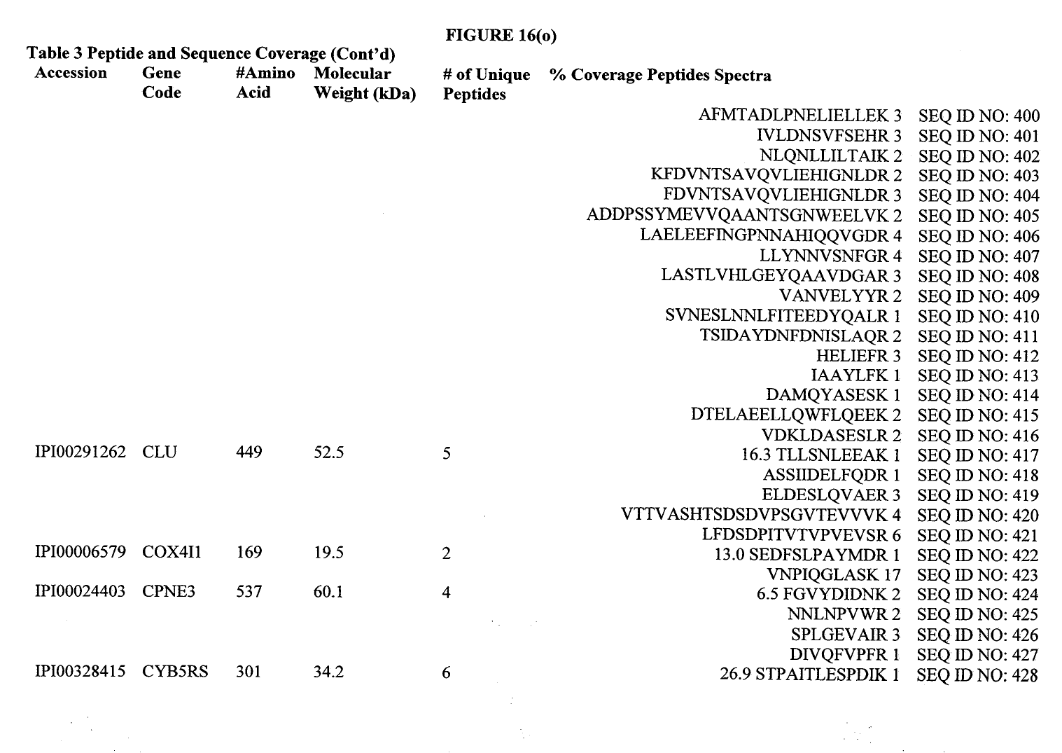

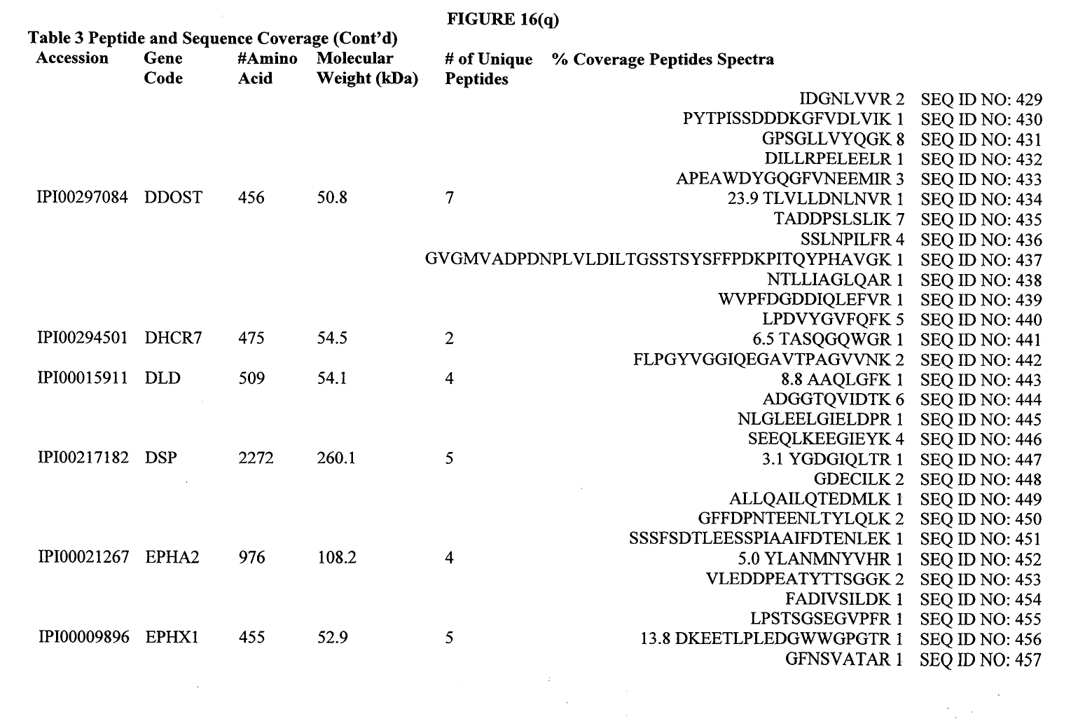

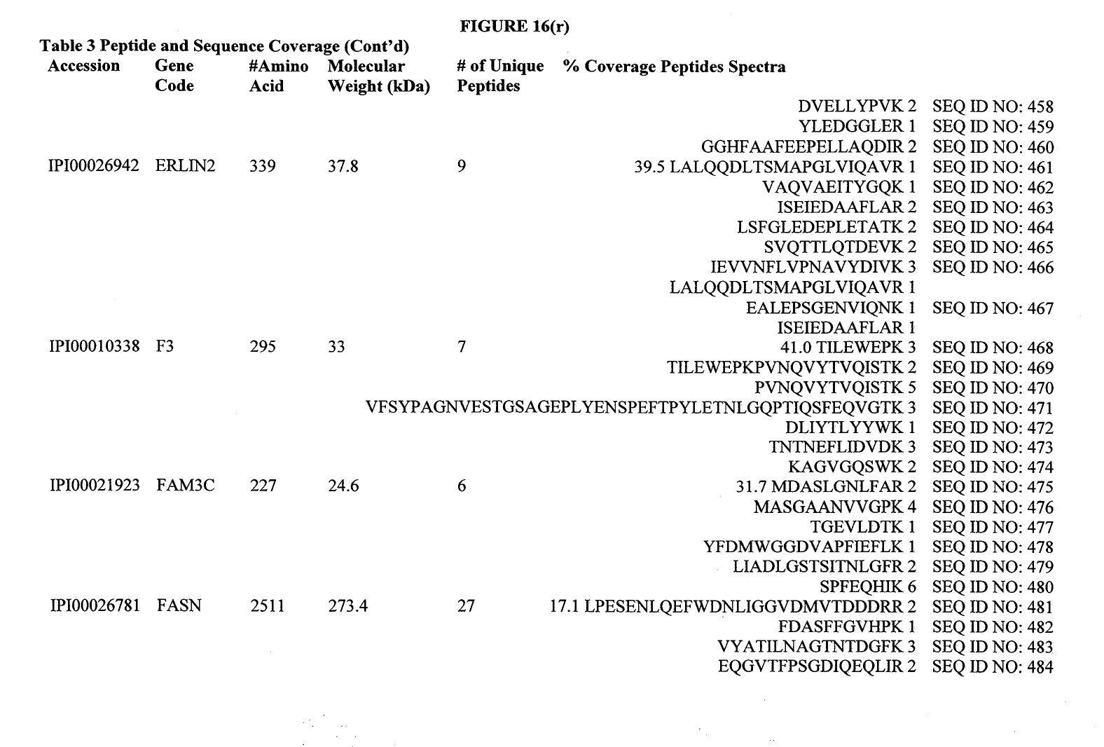

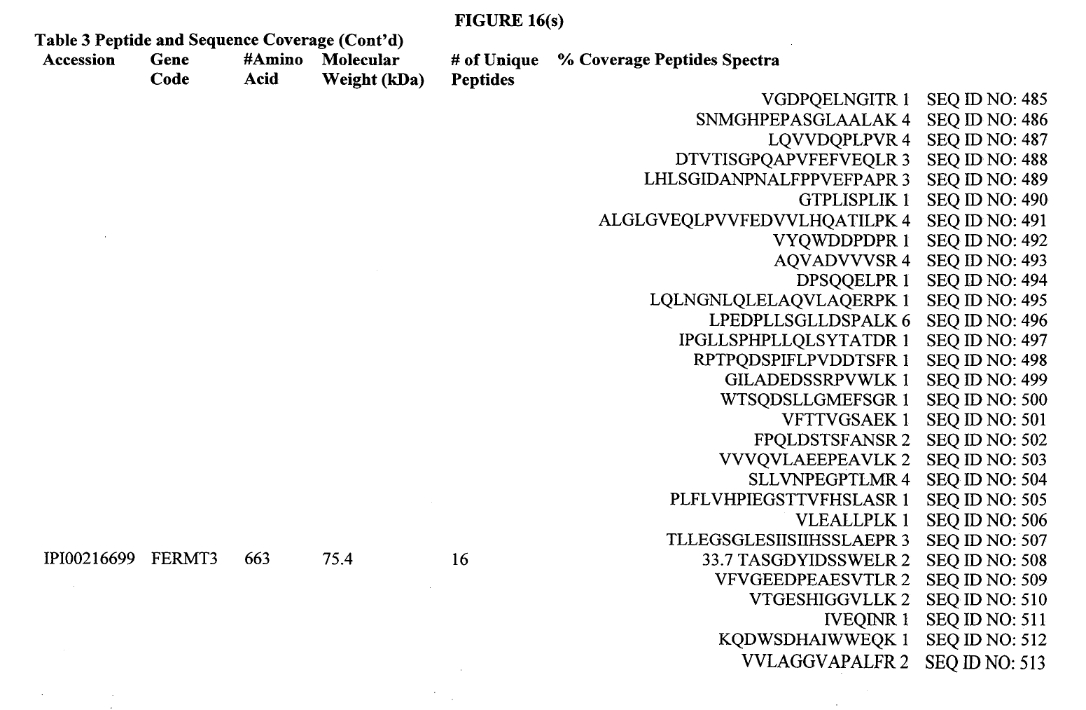

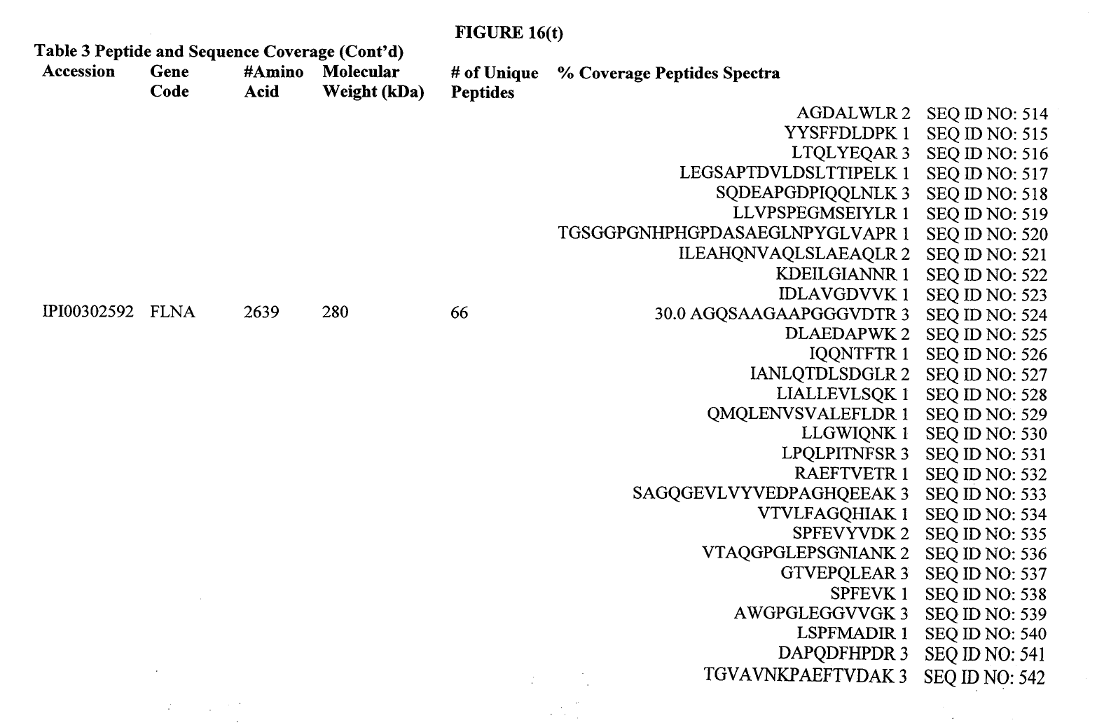

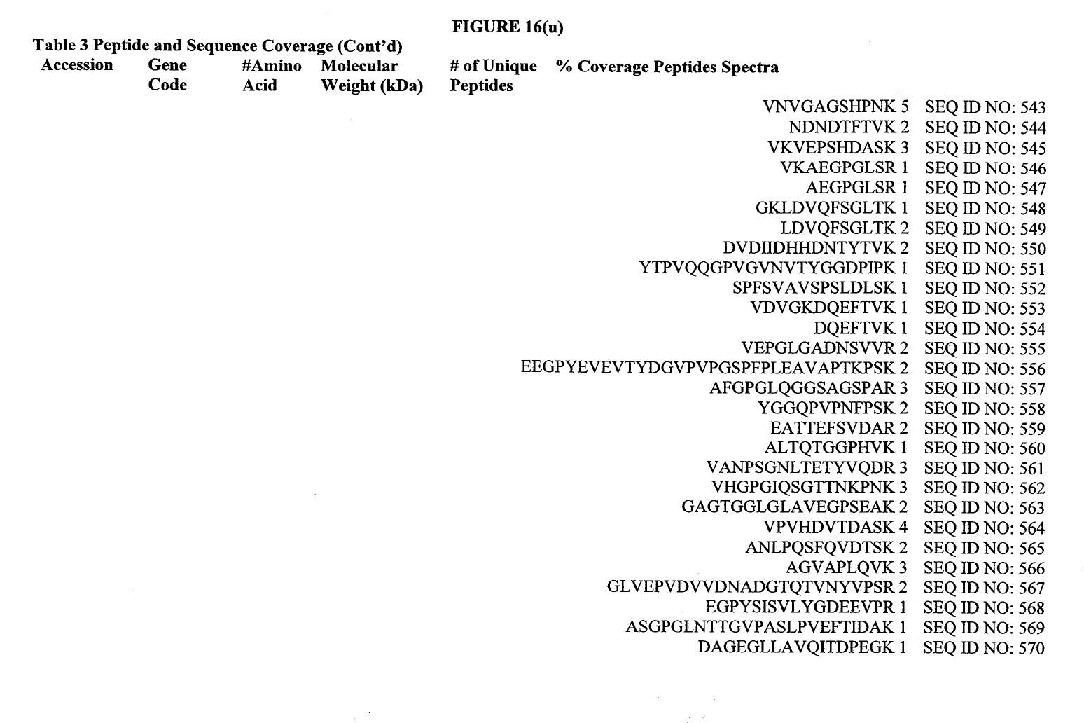

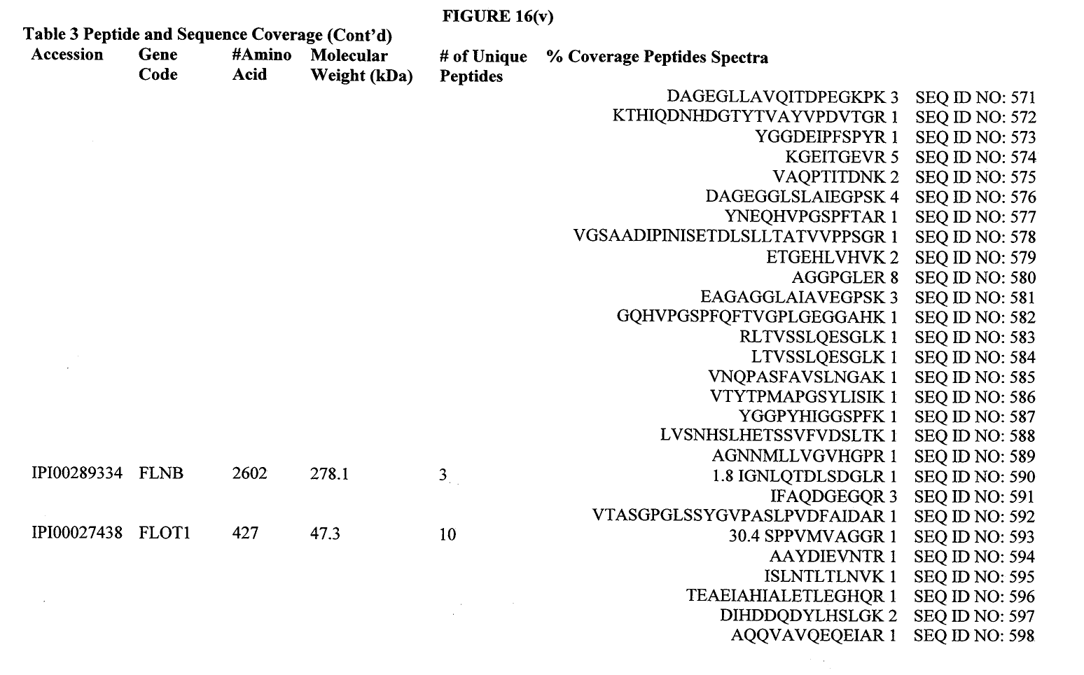

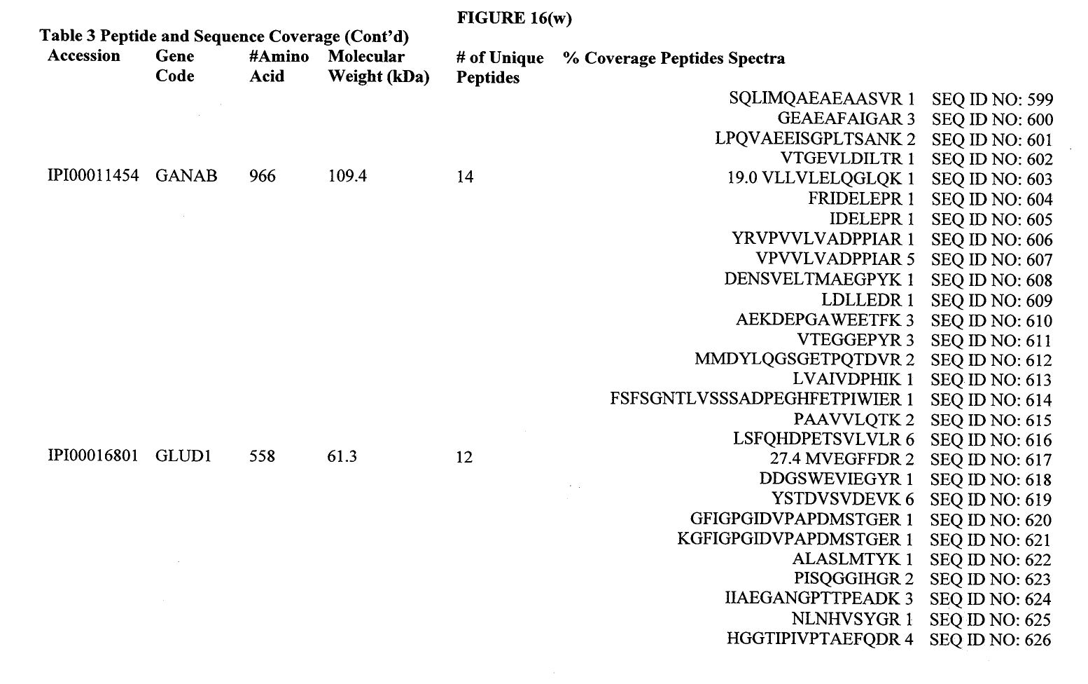

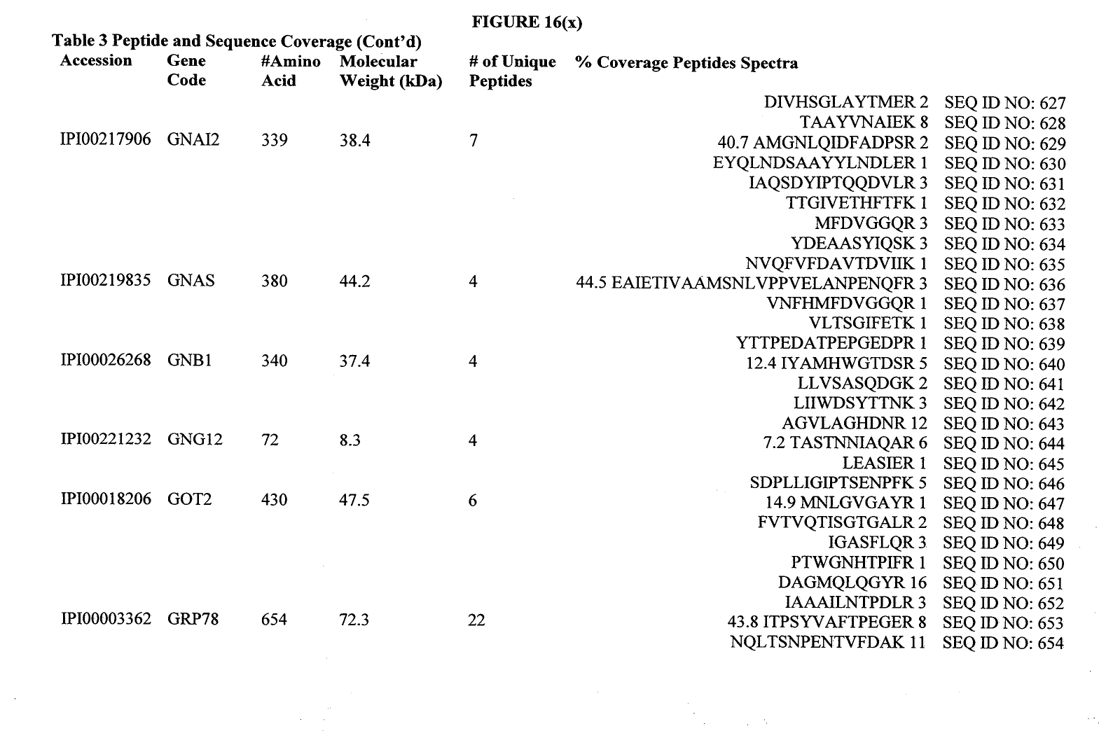

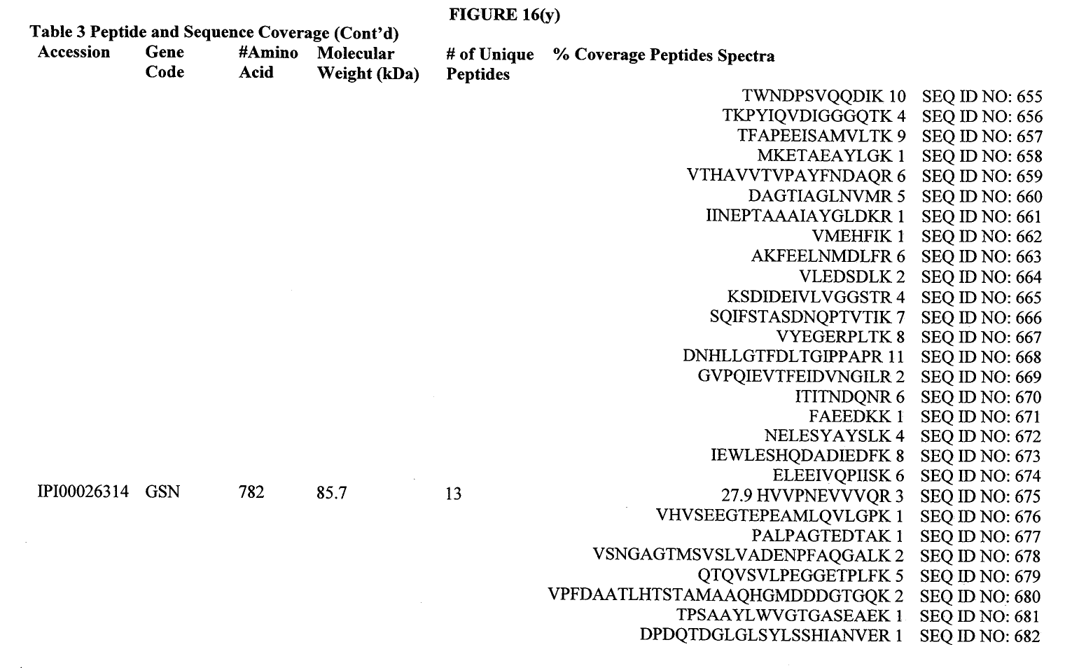

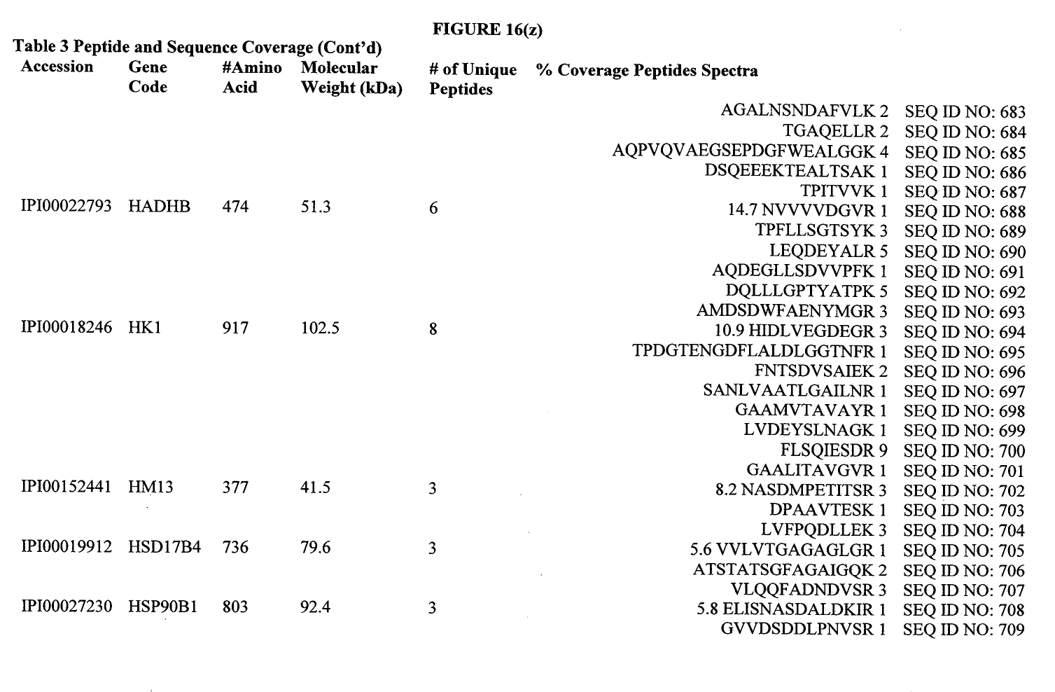

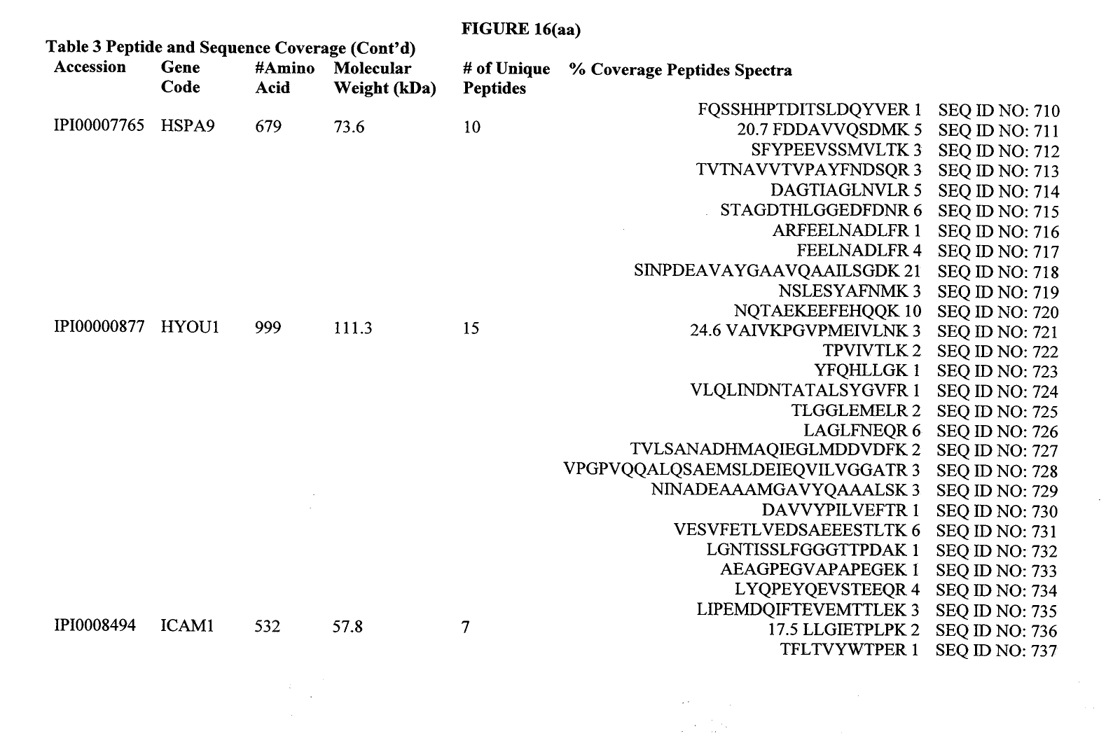

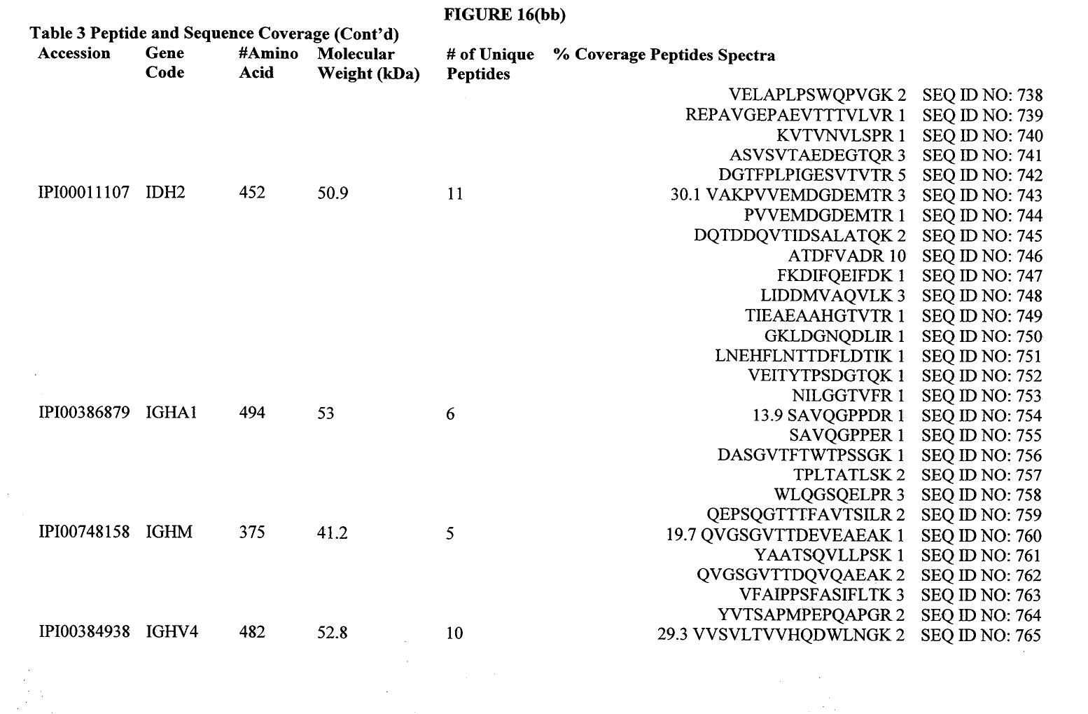

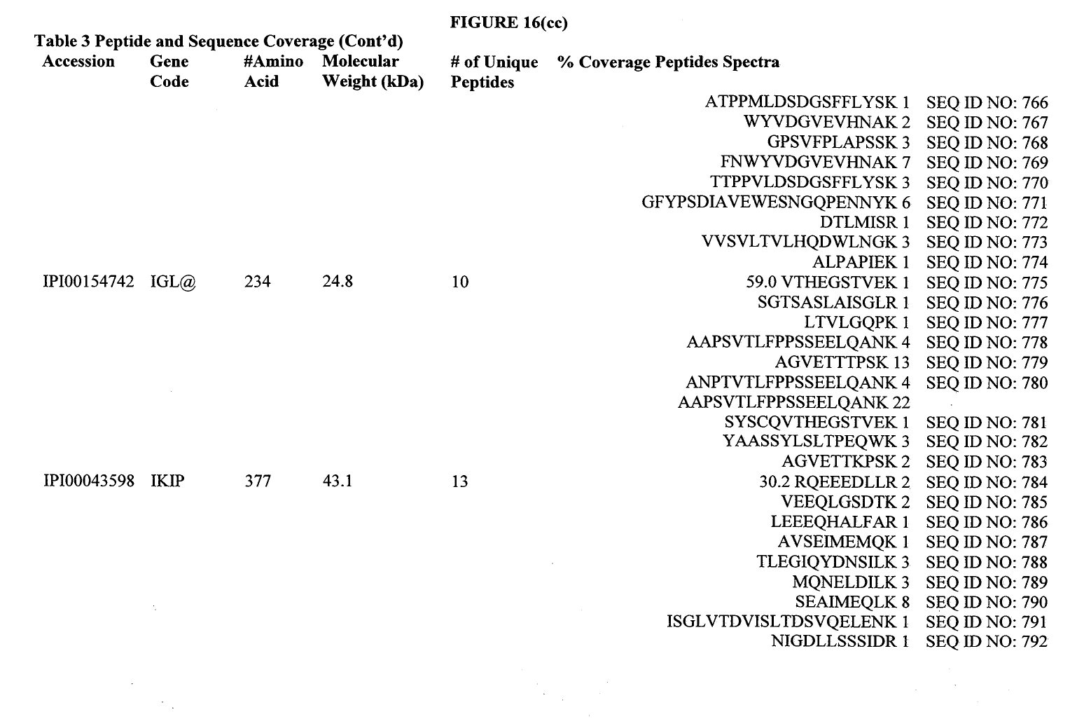

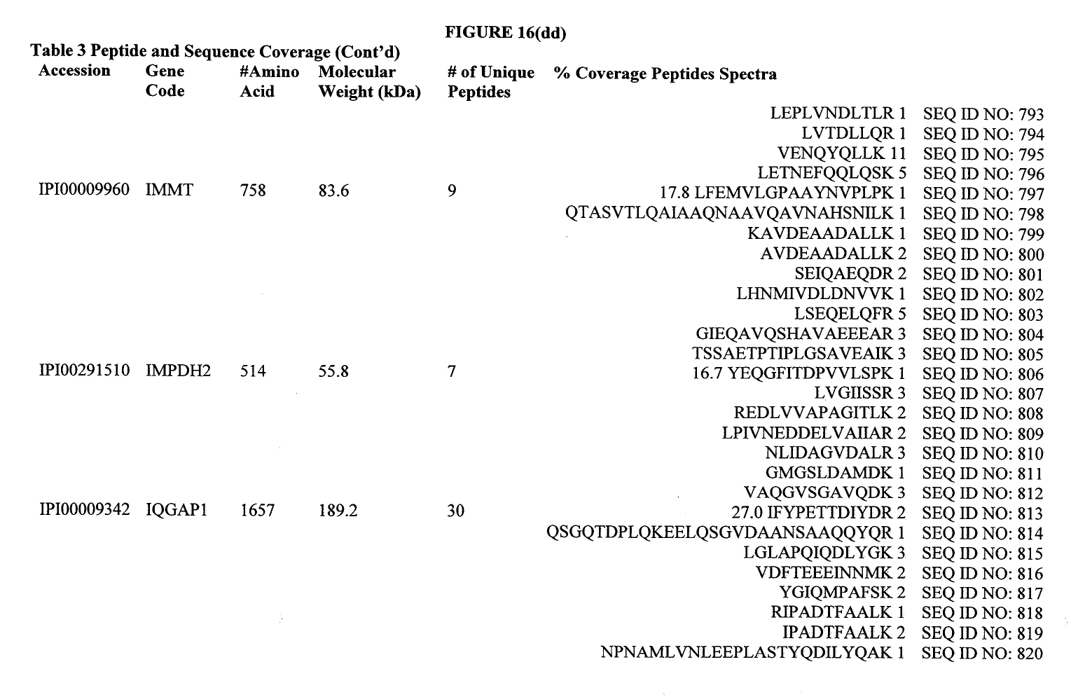

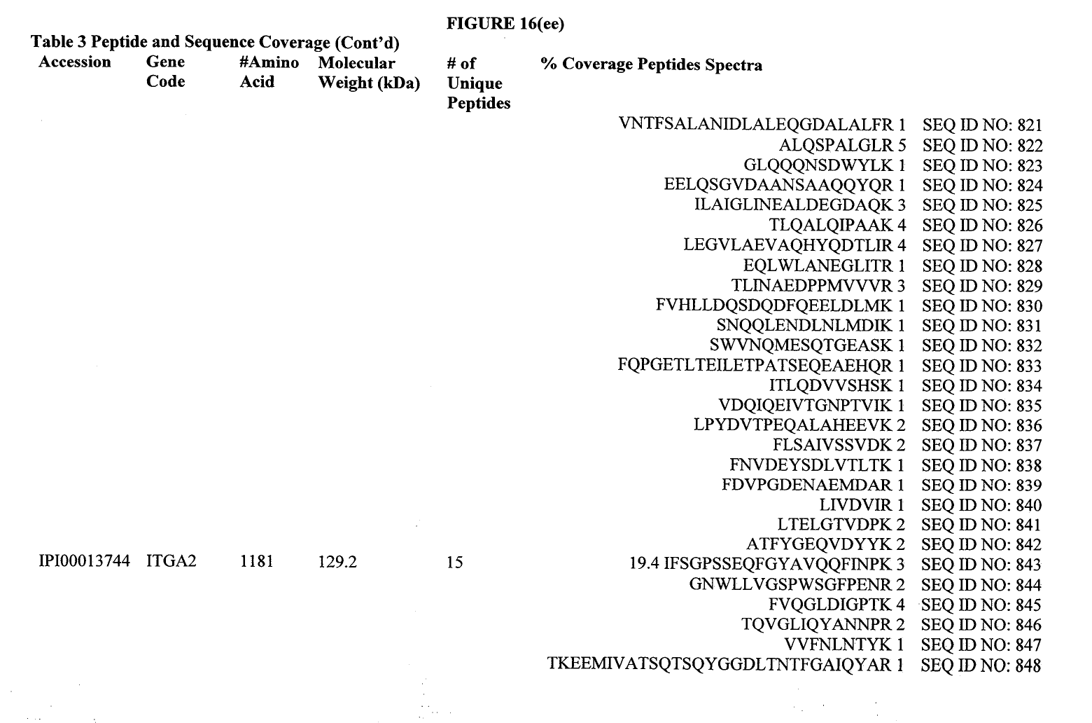

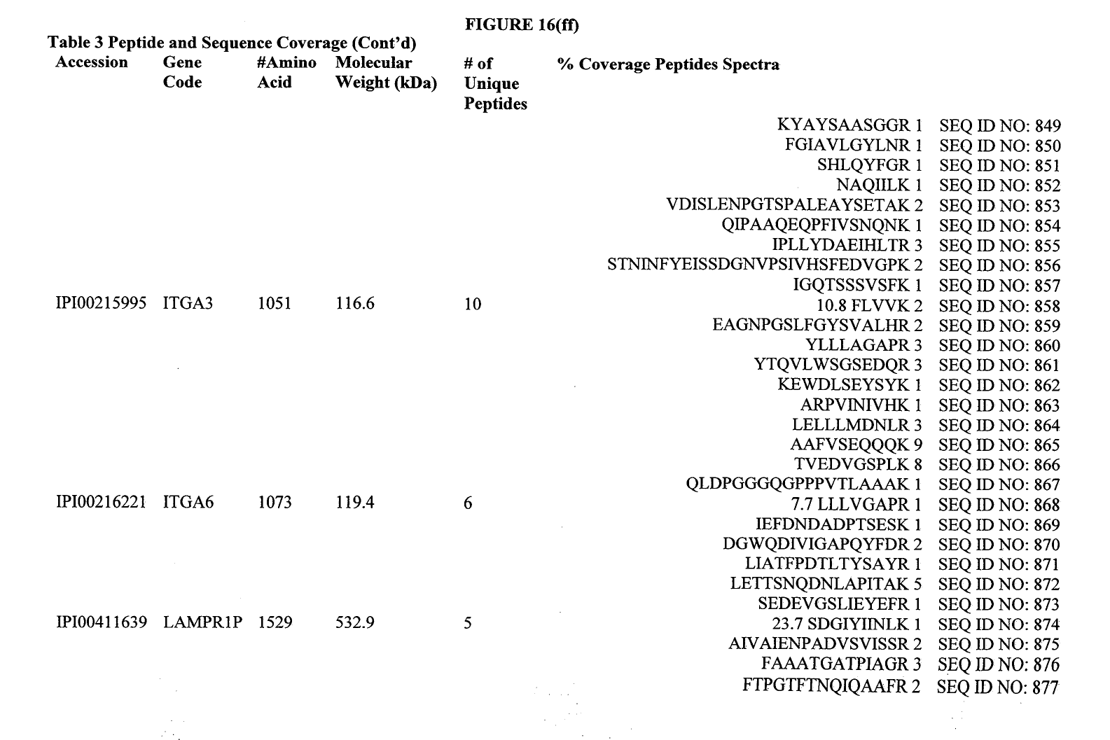

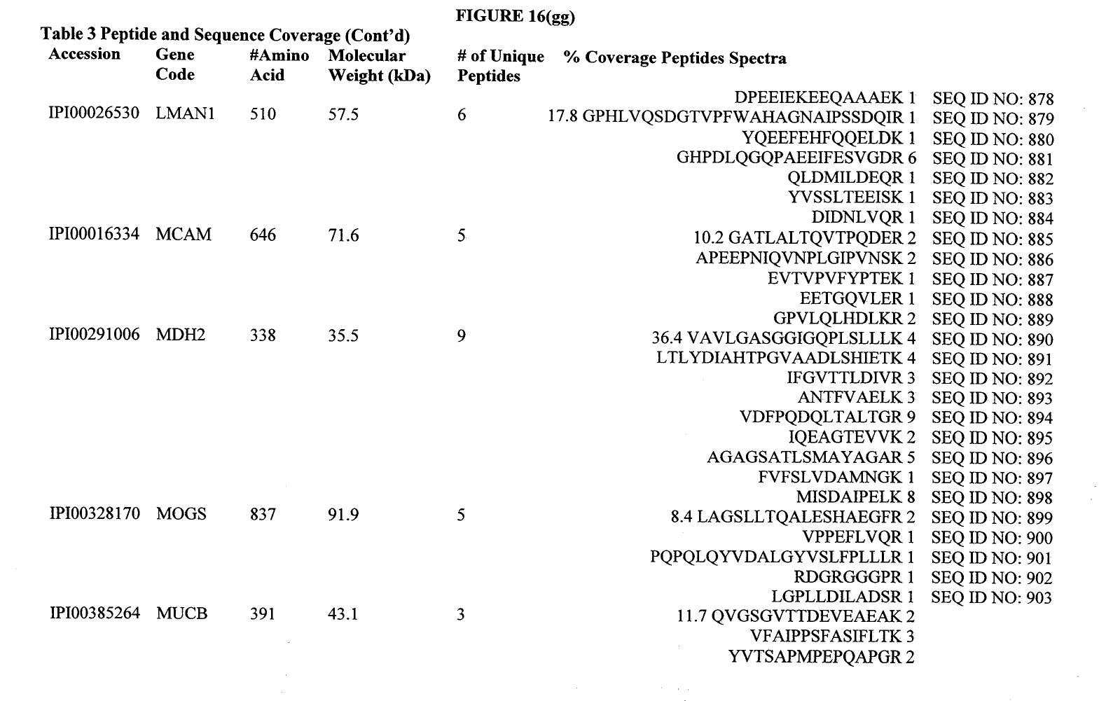

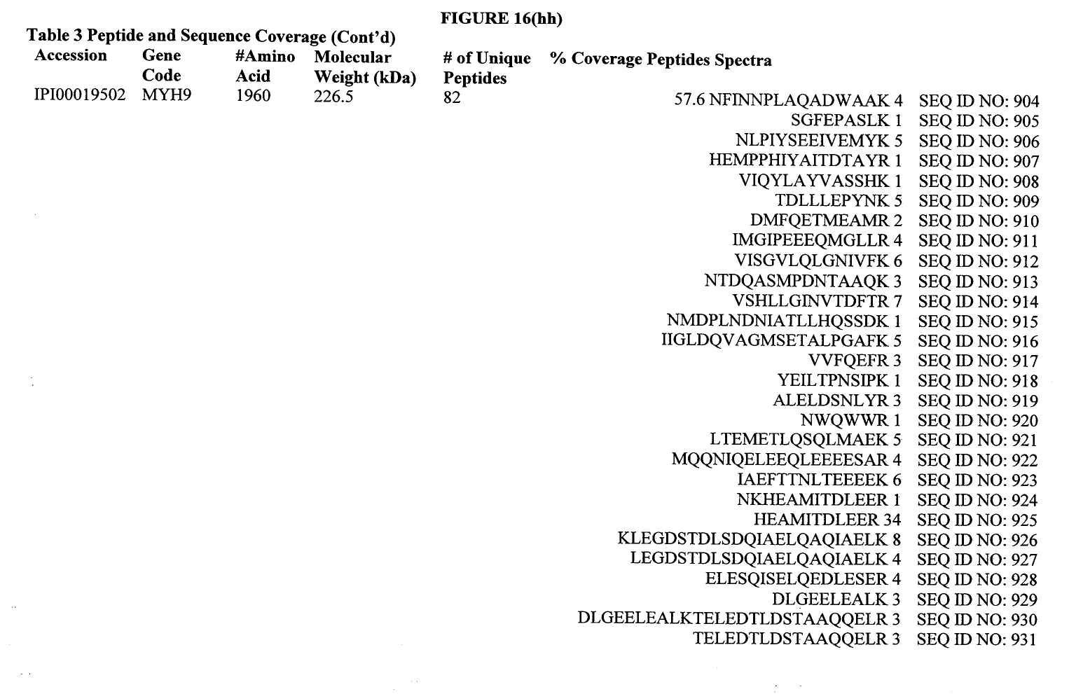

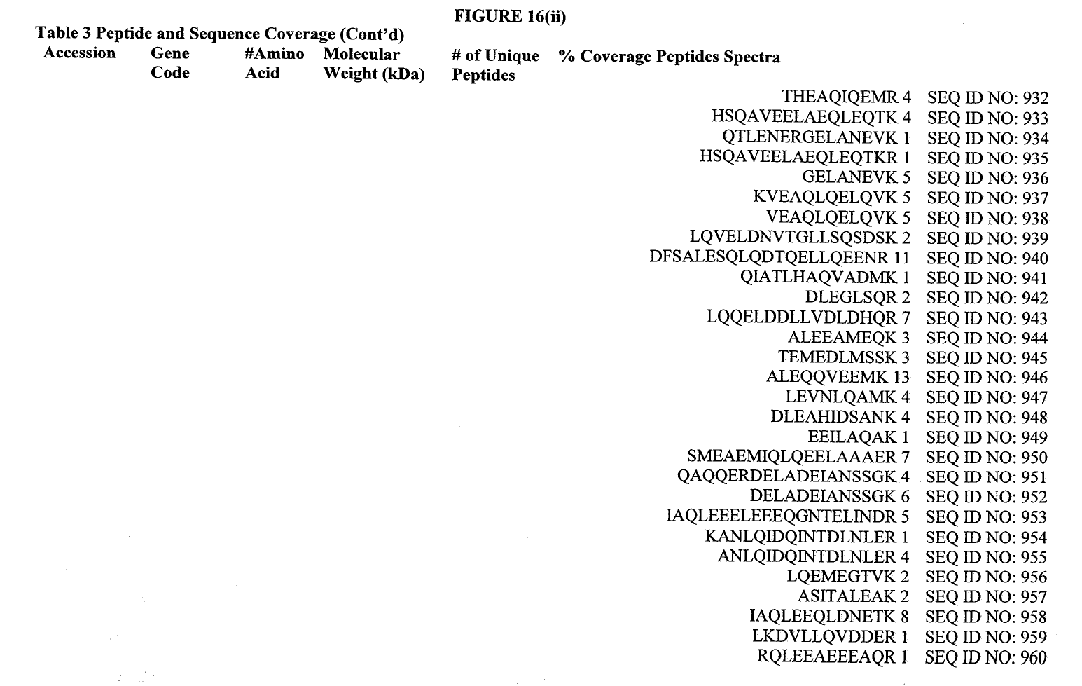

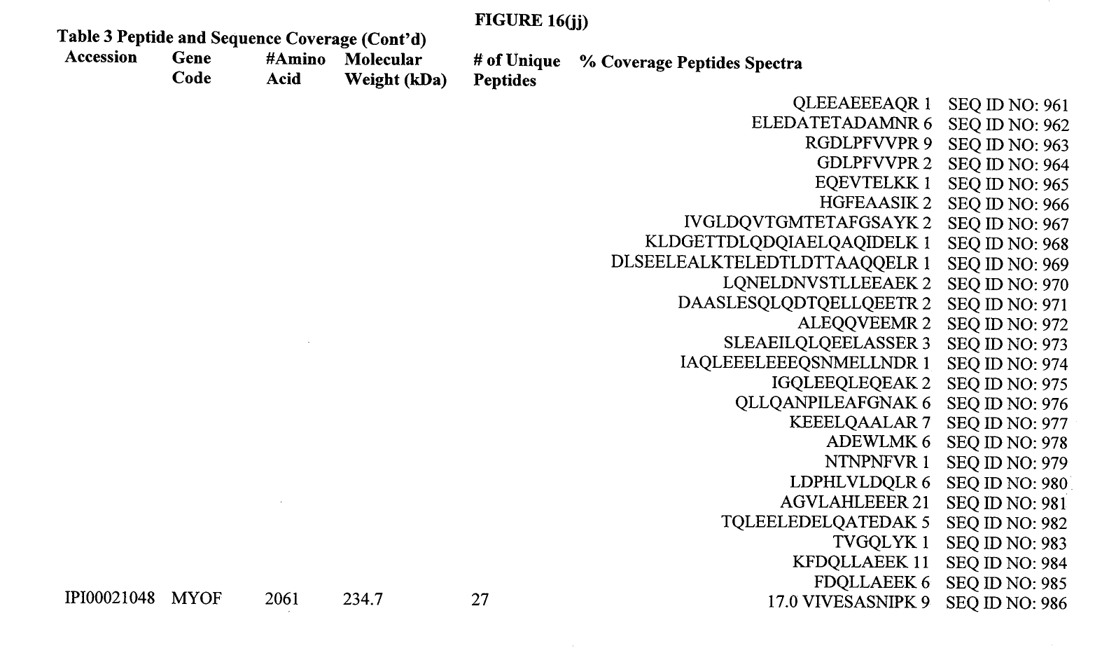

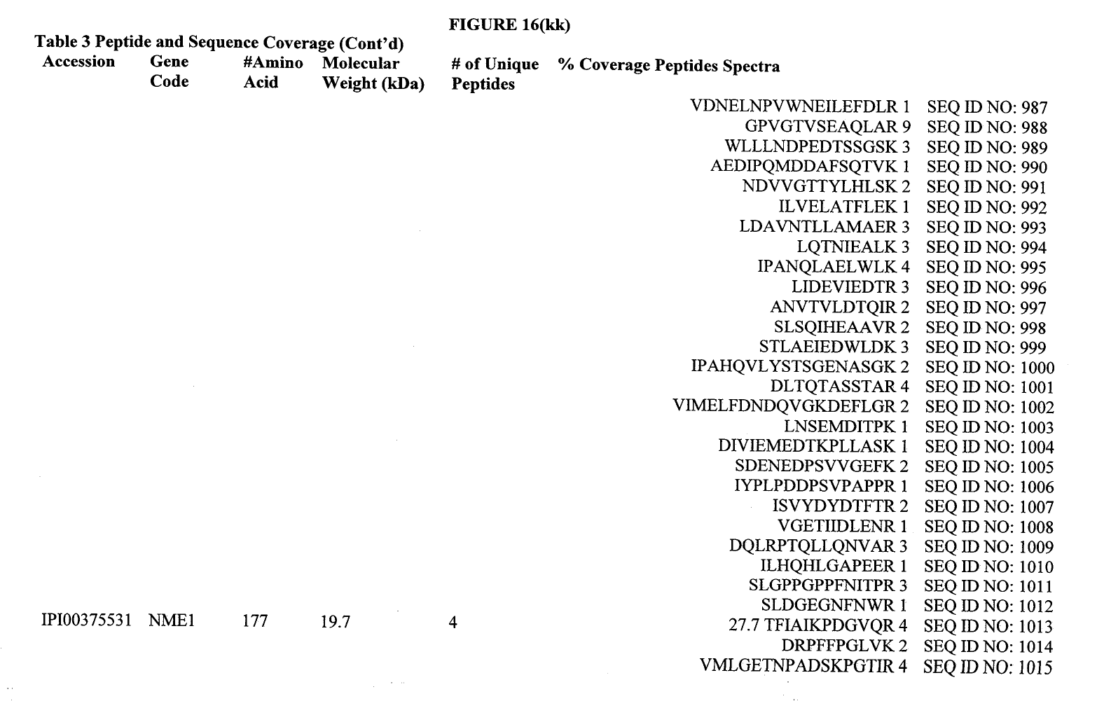

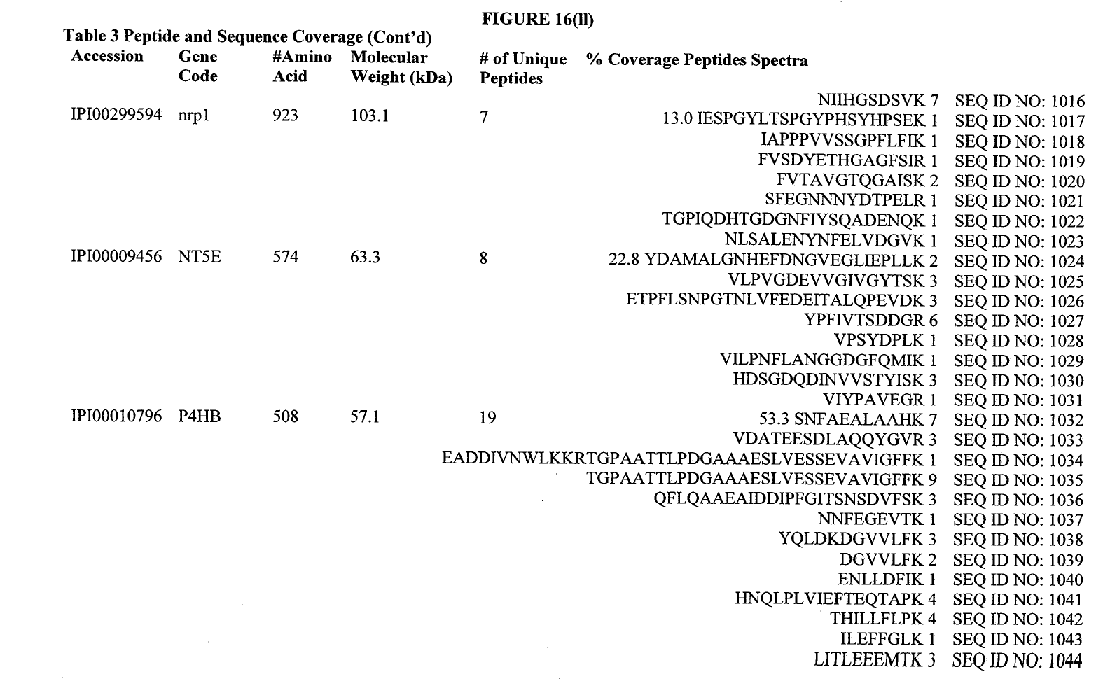

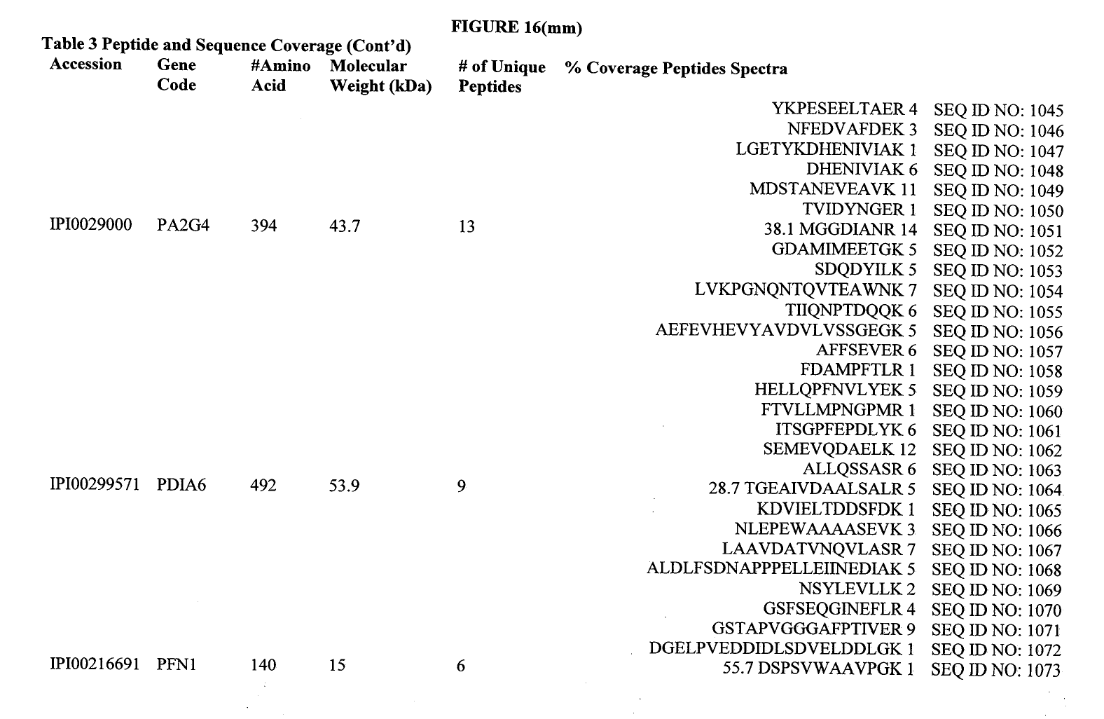

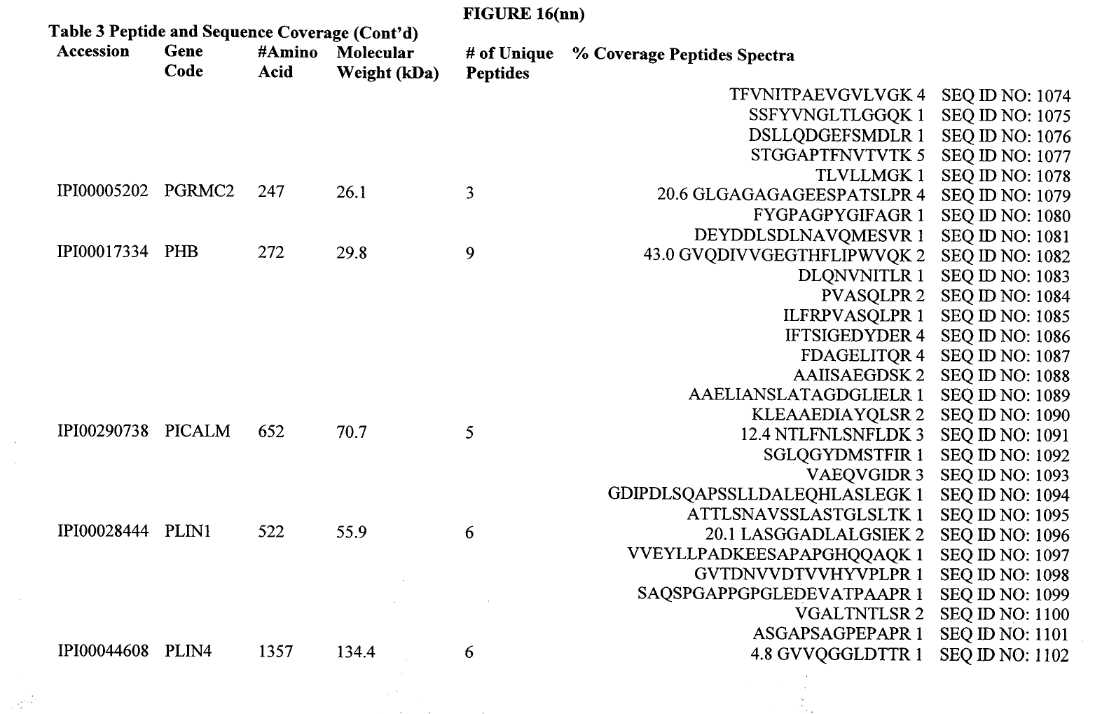

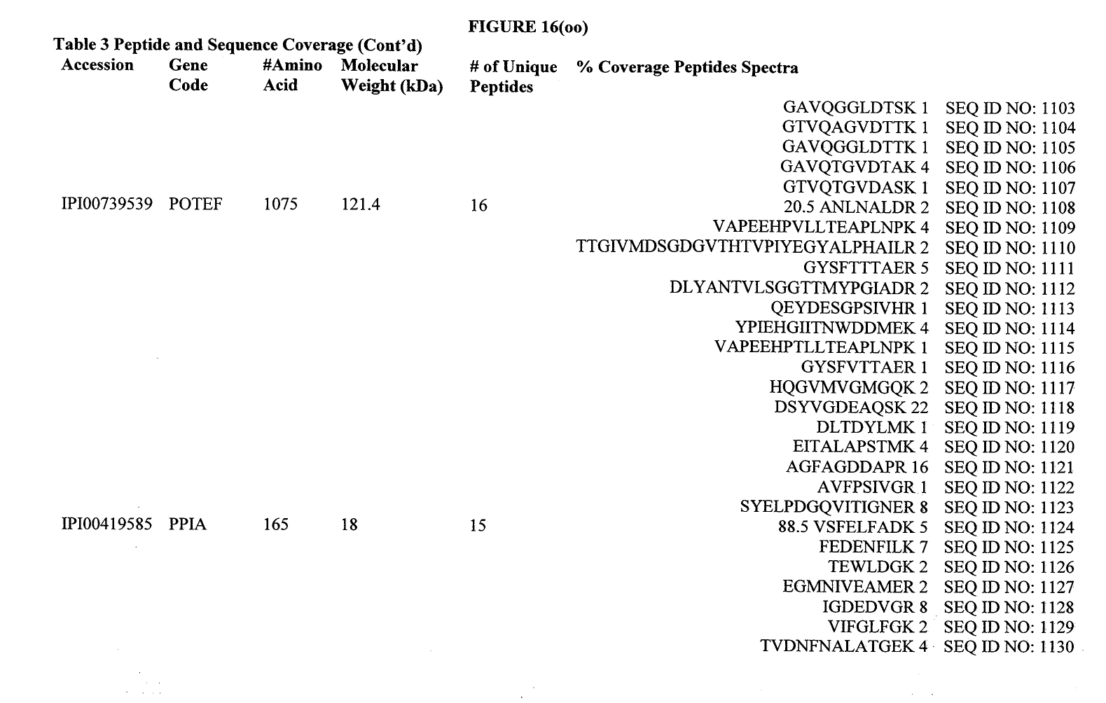

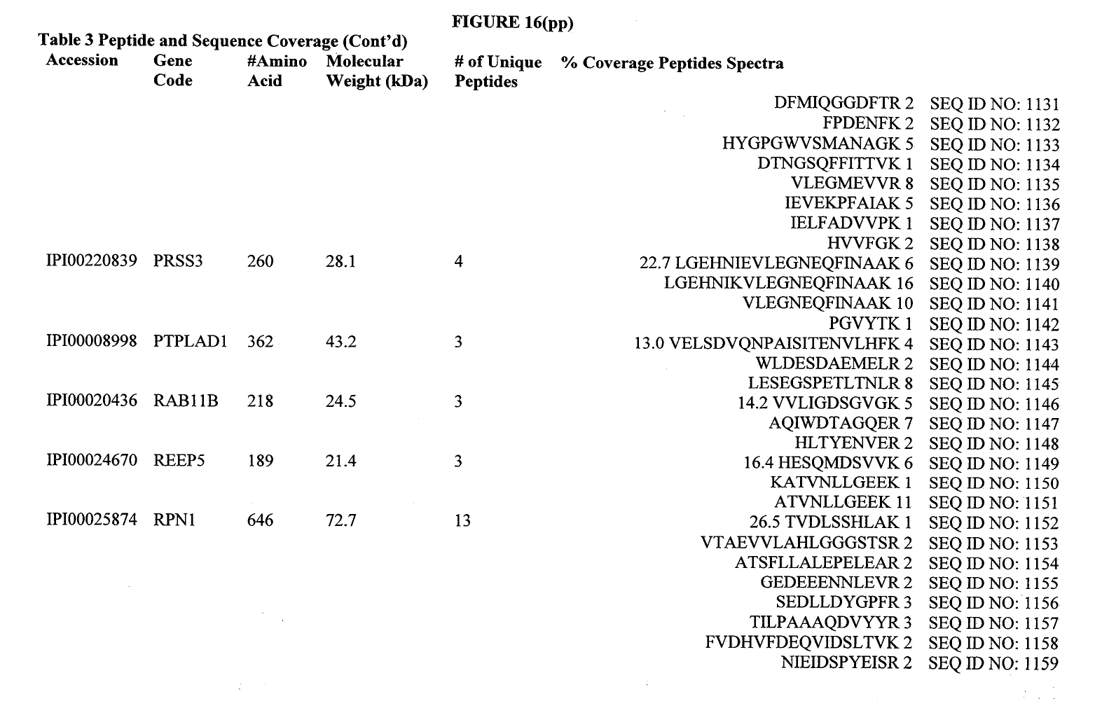

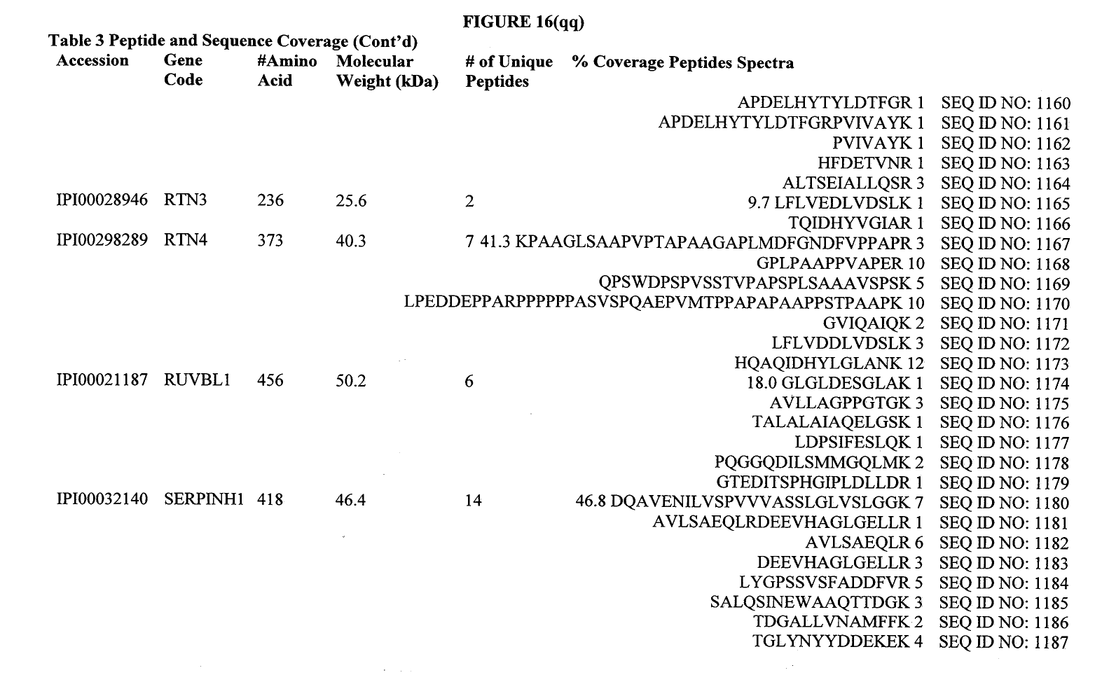

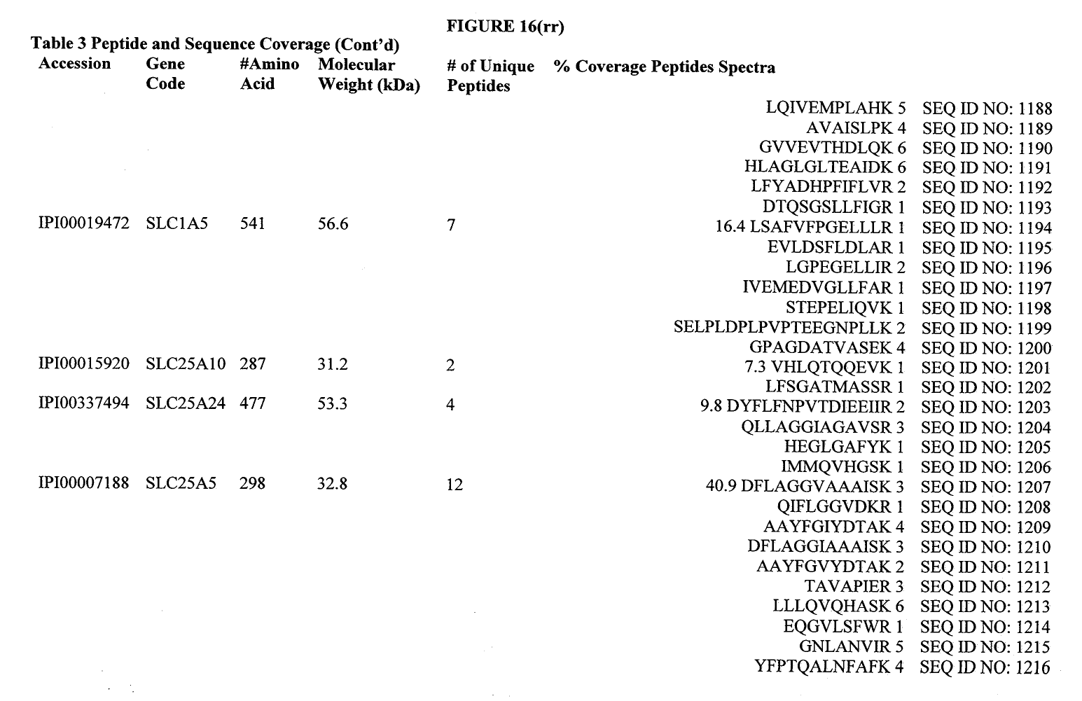

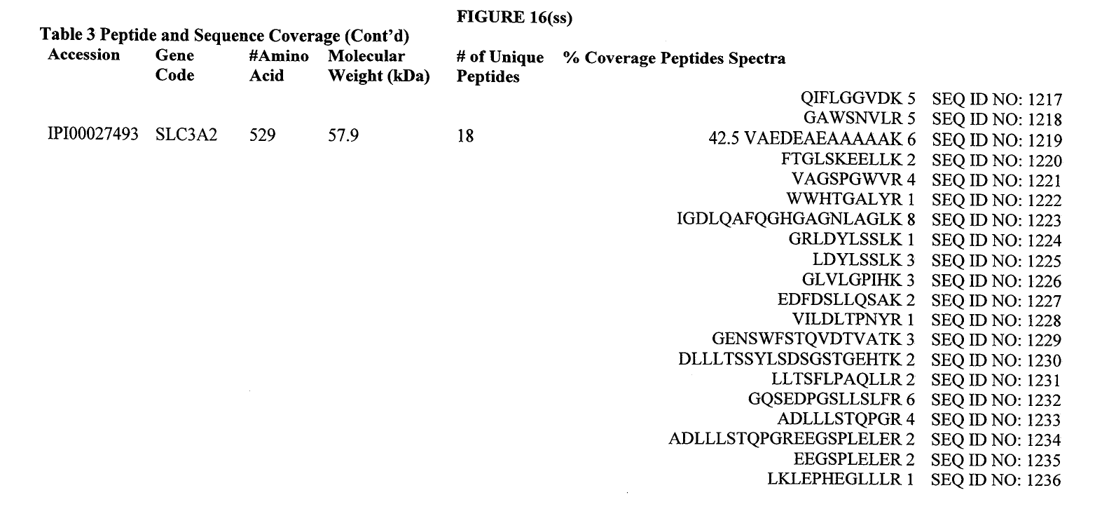

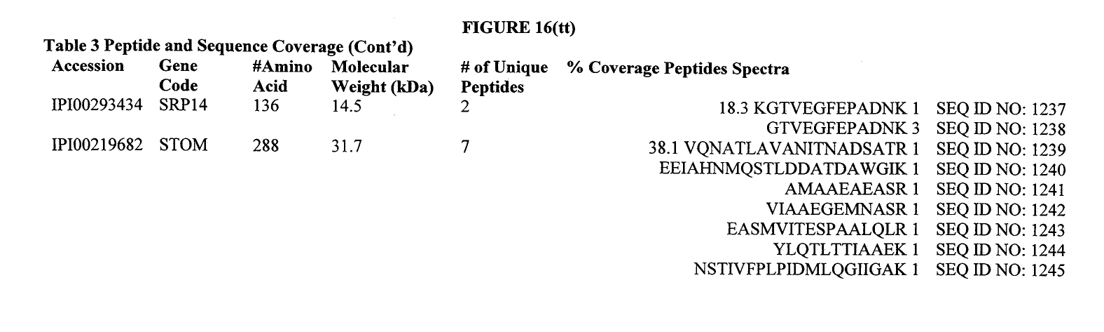

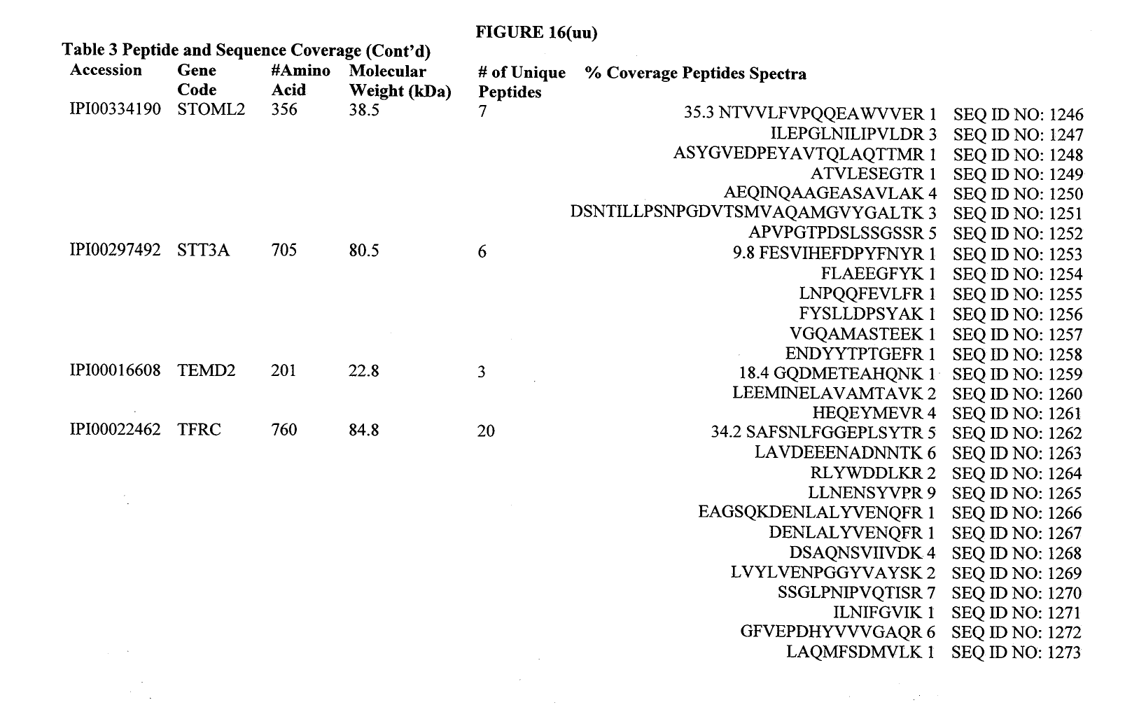

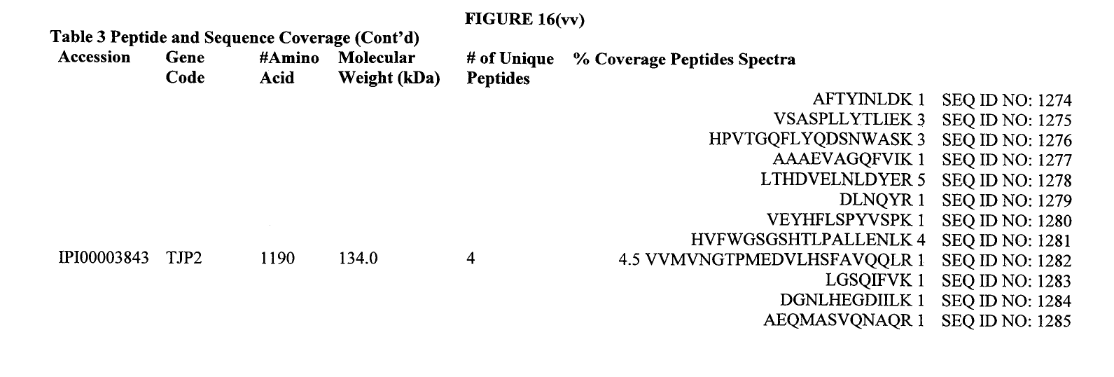

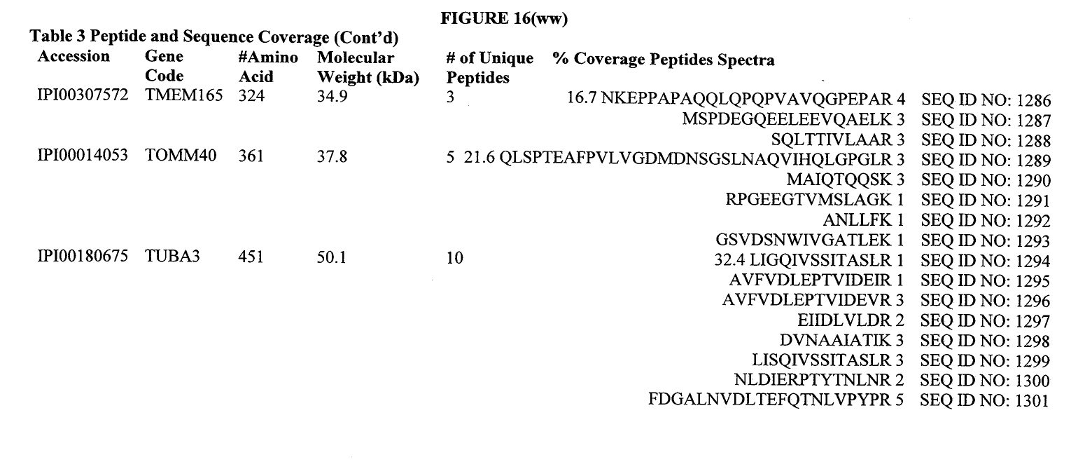

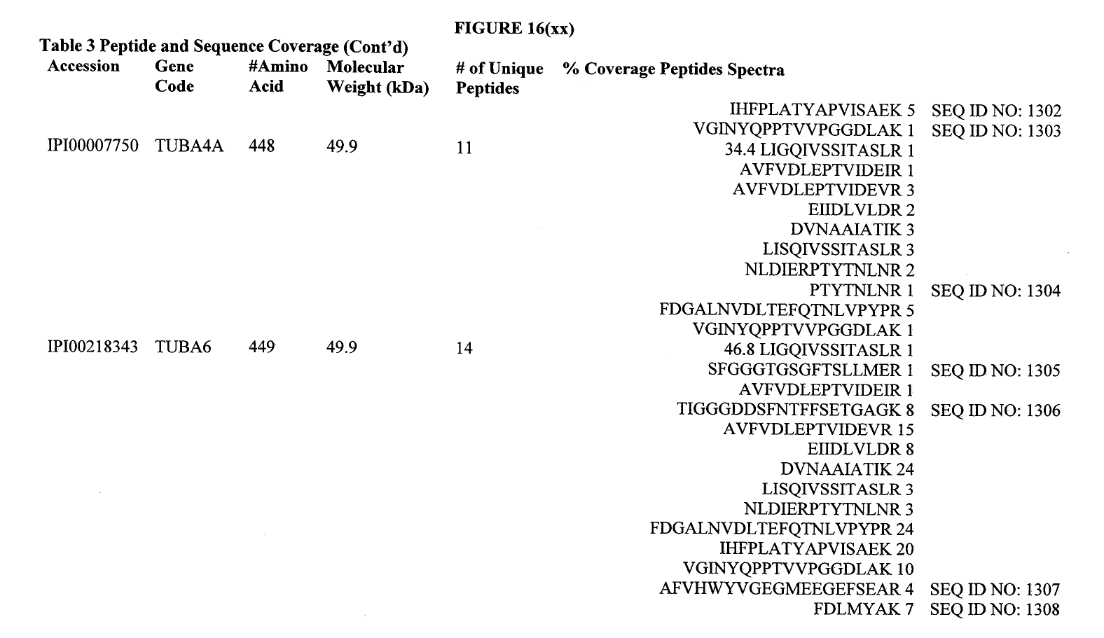

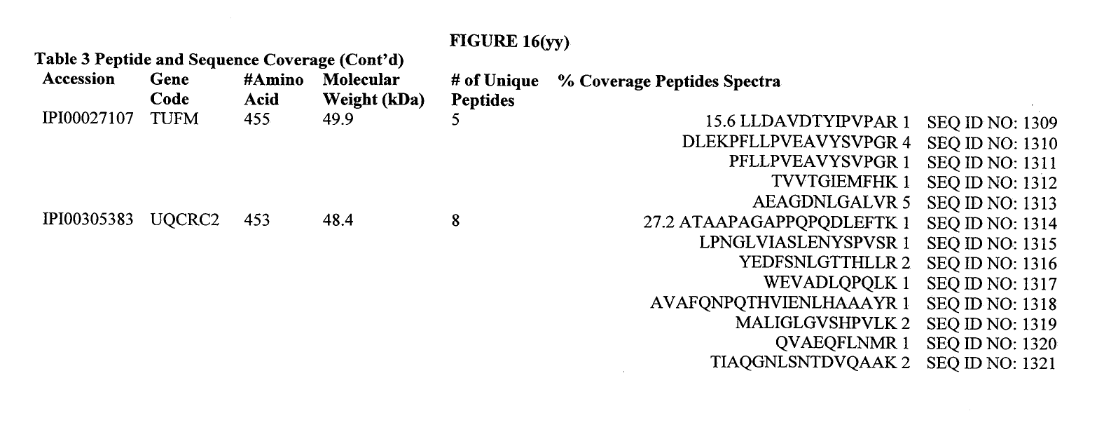

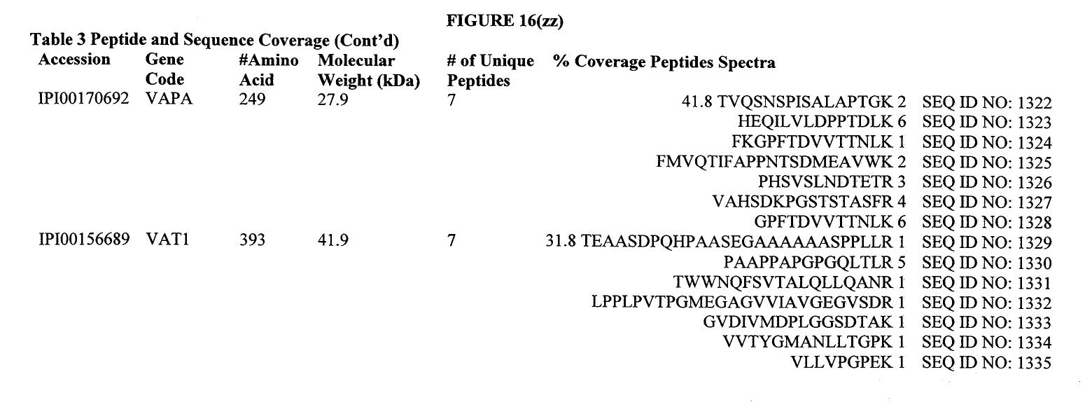

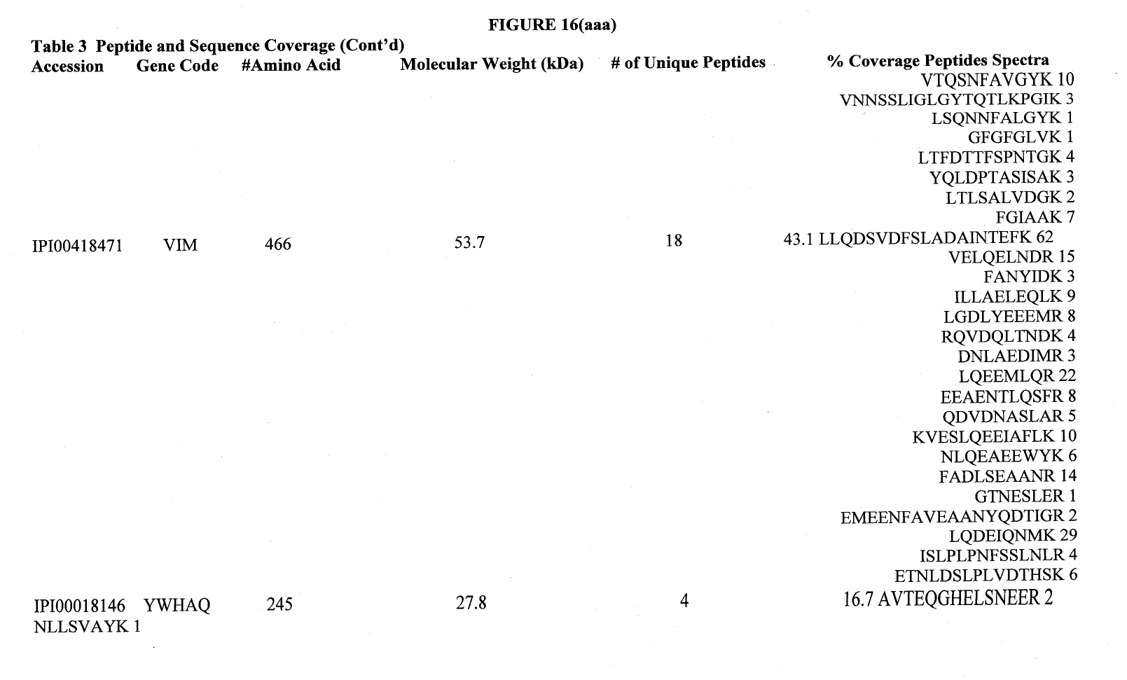

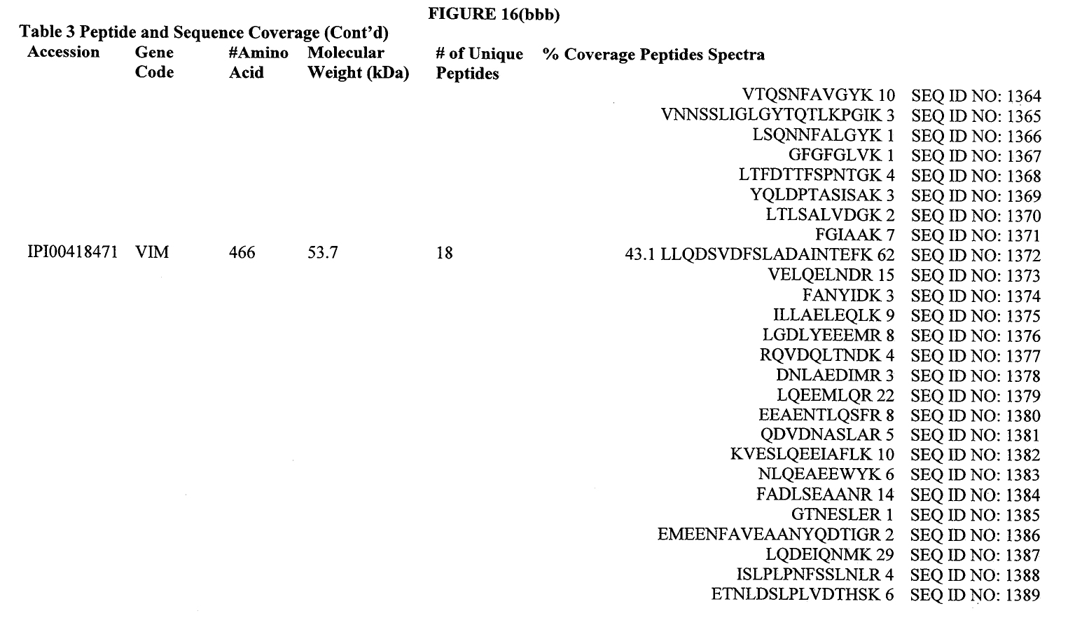

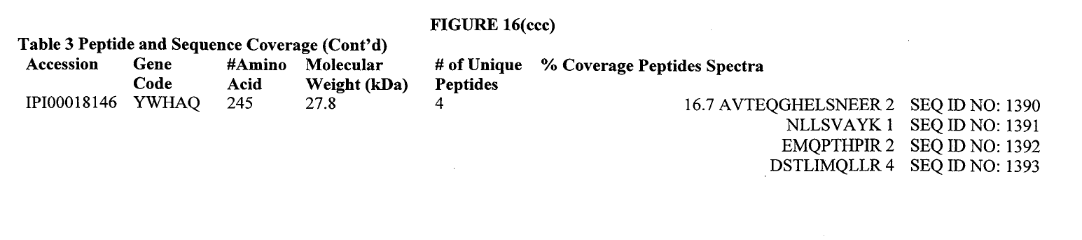

GPI anchored proteins represent valuable therapeutic and/or diagnostic markers due to the fact that they are localized on the surface of malignant cells and certain GPI anchored proteins may be cleaved by phospholipase activity into circulation. Therefore, to identify proteins receiving the GPI anchor in breast carcinoma, we isolated these proteins from human tissue and serum using AT. The proteins identified by nano ESI-RPLC-MS/MS from tissue and serum were compared with data obtained using the human cell lines expressing control siRNA, GPAA1 siRNA, or PIGT siRNA. Cumulatively, over 1,000 individual proteins were identified from the membrane extractions of matched breast cancer tissues (3 cases-stage III ER+/PR+, stage II ER−/PR−, stage III ER+/PR+), cell lines (MCF10A and MDAMB231), and serum analysis (3 cases non-diseased and 3 cases invasive ductal breast carcinoma). As shown in FIG. 11A, total membrane isolation prior to nano ESI-RPLC-MS/MS led to membrane proteins being identified as 48% of the total proteins prior to AT enrichment. This value can be compared to 7-8% of membrane proteins being identified by MS/MS without prior membrane protein enrichment. The distribution of proteins after AT capture (proteins listed in Table 2, FIG. 15) is mainly membrane (>80%) as shown in FIG. 11B. Furthermore, over 50% of the membrane proteins identified binding with AT are localized at the plasma membrane. Therefore, AT enriches for proteins at the cell surface that represent a large pool of potential diagnostic or therapeutic biomarkers. Supplemental Table 2 lists all proteins, in alphabetical order with respect to official gene code, (2 or more peptides assigned for each protein from 2 separate biological samples) detected binding with AT from breast tissues (T), serum (S), and cell lines (CL). The percent coverage and complete peptide list for each protein in Table 2 (FIG. 15) is shown in Table 3 (FIG. 16). Surprisingly, there is an abundance of mitochondrial membrane proteins that are enriched binding to AT from breast cancer samples. We utilized DAVID (Database for Annotation, Visualization, and Integrated Discovery) to annotate the functions of the proteins listed in Table 2 (FIG. 15). Many of the proteins binding with AT function to bind and/or transport molecules such as amino acids, ions, lipids, and nucleotides, examples include: ATP1A, AT2A2, GOT2, RAB11B, SLC1A5, SLC25A24, and SLC25A5. A large number of proteins binding the toxin function in cell signaling and cell communication such as EPHA2, F3, GNAS, GNAI2, ITGA2, ITGA3, MYH9, CAV1, FERMT3 and FLNA. Several have enzymatic activities such as ATPase (ATP5B, ABCDF2, VCP, ATP1A1, RUVBL1, and HSPA8) or oxidoreductase activity (IMPDH2, HSD17B4, MDH2, and UQCRC2). Numerous cell surface glycoproteins binding with AT have IgG domains and function in cell adhesion, protein-protein clustering, integrin activation or antigen presentation such as BCAM, BSG, F3, HM13, IGHM, ITGA2, ITGA3, ITGA6, MUCB, and VCL. Finally, AT enrichment has led to the discovery of a breast cancer-specific uncharacterized membrane protein known as TMEM165 that is annotated to be GPI anchored.

Cluster of Differentiation Markers that Require GPAA1 and PIGT Expression for Membrane Localization are Also Found in Mesenchymal Stem Cell Populations.

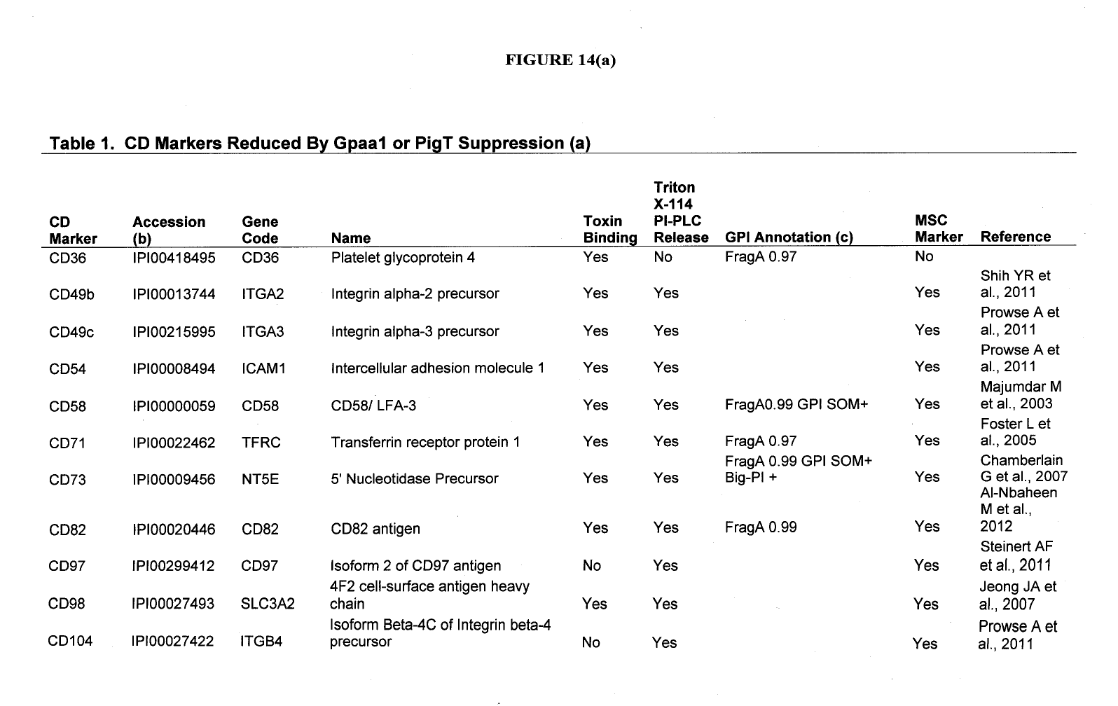

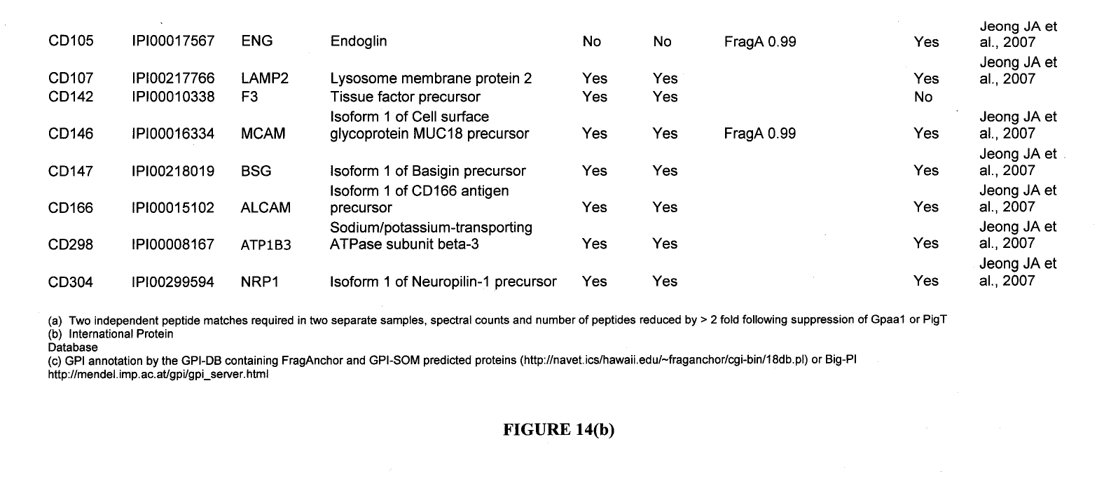

The inventors compared the membrane proteome of MDAMB231 cells expressing control siRNA, GPAA1 siRNA, and PIGT siRNA to identify proteins that change abundance in the membrane in response to changes in GPAA1 and PIGT expression levels. Many of the proteins binding with AT listed in Table 2 (FIG. 15) show GPIT dependent membrane localization. A protein listed as showing “GPIT dependence” indicates that the number of peptides detected and spectral abundance for these peptides identified from MDAMB231 cells were reduced by greater than 2 fold following GPAA1 or PIGT suppression. We identified all cluster of differentiation (CD) receptors that show GPIT dependence (Table 1, FIG. 14). Overwhelmingly, the CD markers listed in Table 1 have been reported in mesenchymal stem cell (MSC) populations (24-31). All of these proteins have tumor-specific expression in breast cancer tissue with the exception of CD36. Many of these proteins partition into the detergent fraction following triton X-114 extraction and can be released into the aqueous phase following PI-PLC treatment (mass spectrometry data). These results demonstrate that the expression of GPAA1 or PIGT in breast carcinoma contributes to the de-differentiation of breast epithelial cells leading to the cell surface expression of CD markers found in mesenchymal stem cell populations. Suppression of GPI addition by reducing the expression of GPAA1 and PIGT reduced the abundance of these CD markers in the cell membrane.

GPAA1 and PIGT Expression Levels Regulate FOXC2 Expression.

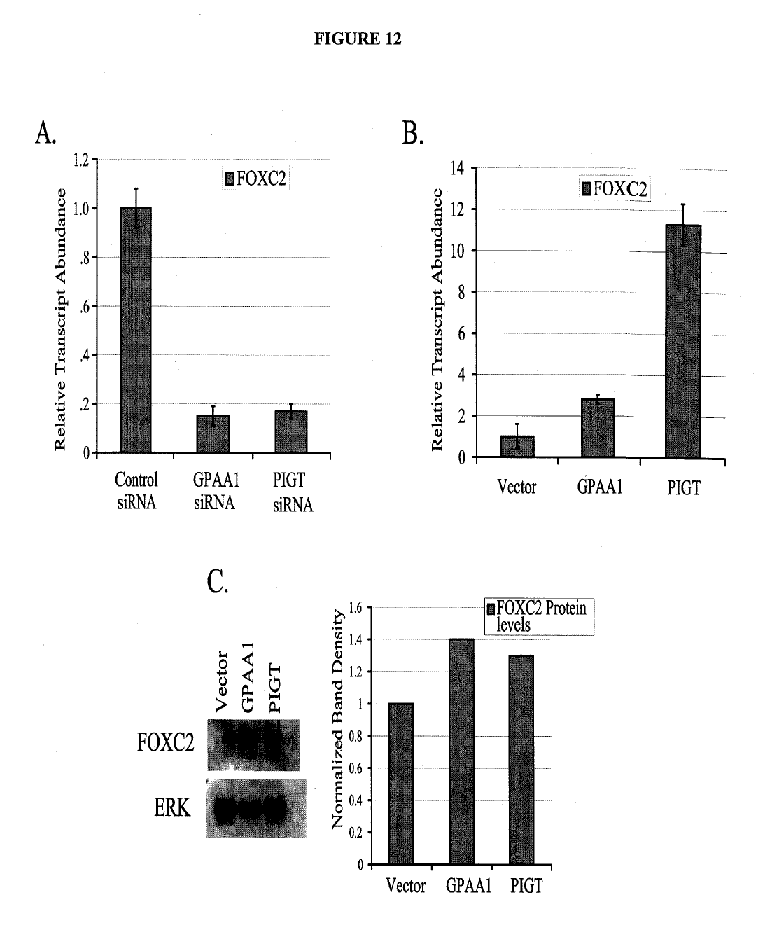

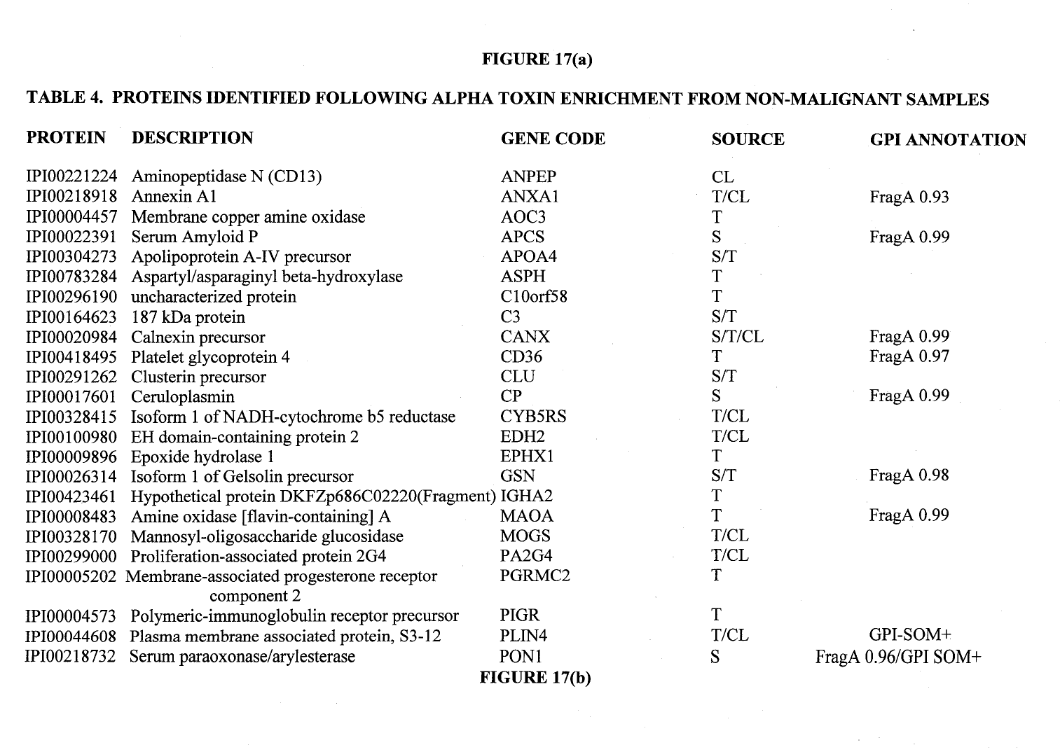

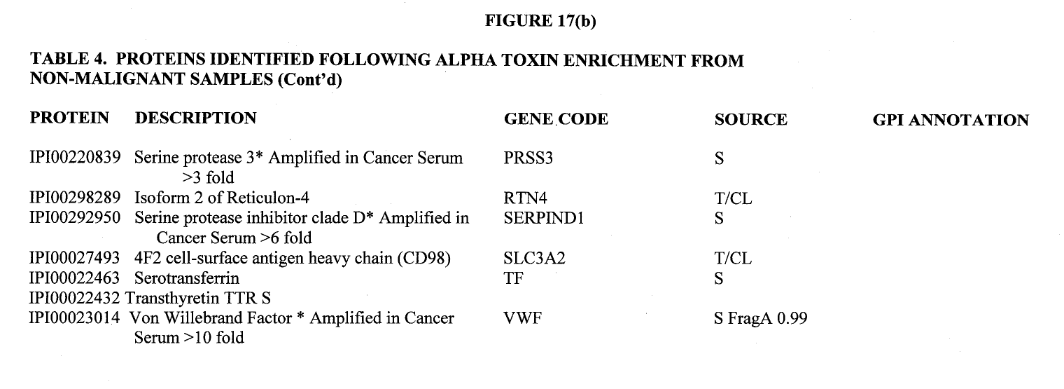

Most of the proteins listed in Table 2 (FIG. 15) and Table 1 (FIG. 14) are not detected by mass spectrometry in non-transformed breast epithelial cultured cells or normal breast tissue and normal serum. In supplemental Table 4 (FIG. 17), we list the AT bound proteins identified in normal MCF10A cells, normal breast tissue, or normal serum. Therefore the proteins in Table 2 (FIG. 15) and Table 1 (FIG. 14) are induced in malignant breast epithelial cells. Furthermore, due to the high prevalence of tumor-specific proteins associated with mesenchymal cell populations, we analyzed the relative expression levels of embryonic transcription factors that may induce mesenchymal gene expression using quantitative real-time PCR. We discovered that FOXC2 levels were decreased by >80% in MDAMB231 cells expressing GPAA1 or PIGT siRNA compared with control siRNA cells (FIG. 12A). This data suggests that FOXC2 expression is dependent on the expression of GPAA1 and PIGT. We wanted to further test for a relationship between the expression level of FOXC2 and GPAA1 or PIGT expression levels. Therefore, we cloned the cDNA for GPAA1 and PIGT into a lentiviral expression vector and expressed these genes in non-transformed MCF10A mammary cells. Stable cell lines express GPAA1 and PIGT mRNA at a 4-fold increase compared with vector only cells (data not shown). Lentiviral expression of either GPAA1 or PIGT in MCF10A cells results in increased levels of FOXC2 mRNA (FIG. 12B). FOXC2 protein levels in these cells were also elevated for GPAA1 and PIGT expressing MCF10A cells compared with control MCF10A cells (FIG. 12C). Therefore, we find a positive correlation between FOXC2 levels and GPAA1 and PIGT levels. Suppression of GPAA1 and PIGT in breast carcinoma significantly reduces FOXC2 expression; while increased expression of GPAA1 and PIGT in non-transformed mammary cells leads to elevated FOXC2 expression.

### Serum Proteins Binding to AT are Potential Biomarkers for the Detection of Breast Carcinoma.

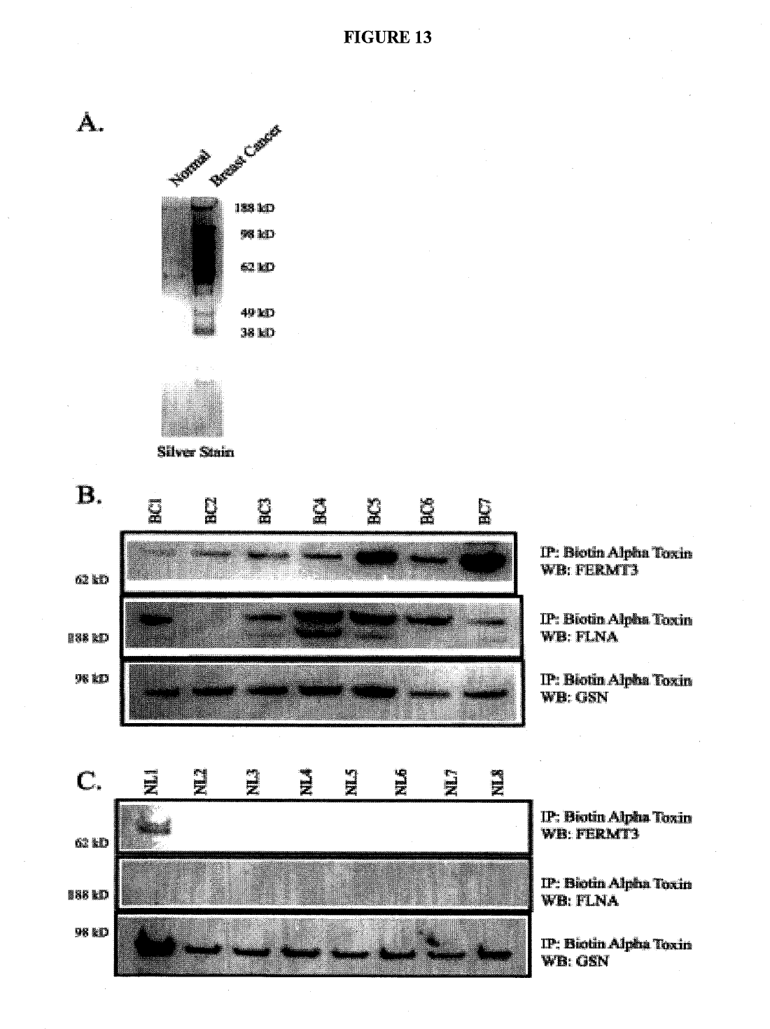

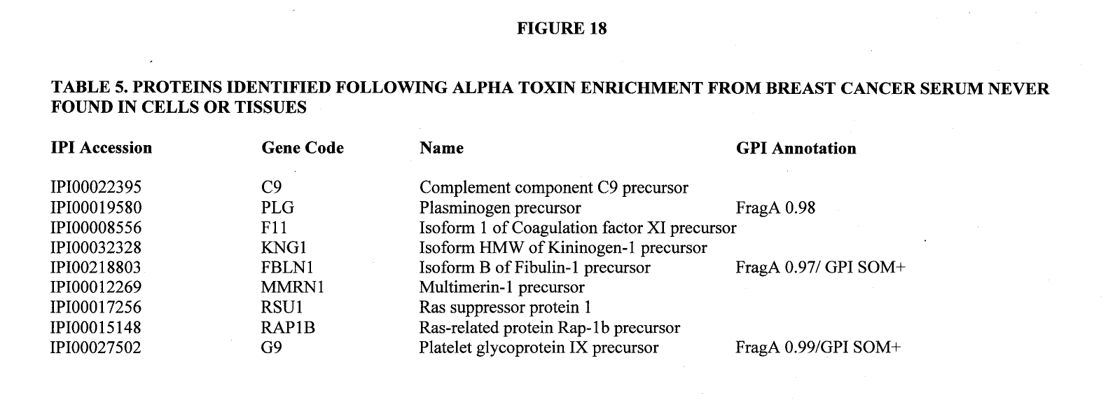

AT can be used to capture GPI anchored proteins from serum. Pooled serum from 5 non-diseased patients was compared with pooled serum from 5 patients with ductal invasive breast carcinoma following AT binding on magnetic beads. The silver stained gel shown in FIG. 13A indicates that GPI anchored proteins are captured using alpha toxin from the sera of breast cancer patients with very few proteins adhering to the toxin from normal serum. The serum proteins binding with AT were identified by nano-ESI-RPLC-MS/MS from 3 non-malignant serum samples and 3 of the serum samples from ductal breast carcinoma patients. Proteins binding with AT that were found in cancer tissue or cells as well as serum, are included in Table 2 (FIG. 15). In Table 5 (FIG. 18), the inventors list serum proteins that were found binding AT from cancer patient serum that were never found in tissues or cells. Interestingly, the inventors found proteins involved in the coagulation pathway (PLG, F11, and KNG1) that were not identified from tissue and cells that bind AT in breast cancer patient serum. How these proteins are connected with breast cancer is unclear; however, these proteins were never identified from non-diseased patient sera after AT pull down.

The inventors chose 2 of the proteins listed in Table 2 (FIG. 15) for further validation based on prevalence in breast cancer tissue and serum cases by nano-ESI-RPLC-MS/MS analysis. The inventors have analyzed by Western blot sera from 15 cases of ductal invasive breast carcinoma and 10 cases of non-malignant sera, including 8 women with benign polycystic breast disease. Selected results shown in FIG. 13B demonstrate that following AT capture, FERMT3 (kindlin 3) and FLNA (filamin A) are detected cumulatively in at least 90% of breast cancer sera analyzed. Results from our analysis of the 10 non-malignant cases, (serum from the polycystic breast disease patients shown in FIG. 13C), indicate that FERMT3 was detected in only 1 of 10 cases with no detection of FLNA in all 10 cases. Gelsolin (GSN) is detected following AT capture from both non-malignant and malignant serum and serves as a control for the input and quality of the serum. Overall, these results evidence that the detection of AT reactive FERMT3 and FLNA are useful for the detection of breast cancer from patient serum.

### Discussion

Mass spectrometry-based comparative membrane proteomics can enable the identification of novel cancer biomarkers by distinguishing proteins that change membrane localization between normal and malignant tissues and cells. The inventors have extended these capabilities by adding an additional selective enrichment using AT from C. septicum to identify proteins receiving a GPI anchor or associating with GPI anchored proteins in normal and malignant breast tissues, cells, and serum. To our knowledge this is the first study that uses AT as an in vitro capture agent and as a probe for GPI anchored proteins in tissue sections. Numerous proteins identified binding with AT (31%) listed in Table 2 (FIG. 15) were annotated to be GPI anchored proteins using the Frag Anchor, GPI SOM, or Big-PI algorithms.

The cell compartment data analysis described in FIG. 4 reveals that the majority of membrane proteins bound by AT are found at the plasma membrane or in membrane vesicles, suggesting possible involvement of the GPI anchor in facilitating protein movement from membrane organelles to the cell surface. GPI anchored proteins have previously been reported in the ER, Golgi, exocytic vesicles, endocytic vesicles, and at the cell surface in analysis of parasite GPI anchored proteins (32). The localization of GPI anchored proteins in lipid rafts has been reported to allow them to serve as platforms to mediate vesicle trafficking and signal transduction. In fact, GPI anchored proteins are probably the most mobile since they can be transferred readily between different cell surfaces (33,34). However, GPI anchored proteins have never been reported in the mitochondria. Our data showing that several mitochondrial membrane proteins isolated from tissues and cells are bound by AT represents the first suggestion of association with GPI anchored proteins or GPI anchor addition for some of these proteins. Four of the top 5 most abundant mitochondrial membrane proteins binding AT (IMMT, HADHB, SLC25A5, and GOT2) are annotated by databases to be GPI anchored. Also, ACO2 a mitochondrial enzyme binds AT in cancer patient serum, tissue, and malignant cells indicating release from the cell. Therefore, these proteins may represent novel biomarkers for breast cancer.

The inventors finding that CD markers associated with mesenchymal stem cell populations are decreased in the cell membrane in response to GPAA1 or PIGT suppression is novel. This is the first report of a link between GPAA1 and PIGT expression and the expression of mesenchymal stem cell markers. The CD44 antigen present on cancer stem cells (CSCs) (CD44^{high}/CD24^{low}) does not change abundance in the membrane in response to GPAA1 or PIGT suppression (data not shown). Moreover, the transcription factors Snail and Twist that often change expression levels in CSC populations are not changing in response to changes in GPAA1 and PIGT expression levels (data not shown). These data suggest that breast cancer cells expressing high levels of GPI anchored proteins may have a distinct mesenchymal stem cell niche, and warrant further investigations.

The inventors also report for the first time that the levels of the embryonic transcription factor Forkhead/Fox FOXC2 are correlated with changes in the expression levels of GPAA1 and PIGT. These results demonstrate that increased GPAA1 and PIGT expression may influence cell signaling pathways that activate FOXC2 expression. The FOXC2 transcription factor has been reported to be elevated in basal-like breast cancers (35). However, a recent study analyzed FOXC2 levels using the T-MTA-6A tissue array and found this transcription factor overexpressed in the majority of breast cancers and colon cancers suggesting a role in tumor progression (36). Ectopic expression of FOXC2 in normal adipose tissue induces mitochondrial biogenesis and increases the metabolic capacity of the cells (37). Our proteomic data indicating an enrichment of mitochondrial membrane proteins supports a possible hypothesis that the induction of FOXC2 in malignant breast epithelial cells due to increased GPIT expression leads to increased mitochondriogenesis and promotes the growth and expansion of dedifferentiated epithelial cells. Mass spectrometry-based proteomic analysis of serum proteins is often an arduous task requiring removal of abundant proteins and multiple steps of protein fractionation. We report that AT can be used to bind and isolate GPI anchored proteins released into serum, thereby simplifying the enrichment and proteomic analysis of potential markers from human serum. Using this method, we have verified that AT bound FERMT3 and FLNA from human serum are potential markers useful for the detection of breast carcinoma. Our analysis of a small set of sera from breast cancer patients and controls indicate that these markers can be bound by AT in 90% of the cancer cases with very low AT binding detected in non-malignant patients. A common obstacle to establishing a serum-based detection assay is the formation of protein complexes in serum and the inability of antibodies to detect certain proteins that may be found in these complexes. We have shown that AT binds very well to proteins from serum in vitro (FIG. 13). Therefore, AT can be used to isolate protein markers before detection using established platforms such as ELISA.

In conclusion, we have developed AT as a reagent useful for the detection and capture of GPI anchored proteins. We have shown that GPI anchored protein expression is elevated in malignant breast epithelial cells. Our mass spectrometry-based comparative membrane proteomic results suggest new roles for GPI anchored proteins in breast cancer progression such as epithelial dedifferentiation and increased mitochondrial protein expression. In addition, we showed that FOXC2 expression is regulated by GPAA1 and PIGT expression levels. These findings indicate that molecular therapeutics targeting GPI biosynthetic machinery or specific GPI anchored cell surface receptors may be useful to control the de-differentiation of breast epithelial cells in malignant disease. Our discovery that AT can be used to capture GPI anchored proteins from serum has led to the discovery of new potential detection markers for breast cancer such as FERMT3 and FLNA. The discovery of AT binding to these proteins in both malignant tissue and cancer patient serum suggests that these proteins may be important in tumor progression and may be useful for the detection and prognostic monitoring of breast cancer using patient serum.

### SUMMARY

The glycosylphosphatidylinositol (GPI) anchor is a lipid and glycan modification added to the C-terminus of certain proteins in the endoplasmic reticulum (ER) by the activity of a multiple subunit enzyme complex known as the GPI transamidase (GPIT). Several subunits of GPIT have increased expression levels in breast carcinoma. In an effort to identify GPI anchored proteins and understand the possible role of these proteins in breast cancer progression, we employed a combination of strategies. First, alpha toxin from Clostridium septicum was used to capture GPI anchored proteins from human breast cancer tissues, cells, and serum for proteomic analysis. We also expressed short interfering RNAs targeting the expression of the GPAA1 and PIGT subunits of GPIT in breast cancer cell lines to identify proteins whose membrane localization is dependent on GPI anchor addition. Comparative membrane proteomics using nano ESI-RPLC-MS/MS led to the discovery of several new potential diagnostic and therapeutic targets for breast cancer. Furthermore, we provide evidence that increased GPI anchor addition in malignant breast epithelial cells promotes the dedifferentiation of malignant breast epithelial cells in part by increasing the levels of cell surface markers associated with mesenchymal stem cells.

### Examples

### Elevated Levels of Glycosylphosphatidylinositol (GPI) Anchored Proteins in Serum from Human Cancers Detected by Alpha Toxin (Third Set of References Applies)

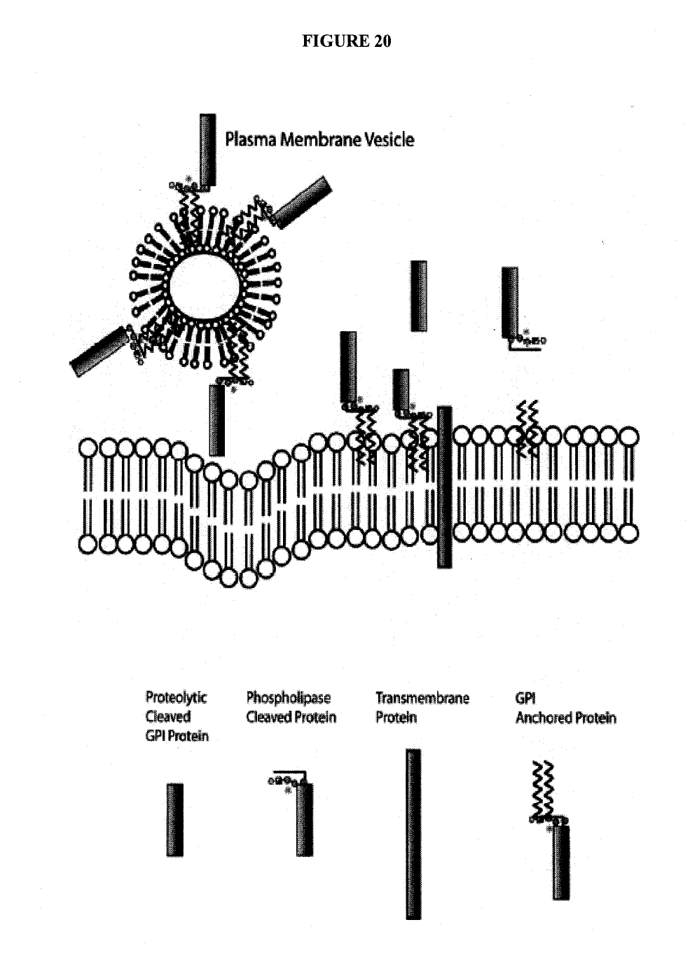

The primary mechanism that allows GPI anchored proteins to enter the circulatory system from tumors is not well understood. GPI anchored proteins can potentially be released from cells by proteolysis (6), GPI-specific phospholipase activities (7, 8), or by exosome vesicular transport from the cell (6) (FIG. 20). The GPI glycan would remain attached to the GPI anchored proteins if the proteins were released by exosome or GPI-specific phospholipase cleavage (FIG. 20). Our goal with the current study is to use alpha toxin to determine if GPI anchored proteins are present at elevated levels with a GPI anchor glycan in serum samples obtained for various human cancers.

### Patients and Methods

### Patients

Blood from non-diseased and patients with ductal invasive breast carcinoma, ovarian cancer, kidney cancer, colon cancer, liver cancer, lung cancer, or brain cancer was collected pre-operatively and in accordance with approved institutional review board human subject guidelines at GHSU or the Ovarian Cancer Institute (Table 1). Serum fractions were stored at −70° C. until use.

### Slot Blot and Alpha Toxin Detection

Serum (5 μl) was mixed with 5 μl laemelli buffer. Samples were heated and applied to nitrocellulose Protran BA85 membrane using a Schleicher and Schuell Minifold I Slot Blot System. The membrane was blocked in 5% milk/1×TBST (Blotto Solution) overnight at 4° C. The blot was incubated with biotin labeled alpha toxin (2 μg/ml) purified and labeled as described previously (4). Bound toxin was detected using a 1:5,000 dilution of streptavidin-HRP (Vector Labs, Burlingame, Calif.) before washing and detection using Western Lightening Plus (Perkin Elmer). Slot blots were then stripped using 0.1M glycine pH 2.9 overnight, blocked again, and detected using anti-alpha 1 glycoprotein antibody (Sigma) to normalize for total protein content. Intensity of alpha toxin binding was determined using ImageJ analysis normalized to total protein band density.

Phospholipase C treatment and detection of CEA5 in LS174T cells. Approximately 10×10⁶ LS174T colon cancer cells were collected by gentle cell scraping. Cells were diluted with 200 μl 1×PBS with Calcium and Magnesium and evenly split into 2 fractions. One fraction received buffer only and one received 1.5 U/ml GPI-specific phospholipase C (Invitrogen) for 1 hour at 37° C. The cells were collected by centrifugation and the supernatants were collected for analysis. Biotin labeled alpha toxin 2 mg/ml was added and the samples were incubated at room temperature for 30 minutes. Streptavidin magnetic beads (Promega) were added for 30 additional minutes at room temperature. Beads were washed 3× with 1×PBS before releasing the proteins with Laemmli buffer. Proteins were separated on a 4-12% Bis-Tris polyacrylamide gel (Invitrogen) and transferred to PVDF for detection of CEA5 (monoclonal antibody COL-1, Invitrogen) or the gel with 10% fractions was fixed and silver stained.

### Results

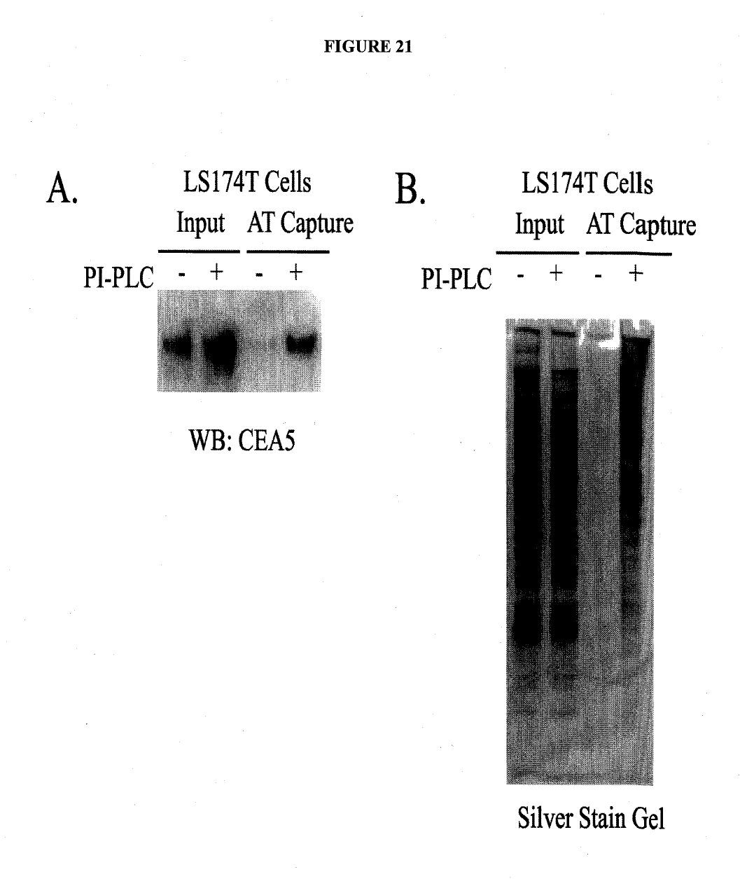

In the previous examples, the inventors suppressed the expression of the GPAA1 and PIGT subunits of the GPIT enabling them to establish that alpha toxin binding required the addition of the GPI anchor to proteins (4). In this example, the inventors show that alpha toxin can bind with GPI anchored proteins that have been cleaved by GPI-specific phospholipase. As shown in FIG. 21A, LS174T colon cancer cells were incubated with or without GPI-specific phospholipase C. Biotin labeled alpha toxin was added to capture GPI anchored proteins. Western blot detection of the GPI anchored protein carcinoembryonic antigen 5 (CEA5) indicate that CEA5 is present in the input supernatant samples from both untreated and PI-PLC treated cells. However, alpha toxin only captures CEA5 from PI-PLC treated cells indicating the at the CEA5 released endogenously from LS174T cells does not contain the GPI anchor glycan and is likely released by proteolysis. Equivalent levels of proteins were present from the supernatant of both reactions (FIG. 21B); therefore, alpha toxin binding is specific for the presence of the GPI anchor glycan attached to GPI anchored proteins.

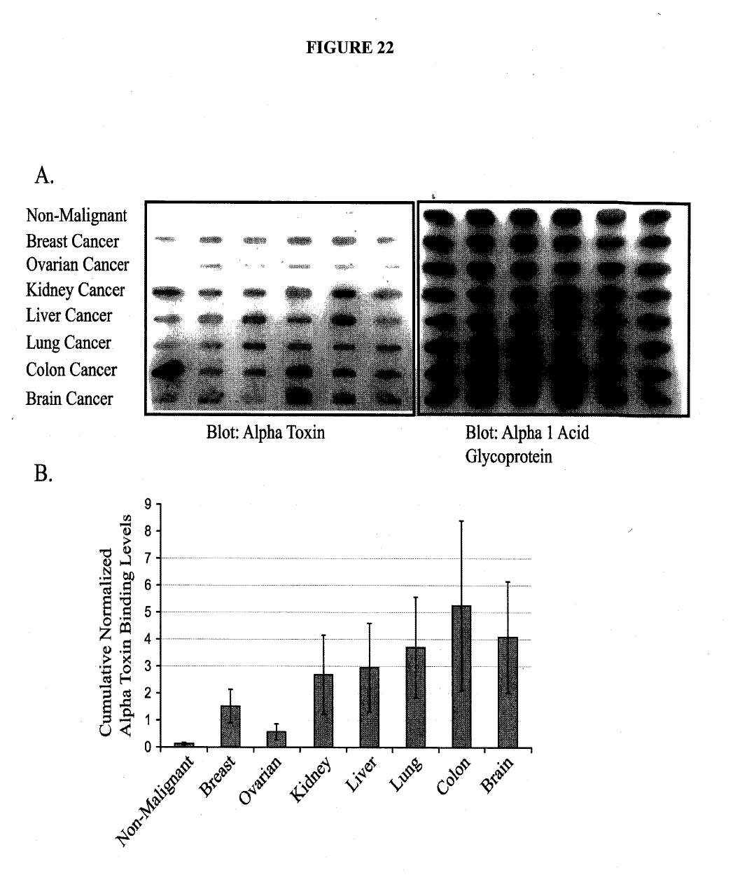

The levels of GPI anchored proteins present in serum can be controlled by many factors such as the levels of GPI anchored protein acceptors, the levels of GPI transamidase subunits expressed in different tumors, the levels of endogenous GPI phospholipase activity, and the levels of protease activity. In addition to these factors, solid tumors in different organs may sequester GPI anchored proteins into highly hydrophobic lipid raft membrane domains that are resistant to enzyme release. Based on our analysis of breast cancer tissue and serum, increased GPI anchored proteins were present in serum from breast cancer patients. Breast cancers have frequent amplifications of chromosomal regions that contain the GPIT subunits. In table 1, we list the subunits of the GPIT and the chromosomal location of each subunit. In addition, we indicate human cancers that have chromosomal amplifications in the regions that contain GPIT subunits (9-19). Furthermore, we list any existing published data indicating increased expression for GPIT subunits in certain human cancers (1-3, 20-22). The information in table 1 indicates that the GPI transamidase is elevated in many human cancers, therefore the inventors hypothesize that increased levels of GPI anchored proteins may be present in serum from patients with these malignancies. The inventors have analyzed serum collected from breast, ovarian, kidney, liver, lung, colon, and brain cancer by slot blot followed by alpha toxin detection (table 2). An example slot blot shown in FIG. 22A indicates that GPI anchored proteins could be detected in serum from breast, ovarian, kidney, liver, lung, colon, and brain cancer with no detection in serum from patients without malignant disease. Each slot contained equivalent levels of serum proteins evidenced by the alpha-1 acid glycoprotein levels (FIG. 22A, right). The inventors have analyzed 12 samples from each type of cancer except ovarian (6 cases) along with 12 serum samples from patients without malignant disease (table 2). Densitometry analysis of slot blots were performed and the cumulative averaged alpha toxin signal intensities normalized to alpha-1 acid glycoprotein levels with SEM for each cancer are shown in FIG. 22. These results indicate that GPI anchored proteins with a GPI anchor glycan attached were detected in the serum from cancer patients at significantly higher levels compared with serum from patients without malignant disease. Variability exists in the levels of GPI anchored proteins detected by alpha toxin in different cancers and between different patients within certain cancer types. The cancers that show the highest variability are colon and brain cancer. Despite the patient variability, cumulative data indicate that GPI anchored proteins were detected in serum at increased levels for all cancers analyzed (FIG. 22B). Therefore, proteomic studies to identify these GPI anchored proteins could lead to the discovery of novel biomarkers for cancer.

### Discussion

GPI anchored proteins are vital for cell viability. However, the levels of GPI anchored proteins in normal cells are under tight control evidenced by the lower levels of GPI transamidase mRNA and protein levels in normal tissues and cells (4). GPIT levels are amplified in human cancers due to chromosomal amplifications acquired during malignant transformation. The impact of how the amplification of GPIT subunits can influence cancer progression is just beginning to be assessed. Breast cancer studies indicate that elevations of GPIT lead to increased levels of GPI anchored proteins (4) and increased levels of tumorigenicity (1). Based on these findings we sought to determine if the levels of GPI anchored proteins in serum for various cancers correlates with previously described amplifications of GPIT levels in the tissues for these cancers. The inventors' results indicate that all cancers that have amplifications of certain GPIT subunits also have elevated alpha toxin binding demonstrating increased levels of GPI anchored proteins in the serum. The inventors were surprised to discover that cancers with high GPIT mRNA and protein expression, such as breast and ovarian, do not have the highest levels of alpha toxin binding. For example, ovarian cancer has been shown to have the highest levels of expression for GPIT subunits, catalytic and non-catalytic (3). Yet, the data presented in this manuscript reveal that while GPI anchored proteins are detected by alpha toxin at higher levels in ovarian cancer compared with control serum, the levels are lower than other cancers. These results illustrate that other factors contribute to release of GPI anchored proteins into serum in a form that can be detected by alpha toxin such as, possible amplification of protease activity, or release by an endogenous GPI phospholipase that may result in a modified GPI anchor glycan region that is not recognized as avidly by alpha toxin. We have proteomic data that indicates high levels of GPI anchored protein expression detected by alpha toxin binding in ovarian cancer tumors (data not shown) and ascites (23); therefore, future studies to detail the differences in the GPI anchor structures of these proteins from tissue, ascites, and serum may offer insight into why alpha toxin binding is lower for ovarian cancer serum.

The functional significance of why GPI anchored proteins are released from the cell is not well understood. Studies from unicellular eukaryotic species have offered some insights into possible roles for releasing GPI anchored proteins (24). Possible explanations include: greater cell to cell communication, a method to control antigenic variability, and control of cell shape influencing growth and migration characteristics of cells. Therefore, it is not difficult to envision how tumor cells would gain an advantage releasing GPI anchored proteins. We have discovered that cancers from the colon and brain have the highest levels of GPI anchored proteins detected in serum. The higher release of GPI anchored proteins from these cancers may reflect a higher level of cell to cell communication such as synaptic activity in the brain, or a greater need to evade immune response such as the adaptive pathogen responses in colonic epithelial cells.

In conclusion, the data documenting elevated levels of GPI anchored proteins in the serum from human cancers indicate that this glycoconjugate is an ideal biomarker useful for the surveillance and detection of human cancers. We have demonstrated that alpha toxin can be used as a GPI lectin to detect GPI anchored proteins in human serum. Therefore, the identification of tumor-specific GPI anchored proteins for cancer can foster the development of novel cancer detection methods and therapeutic strategies utilizing alpha toxin.

### Further Examples

The inventors have optimized the sample preparation and extraction method used in the present invention. In particular, the inventors have performed Triton X-114 phase partitioning using the MDAMB231 breast cancer cell lines, as well as with a pooled sample of the human breast cancer serum analyzed in the original study in order to determine the efficacy of adding this step prior to alpha toxin affinity pull-downs.

Details of the methods used: The Triton X-114 extraction for the cells was performed according to the method of Doering et al., published in Current Protocols (Curr. Protoc. Mol. Biol. 2001 May; Chapter 17: Unit 17.8. The detergent partitioned phase was treated with PI-PLC from B. Cereus (Invitrogen) to release the GPI anchored proteins to the aqueous phase. The aqueous phase was precipitated and either subjected to alpha toxin capture or proteins were separated by SDS-PAGE and extracted by in-gel trypsin digest prior to nano-ESI-RPLC-MS/MS analysis. For serum, we used 200 uL composed of the same cases analyzed previously. The 200 uL serum sample was diluted with equal volume 1×TBS and brought to a final of 2% Triton X-114. Particulates were removed by centrifugation prior to phase separation. The detergent enriched fraction was treated with PI-PLC as described for the cells. These proteins were separated by SDS-PAGE and excised for in-gel trypsin extraction and ESI-RPLC-MS/MS analysis. In total over 25 samples were analyzed by mass spectrometry with subsequent data analysis.

Results: The mass spectrometry data from cell analysis and serum analysis were very similar to the previous data. However, the inventors did discover 2 additional known GPI anchored proteins that were missed without the Triton X-114 extraction; these were added to table 1, CD58/LFA-3 and BMST2. The inventors also included an additional protein CD82 that was identified in the Triton X-114 extraction following PI-PLC cleavage that is annotated by FragAnchor to be GPI anchored. CD82 has been reported as a potential mesenchymal stem cell marker. The inventors have also added to table 1 a column that denotes whether CD markers were released by PI-PLC following Triton x-114 extraction. Several of the CD markers were identified at higher protein coverage following Triton X-114 detergent partitioning and PI-PLC cleavage. These peptides were updated on the supplementary table 2 that lists the peptides identified. The serum analysis revealed that both potential breast cancer markers FERMT3 and FLNA were partitioned in the detergent phase and released with PI-PLC, further demonstrating that these proteins binding with alpha toxin may be GPI anchored.

The inventors could not find a previous analysis of GPI anchored proteins from human breast cancer epithelial cells or non-diseased human breast epithelial cells; therefore, there can be no expectation of specific known GPI anchored proteins from these sources. Table 1 is labeled as “Proteins identified following alpha toxin enrichment of human breast cancer samples”; this does not indicate that these proteins are all GPI anchored. The inventors acknowledge and note that several proteins in the list may be co-associating with GPI anchored proteins, rather than being part of GPI-APs per se. They have, therefore, edited Table 2 and marked in italics proteins that have tryptic peptides identified in their C-termini, making them unlikely to be GPI anchored proteins.

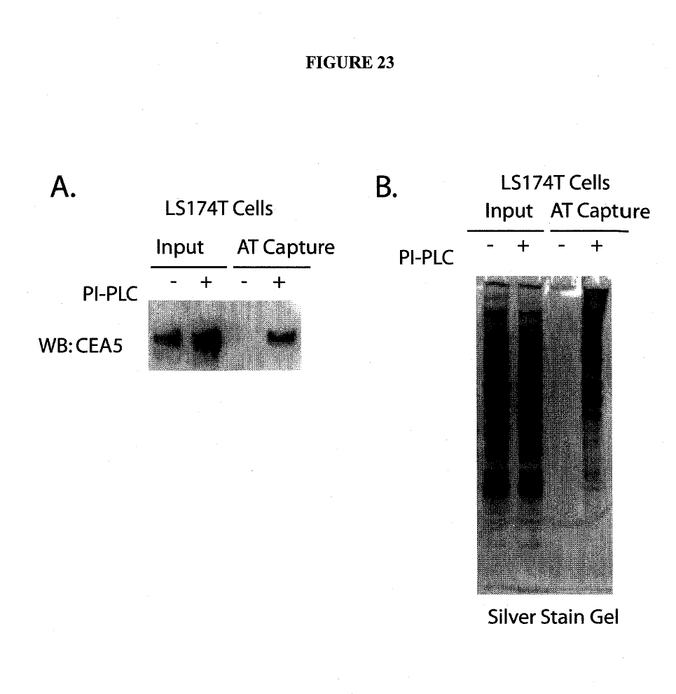

In addition, the inventors have performed validation experiments for FERMT3 and include as a positive control the GFP-GPI (DAF) construct previously used as a control for GPI anchor studies (See Legler, et al., Faseb J, 19(1) 73-75, 2005). The data is not shown. The inventors had performed several optimization experiments using alpha toxin prior to evaluating clinical breast cancer tissues and sera. One of these experiments is shown to demonstrate the specificity of alpha toxin for the GPI anchor. Intact colon cancer cells LS174T were treated with or without GPI specific PI-PLC to release surface GPI anchored proteins prior to alpha toxin capture. The protein carcinoembryonic antigen 5 (CEA5) is present in the sample without PI-PLC treatment (FIG. 23A), yet this form of CEA5 is likely released from the cell and cleaved by proteases leading to removal of the GPI anchor since the alpha toxin only reacts with CEA5 that is PI-PLC released from the cell surface (FIG. 23A). In FIG. 23B the total protein present in each sample used for FIG. 23A is shown by silver stain, demonstrating equivalent inputs in each binding reaction. This experiment provides evidence for the specificity of the toxin for the GPI anchor.

In addition to the experiment shown above demonstrating that alpha toxin can bind with a known GPI anchored protein, we have initiated verification experiments for the marker discovered in this manuscript, FERMT3 in an effort to determine if this protein is GPI anchored in breast cancer. The inventors obtained a FERMT3 plasmid previously published from Dr. Edward Plow. (See Bialkowska, et al., J. Biol. Chem. 285(24) 18640-18649, 2010). This construct allows the expression of GFP fused to the amino-terminal side of FERMT3.

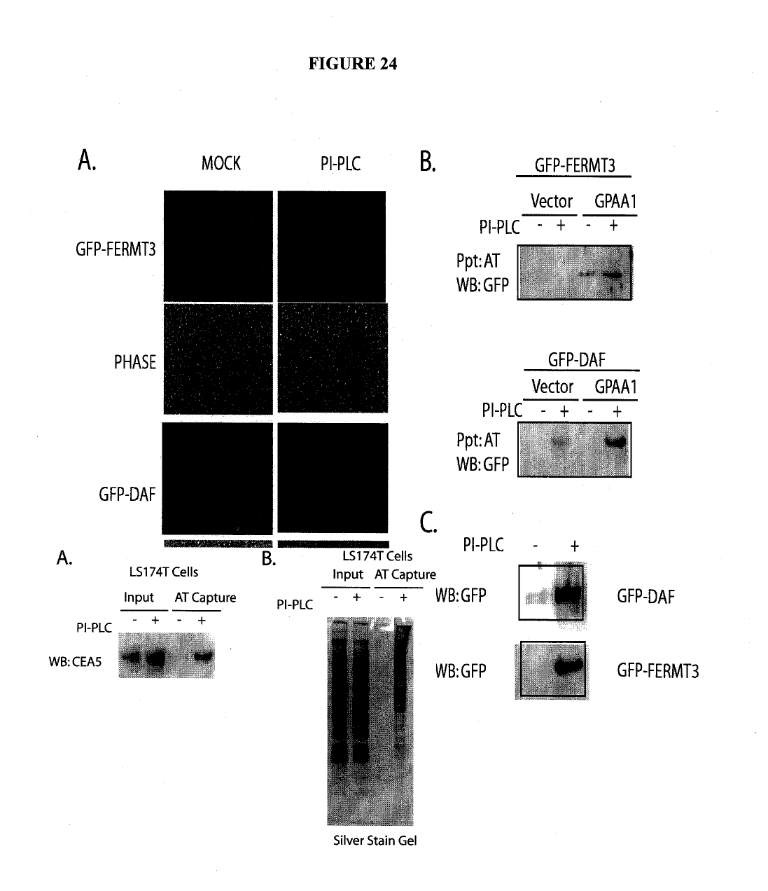

GFP-FERMT3 and GFP-DAF were transiently transfected into HEK cells stably expressing vector only or GPAA1 cDNA. The GPAA1 gene is part of the GPI transamidase and is elevated in breast cancer. The cells were collected and split into 2 tubes and treated with buffer only (mock) or PI-PLC for 1 hour at 37 C. The supernatant was used for alpha toxin capture followed by Western blot detection of GFP (FIG. 24B) and the cells were fluorescently imaged (FIG. 24A—GPAA1 cells shown). Our data indicates that GFP-FERMT3 is not released by PI-PLC in the vector cells (FIG. 24B, top panel) (we also saw no change in fluorescent intensity after PI-PLC—data not shown). However, expression of GPAA1 leads to susceptibility of GFP-FERMT3 to PI-PLC cleavage (FIG. 24A-compare the fluorescence intensity change between mock and PI-PLC treated cells in GFP-DAF and GFP-FERMT3, also note presence in Western blot, FIG. 24B—upper panel). GPAA1 expression also leads to an increase in the level of GFP-DAF control that is released by PI-PLC (FIG. 24B, lower panel compare vector cells and GPAA1 cells). Next, we used Triton X-114 extraction to determine if GFP-FERMT3 is partitioned in the detergent phase and is released by PI-PLC into the aqueous phase. Results shown in FIG. 24C indicate that both the control GFP-DAF and GFP-FERMT3 are partitioned into the detergent fraction and are released upon addition of PI-PLC. This data suggest that the expression of GPAA1, as is observed for breast cancer, increases the levels of GPI anchoring and may alter the specificity of proteins receiving the GPI anchor. FERMT3 is annotated to be an intracellular membrane protein and would not be accessible to intact cell PI-PLC treatment unless it was present on the extracellular side of the membrane. Our data indicates that when GPAA1 is expressed GFP-FERMT3 is susceptible to PI-PLC cleavage indicating extracellular localization. Definitive evidence that FERMT3 is GPI anchored in breast cancer will require mass spectrometry analysis of the GPI anchor and the identification of the peptide sequence with the GPI anchor.

Responses to Specific Points from Reviewers.

### Results:

The inventors have performed Triton X-114 extraction followed by PI-PLC digestion on breast cancer cells and serum prior to in-gel extraction for ESI-RPLC-MS/MS analysis. This method yielded 3 additional annotated GPI anchored proteins found in the cells, 2 known GPI anchored proteins and one annotated by FragAnchor. Therefore, this method of extraction coupled with alpha toxin capture may be useful for increasing the coverage of lower abundance GPI anchored protein detection. Overall, the detergent extraction results were very similar to the previous proteomic data and do not change the conclusions.

Although there were no known GPI anchored proteins identified from serum, the inventors only included serum proteins in table 1 that were also found in breast cancer tissue or cells in an effort to identify potential markers released from breast cancer cells into serum. In the supplement tables 3 and 4 there are several proteins annotated as GPI anchored proteins found in non-malignant samples (example, ceruloplasmin) and cancer patient serum without identification in tissue or serum (example, platelet glycoprotein IX). The inventors performed Triton x-114 extraction followed by PI-PLC release of breast cancer serum. This analysis did not yield different data than the original results. Therefore, the use Triton x-114 phase partitioning to concentrate potential GPI anchored proteins from serum did not increase the identification of GPI anchored proteins from serum and may be viewed as an optional step in separating GPI-APs from cells. The inventors did find that the proteins identified as potential biomarkers, FERMT3 and FLNA, were partitioned into the detergent phase following Triton X-114 and released by PI-PLC.

The inventors also validated mitochondrial protein Triton x-114 partitioning and PI-PLC release. They performed this experiment and found that some mitochondrial proteins did partition to the detergent phase and were released by PI-PLC (determined by mass spectrometry data). To definitively state that these are receiving a GPI anchor in breast cancer will require mass spectrometry analysis of the GPI anchor structure and determination of the GPI anchor attachment site.

### REFERENCES

### First Set

1. Paulick, M. G., and Bertozzi, C. R. (2008). The glycosylphosphatidylinositol anchor: a complex membrane-anchoring structure for proteins. Biochemistry 47, 6991-7000.
2. Simons, M., Friedrichson, T., Schulz, J. B., Pitto, M., Masserini, M., and Kurzchalia, T. V. (1999). Exogenous administration of gangliosides displaces GPI-anchored proteins from lipid microdomains in living cells. Mol Biol Cell 10, 3187-96.
3. Metz, C. N., Brunner, G., Choi-Muira, N. H., Nguyen, H., Gabrilove, J., Caras, I. W., Altszuler, N., Rifkin, D. B., Wilson, E. L., and Davitz, M. A. (1994). Release of GPI-anchored membrane proteins by a cell-associated GPI-specific phospholipase D. Embo J 13, 1741-51.
4. Gordon, V. M., Nelson, K. L., Buckley, J. T., Stevens, V. L., Tweten, R. K., Elwood, P. C., and Leppla, S. H. (1999). Clostridium septicum alpha toxin uses glycosylphosphatidylinositol-anchored protein receptors. J Biol Chem 274, 27274-80.
5. Melton-Witt, J. A., Bentsen, L. M., and Tweten, R. K. (2006). Identification of functional domains of Clostridium septicum alpha toxin. Biochemistry 45, 14347-54.
6. Ikezawa, H., Yamanegi, M., Taguchi, R., Miyashita, T., and Ohyabu, T. (1976). Studies on phosphatidylinositol phosphodiesterase (phospholipase C type) of Bacillus cereus. I. purification, properties and phosphatase-releasing activity. Biochim Biophys Acta 450, 154-64.
7. Elortza, F., Nuhse, T. S., Foster, L. J., Stensballe, A., Peck, S. C., and Jensen, O. N. (2003). Proteomic analysis of glycosylphosphatidylinositol-anchored membrane proteins. Mol Cell Proteomics 2, 1261-70.
8. Elortza, F., Mohammed, S., Bunkenborg, J., Foster, L. J., Nuhse, T. S., Brodbeck, U., Peck, S. C., and Jensen, O. N. (2006). Modification-specific proteomics of plasma membrane proteins: identification and characterization of glycosylphosphatidylinositol-anchored proteins released upon phospholipase D treatment. J Proteome Res 5, 935-43.
9. McConville, M. J., and Ferguson, M. A. (1993). The structure, biosynthesis and function of glycosylated phosphatidylinositols in the parasitic protozoa and higher eukaryotes. Biochem J294 (Pt 2), 305-24.
10. Brewis, I. A., Ferguson, M. A., Mehlert, A., Turner, A. J., and Hooper, N. M. (1995). Structures of the glycosyl-phosphatidylinositol anchors of porcine and human renal membrane dipeptidase. Comprehensive structural studies on the porcine anchor and interspecies comparison of the glycan core structures. J Biol Chem 270, 22946-56.
11. Eisenhaber, B., Maurer-Stroh, S., Novatchkova, M., Schneider, G., and Eisenhaber, F. (2003). Enzymes and auxiliary factors for GPI lipid anchor biosynthesis and posttranslational transfer to proteins. Bioessays 25, 367-85.
12. Kinoshita, T., and Inoue, N. (2000). Dissecting and manipulating the pathway for glycosylphos-phatidylinositol-anchor biosynthesis. Curr Opin Chem Biol 4, 632-8.
13. Hong, Y., Ohishi, K., Inoue, N., Kang, J. Y., Shime, H., Horiguchi, Y., van der Goot, F. G., Sugimoto, N., and Kinoshita, T. (2002). Requirement of N-glycan on GPI-anchored proteins for efficient binding of aerolysin but not Clostridium septicum alpha-toxin. Embo J 21, 5047-56.
14. Shin, D. J., Cho, D., Kim, Y. R., Rhee, J. H., Choy, H. E., Lee, J. J., and Hong, Y. (2006). Diagnosis of paroxysmal nocturnal hemoglobinuria by fluorescent clostridium septicum alpha toxin. J Mol Microbiol Biotechnol 11, 20-7.
15. Wu, G., Guo, Z., Chatterjee, A., Huang, X., Rubin, E., Wu, F., Mambo, E., Chang, X., Osada, M., Sook Kim, M., Moon, C., Califano, J. A., Ratovitski, E. A., Gollin, S. M., Sukumar, S., Sidransky, D., and Trink, B. (2006). Overexpression of glycosylphosphatidylinositol (GPI) transamidase subunits phosphatidylinositol glycan class T and/or GPI anchor attachment 1 induces tumorigenesis and contributes to invasion in human breast cancer. Cancer Res 66, 9829-36.

### REFERENCES

### Second Set

1. Micanovic, R., Kodukula, K., Gerber, L. D., and Udenfriend, S. (1990) Proc Natl Acad Sci USA 87(20), 7939-7943
2. Eisenhaber, B., Bork, P., and Eisenhaber, F. (2001) Protein Eng 14(1), 17-25
3. Yamamoto, Y., Hirakawa, E., Mori, S., Hamada, Y., Kawaguchi, N., and Matsuura, N. (2005) Biochem Biophys Res Commun 333(1), 223-229
4. Argani, P., Iacobuzio-Donahue, C., Ryu, B., Rosty, C., Goggins, M., Wilentz, R. E., Murugesan, S. R., Leach, S. D., Jaffee, E., Yeo, C. J., Cameron, J. L., Kern, S. E., and Hruban, R. H. (2001) Clin Cancer Res 7(12), 3862-3868
5. Han, K. R., Seligson, D. B., Liu, X., Horvath, S., Shintaku, P. I., Thomas, G. V., Said, J. W., and Reiter, R. E. (2004) J Urol 171(3), 1117-1121
6. McCabe, N. P., Angwafo, F. F., 3rd, Zaher, A., Selman, S. H., Kouinche, A., and Jankun, J. (2000) Oncol Rep 7(4), 879-882
7. Eisenhaber, B., Bork, P., and Eisenhaber, F. (1998) Protein Eng 11(12), 1155-1161
8. Poisson, G., Chauve, C., Chen, X., and Bergeron, A. (2007) Genomics Proteomics Bioinformatics 5(2), 121-130
9. Fankhauser, N., and Maser, P. (2005) Bioinformatics 21(9), 1846-1852
10. Eisenhaber, B., Bork, P., and Eisenhaber, F. (1999) J Mol Biol 292(3), 741-758
11. Ikezawa, H. (1991) Cell Biol Int Rep 15(11), 1115-1131
12. Treumann, A., Lifely, M. R., Schneider, P., and Ferguson, M. A. (1995) J Biol Chem 270(11), 6088-6099
13. Gordon, V. M., Nelson, K. L., Buckley, J. T., Stevens, V. L., Tweten, R. K., Elwood, P. C., and Leppla, S. H. (1999) J Biol Chem 274(38), 27274-27280
14. Wu, G., Guo, Z., Chatterjee, A., Huang, X., Rubin, E., Wu, F., Mambo, E., Chang, X., Osada, M., Sook Kim, M., Moon, C., Califano, J. A., Ratovitski, E. A., Gollin, S. M., Sukumar, S., Sidransky, D., and Trink, B. (2006) Cancer Res 66(20), 9829-9836
15. Sellman, B. R., Kagan, B. L., and Tweten, R. K. (1997) Mol Microbiol 23(3), 551-558
16. Doering, T. L., Englund, P. T., and Hart, G. W. (2001) Curr Protoc Protein Sci Chapter 12, Unit 12 15
17. Tabb, D. L., Fernando, C. G., and Chambers, M. C. (2007) JProteome Res 6(2), 654-661
18. Nairn, A. V., dela Rosa, M., and Moremen, K. W. Methods Enzymol 479, 73-91
19. Nairn, A. V., Kinoshita-Toyoda, A., Toyoda, H., Xie, J., Harris, K., Dalton, S., Kulik,
M., Pierce, J. M., Toida, T., Moremen, K. W., and Linhardt, R. J. (2007) J Proteome Res 6(11), 4374-4387
20. Shin, D. J., Choy, H. E., and Hong, Y. (2005) J Microbiol 43(3), 266-271
21. Ashok, A., and Hegde, R. S. (2008) Mol Biol Cell 19(8), 3463-3476
22. Ohishi, K., Inoue, N., Maeda, Y., Takeda, J., Riezman, H., and Kinoshita, T. (2000) Mol Biol Cell 11(5), 1523-1533
23. Ohishi, K., Inoue, N., and Kinoshita, T. (2001) Embo J 20(15), 4088-4098
24. Shih, Y. R., Tseng, K. F., Lai, H. Y., Lin, C. H., and Lee, O. K. J Bone Miner Res 26(4), 730-738
25. Prowse, A. B., Chong, F., Gray, P. P., and Munro, T. P. Stem Cell Res 6(1), 1-12
26. Majumdar, M. K., Keane-Moore, M., Buyaner, D., Hardy, W. B., Moorman, M. A., McIntosh, K. R., and Mosca, J. D. (2003) J Biomed Sci 10(2), 228-241
27. Foster, L. J., Zeemann, P. A., Li, C., Mann, M., Jensen, 0. N., and Kassem, M. (2005) Stem Cells 23(9), 1367-1377
28. Chamberlain, G., Fox, J., Ashton, B., and Middleton, J. (2007) Stem Cells 25(11), 2739-2749
29. Al-Nbaheen, M., Vishnubalaji, R., Ali, D., Bouslimi, A., Al-Jassir, F., Megges, M., Prigione, A., Adjaye, J., Kassem, M., and Aldahmash, A. Stem Cell Rev
30. Steinert, A. F., Kunz, M., Prager, P., Barthel, T., Jakob, F., Noth, U., Murray, M. M., Evans, C. H., and Porter, R. M. Tissue Eng Part A 17(9-10), 1375-1388
31. Jeong, J. A., Ko, K. M., Park, H. S., Lee, J., Jang, C., Jeon, C. J., Koh, G. Y., and Kim, H. (2007) Proteomics 7(22), 4181-4191
32. Weise, F., Stierhof, Y. D., Kuhn, C., Wiese, M., and Overath, P. (2000) J Cell Sci 113 Pt 24, 4587-4603
33. Kooyrnan, D. L., Byrne, G. W., McClellan, S., Nielsen, D., Tone, M., Waldmann, H., Coffman, T. M., McCurry, K. R., Platt, J. L., and Logan, J. S. (1995) Science 269(5220), 89-34. Medof, M. E., Nagaraj an, S., and Tykocinski, M. L. (1996) Faseb J 10(5), 574-586
35. Mani, S. A., Yang, J., Brooks, M., Schwaninger, G., Zhou, A., Miura, N., Kutok, J. L., Hartwell, K., Richardson, A. L., and Weinberg, R. A. (2007) Proc Natl Acad Sci USA 104(24), 10069-10074
36. Sano, H., Leboeuf, J. P., Novitskiy, S. V., Seo, S., Zaja-Milatovic, S., Dikov, M. M., and Kume, T. Biochem Biophys Res Commun 392(2), 201-206
37. Lidell, M. E., Seifert, E. L., Westergren, R., Heglind, M., Gowing, A., Sukonina, V., Arani, Z., Itkonen, P., Wallin, S., Westberg, F., Fernandez-Rodriguez, J., Laakso, M., Nilsson, T., Peng, X. R., Harper, M. E., and Enerback, S. Diabetes 60(2), 427-435

1. A method of isolating GPI anchored proteins (GPI-APs) from cancer cells using labeled alpha-toxin from Clostridium septicum comprising the steps of:
a. providing a sample of cancer cells;
b. lysing said cancer cells, optionally in the presence of a non-ionic detergent, to obtain a mixture of cellular proteins which includes membrane proteins to which are attached GPI-anchor proteins;
c. exposing said mixture of proteins to phospholipase to release membrane proteins from GPI-anchor proteins bound thereto to produce a population of released GPI-anchor proteins;
d. exposing said population of GPI-anchor proteins to labeled C. septicum alpha toxin to produce alpha toxin bound GPI-anchor protein; and
e. isolating and/or purifying said alpha toxin bound GPI-anchor proteins, and optionally releasing alpha toxin from said GPI-anchor protein to produce isolated GPI-APs.

2. The method according to claim 1 wherein said non-ionic detergent is polyethylene glycol tert-octylphenyl ether (Triton X-114).

3. The method according to claim 1 wherein said phospholipase is phospholipase C.

4. The method according to claim 1 wherein said alpha toxin is labeled with biotin.

5. The method according to claim 4 wherein said alpha toxin bound GPI-anchor protein is isolated or purified using streptavidin to bind to biotin on said labeled alpha toxin.

6. The method according to claim 1 wherein said alpha toxin bound GPI-APs are released from alpha toxin to provide alpha toxin released GPI-APs.

7. The method according to claim 1 further comprising the step of releasing proteins from said GPI-APs to produce GPI-released proteins.

8. The method according to claim 6 further comprising the step of analyzing said GPI-APS or said GPI-released proteins to determine the content and quantity of proteins in said population of GPI-anchor proteins.

9. A method for the purification of GPI anchored proteins associated with cancer from a biological sample using labeled alpha-toxin from Clostridium septicum comprising obtaining a biological sample of cancerous tissue from a subject, separating said GPI anchor proteins from said tissue, exposing said separated GPI anchor proteins from said tissue to said alpha-toxin to allow said GPI anchor proteins and said alpha-toxin to bind to form a complex and isolating said GPI anchor protein (GPI-APs) alpha-toxin complex.

10. The method according to claim 9 wherein said alpha toxin is fluorescently labeled.

11. The method according to claim 9 wherein alpha toxin is labeled with biotin.

12. The method according to claim 9 wherein said glycoprotein alpha-toxin complex is treated to remove said alpha-toxin from said GPI-APs and provide isolated GPI-APs.

13. (canceled)

14. The method according to claim 12 wherein said isolated GPI-APs are analyzed.

15. The method according to claim 14 wherein said isolated GPI-APs are analyzed using a mass spectrometer to identify said GPI-APs.

16. (canceled)

17. (canceled)

18. (canceled)

19. (canceled)

20. (canceled)

21. (canceled)

22. (canceled)

23. (canceled)

24. A method of identifying at least one biomarker (GPI-AP) of a cancer cell, wherein said biomarker is expressed selectively by said cancer cells by virtue of such a biomarker being absent on normal cells or said biomarker is expressed in greater numbers on cancer cells in comparison to normal cells, the method comprising:
a. Providing a sample of cancer cells and normal cells from the same tissue;
b. Lysing said cells, optionally in the presence of a non-ionic detergent, to obtain a mixture of cellular proteins which includes a population of membrane proteins;
c. Exposing said cellular proteins or said membrane proteins to phospholipase to release membrane proteins from membranes of said cells to produce a population of membrane-released GPI-anchor proteins;
d. Exposing said population of GPI-anchor proteins to labeled C. septicum alpha toxin to produce alpha toxin bound GPI-anchor protein;
e. Isolating and/or purifying said alpha-toxin bound GPI-anchor protein and releasing alpha toxin from GPI-anchor protein to produce isolated GPI-anchored proteins;
f. Optionally, releasing protein from said GPI-anchor protein by deaminating said protein from the GPI moiety to provide a population of GPI-released proteins to be analyzed;
g. Analyzing by mass spectrometry said GPI-anchor proteins or optionally, said GPI-released proteins to determine the content and quantity of proteins in such population of GPI-anchor proteins for each of said cancer cells and normal cells; and
h. Comparing the content and quantity of proteins in said population of GPI-anchor proteins from said cancer cells with said normal cells, wherein a population of GPI-anchor proteins which is found exclusively on said cancer cells or at an identifiably higher concentration on said cancer cells compared to said normal cells identifies that GPI-anchor protein(s) as a potential selective biomarker for cancer diagnosis and/or treatment.

25. A method of determining whether or not a patient or subject suspected of having cancer has cancer, said method comprising obtaining a biological sample from said patient or subject suspected of being infected with cancer, exposing said sample to at least one labeled antibody which binds to at least one GPI-AP cancer biomarker identified in claim 24, determining the concentration of said at least one GPI-AP cancer biomarker in said sample of said patient and comparing said concentration(s) to a predetermined value, wherein the concentration level of said one or more GPI-AP cancer biomarkers which is above or below said predetermined value is indicative of the existence or absence of cancer in said patient or subject.

26. The method according to claim 25 wherein said antibody is used in combination with alpha toxin or another antibody.

27. The method according to claim 25 wherein said antibody is used in an immunoassay.

28. The method according to claim 27 wherein said immunoassay is a colorimetric or chemiluminescent assay.

29. (canceled)

30. The method according to any of claim 25 wherein said antibody binds to GPI-AP which is bound to alpha toxin.

31. The method according to claim 25 wherein said alpha-toxin is used in a plate assay.

32. (canceled)

33. (canceled)

34. A method of determining whether or not a patient or subject suspected of having breast cancer has breast cancer, said method comprising obtaining a biological sample from said patient or subject suspected of being infected with breast cancer, exposing said sample to at least one antibody, which is optionally labeled and which binds to a GPI-AP cancer biomarker selected from the group consisting of FERMT3/Kindling3 and/or FilamenA (FLNA), determining the concentration of said cancer biomarker(s) in said sample of said patient and comparing said concentration(s) to a predetermined value, wherein the concentration level of said one or more GPI-AP cancer biomarkers which is above or below said predetermined value is indicative of the existence or absence of cancer in said patient or subject.

35. The method according to claim 34 wherein alpha toxin is used in conjunction with said antibody(s), and wherein said alpha toxin is optionally labeled.

36. The method according to claim 34 wherein an additional antibody is used to bind said GPI-AP cancer biomarker.

37. The method according to claim 34 wherein said antibody(s) which is used in an immunoassay.

38. The method according to claim 37 wherein said immunoassay is a colorimetric or chemiluminescent assay.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. A GPI anchored glycoprotein cancer biomarker identified by the method of claim 24.

62. An antibody raised to a complex of a GPI-AP alpha toxin protein.

63. The antibody of claim 62 which is raised to a GPI-AP as set forth in any of Tables 1, 2, 3 or 4.

64. A complex comprising a GPI-AP bound to alpha toxin.

65. The complex of claim 65 wherein said alpha toxin is labeled.

66. (canceled)

67. (canceled)

68. (canceled)

69. (canceled)

70. (canceled)

71. (canceled)

72. (canceled)

73. (canceled)
